(19) United States
(12) Patent Application Publication
(10) Pub. No.: US 2025/0230172 A1
(43) Pub. Date: Jul. 17, 2025

(54) SMALL MOLECULE MODULATORS OF GLUCOCEREBROSIDASE ACTIVITY AND USES THEREOF

(71) Applicant: Vanqua Bio, Inc., Chicago, IL (US)

(72) Inventors: Kevin Hunt, Chicago, IL (US); Jianbin Zheng, Chicago, IL (US); Sida Shen, Chicago, IL (US)

(73) Assignee: Vanqua Bio, Inc., Chicago, IL (US)

(21) Appl. No.: 18/700,617

(22) PCT Filed: Oct. 12, 2022
(86) PCT No.: PCT/US2022/046390

§ 371 Date: Apr. 11, 2024

Related U.S. Application Data
(60) Provisional application No. 63/255,263, filed on Oct. 13, 2021.

(51) Int. Cl. C07D 519/00; A61K 31/444; A61K 31/497; A61K 31/4985; A61K 31/501; A61K 31/506; C07D 471/04; C07D 487/04

(52) U.S. Cl. CPC C07D 519/00; A61K 31/444; A61K 31/497; A61K 31/4985; A61K 31/501; A61K 31/506; C07D 471/04; C07D 487/04

(57) ABSTRACT

Provided herein are compounds that modulate glucocerebrosidase (GCase), an enzyme whose activity is associated with neurological diseases and disorders (e.g., Gaucher's disease, Parkinson's disease). Also provided are pharmaceutical compositions and kits comprising the compounds, and methods of treating GCase-related diseases and disorders (e.g., Gaucher's disease, Parkinson's disease) with the compounds in a subject, by administering the compounds and/or compositions described herein.

### RELATED APPLICATION

This application claims priority under 35 U.S.C. § 119(e) to U.S. Provisional Application 63/255,263 filed Oct. 13, 2021, the entirety of which is incorporated herein by reference.

### BACKGROUND

Glucocerebrosidase (EC 3.2.1.45), also called β-glucocerebrosidase, β-glucosidase, D-glucosyl-N-acylsphingosine glucohydrolase, or GCase, is an enzyme having glucosylceramidase activity. Glucocerebrosidase is required to cleave the beta-glucosidic linkage of the chemical glucocerebroside, which is an intermediate in glycolipid metabolism. Glucocerebrosidase is localized in the lysosome and disabling mutations in the gene for glucocerebrosidase (GBA1) are associated with abnormal accumulation of lipids in lysosomes.

Genetic diseases caused by mutations in GBA1 include neurodegenerative diseases such as Gaucher's disease and Parkinson's disease. Current treatments for diseases such Type 1 Gaucher's disease are limited to enzyme replacement therapy (ERT) administered every two weeks. ERT is very expensive and not effective for neuronopathic forms of Gaucher's disease. Efforts to discover and employ small molecule compounds to activate Gcase have been met with limited success. Thus, there is a need for new compounds that effectively activate Gcase and are useful in the treatment of neurodegenerative diseases (e.g., Gaucher's disease and Parkinson's disease).

### SUMMARY

The present disclosure provides compounds (e.g., any compound delineated herein) that are modulators of GCase. These compounds provide new compositions and methods for the treatment of diseases associated with GCase activity (e.g., neurodegenerative diseases, such as Gaucher's disease and Parkinson's disease).

In one aspect, provided are compounds of Formula (I):

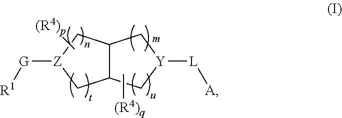

and pharmaceutically acceptable salts, co-crystals, tautomers, stereoisomers, solvates, hydrates, polymorphs, isotopically enriched derivatives, or prodrugs thereof, wherein:
R¹ is substituted or unsubstituted heteroaryl, substituted or unsubstituted aryl, or a nitrogen protecting group;
G is a bond or —O—;
X¹ is N or CR⁴;
Y is N or CR⁴;
Z is N or CR⁴;
L is a bond, —NR^{A}—, or —C(═O)—;
A is

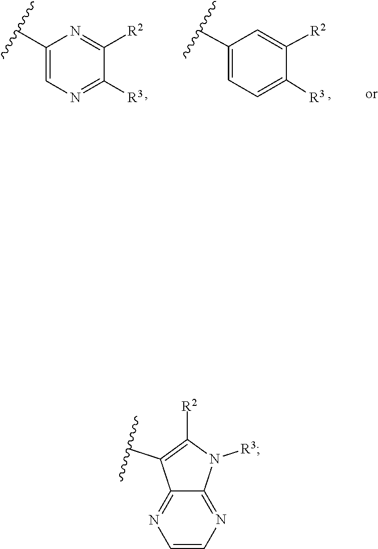

R² and R³ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl;
each R⁴ is independently hydrogen, halogen, substituted or unsubstituted alkyl, substituted or unsubstituted cycloalkyl, substituted or unsubstituted alkoxy, or two instances of R⁴ on the same carbon together with that carbon form a carbonyl;
R^{A} is hydrogen, substituted or unsubstituted alkyl, or a nitrogen protecting group;
p is 0, 1, 2, 3, or 4;
q is 0, 1, 2, 3, or 4;
t is 0, 1 or 2;
u is 0, 1 or 2;
m is 0, 1 or 2; and
n is 0, 1 or 2; provided that the sum of m and u is 2 or 3, and the sum of n and t is 2 or 3.

In certain embodiments, the compounds of Formula (I) are compounds of Formula (I-a), (I-a-1), (I-b), (I-b-1), (I-c), (I-c-1), (I-d), (I-d-1), (I-e), (I-e-1), (I-f), (I-f-1), (I-g), (I-g-1), (I-h), (I-h-1), (I-i), (I-i-1), (I-j), (I-j-1), (I-k), (I-k-1), (I-l), (I-l-1), (I-m), (I-m-1), (I-n), (I-n-1), (I-o), (I-p), (I-q), (I-r), (I-r-4), (I-s), (I-t), (I-u), or (I-v):

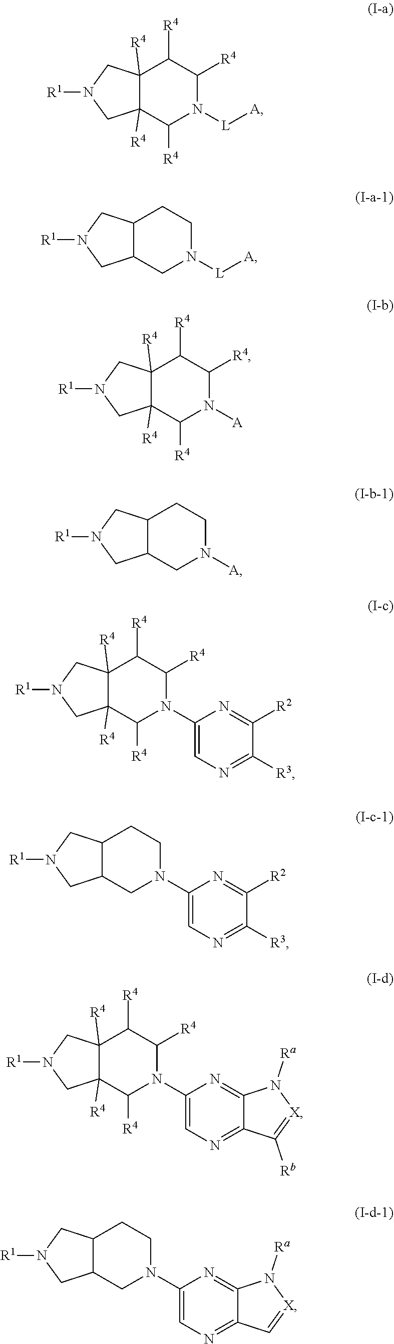

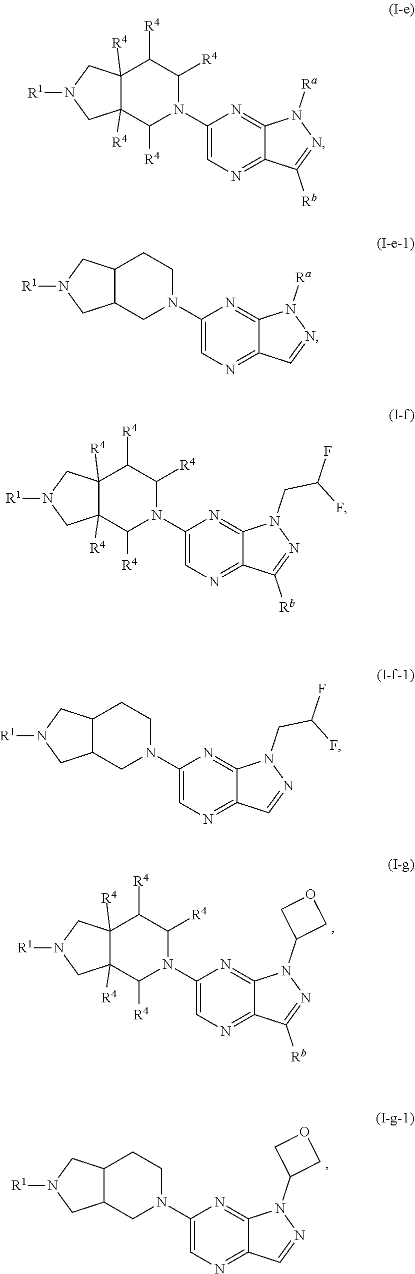

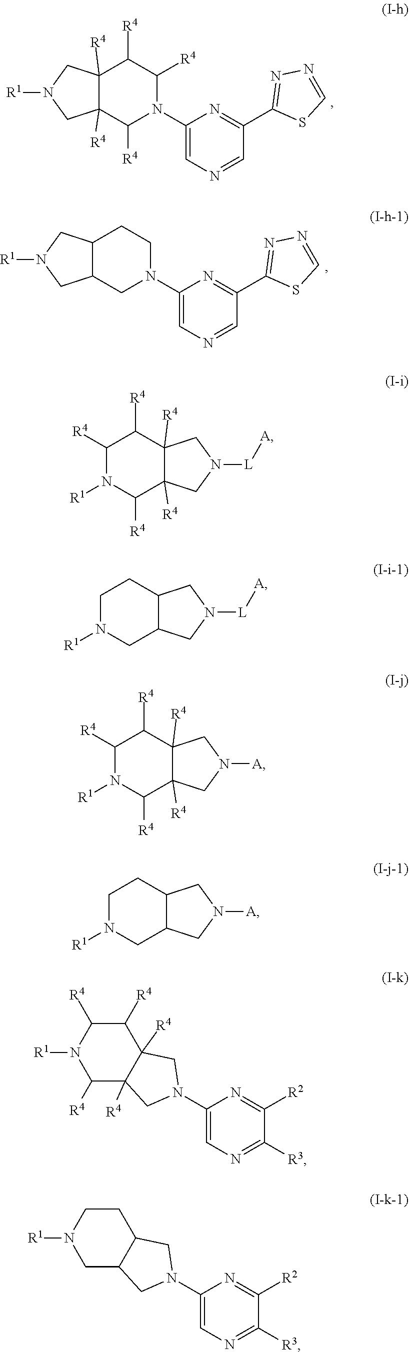

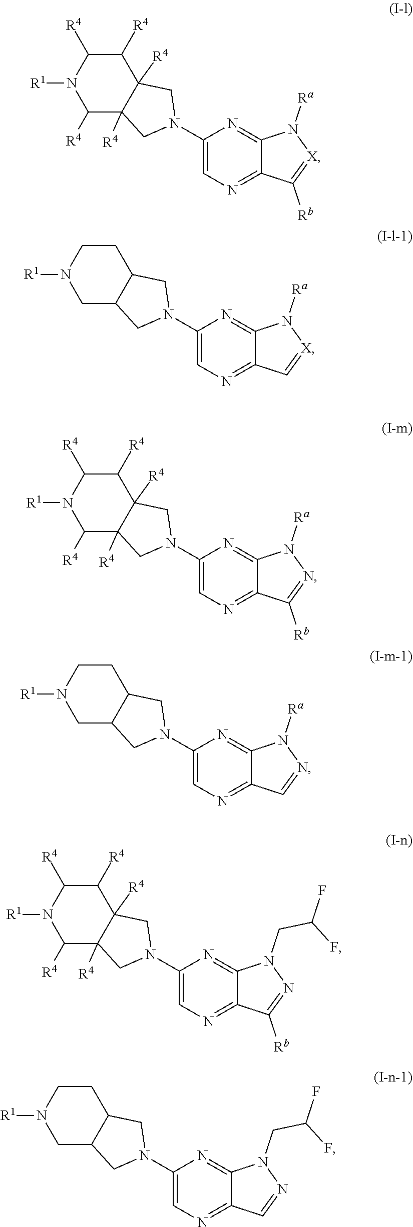

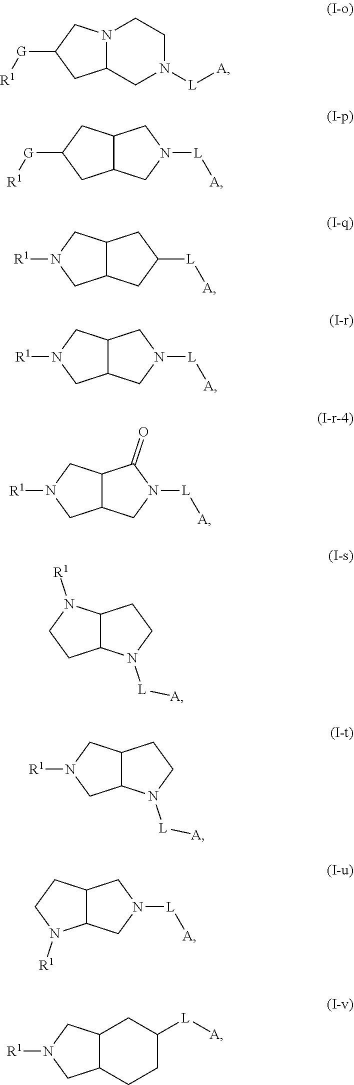

or pharmaceutically acceptable salts thereof; wherein R^{a} and R^{b} are as defined herein.

In another aspect, provided are compounds of Formula (II):

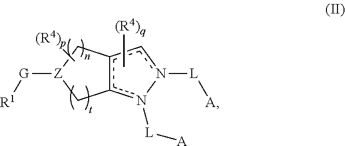

and pharmaceutically acceptable salts, co-crystals, tautomers, stereoisomers, solvates, hydrates, polymorphs, isotopically enriched derivatives, or prodrugs thereof, wherein:
each [symbol] independently represents a single or double bond;
R¹ is substituted or unsubstituted heteroaryl, substituted or unsubstituted aryl, or a nitrogen protecting group;
G is a bond or —O—;
Z is N or CR⁴;
L is absent, a bond, —NR^{A}—, or —C(═O)—;
A is absent,

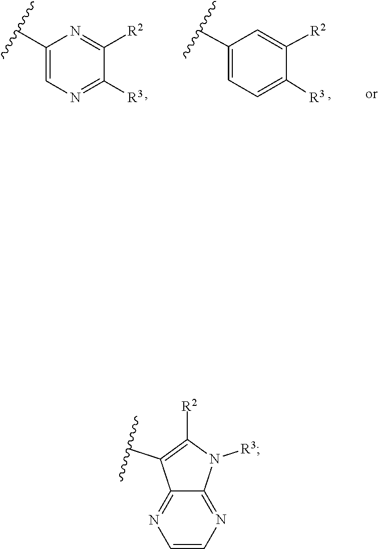

R² and R³ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl;
each R⁴ is independently hydrogen, halogen, substituted or unsubstituted alkyl, substituted or unsubstituted cycloalkyl, substituted or unsubstituted alkoxy, or two instances of R⁴ on the same carbon together with that carbon form a carbonyl;
R^{A} is hydrogen, substituted or unsubstituted alkyl, or a nitrogen protecting group;
p is 0, 1, 2, 3, or 4;
q is 0, 1, 2, or 3;
t is 1 or 2; and
n is 1 or 2; provided that one instance of -L-A is absent.

In certain embodiments, the compounds of Formula (II) are compounds of Formula (II-a), (II-a-1), (II-a-2), (II-b), (II-b-1), or (II-b-2):

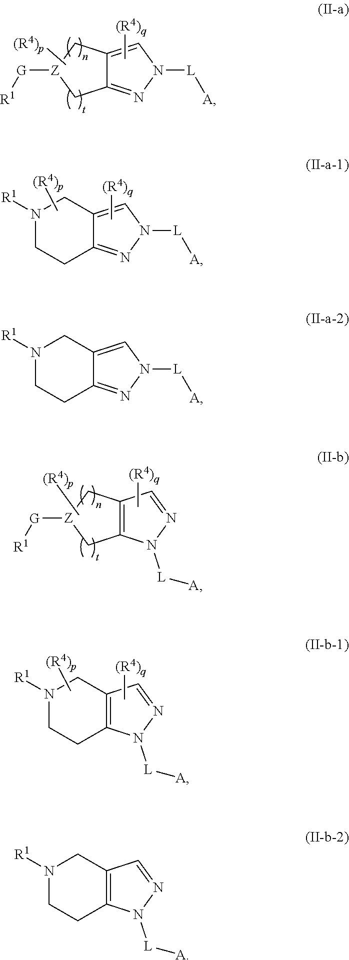

In another aspect, provided are pharmaceutical compositions comprising a compound of Formula (I) or (II), or a pharmaceutically acceptable salt thereof, and optionally a pharmaceutically acceptable excipient.

In another aspect, provided are methods of treating a disease or disorder in a subject in need thereof, the method comprising administering a compound as delineated herein (e.g., Formula (I) or (II)), or a pharmaceutically acceptable salt thereof, or a pharmaceutical composition comprising a compound as delineated herein (e.g., Formula (I) or (II)) to the subject.

In certain embodiments, the disease or disorder is associated with glucocerebrosidase activity. In certain embodiments, the disease or disorder is a neurological disease or disorder. In certain embodiments, the neurological disease or disorder is Parkinson's disease or Gaucher's disease.

In another aspect, provided are methods of activating glucocerebrosidase, the method comprising contacting glucocerebrosidase with an effective amount of a compound of Formula (I) or (II), or a pharmaceutically acceptable salt thereof, or a pharmaceutical composition comprising a compound of Formula (I) or (II) to the subject.

In another aspect, provided are kits comprising a compound of Formula (I) or (II), or a pharmaceutically acceptable salt thereof, or a pharmaceutical composition comprising a compound of Formula (I), or a pharmaceutically acceptable salt thereof. In certain embodiments, the kits further comprise instructions for administration (e.g., human administration).

The details of certain embodiments of the invention are set forth in the Detailed Description of Certain Embodiments, as described below. Other features, objects, and advantages of the invention will be apparent from the Definitions, Examples, and Claims.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers. While compounds may be depicted as racemic or as one or more diastereoisomers, enantiomers, or other isomers, all such racemic, diastereoisomer, enantiomer, or other isomer forms of that depicted are included in the present disclosure.

In a formula, [symbol] is a single bond where the stereochemistry of the moieties immediately attached thereto is not specified, [symbol] is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “alkoxy” refers to an alkyl group, as defined herein, appended to the parent molecular moiety through an oxygen atom. In some embodiments, the alkoxy moiety has 1 to 8 carbon atoms (“C₁₋₈ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋₈ alkoxyalkyl”). In some embodiments, the alkoxyalkyl moiety has 1 to 6 carbon atoms (“C₁₋₆ alkoxyalkyl”). In some embodiments, the alkoxyalkyl moiety has 1 to 4 carbon atoms (“C₁₋₄ alkoxyalkyl”). In some embodiments, the alkoxyalkyl moiety has 1 to 3 carbon atoms (“C₁₋₃ alkoxyalkyl”). In some embodiments, the alkoxyalkyl moiety has 1 to 2 carbon atoms (“C₁₋₂ alkoxyalkyl”).

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and 1 or more heteroatoms within the parent chain (“heteroC₁₋₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀alkenyl. In certain embodiments, the alkenyl group is a substituted C₂₋₁₀alkenyl. In an alkenyl group, a C═C double bond for which the stereochemistry is not specified (e.g., —CH═CHCH₃ or

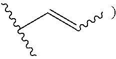

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and lor 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃_cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Arylalkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroarylalkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not intended to be limited in any manner by the exemplary substituents described herein.

When substituted, exemplary carbon atom substituents include, but are not limited to, halogen, —CN, —NO₂, —N₃, —SO₂H, —SO₃H, —OH, —OR—, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{cc})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{f})₂, —N(R^{f})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{f} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)+X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula: —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, or —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined herein.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{c})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), 2-methoxyethyl, methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{cc})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

These and other exemplary substituents are described in more detail in the Detailed Description, Examples, and Claims. The invention is not intended to be limited in any manner by the above exemplary listing of substituents.

### Other Definitions

The following definitions are more general terms used throughout the present application.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and/or animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water molecules. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). Many compounds can adopt a variety of different crystal forms (i.e., different polymorphs). Typically, such different crystalline forms have different X-ray diffraction patterns, infrared spectra, and/or can vary in some or all properties such as melting points, density, hardness, crystal shape, optical and electrical properties, stability, solubility, and bioavailability. Recrystallization solvent, rate of crystallization, storage temperature, and other factors may cause one crystal form to dominate a given preparation. Various polymorphs of a compound can be prepared by crystallization under different conditions.

The term “co-crystal” refers to a crystalline structure composed of at least two components. In certain embodiments, a co-crystal contains a compound of the present disclosure and one or more other component(s), including, but not limited to, atoms, ions, molecules, or solvent molecules. In certain embodiments, a co-crystal contains a compound of the present disclosure and one or more solvent molecules. In certain embodiments, a co-crystal contains a compound of the present disclosure and one or more acid or base. In certain embodiments, a co-crystal contains a compound of the present disclosure and one or more components related to said compound, including, but not limited to, an isomer, tautomer, salt, solvate, hydrate, synthetic precursor, synthetic derivative, fragment, or impurity of said compound.

The term “prodrugs” refers to compounds that have cleavable groups that are removed, by solvolysis or under physiological conditions, to provide the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgaard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇₋₁₂ arylalkyl esters of the compounds described herein may be preferred.

The terms “composition” and “formulation” are used interchangeably.

The term “modulate” means decreasing or inhibiting activity and/or increasing or augmenting activity. For example, modulating glucocerebrosidase activity means decreasing or inhibiting glucocerebrosidase activity and/or increasing or augmenting glucocerebrosidase activity. The compounds disclosed herein may be administered to modulate glucocerebrosidase activity for example, as a chaperone or activator.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, or inhibiting the progress of a disease described herein. In some embodiments, treatment may be administered after one or more signs or symptoms of the disease have developed or have been observed. Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for GCase activation (e.g., at least 5%, at least 10%, at least 20%, at least 30%, at least 40%, at least 50%, at least 60%, at least 70%, at least 80%, at least 90%, at least 95%, at least 100%, at least 150%, at least 200%, at least 250%, at least 300%, or at least 500% increase in the enzymatic activity of GCase). In certain embodiments, a therapeutically effective amount is an amount sufficient for treating a disease or disorder (e.g., neurological disorder). In certain embodiments, a therapeutically effective amount is an amount sufficient for GCase activation and treating a disease or disorder (e.g., neurological disorder).

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for GCase activation. In certain embodiments, a prophylactically effective amount is an amount sufficient for treating a disease or disorder (e.g., neurological disorder). In certain embodiments, a prophylactically effective amount is an amount sufficient for GCase activation and treating a disease or disorder (e.g., neurological disorder).

As used herein, the term “activate” or “activation” in the context of enzymes, for example, in the context of GCase, refers to an increase in the activity of the enzyme. In some embodiments, the term refers to an increase of the level of enzyme activity, e.g., GCase activity, to a level that is statistically significantly higher than an initial level, which may, for example, be a baseline level of enzyme activity (e.g., of wild-type GCase). In some embodiments, the term refers to an increase in the level of enzyme activity, e.g., GCase activity, to a level that is greater than 1%, greater than 5%, greater than 10%, greater than 25%, greater than 50%, greater than 75%, greater than 100%, greater than 150%, greater than 200%, greater than 300%, greater than 400%, greater than 500%, or greater than 1000% of an initial level, which may, for example, be a baseline level of enzyme activity.

The term “immunotherapy” refers to a therapeutic agent that promotes the treatment of disease by inducing, enhancing, or suppressing an immune response. Immunotherapies designed to elicit or amplify an immune response are classified as activation immunotherapies, while immunotherapies that reduce or suppress are classified as suppression immunotherapies. Immunotherapies are typically, but not always, biotherapeutic agents. Numerous immunotherapies are used to treat cancer. These include, but are not limited to, monoclonal antibodies, adoptive cell transfer, cytokines, chemokines, vaccines, and small molecule inhibitors.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities, such as cells and tissues. Biologics may be isolated from a variety of natural sources (e.g., human, animal, microorganism) and may be produced by biotechnological methods and other technologies.

The term “small molecule” or “small molecule therapeutic” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics, or combinations thereof.

### DETAILED DESCRIPTION OF CERTAIN EMBODIMENTS

Provided herein are compounds that are modulators of GCase (e.g., GCase activators). In one aspect, the provided GCase modulators are compounds of Formula (I), and pharmaceutically acceptable salts, solvates, hydrates, polymorphs, co-crystals, tautomers, stereoisomers, isotopically labeled derivatives, prodrugs, and pharmaceutical compositions thereof. In another aspect, the provided GCase modulators are compounds of Formula (II), and pharmaceutically acceptable salts, solvates, hydrates, polymorphs, co-crystals, tautomers, stereoisomers, isotopically labeled derivatives, prodrugs, and pharmaceutical compositions thereof. Accordingly, the compounds are useful for the treatment and/or prevention of diseases and disorders associated with GCase activity (e.g., neurological diseases and disorders) in a subject in need thereof.

The compounds described herein interact with GCase. As described herein, the therapeutic effect may be a result of modulation (e.g., activation), binding, and/or modification of GCase by the compounds described herein. The compounds may be provided for use in any composition, kit, or method described herein as a pharmaceutically acceptable salt, co-crystal, tautomer, stereoisomer, solvate, hydrate, polymorph, isotopically enriched derivative, or prodrug thereof.

### Compounds

In one aspect, disclosed is a compound of Formula (I):

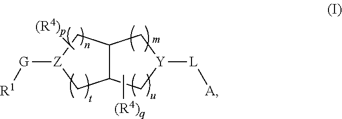

and pharmaceutically acceptable salts, co-crystals, tautomers, stereoisomers, solvates, hydrates, polymorphs, isotopically enriched derivatives, or prodrugs thereof, wherein:
R¹ is substituted or unsubstituted heteroaryl, substituted or unsubstituted aryl, or a nitrogen protecting group;
G is a bond or —O—;
X¹ is N or CR⁴;
Y is N or CR⁴;
Z is N or CR⁴;
L is a bond, —NR^{A}—, or —C(═O)—;
A is

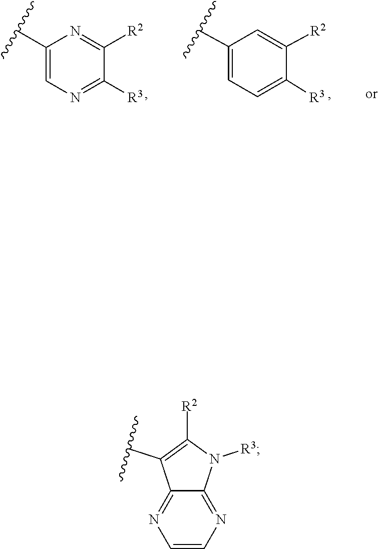

R² and R³ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl;
each R⁴ is independently hydrogen, halogen, substituted or unsubstituted alkyl, substituted or unsubstituted cycloalkyl, substituted or unsubstituted alkoxy, or two instances of R⁴ on the same carbon together with that carbon form a carbonyl;
R^{A} is hydrogen, substituted or unsubstituted alkyl, or a nitrogen protecting group;
p is 0, 1, 2, 3, or 4;
q is 0, 1, 2, 3, or 4;
t is 0, 1 or 2;
u is 0, 1 or 2;
m is 0, 1 or 2; and
n is 0, 1 or 2; provided that the sum of m and u is 2 or 3, and the sum of n and t is 2 or 3.

In certain embodiments, the compound of Formula (I) is a compound of Formula (I-1):

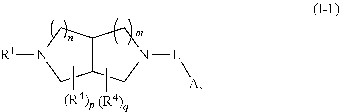

or a pharmaceutically acceptable salt, co-crystal, tautomer, stereoisomer, solvate, hydrate, polymorph, isotopically enriched derivative, or prodrug thereof.

In certain embodiments, the compound of Formula (I) is a compound of Formula (I-1):

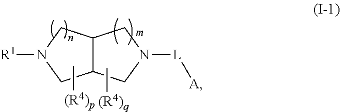

or a pharmaceutically acceptable salt, co-crystal, tautomer, stereoisomer, solvate, hydrate, polymorph, isotopically enriched derivative, or prodrug thereof, wherein:
R¹ is substituted or unsubstituted heteroaryl, substituted or unsubstituted aryl, or a nitrogen protecting group;
L is a bond or —C(═O)—;
A is

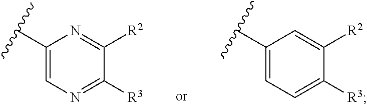

R² and R³ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl;
each R⁴ is independently hydrogen, halogen, substituted or unsubstituted alkyl, substituted or unsubstituted cycloalkyl, substituted or unsubstituted alkoxy, or two instances of R⁴ on the same carbon together with that carbon form a carbonyl;
p is 0, 1, 2, 3, or 4;
q is 0, 1, 2, 3, or 4;
m is 1 or 2; and
n is 1 or 2.

In another aspect, provided are compounds of Formula (II):

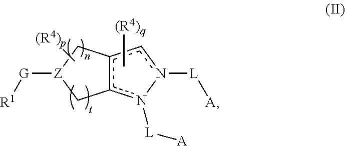

and pharmaceutically acceptable salts, co-crystals, tautomers, stereoisomers, solvates, hydrates, polymorphs, isotopically enriched derivatives, or prodrugs thereof, wherein:
each [symbol] independently represents a single or double bond;
R¹ is substituted or unsubstituted heteroaryl, substituted or unsubstituted aryl, or a nitrogen protecting group;
G is a bond or —O—;
Z is N or CR⁴;
L is absent, a bond, —NR^{A}—, or —C(═O)—;
A is

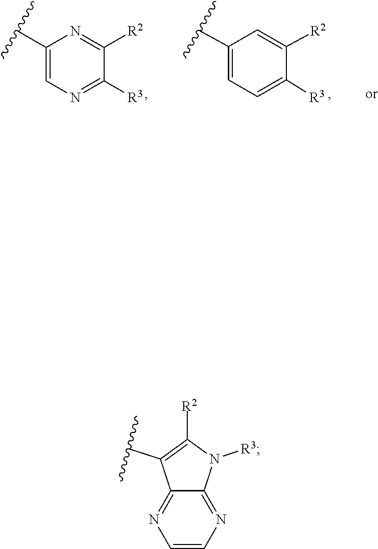

R² and R³ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl;
each R⁴ is independently hydrogen, halogen, substituted or unsubstituted alkyl, substituted or unsubstituted cycloalkyl, substituted or unsubstituted alkoxy, or two instances of R⁴ on the same carbon together with that carbon form a carbonyl;
R^{A} is hydrogen, substituted or unsubstituted alkyl, or a nitrogen protecting group;
p is 0, 1, 2, 3, or 4;
q is 0, 1, 2, or 3;
t is 1 or 2; and
n is 1 or 2; provided that one instance of -L-A is absent.

### R¹

As described herein for Formula (I) and (II), R¹ is substituted or unsubstituted heteroaryl, substituted or unsubstituted aryl, or a nitrogen protecting group. In certain embodiments, R¹ is substituted or unsubstituted heteroaryl, or substituted or unsubstituted aryl. In certain embodiments, R¹ is substituted or unsubstituted heteroaryl, or a nitrogen protecting group.

In certain embodiments, R¹ is a nitrogen protecting group. In certain embodiments, R¹ is a carbamate. In certain embodiments, R¹ is —C(═O)OR^{aa}, wherein R^{aa} is as defined herein. In certain embodiments, R¹ is t-butyl carbamate (i.e., BOC).

In certain embodiments, R¹ is substituted or unsubstituted heteroaryl. In certain embodiments, R¹ is substituted or unsubstituted 6-membered heteroaryl. In certain embodiments, R¹ is substituted or unsubstituted pyridinyl, substituted or unsubstituted pyrazinyl, substituted or unsubstituted pyrimidinyl, or substituted or unsubstituted pyridazinyl.

In certain embodiments, R¹ is substituted heteroaryl. In certain embodiments, R¹ is heteroaryl substituted with at least one instance of halogen, haloalkyl, alkoxy, or haloalkoxy. In certain embodiments, R¹ is heteroaryl substituted with at least one instance of halogen or haloalkyl. In certain embodiments, R¹ is heteroaryl substituted with at least one instance of halogen, C₁₋₄ haloalkyl or C₁₋₄ haloalkoxy. In certain embodiments, R¹ is heteroaryl substituted with at least one instance of halogen or C₁₋₄ haloalkyl. In certain embodiments, R¹ is heteroaryl substituted with at least one instance of fluoro or fluoroalkyl. In certain embodiments, R¹ is heteroaryl substituted with at least one instance of fluoro or C₁₋₄ fluoroalkyl. In certain embodiments, R¹ is heteroaryl substituted with at least one instance of C₁₋₄ fluoroalkyl.

In certain embodiments, R¹ is substituted or unsubstituted pyridinyl. In certain embodiments, R¹ is substituted pyridinyl. In certain embodiments, R¹ is pyridinyl substituted with at least one instance of halogen, haloalkyl or haloalkoxy. In certain embodiments, R¹ is pyridinyl substituted with at least one instance of halogen or haloalkyl. In certain embodiments, R¹ is pyridinyl substituted with at least one instance of halogen or C₁₋₄ haloalkyl or C₁₋₄ haloalkoxy. In certain embodiments, R¹ is pyridinyl substituted with at least one instance of halogen or C₁₋₄ haloalkyl. In certain embodiments, R¹ is pyridinyl substituted with at least one instance of fluoro or fluoroalkyl. In certain embodiments, R¹ is pyridinyl substituted with at least one instance of fluoro or C₁₋₄ fluoroalkyl.

In certain embodiments, R¹ is pyridinyl substituted with at least one instance of haloalkyl. In certain embodiments, R¹ is pyridinyl substituted with at least one instance of C₁. 4 haloalkyl. In certain embodiments, R¹ is pyridinyl substituted with at least one instance of fluoroalkyl. In certain embodiments, R¹ is pyridinyl substituted with at least one instance of C₁₋₄ fluoroalkyl. In certain embodiments, R¹ is pyridinyl substituted with at least one instance of trifluoromethyl, difluoromethyl, or fluoromethyl. In certain embodiments, R¹ is pyridinyl substituted with at least one instance of trifluoromethyl.

In certain embodiments, R¹ is substituted or unsubstituted pyrimidinyl. In certain embodiments, R¹ is substituted pyrimidinyl. In certain embodiments, R¹ is pyrimidinyl substituted with at least one instance of haloalkyl. In certain embodiments, R¹ is pyrimidinyl substituted with at least one instance of C₁₋₄ haloalkyl. In certain embodiments, R¹ is pyrimidinyl substituted with at least one instance of fluoroalkyl. In certain embodiments, R¹ is pyrimidinyl substituted with at least one instance of C₁₋₄ fluoroalkyl. In certain embodiments, R¹ is pyrimidinyl substituted with at least one instance of trifluoromethyl, difluoromethyl, or fluoromethyl. In certain embodiments, R¹ is pyrimidinyl substituted with at least one instance of trifluoromethyl.

In certain embodiments, R¹ is pyrimidinyl substituted with at least one instance of halogen, unsubstituted alkyl, haloalkyl or haloalkoxy. In certain embodiments, R¹ is pyrimidinyl substituted with at least one instance of unsubstituted alkyl or haloalkyl. In certain embodiments, R¹ is pyrimidinyl substituted with at least one instance of unsubstituted C₁₋₄ alkyl or C₁₋₄ haloalkyl. In certain embodiments, R¹ is pyrimidinyl substituted with at least one instance of unsubstituted C₁₋₄ alkyl or C₁₋₄ fluoroalkyl. In certain embodiments, R¹ is pyrimidinyl substituted with at least one instance of methyl or C₁₋₄ fluoroalkyl.

In certain embodiments, R¹ is pyrimidinyl substituted with unsubstituted alkyl and haloalkyl. In certain embodiments, R¹ is pyrimidinyl substituted with unsubstituted C₁₋₄ alkyl and C₁₋₄ haloalkyl. In certain embodiments, R¹ is pyrimidinyl substituted with methyl and fluoroalkyl. In certain embodiments, R¹ is pyrimidinyl substituted with methyl and C₁₋₄ fluoroalkyl. In certain embodiments, R¹ is pyrimidinyl substituted with methyl and trifluoromethyl, difluoromethyl, or fluoromethyl. In certain embodiments, R¹ is pyrimidinyl substituted with methyl and trifluoromethyl.

In certain embodiments, R¹ is substituted or unsubstituted pyrazinyl. In certain embodiments, R¹ is substituted pyrazinyl. In certain embodiments, R¹ is pyrazinyl substituted with at least one instance of haloalkyl or alkoxy. In certain embodiments, R¹ is pyrazinyl substituted with at least one instance of C₁₋₄ haloalkyl or C₁₋₄ alkoxy. In certain embodiments, R¹ is pyrazinyl substituted with at least one instance of fluoroalkyl. In certain embodiments, R¹ is pyrazinyl substituted with at least one instance of C₁₋₄ fluoroalkyl. In certain embodiments, R¹ is pyrazinyl substituted with at least one instance of trifluoromethyl, difluoromethyl, or fluoromethyl. In certain embodiments, R¹ is pyrazinyl substituted with at least one instance of trifluoromethyl. In certain embodiments, R¹ is pyrazinyl substituted with at least one instance of trifluoromethyl or methoxy. In certain embodiments, R¹ is pyrazinyl substituted with at least one instance of methoxy.

In certain embodiments, R¹ is substituted or unsubstituted pyridazinyl. In certain embodiments, R¹ is substituted pyridazinyl. In certain embodiments, R¹ is pyridazinyl substituted with at least one instance of haloalkyl. In certain embodiments, R¹ is pyridazinyl substituted with at least one instance of C₁₋₄ haloalkyl. In certain embodiments, R¹ is pyridazinyl substituted with at least one instance of fluoroalkyl. In certain embodiments, R¹ is pyridazinyl substituted with at least one instance of C₁₋₄ fluoroalkyl. In certain embodiments, R¹ is pyridazinyl substituted with at least one instance of trifluoromethyl, difluoromethyl, or fluoromethyl. In certain embodiments, R¹ is pyridazinyl substituted with at least one instance of trifluoromethyl.

In certain embodiments, R¹ is

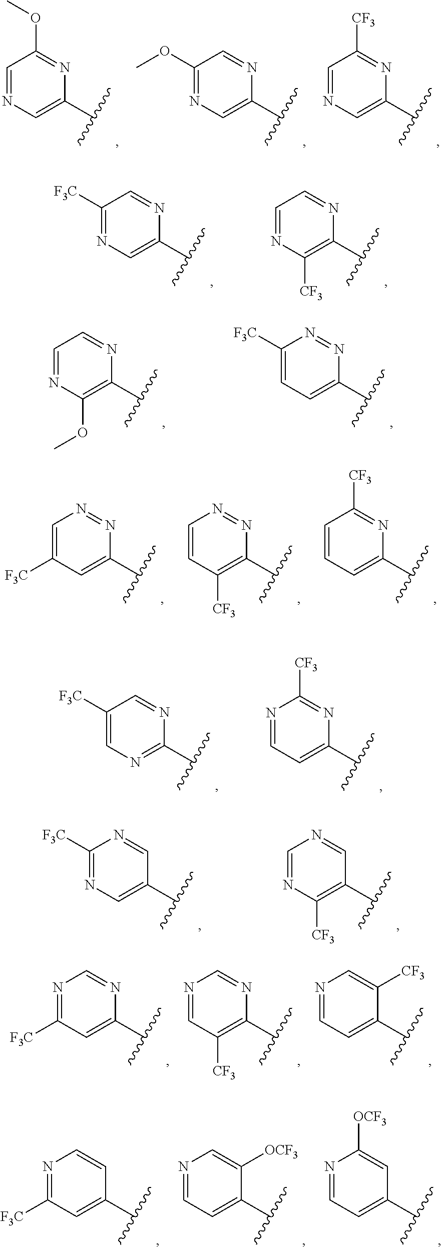

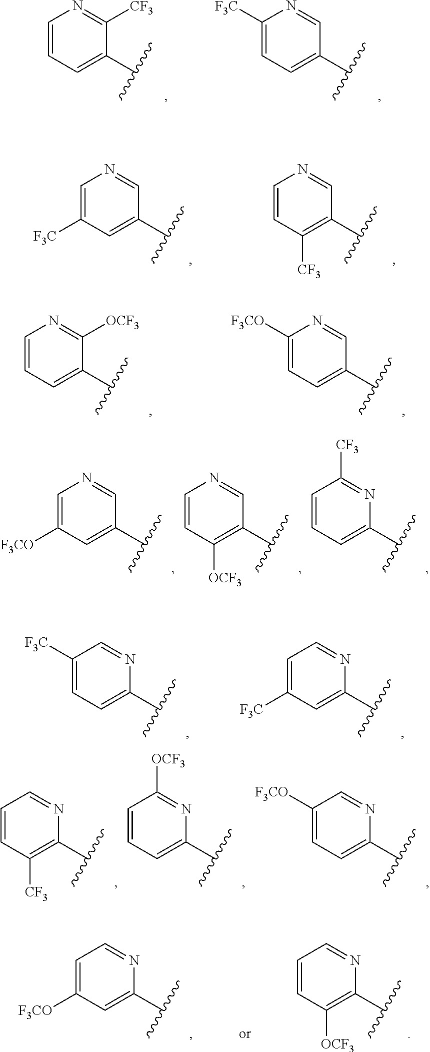

In certain embodiments, R¹ is

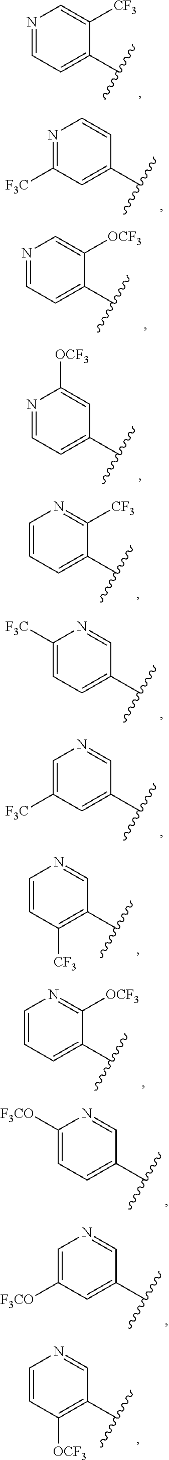

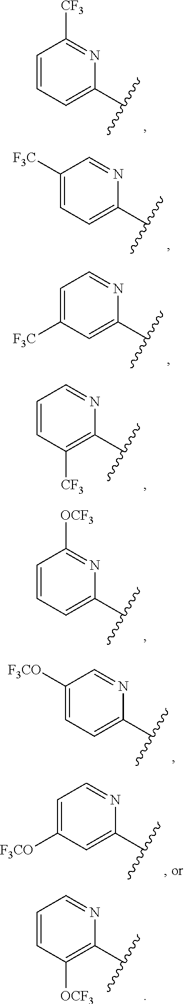

In certain embodiments, R¹ is

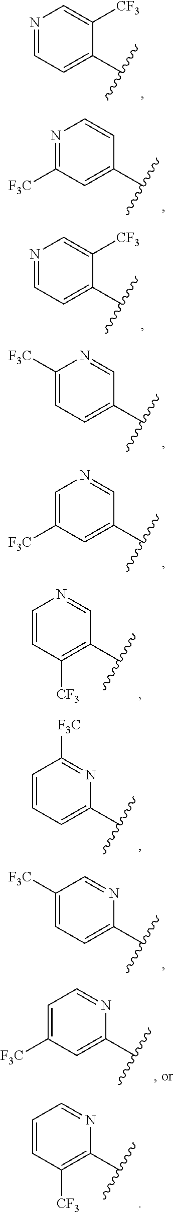

In certain embodiments, R¹ is

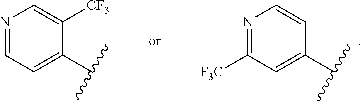 [SMILES: *c1ccncc1C(F)(F)F ; *c1ccnc(C(F)(F)F)c1]

In certain embodiments, R¹ is

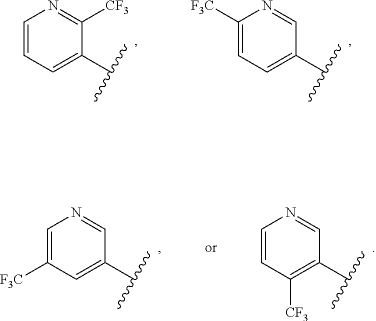 [SMILES: *c1cccnc1C(F)(F)F ; *c1ccc(C(F)(F)F)nc1 ; *c1cncc(C(F)(F)F)c1 ; *c1cnccc1C(F)(F)F]

In certain embodiments, R¹ is

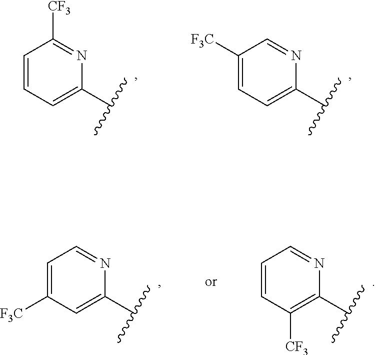 [SMILES: *c1cccc(C(F)(F)F)n1 ; *c1ccc(C(F)(F)F)cn1 ; *c1cc(C(F)(F)F)ccn1 ; *c1ncccc1C(F)(F)F]

In certain embodiments, R¹ is

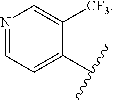 [SMILES: *c1ccncc1C(F)(F)F]

In certain embodiments, R¹ is

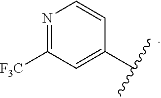 [SMILES: *c1ccnc(C(F)(F)F)c1]

In certain embodiments, R¹ is

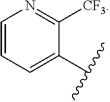 [SMILES: *c1cccnc1C(F)(F)F]

In certain embodiments, R¹ is

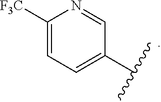 [SMILES: *c1ccc(C(F)(F)F)nc1]

In certain embodiments, R¹ is

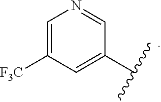 [SMILES: *c1cncc(C(F)(F)F)c1]

In certain embodiments, R¹ is

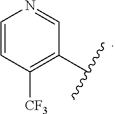 [SMILES: *c1cnccc1C(F)(F)F]

In certain embodiments, R¹ is

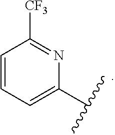 [SMILES: *c1cccc(C(F)(F)F)n1]

In certain embodiments, R¹ is

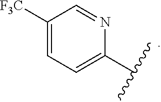 [SMILES: *c1ccc(C(F)(F)F)cn1]

In certain embodiments, R¹ is

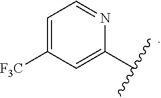 [SMILES: *c1cc(C(F)(F)F)ccn1]

In certain embodiments, R¹ is

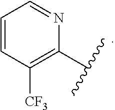 [SMILES: *c1ncccc1C(F)(F)F]

### G

As described herein for Formula (I) and (II), G is a bond or —O—. In certain embodiments, G is a bond. In certain embodiments, G is —O—.

### X¹, Y, and Z

As described herein for Formula (I), X¹ is N or CR⁴. In certain embodiments, X¹ is N. In certain embodiments, X¹ is CR⁴. In certain embodiments, X is CH.

As described herein for Formula (I), Y is N or CR⁴. In certain embodiments, Y is N. In certain embodiments, Y is CR⁴. In certain embodiments, Y is CH.

As described herein for Formula (I) and (II), Z is N or CR⁴. In certain embodiments, Z is N. In certain embodiments, Z is CR⁴. In certain embodiments, Z is CH.

In certain embodiments for Formula (I), X¹ is N; Y is N; and Z is N. In certain embodiments, X¹ is CR⁴; Y is CR⁴; and Z is CR⁴. In certain embodiments, X¹ is CH; Y is CH; and Z is CH. In certain embodiments, X¹ is CR⁴; Y is N; and Z is N. In certain embodiments, X¹ is CH; Y is N; and Z is N. In certain embodiments, X¹ is N; Y is CR⁴; and Z is N. In certain embodiments, X¹ is N; Y is CH; and Z is N. In certain embodiments, X¹ is N; Y is N; and Z is CR⁴. In certain embodiments, X¹ is N; Y is N; and Z is CH. In certain embodiments, X¹ is N; Y is CR⁴; and Z is CR⁴. In certain embodiments, X¹ is CR⁴; Y is N; and Z is CR⁴. In certain embodiments, X¹ is CR⁴; Y is CR⁴; and Z is N. In certain embodiments, X¹ is N; Y is CH; and Z is CH. In certain embodiments, X¹ is CH; Y is N; and Z is CH. In certain embodiments, X¹ is CH; Y is CH; and Z is N.

### L

As described herein for Formula (I), L is a bond, —NR^{A}—, or —C(═O)—. As described herein for Formula (II), L is absent, a bond, —NR^{A}—, or —C(═O)—.

In certain embodiments, L is a bond or —C(═O)—. In certain embodiments, L is a bond. In certain embodiments, L is-C(═O)—. In certain embodiments, when L is —C(═O)—, then A is

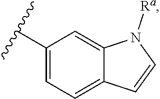

wherein R^{a} is hydrogen or substituted or unsubstituted alkyl. In certain embodiments, L is —NR^{A}—. In certain embodiments, L is —NH—. In certain embodiments, L is —NR^{A}—, wherein R^{A} is substituted or unsubstituted alkyl. In certain embodiments, L is —NR^{A}—, wherein R^{A} is substituted or unsubstituted C₁₋₄ alkyl. In certain embodiments, L is —NR^{A}—, wherein R^{A} is unsubstituted C₁₋₄ alkyl. In certain embodiments, L is —N(CH₃)—.

In each compound of Formula (II), one instance of L is absent. In each compound of Formula (II), one instance of -L-A is absent.

### A

As described herein for Formula (I), A is

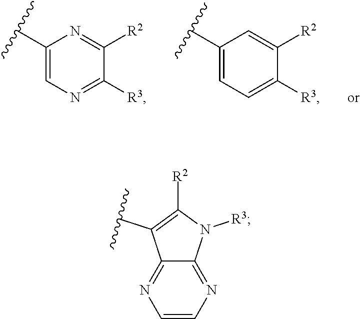

wherein R² and R³ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl.

As described herein for Formula (II), A is absent,

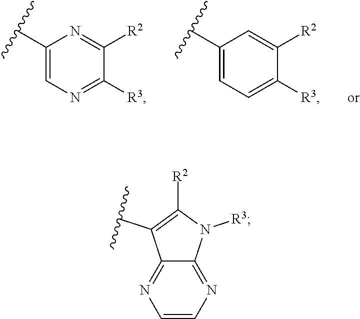

wherein R² and R³ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl. In each compound of Formula (II), one instance of A is absent. In each compound of Formula (II), one instance of -L-A is absent.

In certain embodiments, A is

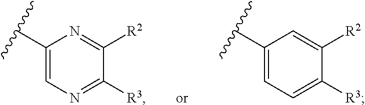

wherein R² and R³ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl.

In certain embodiments, A is

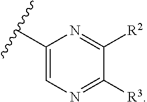

In certain embodiments, A is

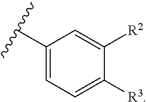

In certain embodiments, R² and R³ are each independently hydrogen or substituted or unsubstituted heteroaryl; or R² and R³ together with the atoms to which they are attached form a substituted or unsubstituted aryl, or substituted or unsubstituted heteroaryl.

In certain embodiments, R² is substituted or unsubstituted heteroaryl. In certain embodiments, R² is unsubstituted heteroaryl. In certain embodiments, R² is substituted or unsubstituted thiadizaolyl. In certain embodiments, R² is unsubstituted thiadizaolyl.

In certain embodiments, R³ is hydrogen.

In certain embodiments, R² is substituted or unsubstituted heteroaryl; and R³ is hydrogen. In certain embodiments, R² is unsubstituted heteroaryl; and R³ is hydrogen. In certain embodiments, R² is substituted or unsubstituted thiadizaolyl; and R³ is hydrogen. In certain embodiments, R² is unsubstituted thiadizaolyl; and R³ is hydrogen.

In certain embodiments, R² and R³ together with the atoms to which they are attached form a substituted or unsubstituted aryl, or substituted or unsubstituted heteroaryl.

In certain embodiments, R² and R³ together with the atoms to which they are attached form a substituted or unsubstituted aryl. In certain embodiments, R² and R³ together with the atoms to which they are attached form a substituted or unsubstituted phenyl. In certain embodiments, R² and R³ together with the atoms to which they are attached form a substituted phenyl. In certain embodiments, R² and R³ together with the atoms to which they are attached form an unsubstituted phenyl.

In certain embodiments, R² and R³ together with the atoms to which they are attached form a substituted or unsubstituted heteroaryl. In certain embodiments, R² and R³ together with the atoms to which they are attached form a substituted or unsubstituted pyrrolyl or substituted or unsubstituted pyrazolyl. In certain embodiments, R² and R³ together with the atoms to which they are attached form a substituted pyrrolyl or substituted pyrazolyl. In certain embodiments, R² and R³ together with the atoms to which they are attached form a substituted pyrrolyl or substituted pyrazolyl, wherein the pyrrolyl or pyrazolyl is substituted with substituted or unsubstituted alkyl, or substituted or unsubstituted heterocyclyl. In certain embodiments, R² and R³ together with the atoms to which they are attached form a substituted pyrrolyl or substituted pyrazolyl, wherein the pyrrolyl or pyrazolyl is substituted with substituted or unsubstituted heterocyclyl, haloalkyl, or substituted or unsubstituted heterocyclylalkyl. In certain embodiments, R² and R³ together with the atoms to which they are attached form a substituted pyrrolyl or substituted pyrazolyl, wherein the pyrrolyl or pyrazolyl is substituted with substituted or unsubstituted heterocyclyl, substituted or unsubstituted heterocyclylalkyl, or C₁₋₄ haloalkyl. In certain embodiments, R² and R³ together with the atoms to which they are attached form a substituted pyrrolyl or substituted pyrazolyl, wherein the pyrrolyl or pyrazolyl is substituted with substituted or unsubstituted heterocyclyl. In certain embodiments, R² and R³ together with the atoms to which they are attached form a substituted pyrrolyl or substituted pyrazolyl, wherein the pyrrolyl or pyrazolyl is substituted with C₁₋₄ haloalkyl. In certain embodiments, R² and R³ together with the atoms to which they are attached form a substituted pyrrolyl or substituted pyrazolyl, wherein the pyrrolyl or pyrazolyl is substituted with substituted or unsubstituted heterocyclylalkyl.

In certain embodiments, R² and R³ together with the atoms to which they are attached form a substituted or unsubstituted pyrazolyl. In certain embodiments, R² and R³ together with the atoms to which they are attached form a substituted pyrazolyl. In certain embodiments, R² and R³ together with the atoms to which they are attached form a substituted pyrazolyl, wherein the pyrazolyl is substituted with substituted or unsubstituted alkyl, or substituted or unsubstituted heterocyclyl. In certain embodiments, R² and R³ together with the atoms to which they are attached form a substituted pyrazolyl, wherein the pyrazolyl is substituted with substituted or unsubstituted heterocyclyl. In certain embodiments, R² and R³ together with the atoms to which they are attached form a substituted pyrazolyl, wherein the pyrazolyl is substituted with substituted or unsubstituted 4-5 membered heterocyclyl. In certain embodiments, R² and R³ together with the atoms to which they are attached form a substituted pyrazolyl, wherein the pyrazolyl is substituted with substituted or unsubstituted oxetanyl. In certain embodiments, R² and R³ together with the atoms to which they are attached form a substituted pyrazolyl, wherein the pyrazolyl is substituted with unsubstituted alkyl or haloalkyl. In certain embodiments, R² and R³ together with the atoms to which they are attached form a substituted pyrazolyl, wherein the pyrazolyl is substituted with unsubstituted C₁₋₄ alkyl or C₁₋₄ haloalkyl. In certain embodiments, R² and R³ together with the atoms to which they are attached form a substituted pyrazolyl, wherein the pyrazolyl is substituted with unsubstituted C₁₋₄ alkyl. In certain embodiments, R² and R³ together with the atoms to which they are attached form a substituted pyrazolyl, wherein the pyrazolyl is substituted with C₁₋₄ haloalkyl.

In certain embodiments, R² and R³ together with the atoms to which they are attached form a substituted or unsubstituted pyrrolyl. In certain embodiments, R² and R³ together with the atoms to which they are attached form a substituted pyrrolyl. In certain embodiments, R² and R³ together with the atoms to which they are attached form a substituted pyrrolyl, wherein the pyrrolyl is substituted with substituted or unsubstituted alkyl. In certain embodiments, R² and R³ together with the atoms to which they are attached form a substituted pyrrolyl, wherein the pyrrolyl is substituted with unsubstituted alkyl or haloalkyl. In certain embodiments, R² and R³ together with the atoms to which they are attached form a substituted pyrrolyl, wherein the pyrrolyl is substituted with unsubstituted C₁₋₄ alkyl or C₁₋₄ haloalkyl. In certain embodiments, R² and R³ together with the atoms to which they are attached form a substituted pyrrolyl, wherein the pyrrolyl is substituted with unsubstituted C₁₋₄ alkyl. In certain embodiments, R² and R³ together with the atoms to which they are attached form a substituted pyrrolyl, wherein the pyrrolyl is substituted with C₁₋₄ haloalkyl. In certain embodiments, R² and R³ together with the atoms to which they are attached form a substituted pyrrolyl, wherein the pyrrolyl is substituted with substituted or unsubstituted heterocyclylalkyl. In certain embodiments, R² and R³ together with the atoms to which they are attached form a substituted pyrrolyl, wherein the pyrrolyl is substituted with substituted or unsubstituted oxetanylalkyl.

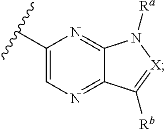

In certain embodiments, A is R^{b}; wherein X is N or CR^{a}; each R^{a} is independently hydrogen, substituted or unsubstituted alkyl, or substituted or unsubstituted heterocyclyl; and R^{b} is hydrogen, halogen, substituted or unsubstituted alkyl, substituted or unsubstituted cycloalkyl, or substituted or unsubstituted alkoxy. In certain embodiments, R^{a} is independently hydrogen, or substituted or unsubstituted alkyl; and R^{b} is hydrogen, halogen, substituted or unsubstituted alkyl, substituted or unsubstituted cycloalkyl, or substituted or unsubstituted alkoxy. In certain embodiments, R^{a} is independently hydrogen, or substituted or unsubstituted alkyl; and R^{b} is halogen, substituted or unsubstituted alkyl, substituted or unsubstituted cycloalkyl, or substituted or unsubstituted alkoxy. In certain embodiments, R^{a} is independently hydrogen, or substituted or unsubstituted alkyl; and R^{b} is substituted or unsubstituted alkyl, substituted or unsubstituted cycloalkyl, or substituted or unsubstituted alkoxy. In certain embodiments, X is N or CH; R^{a} is hydrogen, or substituted or unsubstituted alkyl; and R^{b} is substituted or unsubstituted alkyl, substituted or unsubstituted cycloalkyl, or substituted or unsubstituted alkoxy. In certain embodiments, X is N; R^{a} is hydrogen, or substituted or unsubstituted alkyl; and R^{b} is substituted or unsubstituted alkyl, substituted or unsubstituted cycloalkyl, or substituted or unsubstituted alkoxy. In certain embodiments, X is N; R^{a} is hydrogen, or substituted or unsubstituted alkyl; and R^{b} is hydrogen, substituted or unsubstituted alkyl, substituted or unsubstituted cycloalkyl, or substituted or unsubstituted alkoxy. In certain embodiments, X is N; R^{a} is hydrogen, or substituted or unsubstituted alkyl; and R^{b} is hydrogen, substituted or unsubstituted C₁₋₄ alkyl, substituted or unsubstituted C₃₋₄ cycloalkyl, or substituted or unsubstituted C₁₋₄ alkoxy. In certain embodiments, X is N; R^{a} is hydrogen, or substituted or unsubstituted alkyl; and R^{b} is substituted or unsubstituted C₁₋₄ alkyl, substituted or unsubstituted C₃₋₄ cycloalkyl, or substituted or unsubstituted C₁₋₄ alkoxy. In certain embodiments, X is N; R^{a} is hydrogen, or substituted or unsubstituted alkyl; and R^{b} is unsubstituted C₁₋₄ alkyl, C₁₋₄ haloalkyl, unsubstituted C₃₋₄ cycloalkyl, unsubstituted C₃₋₄ cycloalkylmethyl, C₁₋₄ haloalkoxy, or unsubstituted C₁₋₄ alkoxy. In certain embodiments, X is N; R^{a} is hydrogen, or substituted or unsubstituted alkyl; and R^{b} is hydrogen, unsubstituted C₁₋₄ alkyl, C₁₋₄ haloalkyl, unsubstituted C₃₋₄ cycloalkyl, unsubstituted C₃₋₄ cycloalkylmethyl, C₁₋₄ haloalkoxy, or unsubstituted C₁₋₄ alkoxy. In certain embodiments, X is CH; R^{a} is hydrogen, or substituted or unsubstituted alkyl; and R^{b} is substituted or unsubstituted alkyl, or substituted or unsubstituted alkoxy.

In certain embodiments, R^{b} is hydrogen, halogen, substituted or unsubstituted alkyl, substituted or unsubstituted cycloalkyl, or substituted or unsubstituted alkoxy. In certain embodiments, R^{b} is halogen, substituted or unsubstituted alkyl, substituted or unsubstituted cycloalkyl, or substituted or unsubstituted alkoxy. In certain embodiments, R^{b} is substituted or unsubstituted alkyl, substituted or unsubstituted cycloalkyl, or substituted or unsubstituted alkoxy. In certain embodiments, R^{b} is substituted or unsubstituted alkyl, substituted or unsubstituted cycloalkyl, or substituted or unsubstituted alkoxy. In certain embodiments, R^{b} is hydrogen, substituted or unsubstituted alkyl, substituted or unsubstituted cycloalkyl, or substituted or unsubstituted alkoxy. In certain embodiments, R^{b} is hydrogen, substituted or unsubstituted C₁₋₄ alkyl, substituted or unsubstituted C₃₋₄ cycloalkyl, or substituted or unsubstituted C₁₋₄ alkoxy. In certain embodiments, R^{b} is substituted or unsubstituted C₁₋₄ alkyl, substituted or unsubstituted C₃₋₄ cycloalkyl, or substituted or unsubstituted C₁₋₄ alkoxy. In certain embodiments, R^{b} is unsubstituted C₁₋₄ alkyl, C₁₋₄ haloalkyl, unsubstituted C₃₋₄ cycloalkyl, unsubstituted C₃₋₄ cycloalkylmethyl, C₁₋₄ haloalkoxy, or unsubstituted C₁₋₄ alkoxy. In certain embodiments, R^{b} is hydrogen, unsubstituted C₁₋₄ alkyl, C₁₋₄ haloalkyl, unsubstituted C₃₋₄ cycloalkyl, unsubstituted C₃₋₄ cycloalkylmethyl, C₁₋₄ haloalkoxy, or unsubstituted C₁₋₄ alkoxy. In certain embodiments, R^{b} is hydrogen.

In certain embodiments, A is

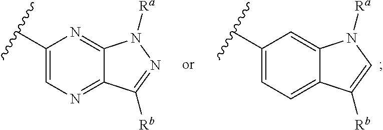

wherein R^{a} is hydrogen, substituted or unsubstituted alkyl, or substituted or unsubstituted heterocyclyl; and R^{b} is hydrogen, halogen, substituted or unsubstituted alkyl, substituted or unsubstituted cycloalkyl, or substituted or unsubstituted alkoxy.

In certain embodiments, A is

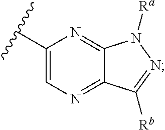

wherein R^{a} is hydrogen, substituted or unsubstituted alkyl, or substituted or unsubstituted heterocyclyl; and R^{b} is hydrogen, halogen, substituted or unsubstituted alkyl, substituted or unsubstituted cycloalkyl, or substituted or unsubstituted alkoxy.

In certain embodiments, A is

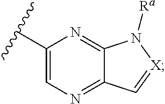

wherein X is N or CR^{a}; and each R^{a} is independently hydrogen, substituted or unsubstituted alkyl, or substituted or unsubstituted heterocyclyl. In certain embodiments, X is N or CH. In certain embodiments, X is N. In certain embodiments, X is CH.

In certain embodiments, A is

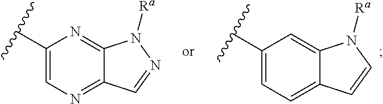

wherein R^{a} is hydrogen, substituted or unsubstituted alkyl, or substituted or unsubstituted heterocyclyl.

In certain embodiments, A is

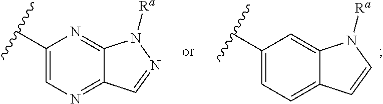

wherein R^{a} is hydrogen, substituted or unsubstituted alkyl, or substituted or unsubstituted 4-5 membered heterocyclyl.

In certain embodiments, A is

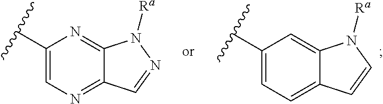

wherein R^{a} is hydrogen, heterocyclylalkyl, haloalkyl, or substituted or unsubstituted 4-5 membered heterocyclyl.

In certain embodiments, A is

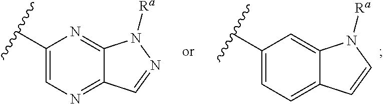

wherein R^{a} is hydrogen, oxetanylalkyl, haloalkyl, or substituted or unsubstituted 4-5 membered heterocyclyl.

In certain embodiments, A is

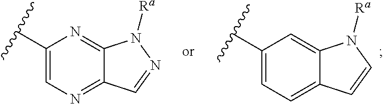

wherein R^{a} is hydrogen, oxetanylalkyl, haloalkyl, or oxetanyl.

In certain embodiments, A is

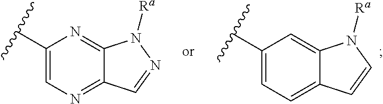

wherein R^{a} is hydrogen, oxetanylalkyl, C₁₋₄ haloalkyl, or oxetanyl.

In certain embodiments, A is

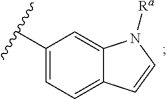

wherein R^{a} is hydrogen, heterocyclyl, or substituted or unsubstituted alkyl.

In certain embodiments, A is

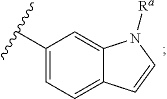

wherein R^{a} is hydrogen, or substituted or unsubstituted alkyl.

In certain embodiments, A is

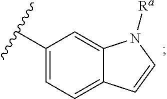

wherein R^{a} is hydrogen, heterocyclyl, or heterocyclylalkyl.

In certain embodiments, A is

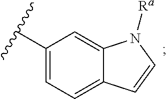

wherein R^{a} is hydrogen or heterocyclylalkyl.

In certain embodiments, A is

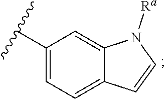

herein R^{a} is hydrogen, oxetanyl, or oxetanylalkyl.

In certain embodiments, A is

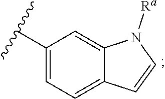

wherein R^{a} is hydrogen or oxetanylalkyl.

In certain embodiments, A is

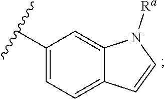

wherein R^{a} is oxetanyl.

In certain embodiments, A is

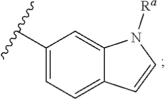

wherein R is hydrogen.

In certain embodiments, A is

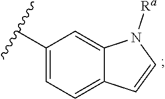

wherein R^{a} is oxetanylalkyl.

In certain embodiments, A is

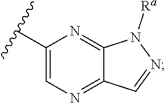

wherein R^{a} is substituted or unsubstituted alkyl, or substituted or unsubstituted heterocyclyl.

In certain embodiments, A is

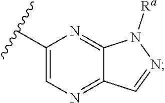

wherein R^{a} is substituted or unsubstituted alkyl, or substituted or unsubstituted 4-5 membered heterocyclyl.

In certain embodiments, A is

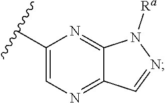

wherein R^{a} is haloalkyl, or substituted or unsubstituted 4-5 membered heterocyclyl.

In certain embodiments, A is

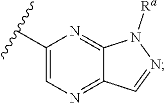

wherein R^{a} is haloalkyl, or substituted or unsubstituted 4-5 membered heterocyclyl.

In certain embodiments, A is

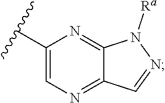

wherein R^{a} is haloalkyl or oxetanyl.

In certain embodiments, A is

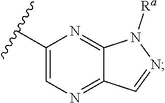

wherein R^{a} is C₁₋₄ haloalkyl or oxetanyl.

In certain embodiments, A is

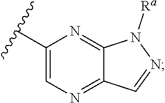

wherein R^{a} is C₁₋₄ haloalkyl.

In certain embodiments, A is

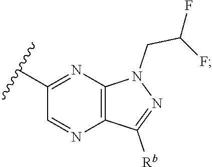

and R^{b} is hydrogen, halogen, substituted or unsubstituted alkyl, substituted or unsubstituted cycloalkyl, or substituted or unsubstituted alkoxy. In certain embodiments, R^{b} is halogen, substituted or unsubstituted alkyl, substituted or unsubstituted cycloalkyl, or substituted or unsubstituted alkoxy. In certain embodiments, R^{b} is substituted or unsubstituted alkyl, substituted or unsubstituted cycloalkyl, or substituted or unsubstituted alkoxy. In certain embodiments, R^{b} is hydrogen, substituted or unsubstituted alkyl, substituted or unsubstituted cycloalkyl, or substituted or unsubstituted alkoxy. In certain embodiments, R^{b} is hydrogen, substituted or unsubstituted C₁₋₄ alkyl, substituted or unsubstituted C₃₋₄ cycloalkyl, or substituted or unsubstituted C₁₋₄ alkoxy. In certain embodiments, R^{b} is substituted or unsubstituted C₁₋₄ alkyl, substituted or unsubstituted C₃₋₄ cycloalkyl, or substituted or unsubstituted C₁₋₄ alkoxy. In certain embodiments, R^{b} is unsubstituted C₁₋₄ alkyl, C₁₋₄ haloalkyl, unsubstituted C₃₋₄ cycloalkyl, unsubstituted C₃₋₄ cycloalkylmethyl, C₁₋₄ haloalkoxy, or unsubstituted C₁₋₄ alkoxy. In certain embodiments, R^{b} is hydrogen, unsubstituted C₁₋₄ alkyl, C₁₋₄ haloalkyl, unsubstituted C₃₋₄ cycloalkyl, unsubstituted C₃₋₄ cycloalkylmethyl, C₁₋₄ haloalkoxy, or unsubstituted C₁₋₄ alkoxy. In certain embodiments, R^{b} is hydrogen, unsubstituted C₁₋₄ alkyl, C₁₋₄ haloalkyl, unsubstituted cyclopropyl, unsubstituted cyclopropylmethyl, C₁₋₄ haloalkoxy, or unsubstituted C₁₋₄ alkoxy. In certain embodiments, R^{b} is substituted or unsubstituted alkyl, or substituted or unsubstituted alkoxy.

In certain embodiments, A is

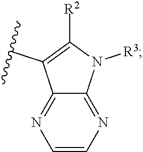

wherein R² and R³ are each independently substituted or unsubstituted alkyl, substituted or unsubstituted carbocyclyl, substituted or unsubstituted heterocyclyl, substituted or unsubstituted aryl, or substituted or unsubstituted heteroaryl. In certain embodiments, A is

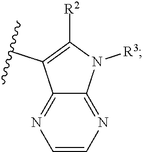

wherein R² and R³ are each independently substituted or unsubstituted alkyl, or substituted or unsubstituted aryl. In certain embodiments, A is

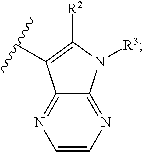

wherein R² is substituted or unsubstituted alkyl, and R³ is substituted or unsubstituted aryl. In certain embodiments, A is

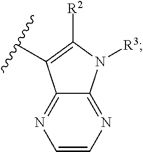

wherein R² is substituted or unsubstituted C₁₋₄ alkyl, and R³ is substituted or unsubstituted phenyl. In certain embodiments, A is

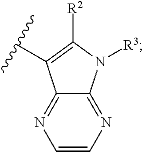

wherein R² is unsubstituted C₁₋₄ alkyl, and R³ is unsubstituted phenyl. In certain embodiments, A is

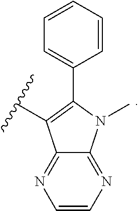 [SMILES: *c1c(-c2ccccc2)n(C)c2nccnc12]

In certain embodiments, A is

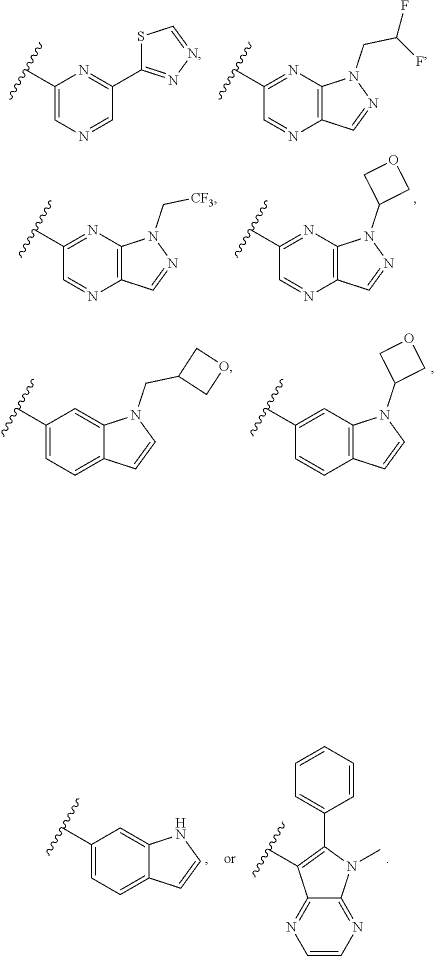

In certain embodiments, A is

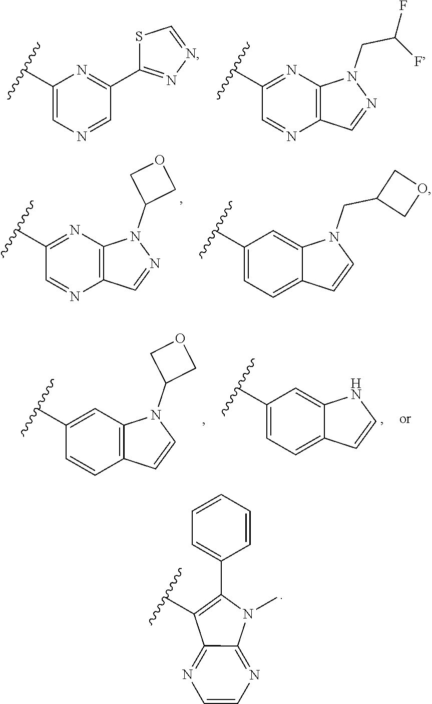

In certain embodiments, A is

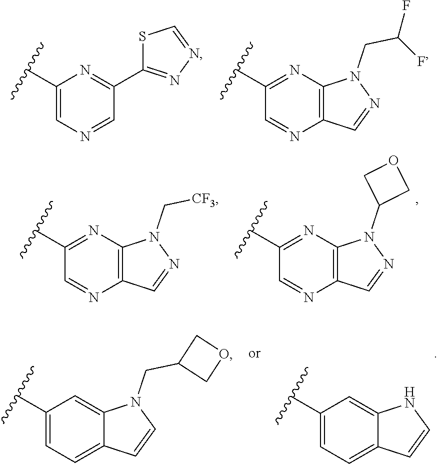

In certain embodiments, A is

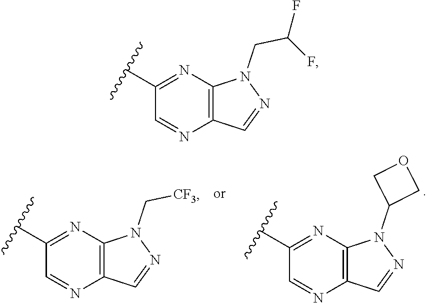 [SMILES: *c1cnc2cnn(CC(F)F)c2n1 ; *c1cnc2cnn(CC(F)(F)F)c2n1 ; "or" ; *c1cnc2cnn(C3COC3)c2n1 ; "."]

In certain embodiments, A is

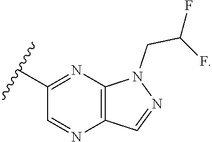 [SMILES: *c1cnc2cnn(CC(F)F)c2n1]

In certain embodiments, A is

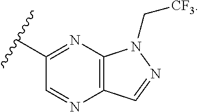 [SMILES: *c1cnc2cnn(CC(F)(F)F)c2n1]

In certain embodiments, A is

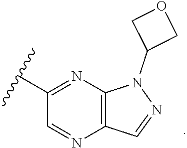 [SMILES: *c1cnc2cnn(C3COC3)c2n1]

In certain embodiments, A is

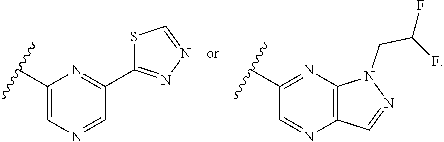 [SMILES: *c1cncc(-c2nncs2)n1 ; *c1cnc2cnn(CC(F)F)c2n1]

In certain embodiments, A is

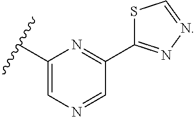 [SMILES: *c1cncc(-c2nncs2)n1]

In certain embodiments, A is

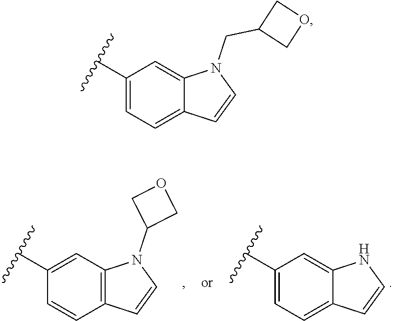 [SMILES: *c1ccc2ccn(CC3COC3)c2c1 ; *c1ccc2ccn(C3COC3)c2c1 ; *c1ccc2cc[nH]c2c1]

In certain embodiments, A is

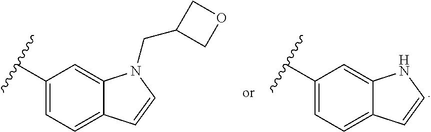 [SMILES: *c1ccc2ccn(CC3COC3)c2c1 ; *c1ccc2cc[nH]c2c1]

In certain embodiments, A is

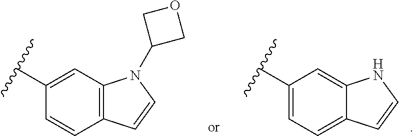 [SMILES: *c1ccc2ccn(C3COC3)c2c1 ; *c1ccc2cc[nH]c2c1]

In certain embodiments, A is

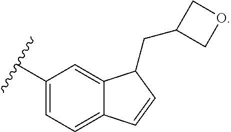 [SMILES: *c1ccc2c(c1)C(CC1COC1)C=C2]

In certain embodiments, A is

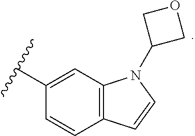 [SMILES: *c1ccc2ccn(C3COC3)c2c1]

In certain embodiments, A is

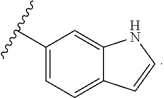 [SMILES: *c1ccc2cc[nH]c2c1]

### R⁴

As described herein for Formula (I), each R⁴ is independently hydrogen, halogen, substituted or unsubstituted alkyl, substituted or unsubstituted cycloalkyl, substituted or unsubstituted alkoxy, or two instances of R⁴ on the same carbon form with that carbon a carbonyl; p is 0, 1, 2, 3, or 4; and q is 0, 1, 2, 3, or 4. In certain embodiments, each R⁴ is independently halogen, substituted or unsubstituted alkyl, substituted or unsubstituted cycloalkyl, substituted or unsubstituted alkoxy, or two instances of R⁴ on the same carbon form with that carbon a carbonyl; p is 0, 1, 2, 3, or 4; and q is 0, 1, 2, 3, or 4.

As described herein for Formula (II), each R⁴ is independently hydrogen, halogen, substituted or unsubstituted alkyl, substituted or unsubstituted cycloalkyl, substituted or unsubstituted alkoxy, or two instances of R⁴ on the same carbon form with that carbon a carbonyl; p is 0, 1, 2, 3, or 4; and q is 0, 1, 2, or 3.

In certain embodiments, each R⁴ is independently halogen, substituted or unsubstituted alkyl, substituted or unsubstituted cycloalkyl, or substituted or unsubstituted alkoxy. In certain embodiments, each R⁴ is independently halogen, substituted or unsubstituted alkyl, substituted or unsubstituted cycloalkyl, or substituted or unsubstituted alkoxy, or two instances of R⁴ on the same carbon form with that carbon a carbonyl. In certain embodiments, each R⁴ is independently substituted or unsubstituted alkyl, substituted or unsubstituted cycloalkyl, or substituted or unsubstituted alkoxy. In certain embodiments, each R⁴ is independently substituted or unsubstituted alkyl, substituted or unsubstituted cycloalkyl, or substituted or unsubstituted alkoxy, or two instances of R⁴ on the same carbon form with that carbon a carbonyl. In certain embodiments, each R⁴ is independently substituted or unsubstituted C₁₋₄ alkyl, substituted or unsubstituted C₃₋₄ cycloalkyl, or substituted or unsubstituted C₁₋₄ alkoxy. In certain embodiments, each R⁴ is independently substituted or unsubstituted C₁₋₄ alkyl, substituted or unsubstituted C₃₋₄ cycloalkyl, or substituted or unsubstituted C₁₋₄ alkoxy, or two instances of R⁴ on the same carbon form with that carbon a carbonyl. In certain embodiments, each R⁴ is independently unsubstituted C₁₋₄ alkyl, C₁₋₄ haloalkyl, unsubstituted C₃₋₄ cycloalkyl, unsubstituted C₃₋₄ cycloalkylmethyl, C₁₋₄ haloalkoxy, or unsubstituted C₁₋₄ alkoxy. In certain embodiments, each R⁴ is independently unsubstituted C₁₋₄ alkyl, C₁₋₄ haloalkyl, unsubstituted C₃₋₄ cycloalkyl, unsubstituted C₃₋₄ cycloalkylmethyl, C₁₋₄ haloalkoxy, or unsubstituted C₁₋₄ alkoxy, or two instances of R⁴ on the same carbon form with that carbon a carbonyl. In certain embodiments, each R⁴ is independently hydrogen, unsubstituted C₁₋₄ alkyl, C₁₋₄ haloalkyl, unsubstituted cyclopropyl, unsubstituted cyclopropylmethyl, C₁₋₄ haloalkoxy, or unsubstituted C₁₋₄ alkoxy. In certain embodiments, each R⁴ is independently hydrogen, unsubstituted C₁₋₄ alkyl, C₁₋₄ haloalkyl, unsubstituted cyclopropyl, unsubstituted cyclopropylmethyl, C₁₋₄ haloalkoxy, or unsubstituted C₁₋₄ alkoxy, or two instances of R⁴ on the same carbon form with that carbon a carbonyl.

In certain embodiments, R⁴ is halogen, or two instances of R⁴ on the same carbon form with that carbon a carbonyl. In certain embodiments, R⁴ is fluoro, or two instances of R⁴ on the same carbon form with that carbon a carbonyl. In certain embodiments, R⁴ is halogen. In certain embodiments, R⁴ is fluoro. In certain embodiments, two instances of R⁴ on the same carbon form with that carbon a carbonyl. In certain embodiments, each R⁴ is hydrogen.

In certain embodiments, p is 0, 1, 2, or 3. In certain embodiments, p is 0, 1, or 2. In certain embodiments, p is 0 or 2. In certain embodiments, p is 0 or 1. In certain embodiments, p is 1 or 2. In certain embodiments, p is 0. In certain embodiments, p is 2. In certain embodiments, p is 1. In certain embodiments, p is 3. In certain embodiments, p is 4. In certain embodiments, q is 0, 1, 2, or 3. In certain embodiments, q is 0, 1, or 2. In certain embodiments, q is 0 or 2. In certain embodiments, q is 0 or 1. In certain embodiments, q is 1 or 2. In certain embodiments, q is 0. In certain embodiments, q is 2. In certain embodiments, q is 1. In certain embodiments, q is 3. In certain embodiments, q is 4.

m, n, t, and u

As described herein for Formula (I), t is 0, 1 or 2; u is 0, 1 or 2; m is 0, 1 or 2; and n is 0, 1 or 2; provided that the sum of m and u is 2 or 3, and the sum of n and t is 2 or 3.

As described herein for Formula (II), t is 1 or 2; and n is 1 or 2.

In certain embodiments, t is 1 or 2; and n is 1 or 2. In certain embodiments, t is 1. In certain embodiments, t is 2. In certain embodiments, n is 1. In certain embodiments, n is 2. In certain embodiments, t is 1; and n is 2. In certain embodiments, t is 2; and n is 1. In certain embodiments, t is 1; and n is 1. In certain embodiments, t is 2; and n is 2.

In certain embodiments, t is 0. In certain embodiments, t is 1. In certain embodiments, t is 2. In certain embodiments, u is 0. In certain embodiments, u is 1. In certain embodiments, u is 2. In certain embodiments, m is 0. In certain embodiments, m is 1. In certain embodiments, m is 2. In certain embodiments, n is 0. In certain embodiments, n is 1. In certain embodiments, n is 2.

In certain embodiments, m is 0; u is 2; n is 0; and t is 2. In certain embodiments, m is 0; u is 2; n is 1; and t is 2. In certain embodiments, m is 0; u is 2; n is 2; and t is 0. In certain embodiments, m is 0; u is 2; n is 2; and t is 1.

In certain embodiments, m is 1; u is 1; n is 0; and t is 2. In certain embodiments, m is 1; u is 1; n is 1; and t is 2. In certain embodiments, m is 1; u is 1; n is 2; and t is 1. In certain embodiments, m is 1; u is 1; n is 2; and t is 0.

In certain embodiments, m is 1; u is 2; n is 0; and t is 2. In certain embodiments, m is 1; u is 2; n is 1; and t is 2. In certain embodiments, m is 1; u is 2; n is 2; and t is 1. In certain embodiments, m is 1; u is 2; n is 2; and t is 0.

In certain embodiments, m is 2; u is 1; n is 0; and t is 2. In certain embodiments, m is 2; u is 1; n is 1; and t is 2. In certain embodiments, m is 2; u is 1; n is 2; and t is 1. In certain embodiments, m is 2; u is 1; n is 2; and t is 0.

In certain embodiments, m is 1 or 2; and n is 1 or 2. In certain embodiments, m is 1. In certain embodiments, m is 2. In certain embodiments, n is 1. In certain embodiments, n is 2. In certain embodiments, m is 1; and n is 2. In certain embodiments, m is 2; and n is 1. In certain embodiments, m is 1; and n is 1. In certain embodiments, m is 2; and n is 2.

### Certain Embodiments

In certain embodiments, the compound of Formula (I) is of Formula (I-a):

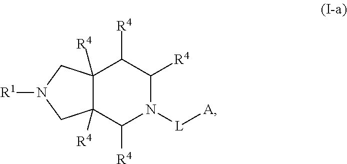

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, the compound of Formula (I-a) is of Formula (I-a-1):

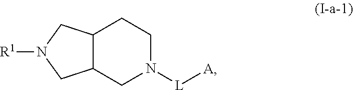

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-a) is of Formula (I-a-2):

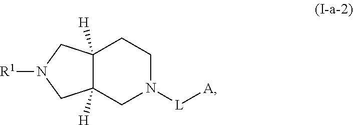

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-a) is of Formula (I-a-3):

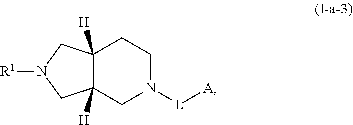

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-a-4):

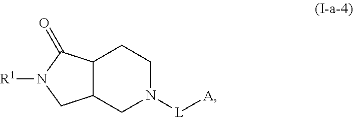

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-a-5):

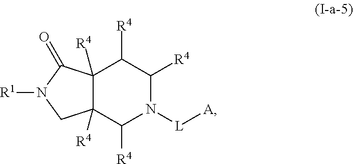

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, the compound of Formula (I) is of Formula (I-a-6):

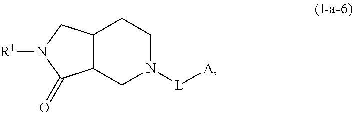

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-a-7):

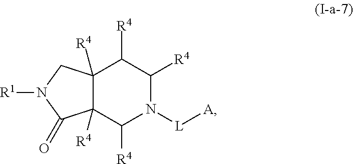

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, the compound of Formula (I) is of Formula (I-a-8):

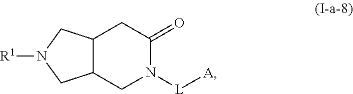

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-b):

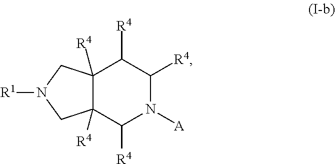

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, the compound of Formula (I-b) is of Formula (I-b-1):

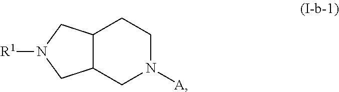

or a pharmaceutically acceptable salt, co-crystal, tautomer, stereoisomer, solvate, hydrate, polymorph, isotopically enriched derivative, or prodrug thereof; wherein R¹ and A are as defined herein.

In certain embodiments, the compound of Formula (I-b) is of Formula (I-b-2):

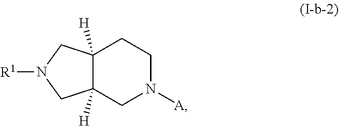

or a pharmaceutically acceptable salt, co-crystal, tautomer, stereoisomer, solvate, hydrate, polymorph, isotopically enriched derivative, or prodrug thereof; wherein R¹ and A are as defined herein.

In certain embodiments, the compound of Formula (I-b) is of Formula (I-b-3):

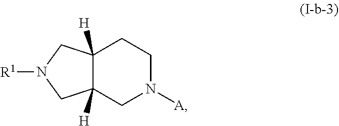

or a pharmaceutically acceptable salt, co-crystal, tautomer, stereoisomer, solvate, hydrate, polymorph, isotopically enriched derivative, or prodrug thereof; wherein R¹ and A are as defined herein.

In certain embodiments, the compound of Formula (I) is of Formula (I-b-4):

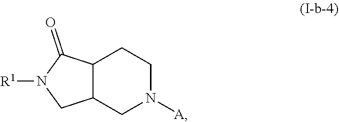

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-b-5):

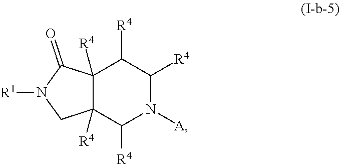

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, the compound of Formula (I) is of Formula (I-b-6):

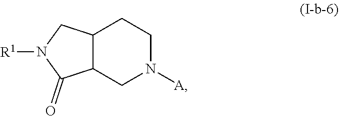

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-b-7):

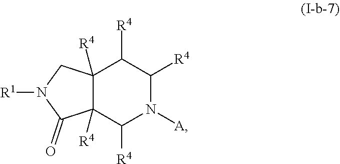

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, the compound of Formula (I) is of Formula (I-c):

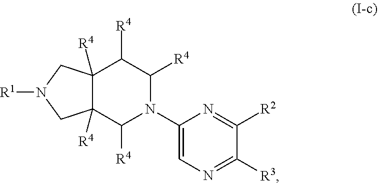

or a pharmaceutically acceptable salt, co-crystal, tautomer, stereoisomer, solvate, hydrate, polymorph, isotopically enriched derivative, or prodrug thereof; wherein R⁴, R¹, R², and R³ are as defined herein.

In certain embodiments, the compound of Formula (I-c) is of Formula (I-c-1):

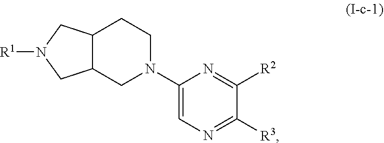

or a pharmaceutically acceptable salt, co-crystal, tautomer, stereoisomer, solvate, hydrate, polymorph, isotopically enriched derivative, or prodrug thereof; wherein R¹, R², and R³ are as defined herein.

In certain embodiments, the compound of Formula (I-c) is of Formula (I-c-2):

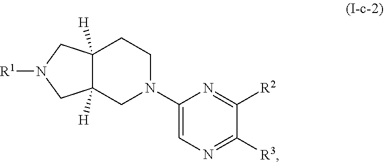

or a pharmaceutically acceptable salt, co-crystal, tautomer, stereoisomer, solvate, hydrate, polymorph, isotopically enriched derivative, or prodrug thereof; wherein R¹, R², and R³ are as defined herein.

In certain embodiments, the compound of Formula (I-c) is of Formula (I-c-3):

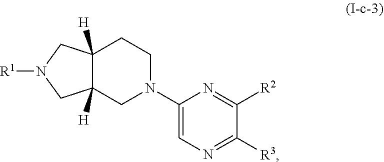

or a pharmaceutically acceptable salt, co-crystal, tautomer, stereoisomer, solvate, hydrate, polymorph, isotopically enriched derivative, or prodrug thereof; wherein R¹, R², and R³ are as defined herein.

In certain embodiments, the compound of Formula (I) is of Formula (I-c-4):

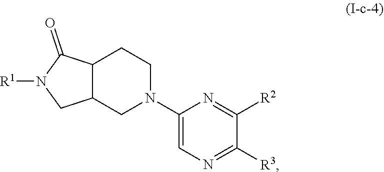

or a pharmaceutically acceptable salt, co-crystal, tautomer, stereoisomer, solvate, hydrate, polymorph, isotopically enriched derivative, or prodrug thereof; wherein R¹, R², and R³ are as defined herein.

In certain embodiments, the compound of Formula (I) is of Formula (I-c-5):

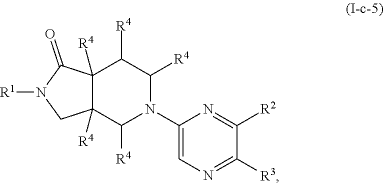

or a pharmaceutically acceptable salt, co-crystal, tautomer, stereoisomer, solvate, hydrate, polymorph, isotopically enriched derivative, or prodrug thereof; wherein R⁴, R¹, R², and R³ are as defined herein.

In certain embodiments, the compound of Formula (I) is of Formula (I-c-6):

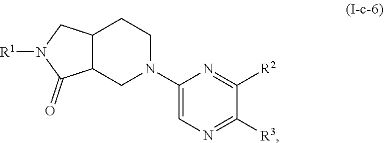

or a pharmaceutically acceptable salt, co-crystal, tautomer, stereoisomer, solvate, hydrate, polymorph, isotopically enriched derivative, or prodrug thereof; wherein R¹, R², and R³ are as defined herein.

In certain embodiments, the compound of Formula (I) is of Formula (I-c-7):

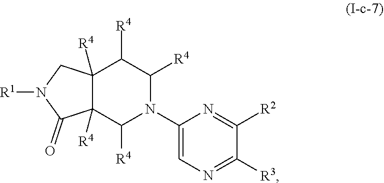

or a pharmaceutically acceptable salt, co-crystal, tautomer, stereoisomer, solvate, hydrate, polymorph, isotopically enriched derivative, or prodrug thereof; wherein R⁴, R¹, R², and R³ are as defined herein.

In certain embodiments, the compound of Formula (I) is of Formula (I-d):

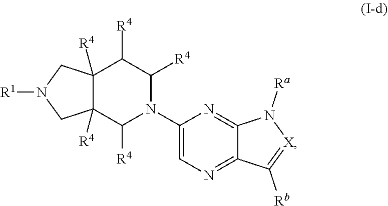

or a pharmaceutically acceptable salt, co-crystal, tautomer, stereoisomer, solvate, hydrate, polymorph, isotopically enriched derivative, or prodrug thereof; wherein R⁴ and R¹ are as defined herein; X is N or CR^{a}; each R^{a} is independently hydrogen, substituted or unsubstituted alkyl or substituted or unsubstituted heterocyclyl; and R^{b} is hydrogen, halogen, substituted or unsubstituted alkyl, substituted or unsubstituted cycloalkyl, or substituted or unsubstituted alkoxy.

In certain embodiments of Formula (I-d), X is N or CH; and R^{a} is hydrogen, haloalkyl, substituted or unsubstituted heterocyclylalkyl, or substituted or unsubstituted heterocyclyl. In certain embodiments, X is N or CH; and R^{a} is hydrogen, fluoroalkyl, oxetanylalkyl, or substituted or unsubstituted oxetanyl. In certain embodiments, X is N or CH; and R^{a} is hydrogen, C₁₋₄ fluoroalkyl, oxetanylalkyl, or unsubstituted oxetanyl. In certain embodiments, X is N; and R^{a} is substituted or unsubstituted alkyl or substituted or unsubstituted heterocyclyl. In certain embodiments, X is N; and R^{a} is haloalkyl or unsubstituted heterocyclyl. In certain embodiments, X is N; and R^{a} is fluoroalkyl or unsubstituted oxetanyl. In certain embodiments, X is N; and R^{a} is C₁₋₄ fluoroalkyl or unsubstituted oxetanyl. In certain embodiments, X is CH; and R^{a} is hydrogen or substituted or unsubstituted alkyl. In certain embodiments, X is CH; and R^{a} is hydrogen or substituted or unsubstituted heterocyclylalkyl. In certain embodiments, X is CH; and R^{a} is hydrogen or substituted or unsubstituted oxetanylalkyl. In certain embodiments, X is CH; and R^{a} is hydrogen. In certain embodiments, X is CH; and R^{a} is substituted or unsubstituted heterocyclylalkyl. In certain embodiments, X is CH; and R^{a} is unsubstituted heterocyclylalkyl. In certain embodiments, X is CH; and R^{a} is unsubstituted oxetanylalkyl.

In certain embodiments, the compound of Formula (I-d) is of Formula (I-d-1):

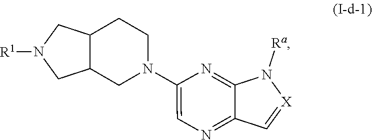

or a pharmaceutically acceptable salt, co-crystal, tautomer, stereoisomer, solvate, hydrate, polymorph, isotopically enriched derivative, or prodrug thereof; wherein R¹ is as defined herein; X is N or CR^{a}; and each R^{a} is independently hydrogen, substituted or unsubstituted alkyl or substituted or unsubstituted heterocyclyl.

In certain embodiments, the compound of Formula (I-d) is of Formula (I-d-2):

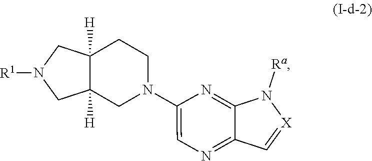

or a pharmaceutically acceptable salt, co-crystal, tautomer, stereoisomer, solvate, hydrate, polymorph, isotopically enriched derivative, or prodrug thereof; wherein R¹ is as defined herein; X is N or CR^{a}; and each R^{a} is independently hydrogen, substituted or unsubstituted alkyl or substituted or unsubstituted heterocyclyl.

In certain embodiments, the compound of Formula (I-d) is of Formula (I-d-3):

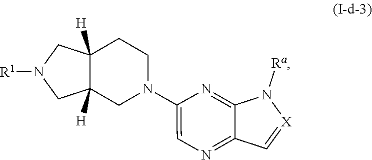

or a pharmaceutically acceptable salt, co-crystal, tautomer, stereoisomer, solvate, hydrate, polymorph, isotopically enriched derivative, or prodrug thereof; wherein R¹ is as defined herein; X is N or CR^{a}; and each R^{a} is independently hydrogen, substituted or unsubstituted alkyl or substituted or unsubstituted heterocyclyl.

In certain embodiments, the compound of Formula (I) is of Formula (I-d-4):

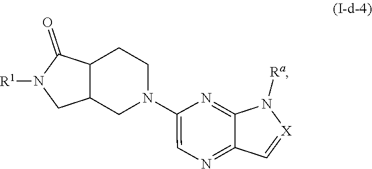

or a pharmaceutically acceptable salt, co-crystal, tautomer, stereoisomer, solvate, hydrate, polymorph, isotopically enriched derivative, or prodrug thereof; wherein R¹ is as defined herein; X is N or CR^{a}; and each R^{a} is independently hydrogen, substituted or unsubstituted alkyl or substituted or unsubstituted heterocyclyl.

In certain embodiments, the compound of Formula (I) is of Formula (I-d-5):

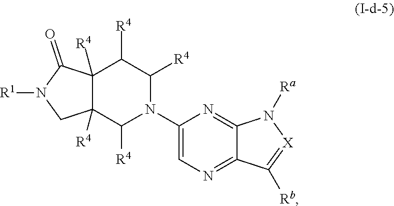

or a pharmaceutically acceptable salt, co-crystal, tautomer, stereoisomer, solvate, hydrate, polymorph, isotopically enriched derivative, or prodrug thereof; wherein R⁴ and R¹ are as defined herein; X is N or CR^{a}; each R^{a} is independently hydrogen, substituted or unsubstituted alkyl or substituted or unsubstituted heterocyclyl; and R^{b} is hydrogen, halogen, substituted or unsubstituted alkyl, substituted or unsubstituted cycloalkyl, or substituted or unsubstituted alkoxy.

In certain embodiments, the compound of Formula (I) is of Formula (I-d-6):

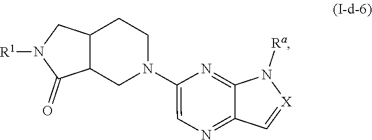

or a pharmaceutically acceptable salt, co-crystal, tautomer, stereoisomer, solvate, hydrate, polymorph, isotopically enriched derivative, or prodrug thereof; wherein R¹ is as defined herein; X is N or CR^{a}; and each R^{a} is independently hydrogen, substituted or unsubstituted alkyl or substituted or unsubstituted heterocyclyl.

In certain embodiments, the compound of Formula (I) is of Formula (I-d-7):

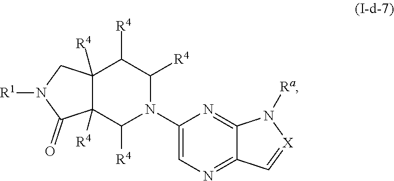

or a pharmaceutically acceptable salt, co-crystal, tautomer, stereoisomer, solvate, hydrate, polymorph, isotopically enriched derivative, or prodrug thereof; wherein R^{b}, R⁴ and R¹ are as defined herein; X is N or CR^{a}; and each R^{a} is independently hydrogen, substituted or unsubstituted alkyl or substituted or unsubstituted heterocyclyl.

In certain embodiments, the compound of Formula (I) is of Formula (I-e):

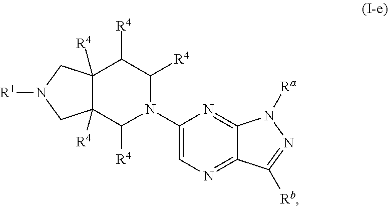

or a pharmaceutically acceptable salt, co-crystal, tautomer, stereoisomer, solvate, hydrate, polymorph, isotopically enriched derivative, or prodrug thereof; wherein R^{b}, R⁴, and R¹ are as defined herein; R^{a} is hydrogen, substituted or unsubstituted alkyl or substituted or unsubstituted heterocyclyl; and R^{b} is hydrogen, halogen, substituted or unsubstituted alkyl, substituted or unsubstituted cycloalkyl, or substituted or unsubstituted alkoxy.

In certain embodiments of Formula (I-e), R^{a} is substituted or unsubstituted alkyl or substituted or unsubstituted heterocyclyl. In certain embodiments, R^{a} is haloalkyl or unsubstituted heterocyclyl. In certain embodiments, R^{a} is fluoroalkyl or unsubstituted oxetanyl. In certain embodiments, R^{a} is C₁₋₄ fluoroalkyl or unsubstituted oxetanyl. In certain embodiments, R^{a} is 2,2-difluoroethyl, 2,2,2-trifluoroethyl or unsubstituted oxetanyl. In certain embodiments, R^{a} is 2,2-difluoroethyl. In certain embodiments, R^{a} is 2,2,2-trifluoroethyl. In certain embodiments, R^{a} is unsubstituted oxetanyl.

In certain embodiments, the compound of Formula (I-e) is of Formula (I-e-1):

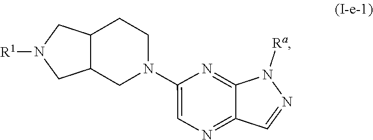

or a pharmaceutically acceptable salt, co-crystal, tautomer, stereoisomer, solvate, hydrate, polymorph, isotopically enriched derivative, or prodrug thereof; wherein R¹ is as defined herein; and R^{a} is hydrogen, substituted or unsubstituted alkyl or substituted or unsubstituted heterocyclyl.

In certain embodiments, the compound of Formula (I-e) is of Formula (I-e-2):

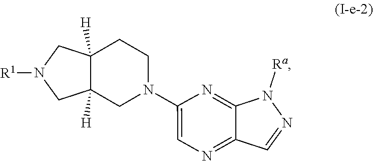

or a pharmaceutically acceptable salt, co-crystal, tautomer, stereoisomer, solvate, hydrate, polymorph, isotopically enriched derivative, or prodrug thereof; wherein R¹ is as defined herein; and R^{a} is hydrogen, substituted or unsubstituted alkyl or substituted or unsubstituted heterocyclyl.

In certain embodiments, the compound of Formula (I-e) is of Formula (I-e-3):

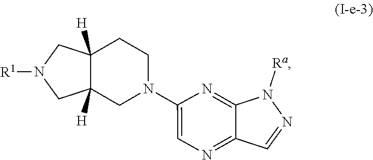

or a pharmaceutically acceptable salt, co-crystal, tautomer, stereoisomer, solvate, hydrate, polymorph, isotopically enriched derivative, or prodrug thereof; wherein R¹ is as defined herein; and R^{a} is hydrogen, substituted or unsubstituted alkyl or substituted or unsubstituted heterocyclyl.

In certain embodiments, the compound of Formula (I) is of Formula (I-e-4):

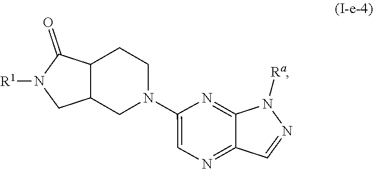

or a pharmaceutically acceptable salt, co-crystal, tautomer, stereoisomer, solvate, hydrate, polymorph, isotopically enriched derivative, or prodrug thereof; wherein R¹ is as defined herein; and R^{a} is hydrogen, substituted or unsubstituted alkyl or substituted or unsubstituted heterocyclyl.

In certain embodiments, the compound of Formula (I) is of Formula (I-e-5):

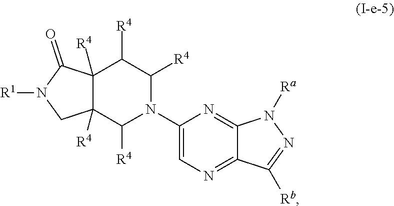

or a pharmaceutically acceptable salt, co-crystal, tautomer, stereoisomer, solvate, hydrate, polymorph, isotopically enriched derivative, or prodrug thereof; wherein R^{b}, R⁴, and R¹ are as defined herein; R^{a} is hydrogen, substituted or unsubstituted alkyl or substituted or unsubstituted heterocyclyl; and R^{b} is hydrogen, halogen, substituted or unsubstituted alkyl, substituted or unsubstituted cycloalkyl, or substituted or unsubstituted alkoxy.

In certain embodiments, the compound of Formula (I) is of Formula (I-e-6):

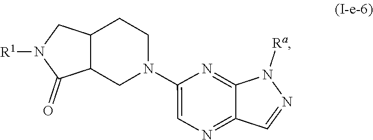

or a pharmaceutically acceptable salt, co-crystal, tautomer, stereoisomer, solvate, hydrate, polymorph, isotopically enriched derivative, or prodrug thereof; wherein R¹ is as defined herein; and R^{a} is hydrogen, substituted or unsubstituted alkyl or substituted or unsubstituted heterocyclyl.

In certain embodiments, the compound of Formula (I) is of Formula (I-e-7):

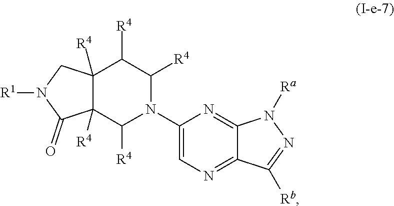

or a pharmaceutically acceptable salt, co-crystal, tautomer, stereoisomer, solvate, hydrate, polymorph, isotopically enriched derivative, or prodrug thereof; wherein R^{b}, R⁴, and R¹ are as defined herein; R^{a} is hydrogen, substituted or unsubstituted alkyl or substituted or unsubstituted heterocyclyl; and R^{b} is hydrogen, halogen, substituted or unsubstituted alkyl, substituted or unsubstituted cycloalkyl, or substituted or unsubstituted alkoxy.

In certain embodiments, the compound of Formula (I) is of Formula (I-f):

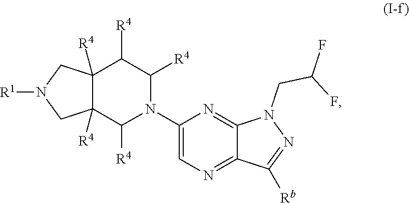

or a pharmaceutically acceptable salt, co-crystal, tautomer, stereoisomer, solvate, hydrate, polymorph, isotopically enriched derivative, or prodrug thereof; wherein R^{b}, R¹ and R⁴ are as defined herein.

In certain embodiments, the compound of Formula (I-f) is of Formula (I-f-1):

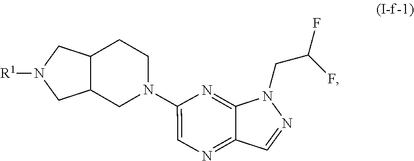

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I-f) is of Formula (I-f-2):

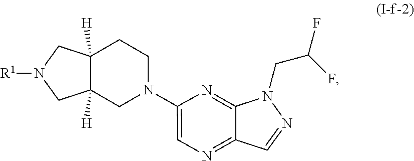

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I-f) is of Formula (I-f-3):

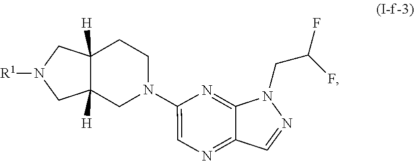

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I) is of Formula (I-f-4):

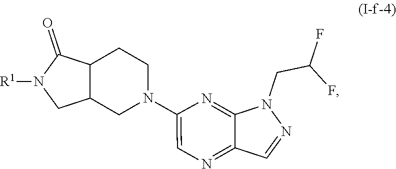

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I) is of Formula (I-f-5):

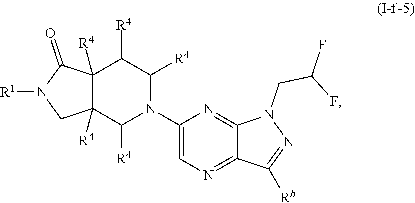

or a pharmaceutically acceptable salt, co-crystal, tautomer, stereoisomer, solvate, hydrate, polymorph, isotopically enriched derivative, or prodrug thereof; wherein R^{b}, R¹ and R⁴ are as defined herein.

In certain embodiments, the compound of Formula (I) is of Formula (I-f-6):

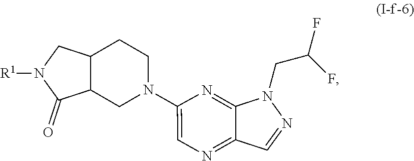

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I) is of Formula (I-f-7):

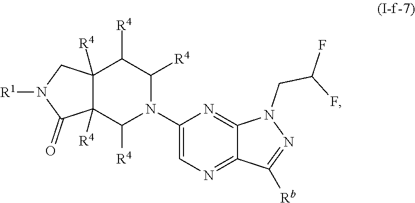

or a pharmaceutically acceptable salt, co-crystal, tautomer, stereoisomer, solvate, hydrate, polymorph, isotopically enriched derivative, or prodrug thereof; wherein R^{b}, R¹ and R⁴ are as defined herein.

In certain embodiments, the compound of Formula (I) is of Formula (I-g):

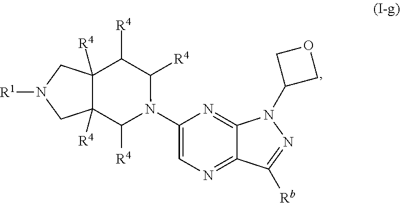

or a pharmaceutically acceptable salt, co-crystal, tautomer, stereoisomer, solvate, hydrate, polymorph, isotopically enriched derivative, or prodrug thereof; wherein R^{b}, R¹ and R⁴ are as defined herein.

In certain embodiments, the compound of Formula (I-g) is of Formula (I-g-1):

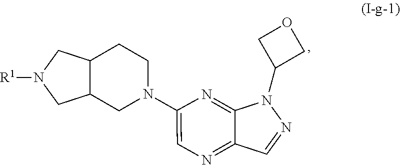

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I-g) is of Formula (I-g-2):

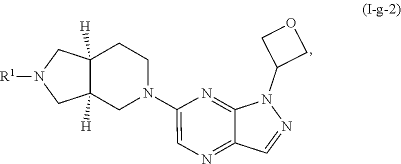

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I-g) is of Formula (I-g-3):

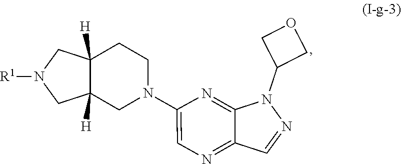

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I) is of Formula (I-h):

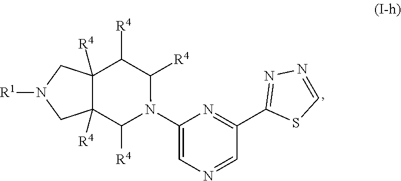

or a pharmaceutically acceptable salt, co-crystal, tautomer, stereoisomer, solvate, hydrate, polymorph, isotopically enriched derivative, or prodrug thereof; wherein R¹ and R⁴ are as defined herein.

In certain embodiments, the compound of Formula (I-h) is of Formula (I-h-1):

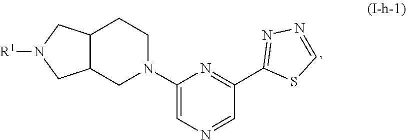

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I-h) is of Formula (I-h-2):

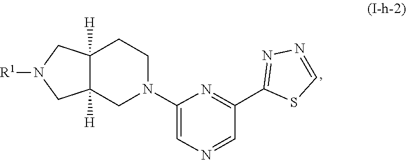

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I-h) is of Formula (I-h-3):

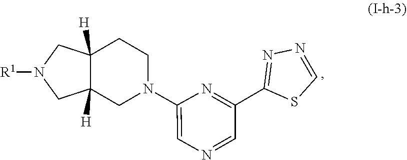

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I) is of Formula (I-i):

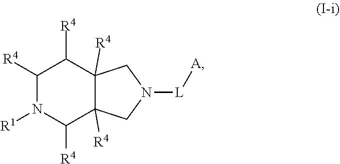

or a pharmaceutically acceptable salt, co-crystal, tautomer, stereoisomer, solvate, hydrate, polymorph, isotopically enriched derivative, or prodrug thereof; wherein A, L, R¹ and R⁴ are as defined herein.

In certain embodiments, the compound of Formula (I-i) is of Formula (I-i-1):

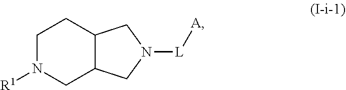

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-i) is of Formula (I-i-2):

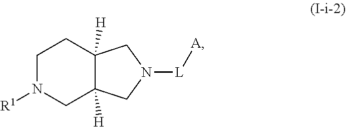

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I-i) is of Formula (I-i-3):

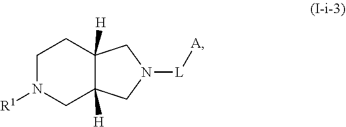

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I) is of Formula (I-j):

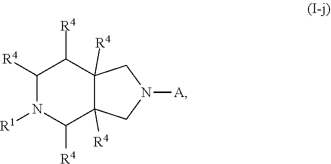

or a pharmaceutically acceptable salt, co-crystal, tautomer, stereoisomer, solvate, hydrate, polymorph, isotopically enriched derivative, or prodrug thereof; wherein A, R¹ and R⁴ are as defined herein.

In certain embodiments, the compound of Formula (I-j) is of Formula (I-j-1):

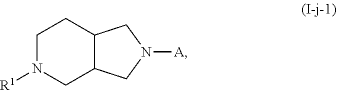

or a pharmaceutically acceptable salt, co-crystal, tautomer, stereoisomer, solvate, hydrate, polymorph, isotopically enriched derivative, or prodrug thereof; wherein R¹ and A are as defined herein.

In certain embodiments, the compound of Formula (I-j) is of Formula (I-j-2):

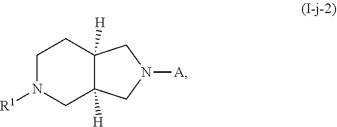

or a pharmaceutically acceptable salt, co-crystal, tautomer, stereoisomer, solvate, hydrate, polymorph, isotopically enriched derivative, or prodrug thereof; wherein R¹ and A are as defined herein.

In certain embodiments, the compound of Formula (I-j) is of Formula (I-j-3):

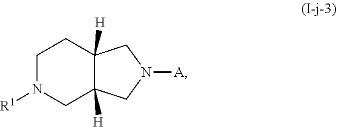

or a pharmaceutically acceptable salt, co-crystal, tautomer, stereoisomer, solvate, hydrate, polymorph, isotopically enriched derivative, or prodrug thereof; wherein R¹ and A are as defined herein.

In certain embodiments, the compound of Formula (I) is of Formula (I-k):

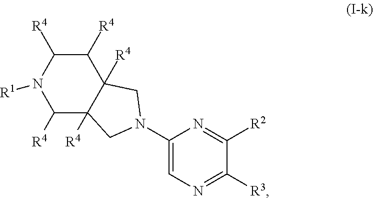

or a pharmaceutically acceptable salt, co-crystal, tautomer, stereoisomer, solvate, hydrate, polymorph, isotopically enriched derivative, or prodrug thereof; wherein R¹, R², R³ and R⁴ are as defined herein.

In certain embodiments, the compound of Formula (I-k) is of Formula (I-k-1):

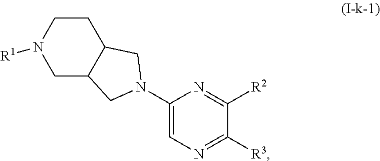

or a pharmaceutically acceptable salt, co-crystal, tautomer, stereoisomer, solvate, hydrate, polymorph, isotopically enriched derivative, or prodrug thereof; wherein R¹, R², and R³ are as defined herein.

In certain embodiments, the compound of Formula (I-k) is of Formula (I-k-2):

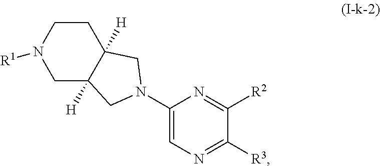

or a pharmaceutically acceptable salt, co-crystal, tautomer, stereoisomer, solvate, hydrate, polymorph, isotopically enriched derivative, or prodrug thereof; wherein R¹, R², and R³ are as defined herein.

In certain embodiments, the compound of Formula (I-k) is of Formula (I-k-3):

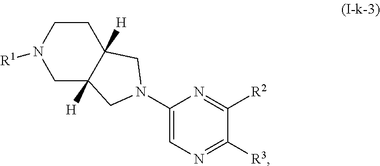

or a pharmaceutically acceptable salt, co-crystal, tautomer, stereoisomer, solvate, hydrate, polymorph, isotopically enriched derivative, or prodrug thereof; wherein R¹, R², and R³ are as defined herein.

In certain embodiments, the compound of Formula (I) is of Formula (I-l):

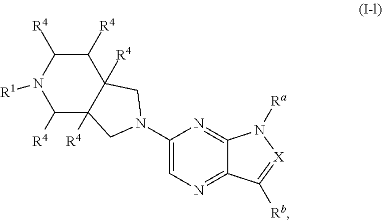

or a pharmaceutically acceptable salt, co-crystal, tautomer, stereoisomer, solvate, hydrate, polymorph, isotopically enriched derivative, or prodrug thereof; wherein R^{b}, R¹ and R⁴ are as defined herein; X is N or CR^{a}; and each R^{a} is independently hydrogen, substituted or unsubstituted alkyl, or substituted or unsubstituted heterocyclyl.

In certain embodiments of Formula (I-l), X is N or CH; and R^{a} is hydrogen, haloalkyl, substituted or unsubstituted heterocyclylalkyl, or substituted or unsubstituted heterocyclyl. In certain embodiments, X is N or CH; and R^{a} is hydrogen, fluoroalkyl, oxetanylalkyl, or substituted or unsubstituted oxetanyl. In certain embodiments, X is N or CH; and R^{a} is hydrogen, C₁₋₄ fluoroalkyl, oxetanylalkyl, or unsubstituted oxetanyl. In certain embodiments, X is N; and R^{a} is substituted or unsubstituted alkyl or substituted or unsubstituted heterocyclyl. In certain embodiments, X is N; and R^{a} is haloalkyl or unsubstituted heterocyclyl. In certain embodiments, X is N; and R^{a} is fluoroalkyl or unsubstituted oxetanyl. In certain embodiments, X is N; and R^{a} is C₁₋₄ fluoroalkyl or unsubstituted oxetanyl. In certain embodiments, X is N; and R^{a} is C₁₋₄ fluoroalkyl. In certain embodiments, X is CH; and R^{a} is hydrogen or substituted or unsubstituted alkyl. In certain embodiments, X is CH; and R^{a} is hydrogen or substituted or unsubstituted heterocyclylalkyl. In certain embodiments, X is CH; and R^{a} is hydrogen or substituted or unsubstituted oxetanylalkyl. In certain embodiments, X is CH; and R^{a} is hydrogen. In certain embodiments, X is CH; and R^{a} is substituted or unsubstituted heterocyclylalkyl. In certain embodiments, X is CH; and R^{a} is unsubstituted heterocyclylalkyl. In certain embodiments, X is CH; and R^{a} is unsubstituted oxetanylalkyl.

In certain embodiments, the compound of Formula (I-l) is of Formula (I-l-1):

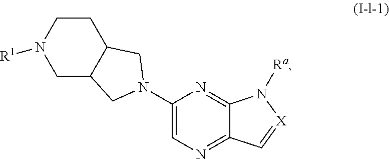

or a pharmaceutically acceptable salt, co-crystal, tautomer, stereoisomer, solvate, hydrate, polymorph, isotopically enriched derivative, or prodrug thereof; wherein R¹ is as defined herein; X is N or CR^{a}; and each R^{a} is independently hydrogen, substituted or unsubstituted alkyl, or substituted or unsubstituted heterocyclyl.

In certain embodiments, the compound of Formula (I-l) is of Formula (I-l-2):

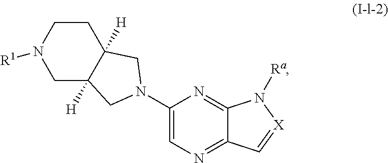

or a pharmaceutically acceptable salt, co-crystal, tautomer, stereoisomer, solvate, hydrate, polymorph, isotopically enriched derivative, or prodrug thereof; wherein R¹ is as defined herein; X is N or CR^{a}; and each R^{a} is independently hydrogen, substituted or unsubstituted alkyl or substituted or unsubstituted heterocyclyl.

In certain embodiments, the compound of Formula (I-l) is of Formula (I-l-3):

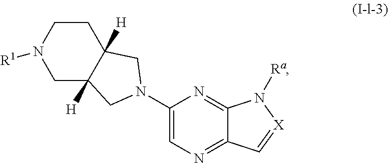

or a pharmaceutically acceptable salt, co-crystal, tautomer, stereoisomer, solvate, hydrate, polymorph, isotopically enriched derivative, or prodrug thereof; wherein R¹ is as defined herein; X is N or CR^{a}; and each R^{a} is independently hydrogen, substituted or unsubstituted alkyl, or substituted or unsubstituted heterocyclyl.

In certain embodiments, the compound of Formula (I) is of Formula (I-m):

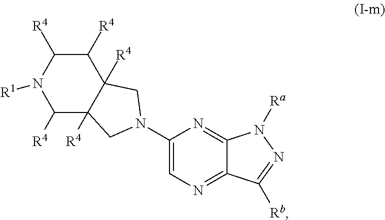

or a pharmaceutically acceptable salt, co-crystal, tautomer, stereoisomer, solvate, hydrate, polymorph, isotopically enriched derivative, or prodrug thereof; wherein R¹ and R⁴ are as defined herein; R^{a} is hydrogen, substituted or unsubstituted alkyl or substituted or unsubstituted heterocyclyl; and R^{b} is hydrogen, halogen, substituted or unsubstituted alkyl, substituted or unsubstituted cycloalkyl, or substituted or unsubstituted alkoxy.

In certain embodiments of Formula (I-m), R^{a} is substituted or unsubstituted alkyl or substituted or unsubstituted heterocyclyl. In certain embodiments, R^{a} is haloalkyl or unsubstituted heterocyclyl. In certain embodiments, R^{a} is fluoroalkyl or unsubstituted oxetanyl. In certain embodiments, R^{a} is C₁₋₄ fluoroalkyl or unsubstituted oxetanyl. In certain embodiments, R^{a} is 2,2-difluoroethyl, 2,2,2-trifluoroethyl or unsubstituted oxetanyl. In certain embodiments, R^{a} is 2,2-difluoroethyl. In certain embodiments, R^{a} is 2,2,2-trifluoroethyl. In certain embodiments, R^{a} is unsubstituted oxetanyl.

In certain embodiments, the compound of Formula (I-m) is of Formula (I-m-1):

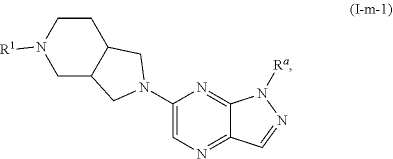

or a pharmaceutically acceptable salt, co-crystal, tautomer, stereoisomer, solvate, hydrate, polymorph, isotopically enriched derivative, or prodrug thereof; wherein R¹ is as defined herein; and R^{a} is hydrogen, substituted or unsubstituted alkyl or substituted or unsubstituted heterocyclyl.

In certain embodiments, the compound of Formula (I-m) is of Formula (I-m-2):

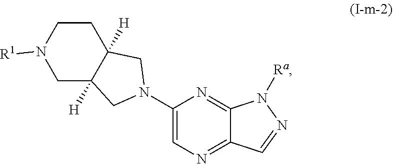

or a pharmaceutically acceptable salt, co-crystal, tautomer, stereoisomer, solvate, hydrate, polymorph, isotopically enriched derivative, or prodrug thereof; wherein R¹ is as defined herein; and R^{a} is hydrogen, substituted or unsubstituted alkyl, or substituted or unsubstituted heterocyclyl.

In certain embodiments, the compound of Formula (I-m) is of Formula (I-m-3):

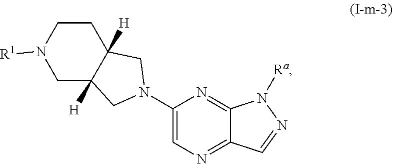

or a pharmaceutically acceptable salt, co-crystal, tautomer, stereoisomer, solvate, hydrate, polymorph, isotopically enriched derivative, or prodrug thereof; wherein R¹ is as defined herein; and R^{a} is hydrogen, substituted or unsubstituted alkyl, or substituted or unsubstituted heterocyclyl.

In certain embodiments, the compound of Formula (I) is of Formula (I-n):

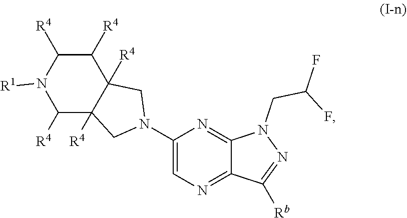

or a pharmaceutically acceptable salt, co-crystal, tautomer, stereoisomer, solvate, hydrate, polymorph, isotopically enriched derivative, or prodrug thereof; wherein R^{b}, R¹ and R⁴ are as defined herein.

In certain embodiments, the compound of Formula (I-n) is of Formula (I-n-1):

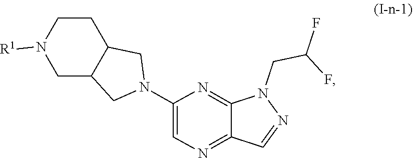

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I-n) is of Formula (I-n-2):

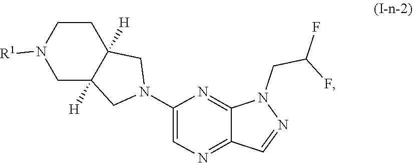

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I-n) is of Formula (I-n-3):

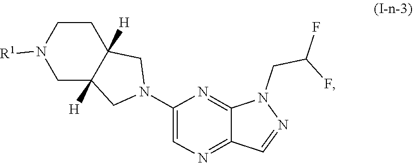

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I) is of Formula (I-o):

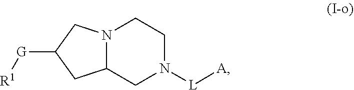

or a pharmaceutically acceptable salt, co-crystal, tautomer, stereoisomer, solvate, hydrate, polymorph, isotopically enriched derivative, or prodrug thereof; wherein R¹, G, L, and A are as defined herein.

In certain embodiments, the compound of Formula (I-o) is of Formula (I-o-1):

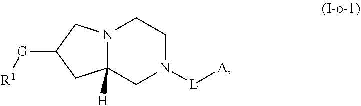

or a pharmaceutically acceptable salt, co-crystal, tautomer, stereoisomer, solvate, hydrate, polymorph, isotopically enriched derivative, or prodrug thereof; wherein R¹, G, L, and A are as defined herein.

In certain embodiments, the compound of Formula (I) is of Formula (I-p):

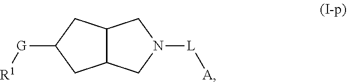

or a pharmaceutically acceptable salt, co-crystal, tautomer, stereoisomer, solvate, hydrate, polymorph, isotopically enriched derivative, or prodrug thereof; wherein R¹, G, L, and A are as defined herein.

In certain embodiments, the compound of Formula (I-p) is of Formula (I-p-1):

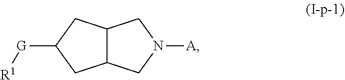

or a pharmaceutically acceptable salt, co-crystal, tautomer, stereoisomer, solvate, hydrate, polymorph, isotopically enriched derivative, or prodrug thereof; wherein R¹, G, and A are as defined herein.

In certain embodiments, the compound of Formula (I) is of Formula (I-q):

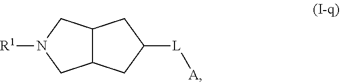

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-q) is of Formula (I-q-1):

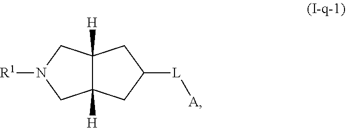

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-q) is of Formula (I-q-2):

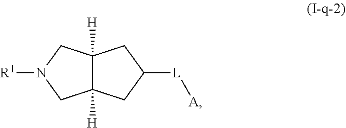

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-r):

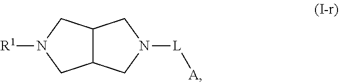

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-r) is of Formula (I-r-1):

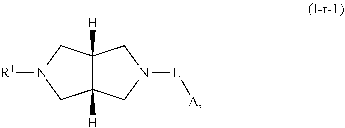

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-r) is of Formula (I-r-2):

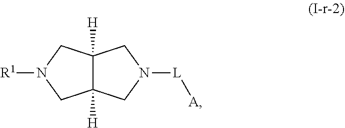

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-r) is of Formula (I-r-3):

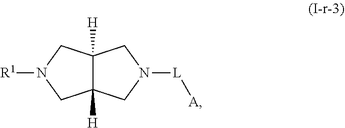

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-r) is of Formula (I-r-4):

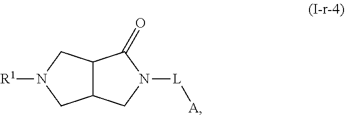

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-r) is of Formula (I-r-5):

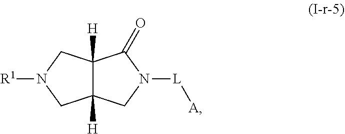

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-r) is of Formula (I-r-6):

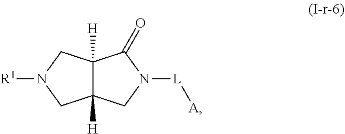

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-r) is of Formula (I-r-7):

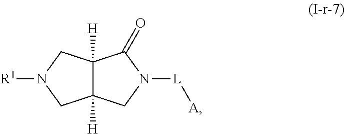

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-s):

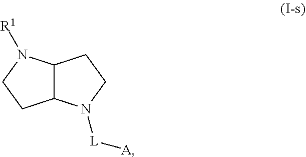

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-s) is of Formula (I-s-1):

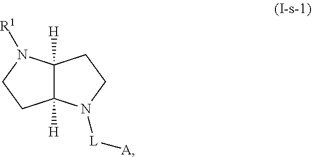

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-t):

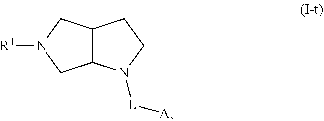

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-t) is of Formula (I-t-1):

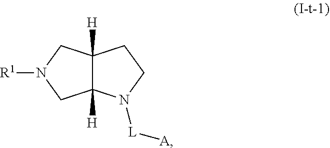

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-u):

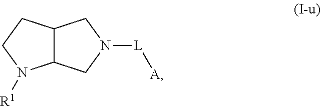

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-u) is of Formula (I-u-1):

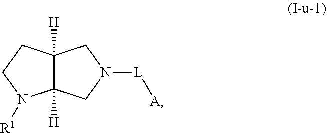

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-v):

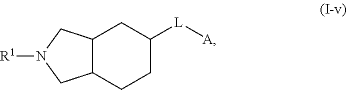

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-v) is of Formula (I-v-1):

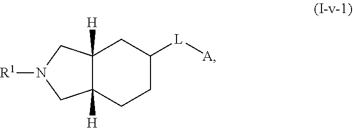

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-v) is of Formula (I-v-2):

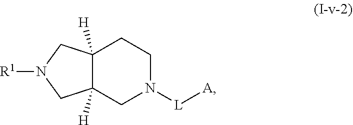

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-w):

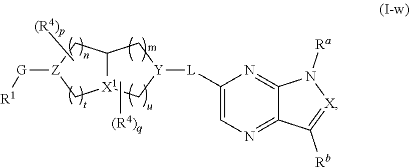

or a pharmaceutically acceptable salt, co-crystal, tautomer, stereoisomer, solvate, hydrate, polymorph, isotopically enriched derivative, or prodrug thereof; wherein R¹, G, Z, X¹, Y, R⁴, L, n, t, m, u, q, p, and R^{b} are as defined herein; X is N or CR^{a}; and each R^{a} is independently hydrogen, substituted or unsubstituted alkyl, or substituted or unsubstituted heterocyclyl.

In certain embodiments of Formula (I-x), X is N or CH; and R^{a} is hydrogen, haloalkyl, substituted or unsubstituted heterocyclylalkyl, or substituted or unsubstituted heterocyclyl. In certain embodiments, X is N or CH; and R^{a} is hydrogen, fluoroalkyl, oxetanylalkyl, or substituted or unsubstituted oxetanyl. In certain embodiments, X is N or CH; and R^{a} is hydrogen, C₁₋₄ fluoroalkyl, oxetanylalkyl, or unsubstituted oxetanyl. In certain embodiments, X is N; and R^{a} is substituted or unsubstituted alkyl or substituted or unsubstituted heterocyclyl. In certain embodiments, X is N; and R^{a} is haloalkyl or unsubstituted heterocyclyl. In certain embodiments, X is N; and R^{a} is fluoroalkyl or unsubstituted oxetanyl. In certain embodiments, X is N; and R^{a} is C₁₋₄ fluoroalkyl or unsubstituted oxetanyl. In certain embodiments, X is N; and R^{a} is C₁₋₄ fluoroalkyl. In certain embodiments, X is CH; and R^{a} is hydrogen or substituted or unsubstituted alkyl. In certain embodiments, X is CH; and R^{a} is hydrogen or substituted or unsubstituted heterocyclylalkyl. In certain embodiments, X is CH; and R^{a} is hydrogen or substituted or unsubstituted oxetanylalkyl. In certain embodiments, X is CH; and R^{a} is hydrogen. In certain embodiments, X is CH; and R^{a} is substituted or unsubstituted heterocyclylalkyl. In certain embodiments, X is CH; and R^{a} is unsubstituted heterocyclylalkyl. In certain embodiments, X is CH; and R^{a} is unsubstituted oxetanylalkyl.

In certain embodiments, the compound of Formula (I-w) is of Formula (I-w-1):

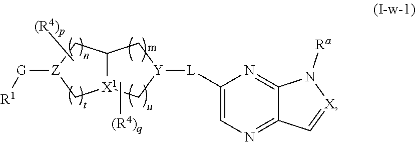

or a pharmaceutically acceptable salt, co-crystal, tautomer, stereoisomer, solvate, hydrate, polymorph, isotopically enriched derivative, or prodrug thereof; wherein R¹, G, Z, X¹, Y, R⁴, L, n, t, m, u, p, and q are as defined herein; X is N or CR^{a}; and each R^{a} is independently hydrogen, substituted or unsubstituted alkyl, or substituted or unsubstituted heterocyclyl.

In certain embodiments, the compound of Formula (I-w) is of Formula (I-w-2):

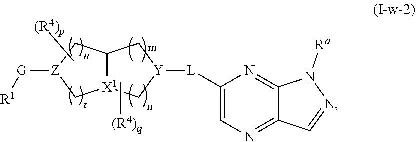

or a pharmaceutically acceptable salt, co-crystal, tautomer, stereoisomer, solvate, hydrate, polymorph, isotopically enriched derivative, or prodrug thereof; wherein R¹, G, Z, X¹, Y, R⁴, L, n, t, m, u, p, and q are as defined herein; and R^{a} is hydrogen, substituted or unsubstituted alkyl or substituted or unsubstituted heterocyclyl.

In certain embodiments, the compound of Formula (I-w) is of Formula (I-w-3):

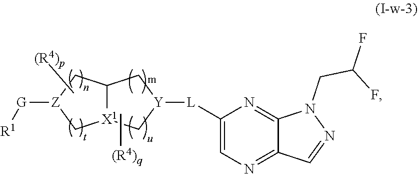

or a pharmaceutically acceptable salt, co-crystal, tautomer, stereoisomer, solvate, hydrate, polymorph, isotopically enriched derivative, or prodrug thereof; wherein R¹, G, Z, X¹, Y, R⁴, L, n, t, m, u, p, and q are as defined herein.

In certain embodiments, the compound of Formula (I) is one of the following compounds, or a pharmaceutically acceptable salt, co-crystal, tautomer, stereoisomer, solvate, hydrate, polymorph, isotopically enriched derivative, or prodrug thereof. The designation “(rac)” denotes a racemic mixture. While compounds may be depicted as racemic or as one or more diastereoisomers, enantiomers, or other isomers, all such racemic, diastereoisomer, enantiomer, or other isomer forms of that depicted are included in the present disclosure.

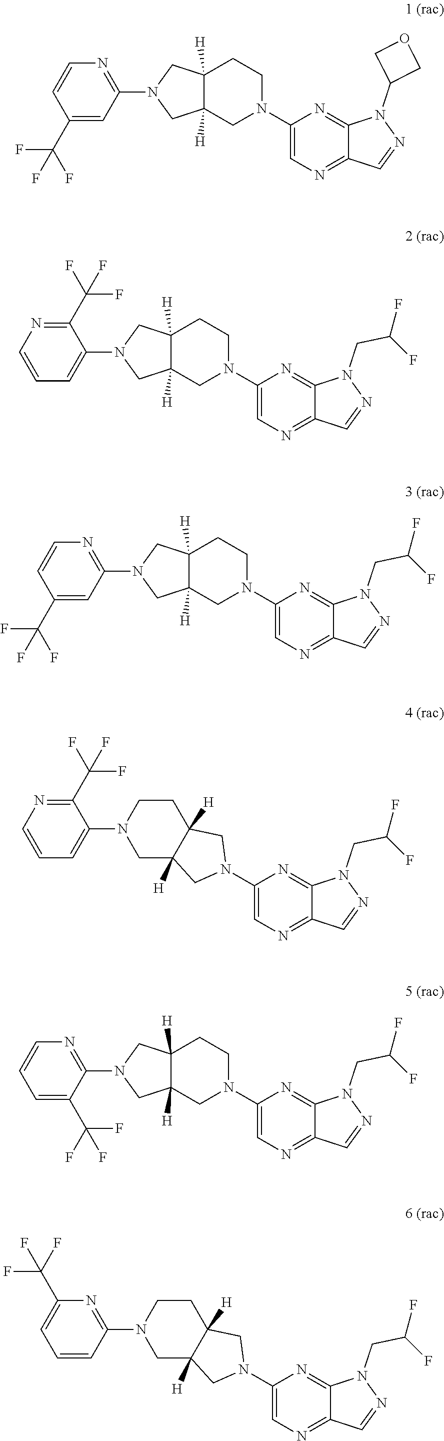 [SMILES: FC(F)(F)c1ccnc(N2C[C@H]3CCN(c4cnc5cnn(C6COC6)c5n4)C[C@H]3C2)c1 ; FC(F)Cn1ncc2ncc(N3CC[C@@H]4CN(c5cccnc5C(F)(F)F)C[C@@H]4C3)nc21 ; FC(F)Cn1ncc2ncc(N3CC[C@@H]4CN(c5cc(C(F)(F)F)ccn5)C[C@@H]4C3)nc21 ; FC(F)Cn1ncc2ncc(N3C[C@H]4CCN(c5cccnc5C(F)(F)F)C[C@H]4C3)nc21 ; FC(F)Cn1ncc2ncc(N3CC[C@H]4CN(c5ncccc5C(F)(F)F)C[C@H]4C3)nc21 ; FC(F)Cn1ncc2ncc(N3C[C@H]4CCN(c5cccc(C(F)(F)F)n5)C[C@H]4C3)nc21]

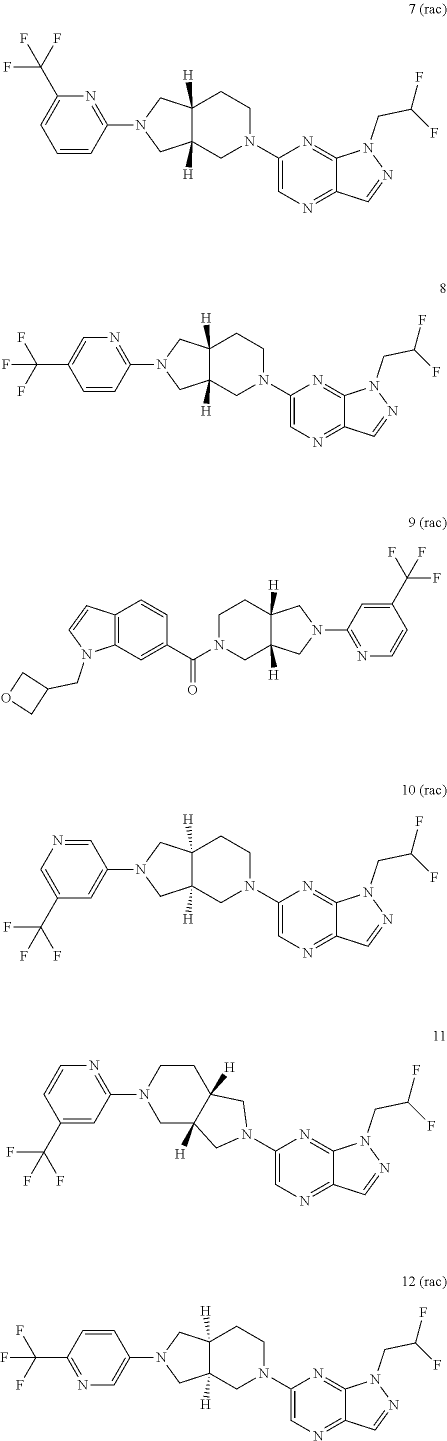 [SMILES: FC(F)Cn1ncc2ncc(N3CC[C@H]4CN(c5cccc(C(F)(F)F)n5)C[C@H]4C3)nc21 ; FC(F)Cn1ncc2ncc(N3CC[C@H]4CN(c5ccc(C(F)(F)F)cn5)C[C@H]4C3)nc21 ; O=C(c1ccc2ccn(CC3COC3)c2c1)N1CC[C@@H]2CN(c3cc(C(F)(F)F)ccn3)C[C@@H]2C1 ; FC(F)Cn1ncc2ncc(N3CC[C@@H]4CN(c5cncc(C(F)(F)F)c5)C[C@@H]4C3)nc21 ; FC(F)Cn1ncc2ncc(N3C[C@H]4CCN(c5cc(C(F)(F)F)ccn5)C[C@H]4C3)nc21 ; FC(F)Cn1ncc2ncc(N3CC[C@@H]4CN(c5ccc(C(F)(F)F)nc5)C[C@@H]4C3)nc21]

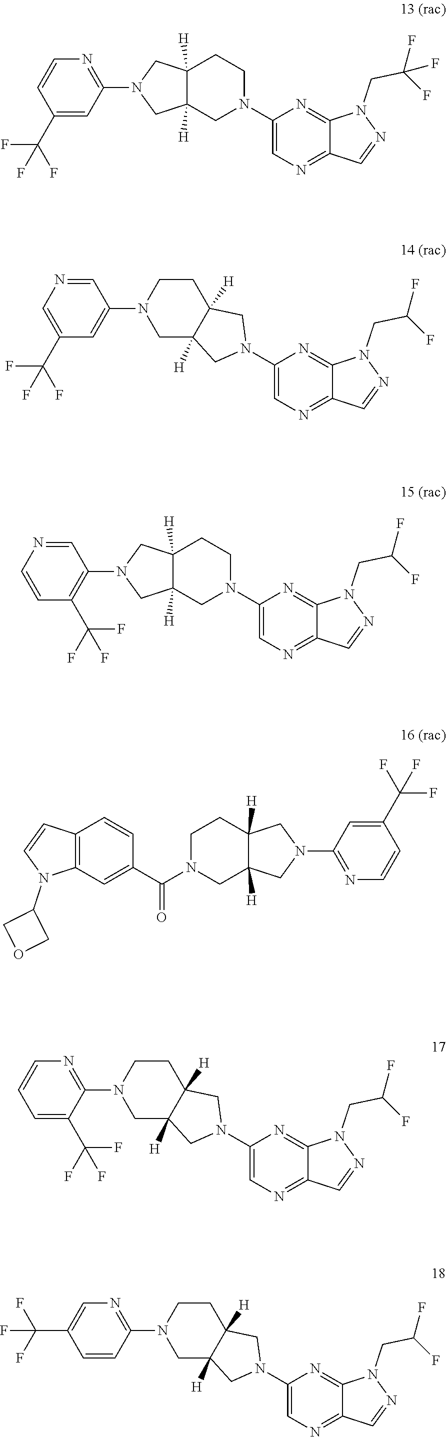 [SMILES: FC(F)(F)Cn1ncc2ncc(N3CC[C@@H]4CN(c5cc(C(F)(F)F)ccn5)C[C@@H]4C3)nc21 ; FC(F)Cn1ncc2ncc(N3C[C@H]4CN(c5cncc(C(F)(F)F)c5)CC[C@H]4C3)nc21 ; FC(F)Cn1ncc2ncc(N3CC[C@@H]4CN(c5cnccc5C(F)(F)F)C[C@@H]4C3)nc21 ; O=C(c1ccc2ccn(C3COC3)c2c1)N1CC[C@@H]2CN(c3cc(C(F)(F)F)ccn3)C[C@@H]2C1 ; FC(F)Cn1ncc2ncc(N3C[C@H]4CCN(c5ncccc5C(F)(F)F)C[C@H]4C3)nc21 ; FC(F)Cn1ncc2ncc(N3C[C@H]4CCN(c5ccc(C(F)(F)F)cn5)C[C@H]4C3)nc21]

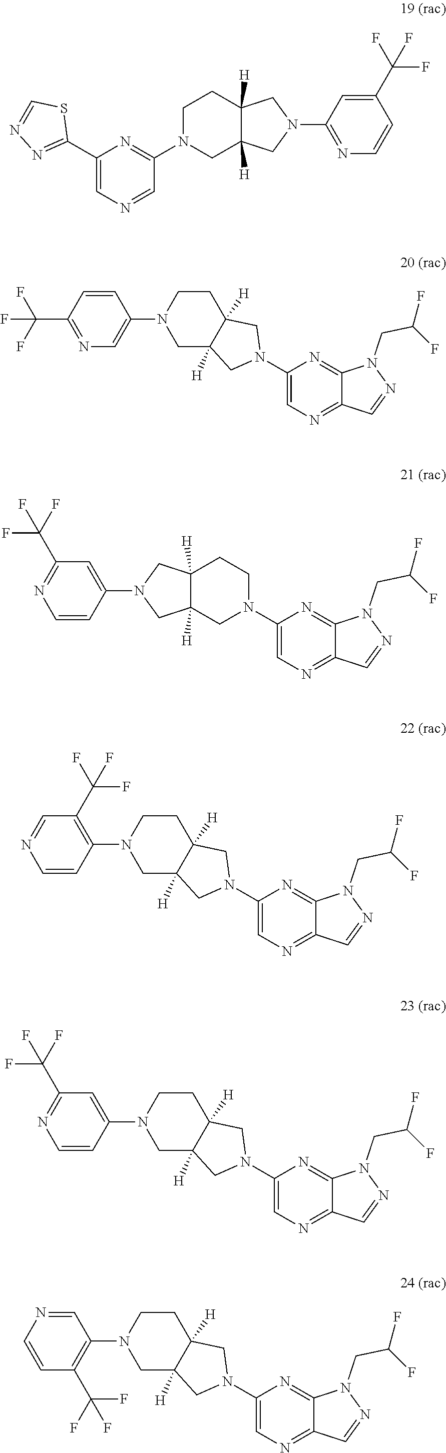 [SMILES: FC(F)(F)c1ccnc(N2C[C@H]3CCN(c4cncc(-c5nncs5)n4)C[C@H]3C2)c1 ; FC(F)Cn1ncc2ncc(N3C[C@H]4CN(c5ccc(C(F)(F)F)nc5)CC[C@H]4C3)nc21 ; FC(F)Cn1ncc2ncc(N3CC[C@@H]4CN(c5ccnc(C(F)(F)F)c5)C[C@@H]4C3)nc21 ; FC(F)Cn1ncc2ncc(N3C[C@@H]4CCN(c5ccncc5C(F)(F)F)C[C@@H]4C3)nc21 ; FC(F)Cn1ncc2ncc(N3C[C@H]4CN(c5ccnc(C(F)(F)F)c5)CC[C@H]4C3)nc21 ; FC(F)Cn1ncc2ncc(N3C[C@@H]4CCN(c5cnccc5C(F)(F)F)C[C@@H]4C3)nc21]

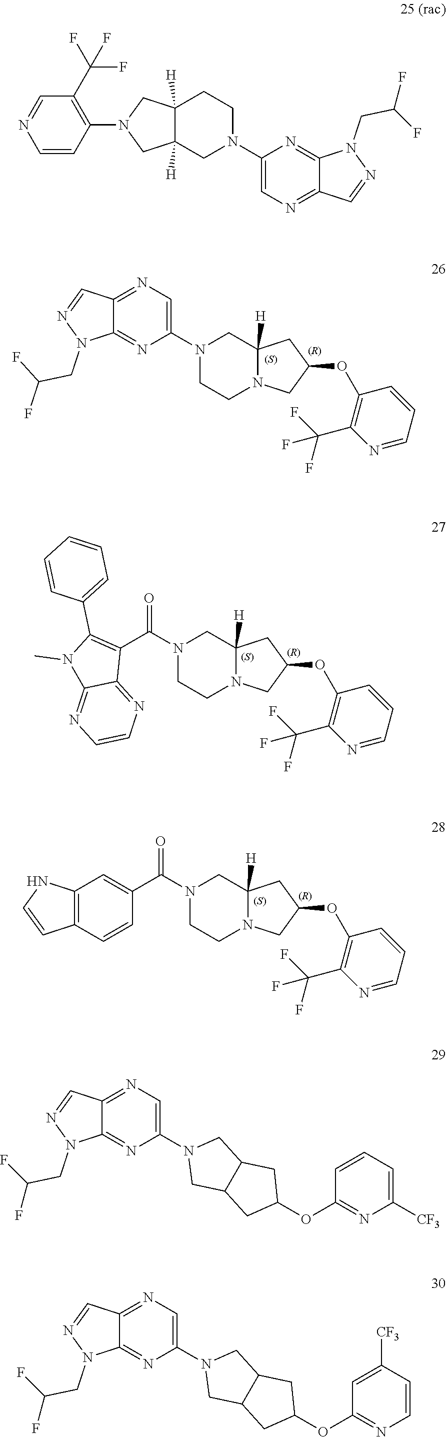

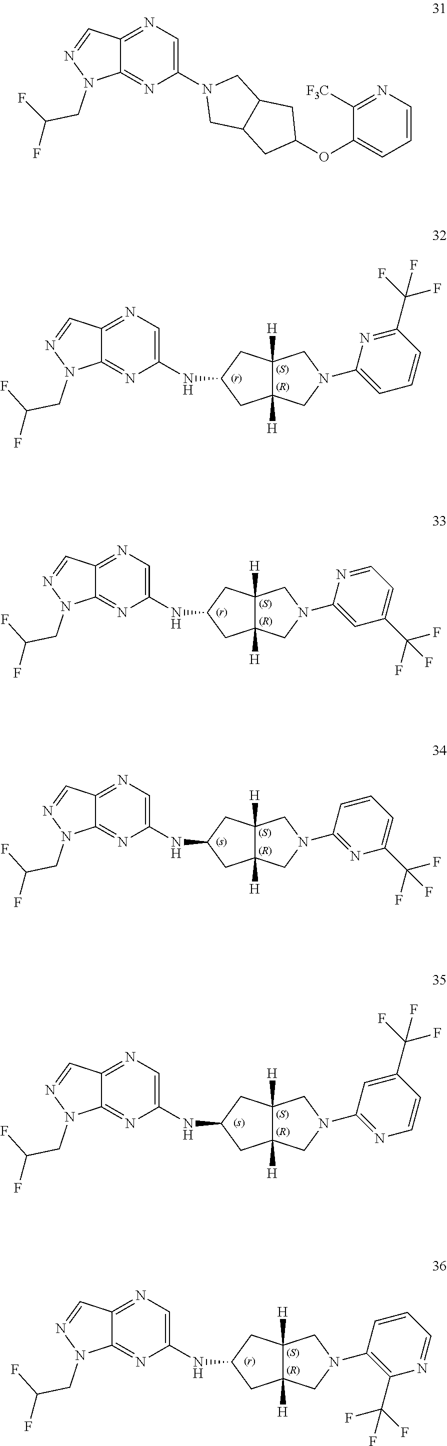

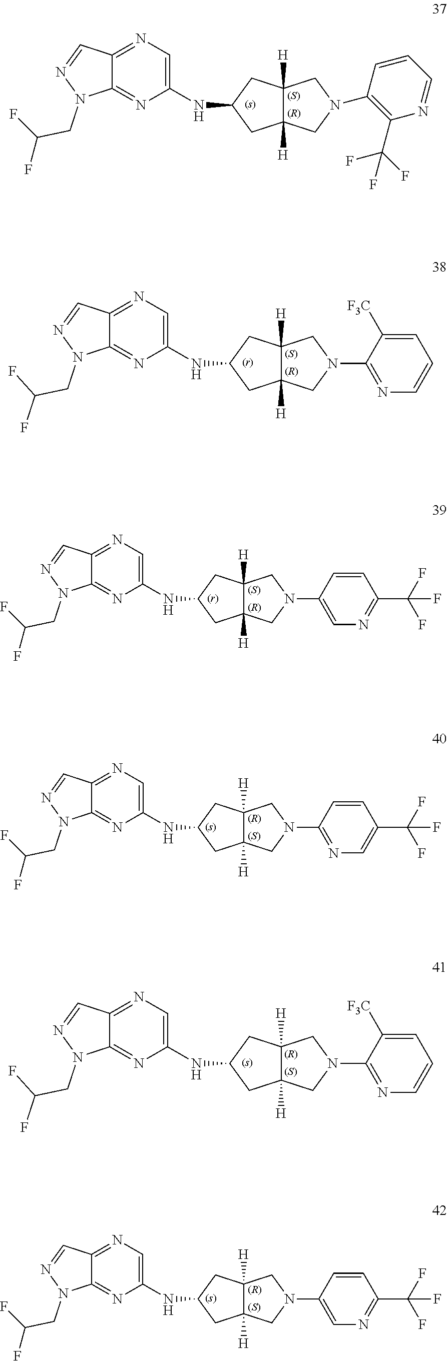

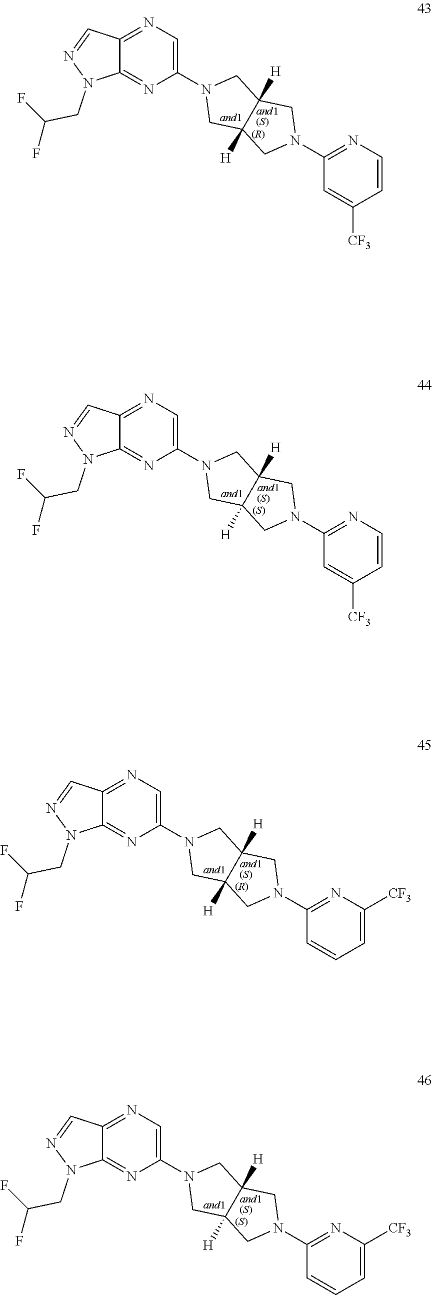

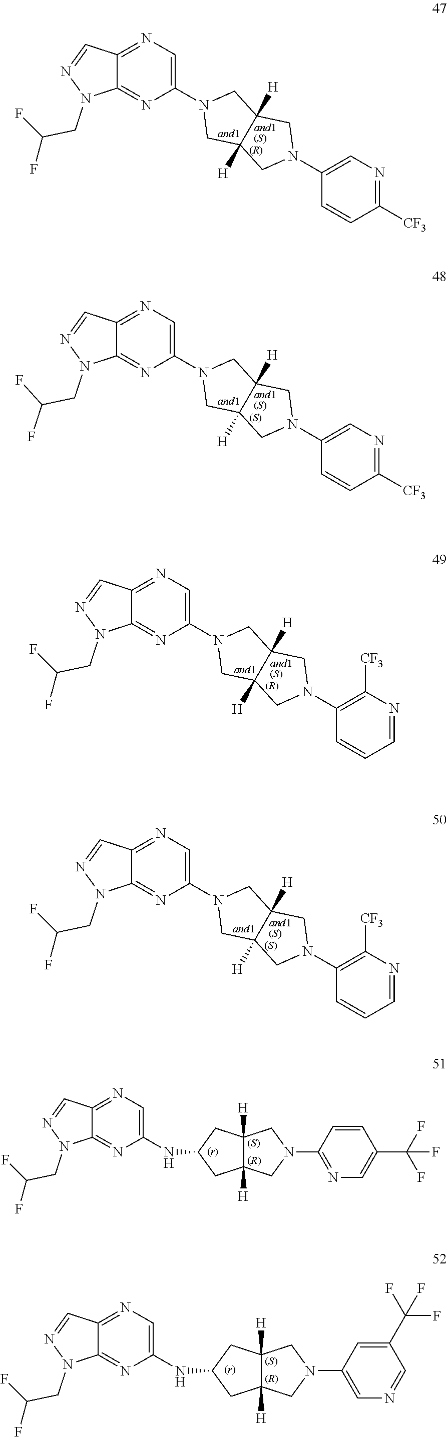

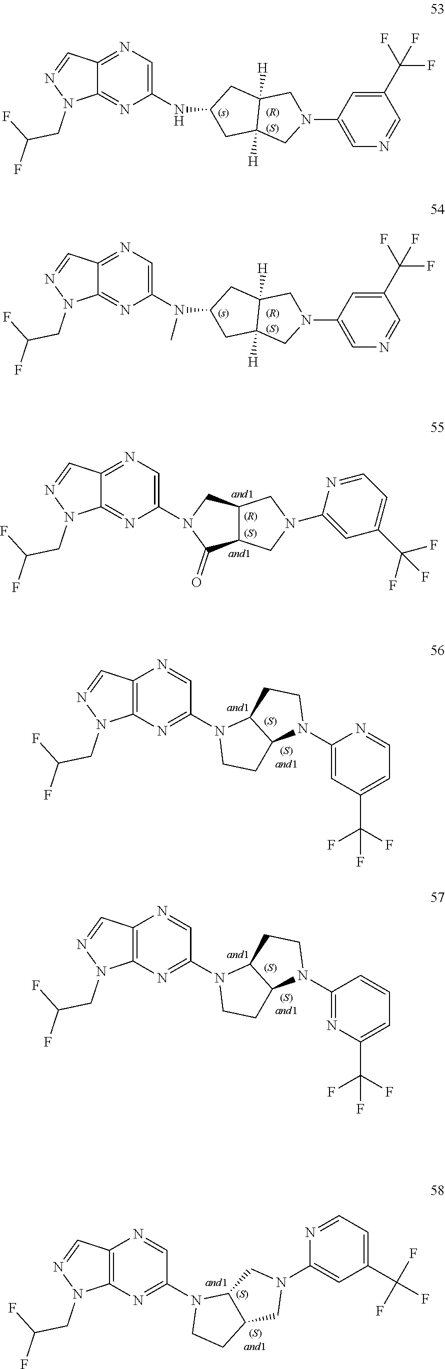

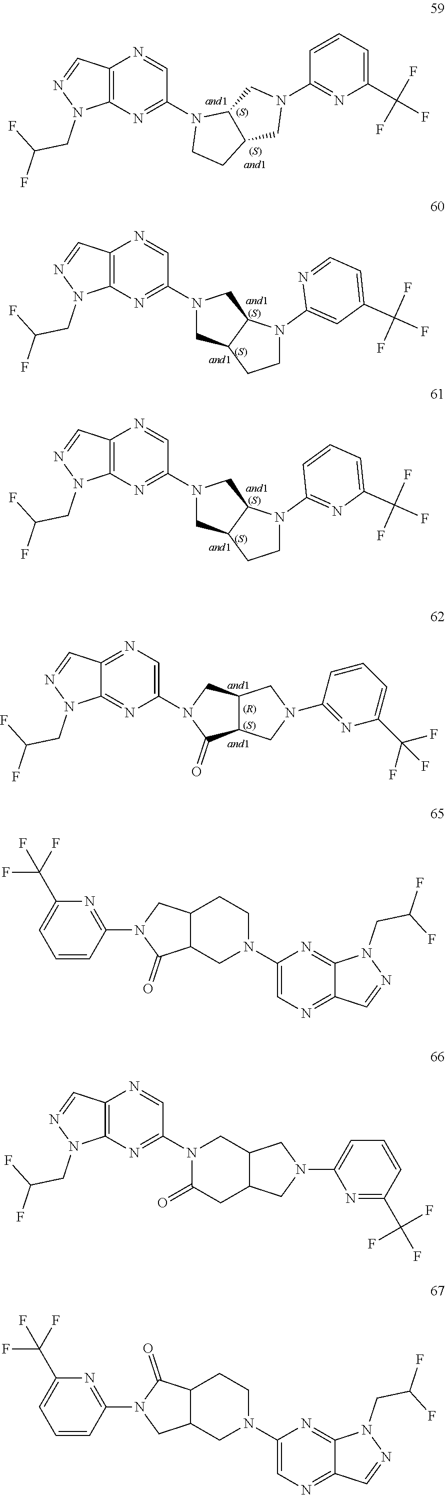

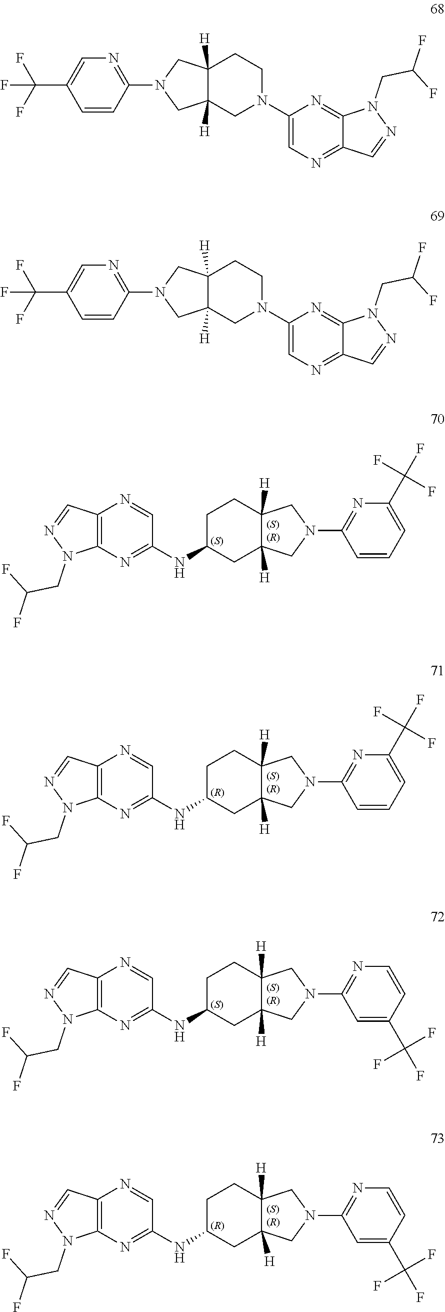

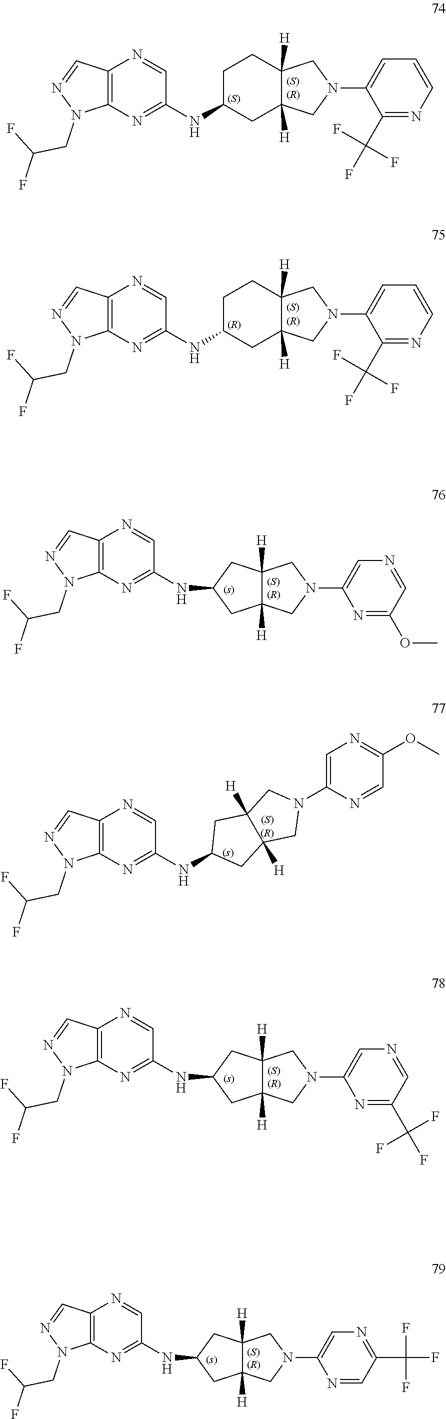

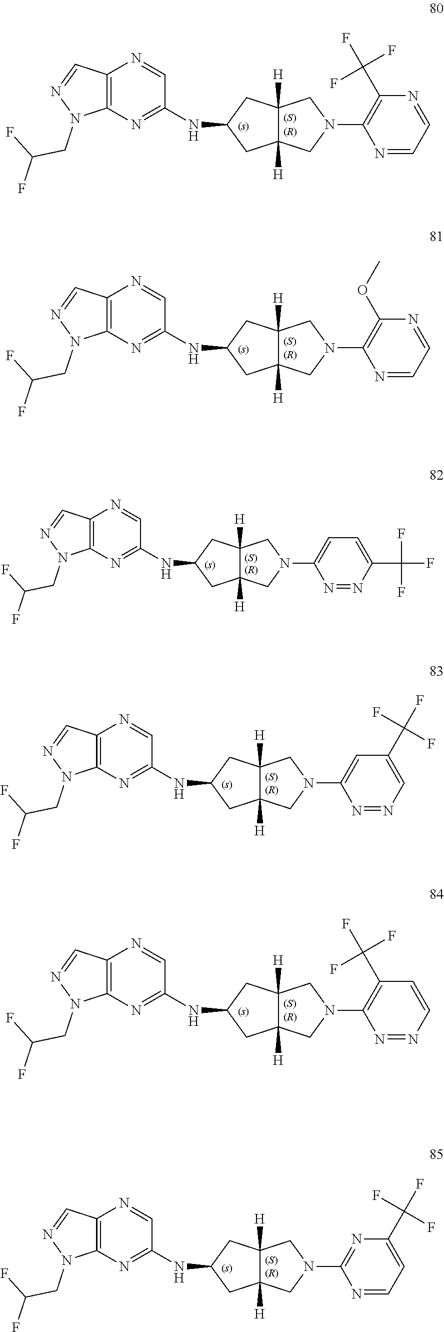

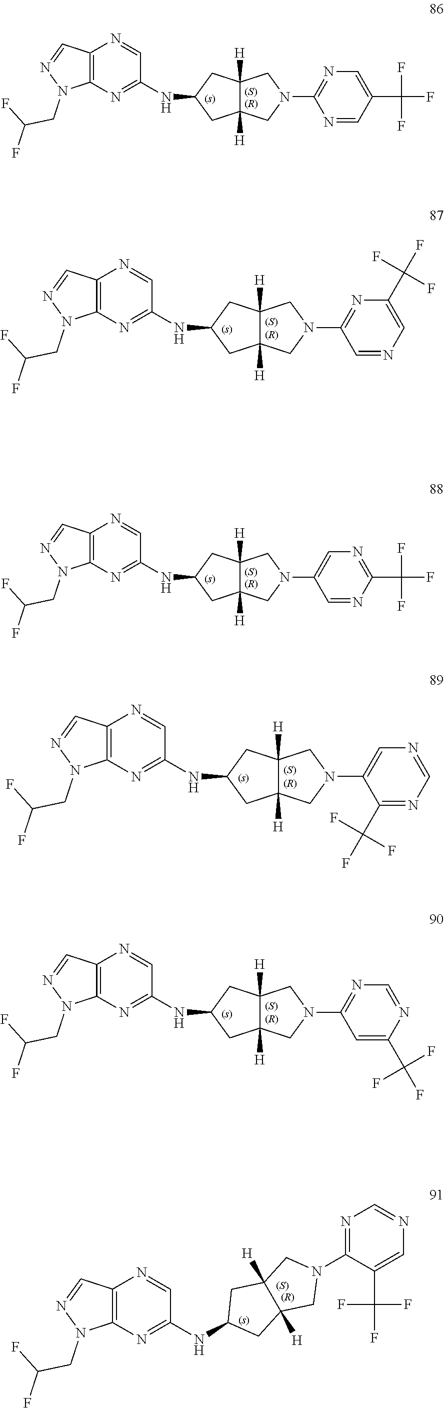

In certain embodiments, the compound of Formula (II) is of Formula (II-a):

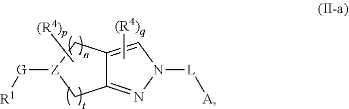

or a pharmaceutically acceptable salt, co-crystal, tautomer, stereoisomer, solvate, hydrate, polymorph, isotopically enriched derivative, or prodrug thereof; wherein R¹, G, Z, R⁴, p, q, n, t, L, and A are as defined herein.

In certain embodiments, the compound of Formula (II) is of Formula (II-a-1):

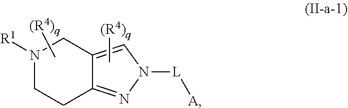

or a pharmaceutically acceptable salt, co-crystal, tautomer, stereoisomer, solvate, hydrate, polymorph, isotopically enriched derivative, or prodrug thereof; wherein R¹, R⁴, p, q, L, and A are as defined herein.

In certain embodiments, the compound of Formula (II) is of Formula (II-a-2):

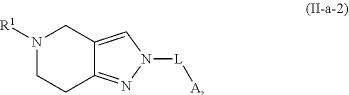

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (II) is of Formula (II-b):

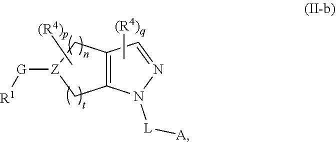

or a pharmaceutically acceptable salt, co-crystal, tautomer, stereoisomer, solvate, hydrate, polymorph, isotopically enriched derivative, or prodrug thereof; wherein R¹, G, Z, R⁴, p, q, n, t, L, and A are as defined herein.

In certain embodiments, the compound of Formula (II) is of Formula (II-b-1):

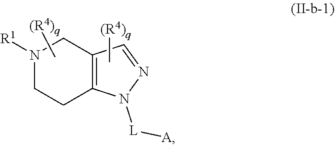

or a pharmaceutically acceptable salt, co-crystal, tautomer, stereoisomer, solvate, hydrate, polymorph, isotopically enriched derivative, or prodrug thereof; wherein R¹, R⁴, p, q, L, and A are as defined herein.

In certain embodiments, the compound of Formula (II) is of Formula (II-b-2):

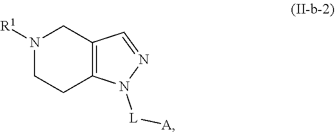

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (II) is of Formula (II-c):

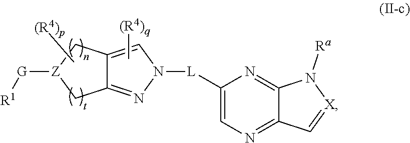

or a pharmaceutically acceptable salt, co-crystal, tautomer, stereoisomer, solvate, hydrate, polymorph, isotopically enriched derivative, or prodrug thereof; wherein R¹, G, Z, R⁴, L, n, t, p, and q are as defined herein; X is N or CR^{a}; and each R^{a} is independently hydrogen, substituted or unsubstituted alkyl, or substituted or unsubstituted heterocyclyl.

In certain embodiments, the compound of Formula (II) is of Formula (II-c-1):

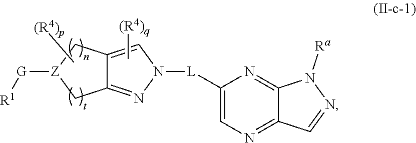

or a pharmaceutically acceptable salt, co-crystal, tautomer, stereoisomer, solvate, hydrate, polymorph, isotopically enriched derivative, or prodrug thereof; wherein R¹, G, Z, R⁴, L, n, t, p, and q are as defined herein; and R^{a} is hydrogen, substituted or unsubstituted alkyl or substituted or unsubstituted heterocyclyl.

In certain embodiments, the compound of Formula (II) is of Formula (II-c-2):

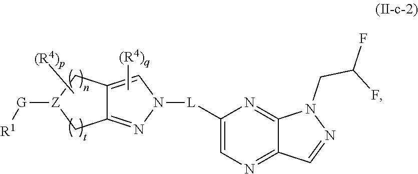

or a pharmaceutically acceptable salt, co-crystal, tautomer, stereoisomer, solvate, hydrate, polymorph, isotopically enriched derivative, or prodrug thereof; wherein R¹, G, Z, R⁴, L, n, t, p, and q are as defined herein.

In certain embodiments, the compound of Formula (II) is of Formula (II-d):

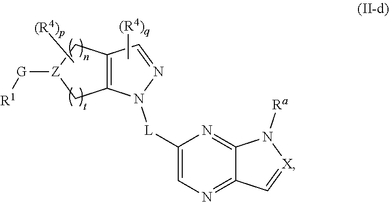

or a pharmaceutically acceptable salt, co-crystal, tautomer, stereoisomer, solvate, hydrate, polymorph, isotopically enriched derivative, or prodrug thereof; wherein R¹, G, Z, R⁴, L, n, t, p, and q are as defined herein; X is N or CR^{a}; and each R^{a} is independently hydrogen, substituted or unsubstituted alkyl, or substituted or unsubstituted heterocyclyl.

In certain embodiments, the compound of Formula (II) is of Formula (II-d-1):

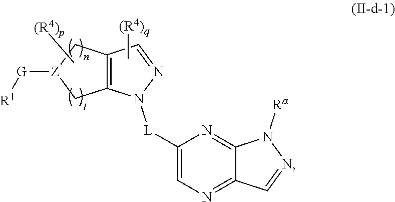

or a pharmaceutically acceptable salt, co-crystal, tautomer, stereoisomer, solvate, hydrate, polymorph, isotopically enriched derivative, or prodrug thereof; wherein R¹, G, Z, R⁴, L, n, t, p, and q are as defined herein; and R^{a} is hydrogen, substituted or unsubstituted alkyl or substituted or unsubstituted heterocyclyl.

In certain embodiments, the compound of Formula (II) is of Formula (II-d-2):

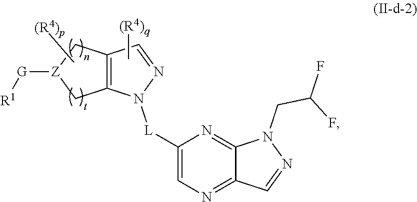

or a pharmaceutically acceptable salt, co-crystal, tautomer, stereoisomer, solvate, hydrate, polymorph, isotopically enriched derivative, or prodrug thereof; wherein R¹, G, Z, R⁴, L, n, t, p, and q are as defined herein.

In certain embodiments, the compound of Formula (II) is one of the following compounds, or a pharmaceutically acceptable salt, co-crystal, tautomer, stereoisomer, solvate, hydrate, polymorph, isotopically enriched derivative, or prodrug thereof:

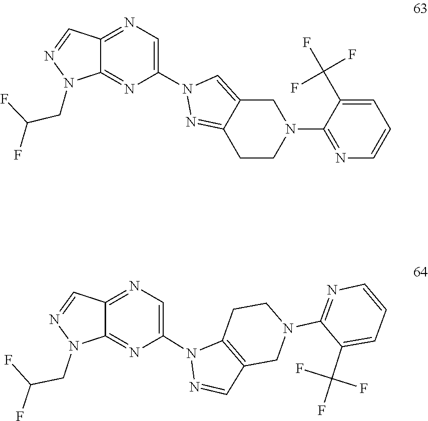 [SMILES: FC(F)Cn1ncc2ncc(-n3cc4c(n3)CCN(c3ncccc3C(F)(F)F)C4)nc21 ; FC(F)Cn1ncc2ncc(-n3ncc4c3CCN(c3ncccc3C(F)(F)F)C4)nc21]

In certain embodiments, the provided compounds (e.g., compounds of Formula (I) and (II)), activate GCase with an EC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

### Pharmaceutical Compositions, Kits, and Administration

The present disclosure provides pharmaceutical compositions comprising a disclosed compound (e.g., a compound of Formula (I) or (II)), or a pharmaceutically acceptable salt, co-crystal, tautomer, stereoisomer, solvate, hydrate, polymorph, isotopically enriched derivative, or prodrug thereof, and optionally a pharmaceutically acceptable excipient. In certain embodiments, the pharmaceutical composition described herein comprises a compound of Formula (I) or (II), or a pharmaceutically acceptable salt thereof, and a pharmaceutically acceptable excipient.

In certain embodiments, the compound of Formula (I) or (II) is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a disease or disorder in a subject in need thereof. In certain embodiments, the effective amount is an amount effective for treating a neurological disease or disorder in a subject in need thereof. In certain embodiments, the effective amount is an amount effective for preventing a neurological disease or disorder in a subject in need thereof.

In certain embodiments, the effective amount is an amount effective for reducing the risk of developing a disease (e.g., neurological disease or disorder) in a subject in need thereof.

In certain embodiments, the effective amount is an amount effective for increasing the activity of GCase in a subject, tissue, biological sample, or cell.

In certain embodiments, the subject being treated or administered a compound described herein is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for increasing the activity of GCase by at least about 10%, at least about 20%, at least about 30%, at least about 40%, at least about 50%, at least about 60%, at least about 70%, at least about 80%, at least about 90%, at least about 95%, at least about 100%, at least about 150%, at least about 200%, at least about 250%, at least about 300%, at least about 400%, at least about 500%, or at least about 1000%. In certain embodiments, the effective amount is an amount effective for increasing the activity of GCase by a range between a percentage described in this paragraph and another percentage described in this paragraph, inclusive.

The present disclosure provides pharmaceutical compositions comprising a compound that interacts with (e.g., activates) GCase for use in treating a GCase-related disease or disorder in a subject in need thereof. The present disclosure provides pharmaceutical compositions comprising a compound that interacts with (e.g., activates) GCase for use in treating a disease or disorder associated with aberrant activity of GCase in a subject in need thereof. The present disclosure provides pharmaceutical compositions comprising a compound that interacts with (e.g., activates) GCase for use in treating a disease or disorder associated with mutated GCase in a subject in need thereof.

In certain embodiments, the composition is for use in treating a disease or disorder. In certain embodiments, the composition is for use in treating a neurological disease or disorder. In certain embodiments, the composition is for use in treating Gaucher's disease or Parkinson's disease. In certain embodiments, the composition is for use in treating Gaucher's disease. In certain embodiments, the composition is for use in treating Parkinson's disease.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent exhibit a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., neurological disease or disorder). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the compound or pharmaceutical composition is a solid. In certain embodiments, the compound or pharmaceutical composition is a powder. In certain embodiments, the compound or pharmaceutical composition can be dissolved in a liquid to make a solution. In certain embodiments, the compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

After formulation with an appropriate pharmaceutically acceptable excipient in a desired dosage, the pharmaceutical compositions of the present disclosure can be administered to humans and other animals orally, parenterally, intracisternally, intraperitoneally, topically, bucally, or the like, depending on the disease or condition being treated.

In certain embodiments, a pharmaceutical composition comprising a compound of Formula (I) or (II) is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Pharmaceutical compositions described herein can be prepared by any method known in the art of pharmacology. In general, such preparatory methods include the steps of bringing the composition comprising a compound of Formula (I) or (II) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. Cremophor™), polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazelnut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the invention are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this invention. Additionally, the present disclosure contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the carrier for a topical formulation can be in the form of a hydroalcoholic system (e.g., liquids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., neurological disease or disorder) in a subject in need thereof. In certain embodiments, the kits are useful for preventing a disease (e.g., neurological disease or disorder) in a subject in need thereof. In certain embodiments, the kits are useful for reducing the risk of developing a disease (e.g., neurological disease or disorder) in a subject in need thereof. In certain embodiments, the kits are useful for increasing the activity of GCase in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., neurological disease or disorder) in a subject in need thereof. In certain embodiments, the kits and instructions provide for preventing a disease (e.g., neurological disease or disorder) in a subject in need thereof. In certain embodiments, the kits and instructions provide for reducing the risk of developing a disease (e.g., neurological disease or disorder) in a subject in need thereof. In certain embodiments, the kits and instructions provide for increasing the activity of GCase in a subject or cell. A kit described herein may include one or more additional pharmaceutical agents described herein as a separate composition.

### Methods of Treatment

The present disclosure provides methods for treating a disease or disorder in a subject in need thereof. In certain embodiments, the present disclosure provides methods for treating a disease or disorder associated with GCase activity. In certain embodiments, the application provides a method of treating a neurological disease or disorder. In certain embodiments, the application provides a method of treating Gaucher's disease or Parkinson's disease. In certain embodiments, the application provides a method of treating Gaucher's disease. In certain embodiments, the application provides a method of treating Parkinson's disease.

The present disclosure provides a method of activating GCase. The present disclosure provides a method of increasing the activity of GCase. In certain embodiments, the application provides a method of activating GCase (e.g., increasing the activity of GCase) in vitro. In certain embodiments, the application provides a method of activating GCase (e.g., increasing the activity of GCase) in vivo. In certain embodiments, the application provides a method of increasing the activity of GCase in a cell. In certain embodiments, the application provides a method of increasing the activity of GCase in a human cell.

In certain embodiments, the methods comprise administering to a subject in need thereof (e.g., a subject with a neurological disease or disorder) a compound that interacts with GCase, for example, a compound that is a modulator of GCase (e.g., an activator of GCase), a binder of GCase, or a compound that modifies GCase. In certain embodiments, the methods comprise administering a compound of the disclosure (e.g., a compound of Formula (I) or (II)), or a pharmaceutically acceptable salt, co-crystal, tautomer, stereoisomer, solvate, hydrate, polymorph, isotopically enriched derivative, or prodrug, or composition thereof, to a subject in need thereof. In some embodiments, the method comprises administering a pharmaceutical composition comprising a compound of the disclosure (e.g., a compound of Formula (I) or (II)), or a pharmaceutically acceptable salt, co-crystal, tautomer, stereoisomer, solvate, hydrate, polymorph, isotopically enriched derivative, or prodrug, or composition thereof, to a subject in need thereof.

Another object of the present disclosure is the use of a compound as described herein (e.g., of any formulae herein) in the manufacture of a medicament for use in the treatment of a disorder or disease described herein. Another object of the present disclosure is the use of a compound as described herein (e.g., of any formulae herein) for use in the treatment of a disorder or disease described herein.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Synthetic Methods

Compounds of Formula (I) and (II) were prepared following the synthetic schemes and procedures described in detail below. The examples described in this application are offered to illustrate the compounds, pharmaceutical compositions, and methods provided herein and are not to be construed in any way as limiting their scope. Compounds of the disclosure that are not explicitly described in the following procedures may be prepared by analogous methods. Those having ordinary skill in the art would understand how to make such compounds from the disclosure provided herein and by means known in the art of organic synthesis. For example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof are representative and instructive. Methods for optimizing reaction conditions, if necessary minimizing competing by products, are known in the art. Embodiments of this disclosure include methods of synthesizing compounds delineated herein using any of the compounds, reactants, and/or processes delineated herein.

In the naming of certain compounds, the word “assumed” is written immediately after the name of the compound. This is meant to designate that the assigned stereochemical centers of that compound are assumed to be the orientation named in the compound name.

### General Procedure A

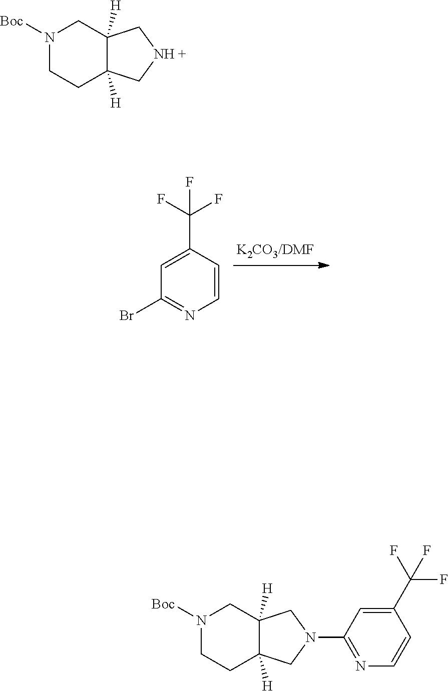

Rac-tert-butyl (3aR,7aS)-2-[4-(trifluoromethyl)pyridin-2-yl]-octahydro-1H-pyrrolo[3,4-c]pyridine-5-carboxylate: A mixture of rac-tert-butyl (3aR,7aS)-octahydro-1H-pyrrolo[3,4-c]pyridine-5-carboxylate (50 mg, 221 μmol, 1.0 equiv.), 2-bromo-4-(trifluoromethyl)pyridine (50 mg, 221 μmol, 1.0 equiv.), and K₂CO₃ (61.1 mg, 442 μmol, 2.0 equiv.) in DMF (1 mL) was heated at 75° C. overnight. The reaction was monitored by LCMS. The mixture was diluted with water (5 mL) and extracted with EtOAc (5 mL×2). The organic layer was combined, washed with brine, dried, evaporated, and purified with a silico gel column, eluted with gradient of Hexane/EtOAc. The fractions were collected, and concentrated to give rac-tert-butyl (3aR,7aS)-2-[4-(trifluoromethyl)pyridin-2-yl]-octahydro-1H-pyrrolo[3,4-c]pyridine-5-carboxylate (50 mg, 61%) as an off-white solid.

### General Procedure B

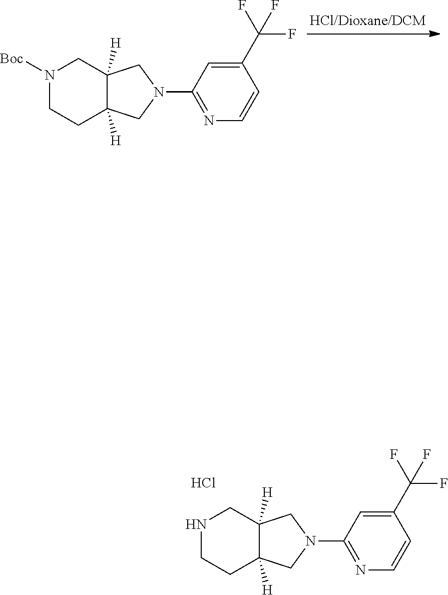 [SMILES: CC(C)(C)OC(=O)N1CC[C@@H]2CN(c3cc(C(F)(F)F)ccn3)C[C@@H]2C1 ; Cl ; FC(F)(F)c1ccnc(N2C[C@H]3CCNC[C@H]3C2)c1]

Rac-2-[(3aR,7aR)-octahydro-1H-pyrrolo[3,4-c]pyridin-2-yl]-4-(trifluoromethyl)pyridine hydrochloride: To a stirred solution of rac-tert-butyl (3aR,7aS)-2-[4-(trifluoromethyl)pyridin-2-yl]-octahydro-1H-pyrrolo[3,4-c]pyridine-5-carboxylate (50 mg, 135 μmol, 1.0 equiv.) in DCM (2.5 mL) was added HCl (gas) in dioxane (4M, 670 μL, 20 equiv.) in a ice bath. The mixture was allowed to stir at room temperature for 2 h. After removing the solvent, the crude product rac-2-[(3aR,7aR)-octahydro-1H-pyrrolo[3,4-c]pyridin-2-yl]-4-(trifluoromethyl)pyridine hydrochloride (35 mg) was directly used in next step without further purification.

### General Procedure C

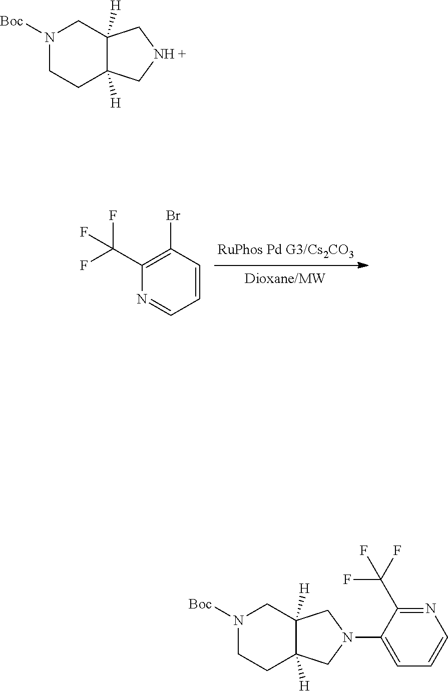

Rac-tert-butyl (3aR,7aS)-2-[2-(trifluoromethyl)pyridin-3-yl]-octahydro-1H-pyrrolo[3,4-c]pyridine-5-carboxylate: A mixture of rac-tert-butyl (3aR,7aS)-octahydro-1H-pyrrolo[3,4-c]pyridine-5-carboxylate (50 mg, 221 μmol, 1.0 equiv), 3-bromo-2-(trifluoromethyl)pyridine (49.9 mg, 221 μmol, 1.0 equiv.), RuPhos Pd G3 (18.5 mg, 22 μmol, 0.1 equiv.), and Cs₂CO₃ (144 mg, 442 μmol, 2.0 equiv.) in 1,4-dioxane (2 mL) was heated in a microwave reactor at 150° C. for 20 min. The reaction was monitored by LCMS. The mixture was filtered through Celite, and washed with EtOAc (10 mL). The organic layer was combined, evaporated, and purified with a silico gel column, eluted with gradient of Hexane/EtOAc. The fractions were collected, and concentrated to give rac-tert-butyl (3aR,7aS)-2-[2-(trifluoromethyl)pyridin-3-yl]-octahydro-1H-pyrrolo[3,4-c]pyridine-5-carboxylate (50 mg, 61%) as a white solid.

### General Procedure D

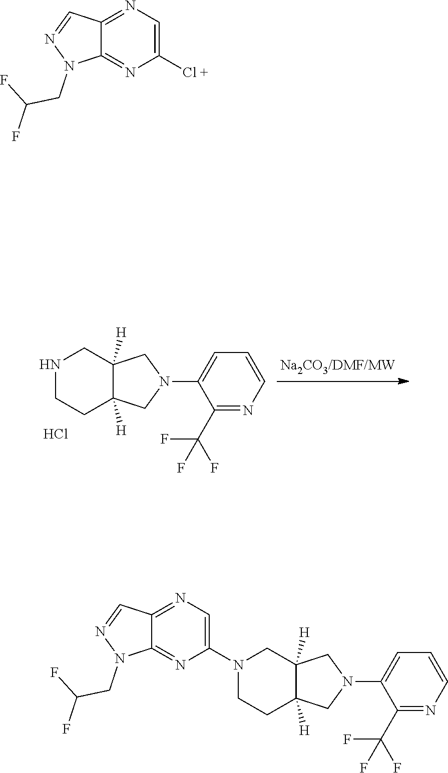

Rac-3-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-2-(trifluoromethyl)pyridine: A mixture of 6-chloro-1-(2,2-difluoroethyl)-1H-pyrazolo[3,4-b]pyrazine (20 mg, 92 umol, 1.0 equiv.), rac-3-[(3aR,7aR)-octahydro-1H-pyrrolo[3,4-c]pyridin-2-yl]-2-(trifluoromethyl)pyridine hydrochloride (28.4 mg, 92 μmol, 1.0 equiv), and Na₂CO₃ (29.3 mg, 280 μmol, 3.0 equiv.) in DMF (1 mL) was heated in a microwave reactor at 150° C. for 30 min. The reaction was monitored by LCMS. The mixture was diluted with water (5 mL), and extracted with EtOAc (5 mL×2). The organic layer was combined, evaporated, and purified with a silico gel column. The fractions were collected, and concentrated to give rac-3-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-2-(trifluoromethyl)pyridine (8 mg, 19%) as an off-white foam.

### General Procedure E

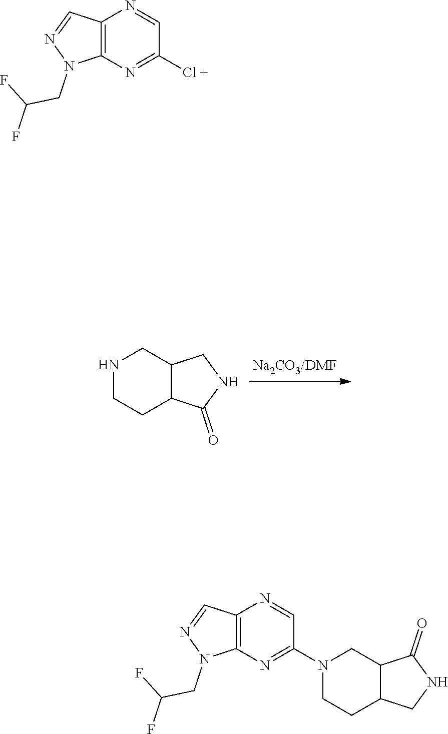

5-(1-(2,2-difluoroethyl)-1H-pyrazolo[3,4-b]pyrazin-6-yl)octahydro-3H-pyrrolo[3,4-c]pyridin-3-one: To a stirred solution of octahydro-3H-pyrrolo[3,4-c]pyridin-3-one (38.5 mg, 0.275 mmol, 1.00 equiv.) and 6-chloro-1-(2,2-difluoroethyl)-1H-pyrazolo[3,4-b]pyrazine (60 mg, 0.275 mmol, 1.00 equiv.) in DMF (1 mL) was added Na₂CO₃ (87.4 mg, 0.825 mmol, 3.0 equiv). The resulting mixture was stirred for 2 h at 100° C. Desired product could be detected by LCMS. The mixture was diluted with EtOAc (15 mL), washed with water and brine, dried, evaporated, and purified with Combi-flash (40 g silico gel column), eluted with gradient of Hex:EtOAc. The fractions were collected, and concentrated to give 5-(1-(2,2-difluoroethyl)-1H-pyrazolo[3,4-b]pyrazin-6-yl)octahydro-3H-pyrrolo[3,4-c]pyridin-3-one (60 mg, 67%) as a white solid.

### General Procedure F:

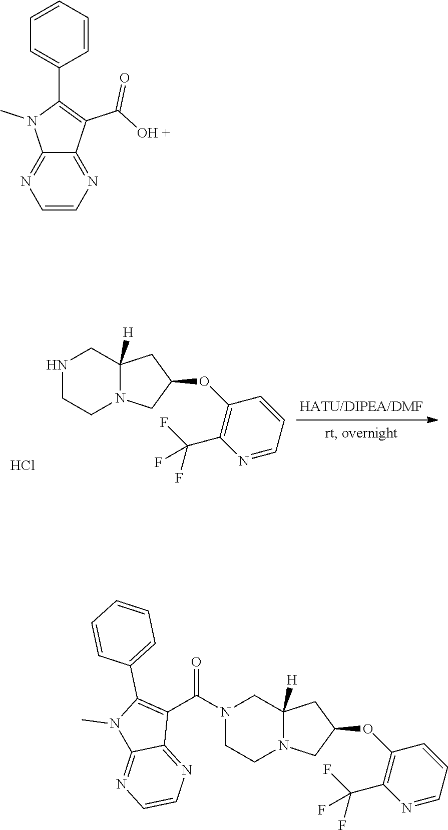

A mixture of 5-methyl-6-phenyl-5H-pyrrolo[2,3-b]pyrazine-7-carboxylic acid (14 mg, 55.6 μmol, 1 equiv.) and HATU (23.3 mg, 61.2 μmol, 1.1 equiv.) were added to DMF (1.00 mL) followed by DIPEA (29.5 μL, 167 μmol, 3 equiv.) and 3-[(7R,8aS)-octahydropyrrolo[1,2-a]pyrazin-7-yloxy]-2-(trifluoromethyl)pyridine hydrochloride (18 mg, 55.6 μmol, 1 equiv.) at room temperature. The mixture was stirred at room temperature for 16 h. The reaction was monitored by LCMS. The mixture was diluted with water (15 mL) and extracted with EtOAc (15 mL×2). The organic layers were combined, washed with brine, dried, evaporated, and purified with Combi-flash (4 g silico gel column), eluted with Hex:EtOAc. The fractions were collected, and concentrated to afford the product 3-{[(7R,8aS)-2-{5-methyl-6-phenyl-5H-pyrrolo[2,3-b]pyrazine-7-carbonyl}-octahydropyrrolo[1,2-a]pyrazin-7-yl]oxy}-2-(trifluoromethyl)pyridine (13 mg, 45%) as a white solid.

### rac-2-[(3aR,7aS)-5-[1-(oxetan-3-yl)-1H-pyrazolo[3,4-b]pyrazin-6-yl]-octahydro-1H-pyrrolo[3,4-c]pyridin-2-yl]-4-(trifluoromethyl)pyridine (1)

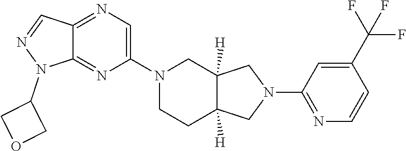 [SMILES: FC(F)(F)c1ccnc(N2C[C@H]3CCN(c4cnc5cnn(C6COC6)c5n4)C[C@H]3C2)c1]

Step 1: Rac-tert-butyl (3aR,7aS)-2-[4-(trifluoromethyl)pyridin-2-yl]-octahydro-1H-pyrrolo[3,4-c]pyridine-5-carboxylate: Followed the general procedure A using rac-tert-butyl (3aR,7aS)-octahydro-1H-pyrrolo[3,4-c]pyridine-5-carboxylate (50 mg, 221 μmol, 1.0 equiv) and 2-bromo-4-(trifluoromethyl)pyridine (50 mg, 221 μmol, 1.0 equiv.) as the starting materials to give rac-tert-butyl (3aR,7aS)-2-[4-(trifluoromethyl)pyridin-2-yl]-octahydro-1H-pyrrolo[3,4-c]pyridine-5-carboxylate (50 mg, 61%) as an off-white solid. MS m/z: 372 [M+H]⁺.

Step 2: rac-2-[(3aR,7aR)-octahydro-1H-pyrrolo[3,4-c]pyridin-2-yl]-4-(trifluoromethyl)pyridine hydrochloride: Followed the general procedure B using (3aR,7aS)-2-[4-(trifluoromethyl)pyridin-2-yl]-octahydro-1H-pyrrolo[3,4-c]pyridine-5-carboxylate (50 mg) as the starting material to give the crude product rac-2-[(3aR,7aR)-octahydro-1H-pyrrolo[3,4-c]pyridin-2-yl]-4-(trifluoromethyl)pyridine hydrochloride (35 mg). MS m/z: 272 [M+H]⁺.

Step 3: rac-2-[(3aR,7aS)-5-[1-(oxetan-3-yl)-1H-pyrazolo[3,4-b]pyrazin-6-yl]-octahydro-1H-pyrrolo[3,4-c]pyridin-2-yl]-4-(trifluoromethyl)pyridine: Followed the general procedure A using rac-2-[(3aR,7aR)-octahydro-1H-pyrrolo[3,4-c]pyridin-2-yl]-4-(trifluoromethyl)pyridine hydrochloride (29.2 mg, 95 μmol, 1.0 equiv) and 6-chloro-1-(oxetan-3-yl)-1H-pyrazolo[3,4-b]pyrazine (20 mg, 95 μmol, 1.0 equiv) as the starting materials to give rac-2-[(3aR,7aS)-5-[1-(oxetan-3-yl)-1H-pyrazolo[3,4-b]pyrazin-6-yl]-octahydro-1H-pyrrolo[3,4-c]pyridin-2-yl]-4-(trifluoromethyl)pyridine (13 mg, 31%) as a colorless oil. ¹H NMR (500 MHz, DMSO-d₆) δ 8.47 (s, 1H), 8.26 (d, J=5.2 Hz, 1H), 8.18 (s, 1H), 6.77 (d, J=5.2 Hz, 1H), 6.63 (s, 1H), 5.95-5.86 (m, 1H), 5.06 (q, J=6.3 Hz, 2H), 5.01-4.95 (m, 2H), 4.08-4.02 (m, 1H), 3.94 (dd, J=14.0, 5.4 Hz, 1H), 3.83 (dd, J=13.8, 4.1 Hz, 1H), 3.63-3.43 (m, 4H), 3.25 (dd, J=10.5, 6.0 Hz, 1H), 2.63-2.55 (m, 2H), 1.90-1.80 (m, 1H), 1.61-1.51 (m, 1H). MS m/z: 446 [M+H]⁺.

### rac-3-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-2-(trifluoromethyl)pyridine (2)

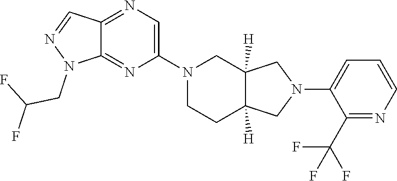 [SMILES: FC(F)Cn1ncc2ncc(N3CC[C@@H]4CN(c5cccnc5C(F)(F)F)C[C@@H]4C3)nc21]

Step 1: rac-tert-butyl (3aR,7aS)-2-[2-(trifluoromethyl)pyridin-3-yl]-octahydro-1H-pyrrolo[3,4-c]pyridine-5-carboxylate: Followed the general procedure C using rac-tert-butyl (3aR,7aS)-octahydro-1H-pyrrolo[3,4-c]pyridine-5-carboxylate (50 mg, 221 μmol, 1.0 equiv.) and 3-bromo-2-(trifluoromethyl)pyridine (49.9 mg, 221 μmol, 1.0 equiv.) as the starting materials to afford rac-tert-butyl (3aR,7aS)-2-[2-(trifluoromethyl)pyridin-3-yl]-octahydro-1H-pyrrolo[3,4-c]pyridine-5-carboxylate (26 mg, 32%) as a white solid. MS m/z: 372 [M+H]⁺.

Step 2: rac-3-[(3aR,7aR)-octahydro-1H-pyrrolo[3,4-c]pyridin-2-yl]-2-(trifluoromethyl)pyridine hydrochloride: Followed the general procedure B using rac-tert-butyl (3aR,7aS)-2-[2-(trifluoromethyl)pyridin-3-yl]-octahydro-1H-pyrrolo[3,4-c]pyridine-5-carboxylate (26 mg) to give the crude product rac-3-[(3aR,7aR)-octahydro-1H-pyrrolo[3,4-c]pyridin-2-yl]-2-(trifluoromethyl)pyridine hydrochloride (20 mg). MS m/z: 272 [M+H]⁺.

Step 3: rac-2-[(3aR,7aS)-5-[1-(oxetan-3-yl)-1H-pyrazolo[3,4-b]pyrazin-6-yl]-octahydro-1H-pyrrolo[3,4-c]pyridin-2-yl]-4-(trifluoromethyl)pyridine: Followed the general procedure D using 6-chloro-1-(2,2-difluoroethyl)-1H-pyrazolo[3,4-b]pyrazine (20 mg, 92 μmol, 1.0 equiv.) and rac-3-[(3aR,7aR)-octahydro-1H-pyrrolo[3,4-c]pyridin-2-yl]-2-(trifluoromethyl)pyridine hydrochloride (28.4 mg, 92 μmol, 1.0 equiv) as the starting materials to afford rac-3-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-2-(trifluoromethyl)pyridine (8 mg, 19%) as an off-white foam. ¹H NMR (500 MHz, DMSO-d₆) δ 8.47 (s, 1H), 8.13 (s, 1H), 8.04 (dd, J=3.4, 2.1 Hz, 1H), 7.46-7.40 (m, 2H), 6.43 (tt, J=54.9, 3.8 Hz, 1H), 4.78-4.63 (m, 2H), 4.12-4.01 (m, 1H), 3.95 (dd, J=13.9, 5.9 Hz, 1H), 3.80 (dd, J=13.9, 4.5 Hz, 1H), 3.54 (ddt, J=16.0, 10.0, 5.1 Hz, 2H), 3.41 (t, J=8.5 Hz, 1H), 3.27 (dd, J=9.6, 4.3 Hz, 1H), 3.21 (dd, J=9.7, 6.7 Hz, 1H), 2.65-2.51 (m, 2H), 1.88-1.75 (m, 1H), 1.57 (dtd, J=12.9, 8.7, 3.8 Hz, 1H). MS m/z: 454 [M+H]⁺.

### rac-2-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-4-(trifluoromethyl)pyridine (3)

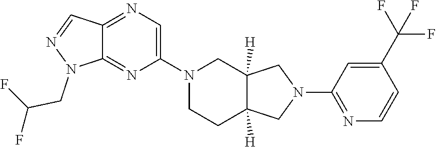 [SMILES: FC(F)Cn1ncc2ncc(N3CC[C@@H]4CN(c5cc(C(F)(F)F)ccn5)C[C@@H]4C3)nc21]

Followed the general procedure A using 6-chloro-1-(2,2-difluoroethyl)-1H-pyrazolo[3,4-b]pyrazine (20.8 mg, 95 μmol, 1.0 equiv.) and rac-2-[(3aR,7aR)-octahydro-1H-pyrrolo[3,4-c]pyridin-2-yl]-4-(trifluoromethyl)pyridine hydrochloride (29.2 mg, 95 μmol, 1.0 equiv) as the starting materials to afford rac-2-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-4-(trifluoromethyl)pyridine (11 mg, 25%) as an off-white foam. ¹H NMR (500 MHz, DMSO-d₆) δ 8.49 (s, 1H), 8.26 (d, J=5.2 Hz, 1H), 8.13 (s, 1H), 6.77 (d, J=5.2 Hz, 1H), 6.62 (s, 1H), 6.45 (tt, J=54.9, 3.8 Hz, 1H), 4.70 (tdd, J=15.1, 3.9, 1.7 Hz, 2H), 4.09-4.03 (m, 1H), 3.97 (dd, J=14.0, 5.3 Hz, 1H), 3.83 (dd, J=13.9, 4.1 Hz, 1H), 3.66-3.52 (m, 3H), 3.51-3.42 (m, 1H), 3.29-3.20 (m, 1H), 2.62 (d, J=19.9 Hz, 2H), 1.90-1.80 (m, 1H), 1.60-1.49 (m, 1H). MS m/z: 454 [M+H]⁺.

### rac-3-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-2-(trifluoromethyl)pyridine (4)

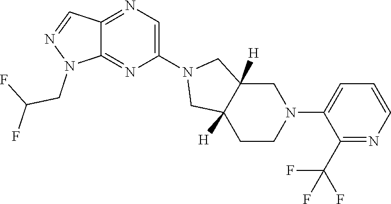 [SMILES: FC(F)Cn1ncc2ncc(N3C[C@H]4CCN(c5cccnc5C(F)(F)F)C[C@H]4C3)nc21]

Step 1: rac-tert-butyl (3aR,7aR)-5-[2-(trifluoromethyl)pyridin-3-yl]-octahydro-1H-pyrrolo[3,4-c]pyridine-2-carboxylate: Followed the general procedure C using rac-tert-butyl (3aR,7aR)-octahydro-1H-pyrrolo[3,4-c]pyridine-2-carboxylate (100 mg, 0.44 mmol, 1.0 equiv.) and 3-bromo-2-(trifluoromethyl)pyridine (150 mg, 0.66 mmol, 1.5 equiv.) as starting materials to afford rac-tert-butyl (3aR,7aR)-5-[2-(trifluoromethyl)pyridin-3-yl]-octahydro-1H-pyrrolo[3,4-c]pyridine-2-carboxylate (45 mg, 27%) as a light-yellow oil. MS m/z: 372 [M+H]⁺.

Step 2: rac-3-[(3aR,7aS)-octahydro-1H-pyrrolo[3,4-c]pyridin-5-yl]-2-(trifluoromethyl)pyridine hydrochloride: Followed the general procedure B using rac-tert-butyl (3aR,7aR)-5-[2-(trifluoromethyl)pyridin-3-yl]-octahydro-1H-pyrrolo[3,4-c]pyridine-2-carboxylate (45 mg) as the starting material to give the crude product rac-3-[(3aR,7aS)-octahydro-1H-pyrrolo[3,4-c]pyridin-5-yl]-2-(trifluoromethyl)pyridine hydrochloride (35 mg). MS m/z: 272 [M+H]⁺.

Step 3: rac-3-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-2-(trifluoromethyl)pyridine: Followed the general procedure A using 6-chloro-1-(2,2-difluoroethyl)-1H-pyrazolo[3,4-b]pyrazine (12 mg, 55 umol, 1.0 equiv.) and rac-3-[(3aR,7aS)-octahydro-1H-pyrrolo[3,4-c]pyridin-5-yl]-2-(trifluoromethyl)pyridine hydrochloride (16.9 mg, 55 umol, 1.0 equiv) as the starting materials to afford rac-3-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-2-(trifluoromethyl)pyridine (21 mg, 84%) as an off-white foam. ¹H NMR (500 MHz, DMSO-d₆) δ 8.46 (dd, J=4.5, 1.4 Hz, 1H), 8.13 (s, 1H), 8.08 (br s, 1H), 8.06 (d, J=7.7 Hz, 2H), 7.69 (dd, J=8.3, 4.5 Hz, 1H), 6.45 (tt, J=54.9, 4.1 Hz, 1H), 4.68 (td, J=14.9, 3.8 Hz, 2H), 3.76-3.55 (m, 4H), 3.10 (dd, J=12.0, 4.3 Hz, 1H), 3.00-2.93 (m, 2H), 2.82 (ddd, J=11.7, 8.3, 3.3 Hz, 1H), 2.63 (p, J=1.8 Hz, 2H), 1.92-1.84 (m, 1H), 1.72-1.66 (m, 1H). MS m/z: 454 [M+H]⁺.

### rac-2-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-3-(trifluoromethyl)pyridine (5)

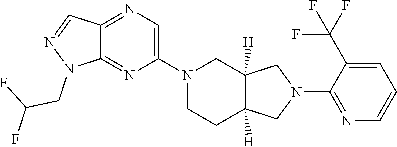 [SMILES: FC(F)Cn1ncc2ncc(N3CC[C@@H]4CN(c5ncccc5C(F)(F)F)C[C@@H]4C3)nc21]

Step 1: rac-tert-butyl (3aR,7aR)-5-[1-(2,2-difluoroethyl)-1H-pyrazolo[3,4-b]pyrazin-6-yl]-octahydro-1H-pyrrolo[3,4-c]pyridine-2-carboxylate: Followed the general procedure D using rac-tert-butyl (3aR,7aR)-octahydro-1H-pyrrolo[3,4-c]pyridine-2-carboxylate (104 mg, 0.46 mmol, 1.0 equiv) and 6-chloro-1-(2,2-difluoroethyl)-1H-pyrazolo[3,4-b]pyrazine (100 mg, 0.46 mmol, 1.0 equiv) as the starting materials to afford rac-tert-butyl (3aR,7aR)-5-[1-(2,2-difluoroethyl)-1H-pyrazolo[3,4-b]pyrazin-6-yl]-octahydro-1H-pyrrolo[3,4-c]pyridine-2-carboxylate (125 mg, 67%) as a light-yellow oil. MS m/z: 409 [M+H]⁺.

Step 2: rac-6-[(3aR,7aS)-octahydro-1H-pyrrolo[3,4-c]pyridin-5-yl]-1-(2,2-difluoroethyl)-1H-pyrazolo[3,4-b]pyrazine hydrochloride: Followed the general procedure B using rac-tert-butyl (3aR,7aR)-5-[1-(2,2-difluoroethyl)-1H-pyrazolo[3,4-b]pyrazin-6-yl]-octahydro-1H-pyrrolo[3,4-c]pyridine-2-carboxylate (120 mg) as the starting material to give the crude product rac-6-[(3aR,7aS)-octahydro-1H-pyrrolo[3,4-c]pyridin-5-yl]-1-(2,2-difluoroethyl)-1H-pyrazolo[3,4-b]pyrazine hydrochloride (90 mg). MS m/z: 309 [M+H]⁺.

Step 3: rac-2-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-3-(trifluoromethyl)pyridine: Followed the general procedure A using rac-6-[(3aR,7aS)-octahydro-1H-pyrrolo[3,4-c]pyridin-5-yl]-1-(2,2-difluoroethyl)-1H-pyrazolo[3,4-b]pyrazine hydrochloride (33.5 mg, 97 μmol, 1.0 equiv.) and 2-bromo-3-(trifluoromethyl)pyridine (22 mg, 97 μmol, 1.0 equiv) as the starting materials to afford rac-2-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-3-(trifluoromethyl)pyridine (21 mg, 48%) as an off-white solid. ¹H NMR (500 MHz, DMSO-d₆) δ 8.48 (s, 1H), 8.29 (dd, J=4.7, 1.8 Hz, 1H), 8.13 (s, 1H), 7.89 (dd, J=7.8, 1.8 Hz, 1H), 6.77 (dd, J=7.7, 4.7 Hz, 1H), 6.43 (tt, J=54.9, 3.8 Hz, 1H), 4.69 (tdd, J=15.0, 3.9, 1.7 Hz, 2H), 4.11 (dq, J=10.2, 3.9, 3.2 Hz, 1H), 4.03 (dd, J=13.9, 5.4 Hz, 1H), 3.74 (dd, J=13.9, 4.3 Hz, 1H), 3.69 (dd, J=10.8, 6.4 Hz, 1H), 3.57 (dd, J=10.9, 7.3 Hz, 1H), 3.53-3.43 (m, 2H), 3.38 (dd, J=10.9, 7.4 Hz, 1H), 2.58-2.54 (m, 2H), 1.84-1.79 (m, 1H), 1.52-1.45 (m, 1H).). MS m/z: 454 [M+H]⁺.

### rac-2-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-6-(trifluoromethyl)pyridine (6)

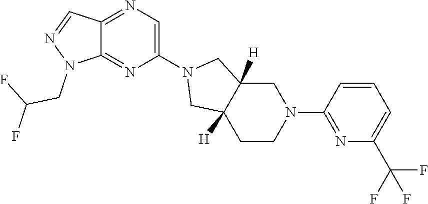 [SMILES: FC(F)Cn1ncc2ncc(N3C[C@H]4CCN(c5cccc(C(F)(F)F)n5)C[C@H]4C3)nc21]

Step 1: rac-tert-butyl (3aR,7aS)-2-[1-(2,2-difluoroethyl)-1H-pyrazolo[3,4-b]pyrazin-6-yl]-octahydro-1H-pyrrolo[3,4-c]pyridine-5-carboxylate: Followed the general procedure D using rac-tert-butyl (3aR,7aS)-octahydro-1H-pyrrolo[3,4-c]pyridine-5-carboxylate (104 mg, 0.46 mmol, 1.0 equiv) and 6-chloro-1-(2,2-difluoroethyl)-1H-pyrazolo[3,4-b]pyrazine (100 mg, 0.46 mmol, 1.0 equiv) as the starting materials to afford rac-tert-butyl (3aR,7aS)-2-[1-(2,2-difluoroethyl)-1H-pyrazolo[3,4-b]pyrazin-6-yl]-octahydro-1H-pyrrolo[3,4-c]pyridine-5-carboxylate (120 mg, 64%) as a yellow oil. MS m/z: 409 [M+H]⁺.

Step 2: rac-6-[(3aR,7aR)-octahydro-1H-pyrrolo[3,4-c]pyridin-2-yl]-1-(2,2-difluoroethyl)-1H-pyrazolo[3,4-b]pyrazine hydrochloride: Followed the general procedure B using rac-tert-butyl (3aR,7aS)-2-[1-(2,2-difluoroethyl)-1H-pyrazolo[3,4-b]pyrazin-6-yl]-octahydro-1H-pyrrolo[3,4-c]pyridine-5-carboxylate (120 mg) as the starting material to give the crude product rac-6-[(3aR,7aR)-octahydro-1H-pyrrolo[3,4-c]pyridin-2-yl]-1-(2,2-difluoroethyl)-1H-pyrazolo[3,4-b]pyrazine hydrochloride (100 mg). MS m/z: 309 [M+H]⁺.

Step 3: rac-2-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-6-(trifluoromethyl)pyridine: Followed the general procedure A using rac-6-[(3aR,7aR)-octahydro-1H-pyrrolo[3,4-c]pyridin-2-yl]-1-(2,2-difluoroethyl)-1H-pyrazolo[3,4-b]pyrazine hydrochloride (33.5 mg, 97 umol, 1.0 equiv.) and 2-bromo-6-(trifluoromethyl)pyridine (22 mg, 97 μmol, 1.0 equiv) as starting materials to afford rac-2-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-6-(trifluoromethyl)pyridine (7.2 mg, 16%) as a white solid. ¹H NMR (500 MHz, DMSO-d₆) δ 8.11 (s, 1H), 8.06 (s, 1H), 7.73 (ddd, J=8.9, 7.2, 0.9 Hz, 1H), 7.17 (d, J=8.8 Hz, 1H), 6.99 (d, J=7.2 Hz, 1H), 6.44 (tt, J=54.7, 3.7 Hz, 1H), 4.65 (td, J=14.9, 3.9 Hz, 2H), 3.91-3.74 (m, 2H), 3.73-3.67 (m, 3H), 3.60 (dd, J=10.9, 4.7 Hz, 1H), 3.45 (ddd, J=12.7, 8.3, 3.6 Hz, 1H), 3.41-3.35 (m, 1H), 2.58 (br s, 2H), 1.86-1.77 (m, 1H), 1.54 (m, 1H). MS m/z: 454 [M+H]⁺.

### rac-2-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-6-(trifluoromethyl)pyridine (7)

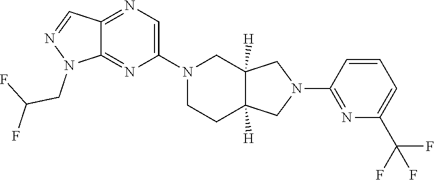 [SMILES: FC(F)Cn1ncc2ncc(N3CC[C@@H]4CN(c5cccc(C(F)(F)F)n5)C[C@@H]4C3)nc21]

Followed the general procedure A using rac-6-[(3aR,7aS)-octahydro-1H-pyrrolo[3,4-c]pyridin-5-yl]-1-(2,2-difluoroethyl)-1H-pyrazolo[3,4-b]pyrazine hydrochloride (33.5 mg, 97 μmol, 1.0 equiv.) and 2-bromo-6-(trifluoromethyl)pyridine (22 mg, 97 umol, 1.0 equiv) as the starting materials to afford rac-2-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-6-(trifluoromethyl)pyridine (19 mg, 43%) as an off-white solid. ¹H NMR (500 MHz, DMSO-d₆) δ 8.49 (s, 1H), 8.12 (s, 1H), 7.66 (dd, J=8.6, 7.3 Hz, 1H), 6.93 (d, J=7.1 Hz, 1H), 6.69 (d, J=8.6 Hz, 1H), 6.44 (tt, J=54.9, 3.8 Hz, 1H), 4.69 (td, J=14.9, 3.8 Hz, 2H), 4.07-3.99 (m, 1H), 3.93 (dd, J=14.0, 5.8 Hz, 1H), 3.85 (dd, J=13.9, 4.2 Hz, 1H), 3.61 (ddd, J=12.8, 8.4, 3.5 Hz, 1H), 3.58-3.52 (m, 2H), 3.47-3.40 (m, 1H), 3.22-3.19 (m, 1H), 2.63-2.54 (m, 2H), 1.90-1.80 (m, 1H), 1.61-1.52 (m, 1H). MS m/z: 454 [M+H]⁺.

### rac-2-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-5-(trifluoromethyl)pyridine (8)

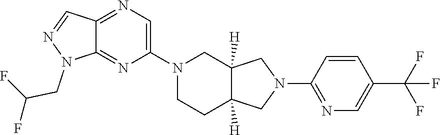 [SMILES: FC(F)Cn1ncc2ncc(N3CC[C@@H]4CN(c5ccc(C(F)(F)F)cn5)C[C@@H]4C3)nc21]

Followed the general procedure A using rac-6-[(3aR,7aS)-octahydro-1H-pyrrolo[3,4-c]pyridin-5-yl]-1-(2,2-difluoroethyl)-1H-pyrazolo[3,4-b]pyrazine (30 mg, 97 μmol, 1.0 equiv) and 2-bromo-5-(trifluoromethyl)pyridine (22 mg, 97 μmol, 1.0 equiv) as the starting materials to afford rac-2-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-5-(trifluoromethyl)pyridine as white solid. ¹H NMR (500 MHz, DMSO-d₆) δ 8.48 (s, 1H), 8.34 (s, 1H), 8.12 (s, 1H), 7.72 (dd, J=9.0, 2.6 Hz, 1H), 6.53 (d, J=8.9 Hz, 1H), 6.44 (tt, J=54.9, 3.8 Hz, 1H), 4.69 (td, J=15.0, 3.9 Hz, 2H), 4.08-3.96 (m, 1H), 3.97 (dd, J=14.0, 5.5 Hz, 1H), 3.82 (dd, J=14.0, 4.2 Hz, 1H), 3.64-3.52 (m, 3H), 3.47-3.45 (m, 1H), 3.28-3.17 (m, 1H), 2.60 (br s, 2H), 1.86-1.83 (m, 1H), 1.57-1.51 (m, 1H). MS m/z: 454 [M+H]⁺.

### rac-6-[(3aR,7aS)-2-[4-(trifluoromethyl)pyridin-2-yl]-octahydro-1H-pyrrolo[3,4-c]pyridine-5-carbonyl]-1-[(oxetan-3-yl)methyl]-1H-indole (9)

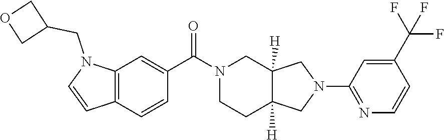 [SMILES: O=C(c1ccc2ccn(CC3COC3)c2c1)N1CC[C@@H]2CN(c3cc(C(F)(F)F)ccn3)C[C@@H]2C1]

A mixture of 1-[(oxetan-3-yl)methyl]-1H-indole-6-carboxylic acid (21.3 mg, 92 μmol, 1.0 equiv.) and HATU (51.9 mg, 137 μmol, 1.1 equiv.) were added into DMF (1.00 mL) followed by DIPEA (64.9 μL, 372 μmol, 3 equiv.) and rac-2-[(3aR,7aR)-octahydro-1H-pyrrolo[3,4-c]pyridin-2-yl]-4-(trifluoromethyl)pyridine (15 mg, 92 μmol, 1.0 equiv.) at room temperature. The mixture was stirred at room temperature for 16 h. The reaction was monitored by LCMS. The mixture was diluted with water (15 mL), and extracted with EtOAc (15 mL×2). The organic layer was combined, washed with brine, dried, evaporated, and purified with Combi-flash (4 g silico gel column), eluted with Hexane/EtOAc. The fractions were collected, and concentrated to afford rac-6-[(3aR,7aS)-2-[4-(trifluoromethyl)pyridin-2-yl]-octahydro-1H-pyrrolo[3,4-c]pyridine-5-carbonyl]-1-[(oxetan-3-yl)methyl]-1H-indole as a white solid. ¹H NMR (500 MHz, DMSO-d₆) δ 8.28 (d, J=5.2 Hz, 1H), 7.62 (s, 1H), 7.58 (d, J=8.1 Hz, 1H), 7.54 (d, J=3.2 Hz, 1H), 7.05 (d, J=7.8 Hz, 1H), 6.79 (d, J=4.2 Hz, 1H), 6.64 (s, 1H), 6.48 (d, J=3.1 Hz, 1H), 4.60 (t, J=7.0 Hz, 2H), 4.52 (d, J=7.4 Hz, 2H), 4.40 (t, J=6.2 Hz, 2H), 3.66-3.37 (m, 6H), 3.33-3.23 (m, 2H), 2.53 (br s, 2H, overlapped with DMSO solvent peak), 1.74 (br s, 1H), 1.48 (br s, 1H). MS m/z: 485 [M+H]⁺.

### rac-3-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-5-(trifluoromethyl)pyridine (10)

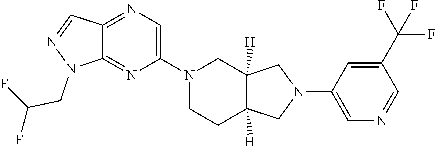 [SMILES: FC(F)Cn1ncc2ncc(N3CC[C@@H]4CN(c5cncc(C(F)(F)F)c5)C[C@@H]4C3)nc21]

Step 1: rac-tert-butyl (3aR,7aS)-2-[5-(trifluoromethyl)pyridin-3-yl]-octahydro-1H-pyrrolo[3,4-c]pyridine-5-carboxylate: Followed the general procedure C using rac-tert-butyl (3aR,7aS)-octahydro-1H-pyrrolo[3,4-c]pyridine-5-carboxylate (50 mg, 221 μmol, 1.0 equiv) and 3-bromo-5-(trifluoromethyl)pyridine (49.9 mg, 221 μmol, 1.0 equiv) as the starting materials to afford rac-tert-butyl (3aR,7aS)-2-[5-(trifluoromethyl)pyridin-3-yl]-octahydro-1H-pyrrolo[3,4-c]pyridine-5-carboxylate (60 mg, 73%) as a light-yellow oil. MS m/z: 372 [M+H]⁺.

Step 2: rac-3-[(3aR,7aR)-octahydro-1H-pyrrolo[3,4-c]pyridin-2-yl]-5-(trifluoromethyl)pyridine hydrochloride: Followed the general procedure B using rac-tert-butyl (3aR,7aS)-2-[5-(trifluoromethyl)pyridin-3-yl]-octahydro-1H-pyrrolo[3,4-c]pyridine-5-carboxylate (60 mg) as starting material to give the crude product rac-3-[(3aR,7aR)-octahydro-1H-pyrrolo[3,4-c]pyridin-2-yl]-5-(trifluoromethyl)pyridine hydrochloride (60 mg). MS m/z: 272 [M+H]⁺.

Step 3: rac-3-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-5-(trifluoromethyl)pyridine: Followed the general procedure D using rac-3-[(3aR,7aR)-octahydro-1H-pyrrolo[3,4-c]pyridin-2-yl]-5-(trifluoromethyl)pyridine hydrochloride (60 mg, 221 μmol, 1.0 equiv.) and 6-chloro-1-(2,2-difluoroethyl)-1H-pyrazolo[3,4-b]pyrazine (48.3 mg, 221 μmol, 1.0 equiv) as the starting materials to afford rac-3-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-5-(trifluoromethyl)pyridine (28 mg, 28%) as an off-white foam. ¹H NMR (500 MHz, DMSO-d₆) δ 8.47 (s, 1H), 8.15 (d, J=2.8 Hz, 1H), 8.12 (s, 1H), 8.10 (s, 1H), 7.04 (t, J=2.4 Hz, 1H), 6.44 (tt, J=54.9, 3.8 Hz, 1H), 4.70 (tdd, J=15.1, 3.8, 2.0 Hz, 2H), 4.02 (ddd, J=13.4, 6.9, 3.7 Hz, 1H), 3.93 (dd, J=13.9, 5.8 Hz, 1H), 3.84 (dd, J=13.9, 4.3 Hz, 1H), 3.66-3.57 (m, 1H), 3.47 (ddd, J=16.4, 9.9, 6.8 Hz, 2H), 3.38-3.35 (m, 1H, overlapped with water solvent peak), 3.16 (dd, J=10.0, 5.9 Hz, 1H), 2.67-2.55 (m, 2H), 1.91-1.78 (m, 1H), 1.64-1.53 (m, 1H). MS m/z: 454 [M+H]⁺.

### rac-2-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-4-(trifluoromethyl)pyridine (11)

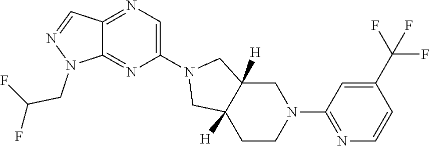 [SMILES: FC(F)Cn1ncc2ncc(N3C[C@H]4CCN(c5cc(C(F)(F)F)ccn5)C[C@H]4C3)nc21]

Followed the general procedure A using rac-6-[(3aR,7aR)-octahydro-1H-pyrrolo[3,4-c]pyridin-2-yl]-1-(2,2-difluoroethyl)-1H-pyrazolo[3,4-b]pyrazine hydrochloride (70 mg, 203 μmol, 1.0 equiv.) and 2-bromo-4-(trifluoromethyl)pyridine (45.9 mg, 203 μmol, 1.0 equiv) as the starting materials to afford rac-2-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-4-(trifluoromethyl)pyridine (25 mg, 27%) as a colorless oil. ¹H NMR (500 MHz, DMSO-d₆) δ 8.31 (d, J=5.1 Hz, 1H), 8.11 (s, 1H), 8.06 (s, 1H), 7.13 (s, 1H), 6.83 (dd, J=5.1, 1.4 Hz, 1H), 6.44 (tt, J=54.9, 3.9 Hz, 1H), 4.66 (td, J=14.9, 3.9 Hz, 2H), 3.93-3.77 (m, 2H), 3.70 (td, J=13.7, 12.9, 5.3 Hz, 3H), 3.60 (dd, J=10.9, 4.8 Hz, 1H), 3.50 (ddd, J=13.2, 8.1, 3.6 Hz, 1H), 3.40-3.34 (m, 2H), 2.57 (s, 2H), 1.86-1.75 (m, 1H), 1.62-1.48 (m, 1H). MS m/z: 454 [M+H]⁺.

### rac-5-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-2-(trifluoromethyl)pyridine (12)

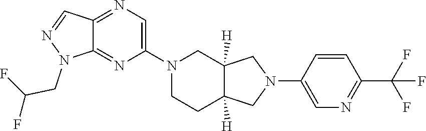 [SMILES: FC(F)Cn1ncc2ncc(N3CC[C@@H]4CN(c5ccc(C(F)(F)F)nc5)C[C@@H]4C3)nc21]

Followed the general procedure C using rac-6-[(3aR,7aS)-octahydro-1H-pyrrolo[3,4-c]pyridin-5-yl]-1-(2,2-difluoroethyl)-1H-pyrazolo[3,4-b]pyrazine hydrochloride (20 mg, 58 μmol, 1.0 equiv) 5-bromo-2-(trifluoromethyl)pyridine (13.1 mg, 58 μmol, 1.0 equiv) as the starting materials to afford rac-5-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-2-(trifluoromethyl)pyridine (4.2 mg, 16%) as a colorless oil. ¹H NMR (500 MHz, DMSO-d₆) δ 8.48 (s, 1H), 8.13 (s, 1H), 7.99 (d, J=2.8 Hz, 1H), 7.56 (d, J=8.7 Hz, 1H), 6.95 (dd, J=8.8, 2.8 Hz, 1H), 6.44 (tt, J=54.9, 3.8 Hz, 1H), 4.70 (td, J=15.2, 3.8 Hz, 2H), 4.02 (ddd, J=13.4, 7.0, 3.7 Hz, 1H), 3.94 (dd, J=14.0, 5.8 Hz, 1H), 3.85 (dd, J=13.9, 4.4 Hz, 1H), 3.62 (ddd, J=13.5, 8.4, 3.6 Hz, 1H), 3.48 (ddd, J=16.8, 10.1, 6.8 Hz, 2H), 3.15 (dd, J=10.2, 5.9 Hz, 1H), 2.66-2.51 (m, 2H), 2.52 (d, J=2.8 Hz, 1H), 1.92-1.81 (m, 1H), 1.62-1.50 (m, 1H). MS m/z: 454 [M+H]⁺.

### rac-2-[(3aR,7aS)-5-[1-(2,2,2-trifluoroethyl)-1H-pyrazolo[3,4-b]pyrazin-6-yl]-octahydro-1H-pyrrolo[3,4-c]pyridin-2-yl]-4-(trifluoromethyl)pyridine (13)

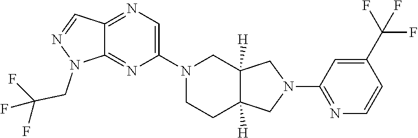 [SMILES: FC(F)(F)Cn1ncc2ncc(N3CC[C@@H]4CN(c5cc(C(F)(F)F)ccn5)C[C@@H]4C3)nc21]

Followed the general procedure D using 6-chloro-1-(2,2,2-trifluoroethyl)-1H-pyrazolo[3,4-b]pyrazine (46.9 mg, 198 μmol, 1.0 equiv) and rac-2-[(3aR,7aR)-octahydro-1H-pyrrolo[3,4-c]pyridin-2-yl]-4-(trifluoromethyl)pyridine (61 mg, 198 μmol, 1.0 equiv) as the starting materials to afford rac-2-[(3aR,7aS)-5-[1-(2,2,2-trifluoroethyl)-1H-pyrazolo[3,4-b]pyrazin-6-yl]-octahydro-1H-pyrrolo[3,4-c]pyridin-2-yl]-4-(trifluoromethyl)pyridine (56 mg, 60%) as a colorless oil. ¹H NMR (500 MHz, DMSO-d₆) δ 8.50 (s, 1H), 8.25 (d, J=5.2 Hz, 1H), 8.17 (s, 1H), 6.76 (dd, J=5.3, 1.5 Hz, 1H), 6.59 (s, 1H), 5.16 (qd, J=9.2, 3.1 Hz, 2H), 4.09-3.99 (m, 2H), 3.81 (dd, J=13.8, 4.1 Hz, 1H), 3.66-3.42 (m, 4H), 3.30-3.14 (m, 1H), 2.63-2.56 (m, 2H), 1.88-1.75 (m, 1H), 1.58-1.38 (m, 1H). MS m/z: 454 [M+H]⁺.

### rac-3-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-5-(trifluoromethyl)pyridine (14)

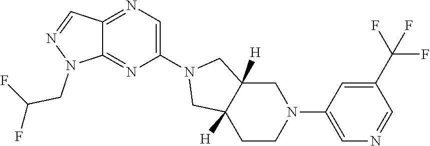 [SMILES: FC(F)Cn1ncc2ncc(N3C[C@H]4CCN(c5cncc(C(F)(F)F)c5)C[C@H]4C3)nc21]

Step 1: rac-tert-butyl (3aR,7aR)-5-[5-(trifluoromethyl)pyridine-3-yl]-octahydro-1H-pyrrolo[3,4-c]pyridine-2-carboxylate: Followed the general procedure C using rac-tert-butyl (3aR,7aR)-octahydro-1H-pyrrolo[3,4-c]pyridine-2-carboxylate (50 mg, 221 μmol, 1.0 equiv) and 3-bromo-5-(trifluoromethyl)pyridine (49.9 mg, 221 μmol, 1.0 equiv) as starting materials to afford rac-tert-butyl (3aR,7aR)-5-[5-(trifluoromethyl)pyridin-3-yl]-octahydro-1H-pyrrolo[3,4-c]pyridine-2-carboxylate (48 mg, 59%) as a light-yellow oil. MS m/z: 372 [M+H]⁺.

Step 2: rac-3-[(3aR,7aS)-octahydro-1H-pyrrolo[3,4-c]pyridin-5-yl]-5-(trifluoromethyl)pyridine: Followed the general procedure B using rac-tert-butyl (3aR,7aR)-5-[5-(trifluoromethyl)pyridin-3-yl]-octahydro-1H-pyrrolo[3,4-c]pyridine-2-carboxylate (48 mg) as the starting material to give the crude product rac-3-[(3aR,7aS)-octahydro-1H-pyrrolo[3,4-c]pyridin-5-yl]-5-(trifluoromethyl)pyridine hydrochloride (53 mg). MS m/z: 272 [M+H]⁺.

Step 3: rac-3-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-5-(trifluoromethyl)pyridine: Followed the general procedure A using rac-3-[(3aR,7aS)-octahydro-1H-pyrrolo[3,4-c]pyridin-5-yl]-5-(trifluoromethyl)pyridine hydrochloride (48 mg, 156 umol, 1.0 equiv.) and 6-chloro-1-(2,2-difluoroethyl)-1H-pyrazolo[3,4-b]pyrazine (34.1 mg, 156 umol, 1.0 equiv) as starting materials to afford rac-3-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-5-(trifluoromethyl)pyridine (49 mg, 69%) as an off-white solid. ¹H NMR (500 MHz, DMSO-d₆) δ 8.61 (d, J=2.8 Hz, 1H), 8.24 (s, 1H), 8.12 (s, 1H), 8.07 (s, 1H), 7.60 (t, J=2.4 Hz, 1H), 6.44 (tt, J=54.9, 3.9 Hz, 1H), 4.66 (td, J=14.9, 3.9 Hz, 2H), 3.73-3.68 (m, 2H), 3.65-3.52 (m, 2H), 3.49 (dd, J=12.0, 5.6 Hz, 3H), 3.27 (ddd, J=12.1, 7.8, 3.4 Hz, 1H), 2.67-2.51 (m, 2H), 1.92-1.81 (m, 1H), 1.68-1.51 (m, 1H). MS m/z: 454 [M+H]⁺.

### rac-3-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-4-(trifluoromethyl)pyridine (15)

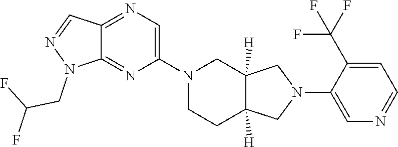 [SMILES: FC(F)Cn1ncc2ncc(N3CC[C@@H]4CN(c5cnccc5C(F)(F)F)C[C@@H]4C3)nc21]

Step 1: rac-tert-butyl (3aR,7aS)-2-[4-(trifluoromethyl)pyridin-3-yl]-octahydro-1H-pyrrolo[3,4-c]pyridine-5-carboxylate: Followed the general procedure C using rac-tert-butyl (3aR,7aS)-octahydro-1H-pyrrolo[3,4-c]pyridine-5-carboxylate (50 mg, 221 μmol, 1.0 equiv) and 3-bromo-4-(trifluoromethyl)pyridine (49.9 mg, 221 μmol, 1.0 equiv) as the starting materials to afford rac-tert-butyl (3aR,7aS)-2-[4-(trifluoromethyl)pyridin-3-yl]-octahydro-1H-pyrrolo[3,4-c]pyridine-5-carboxylate (26 mg, 32%) as a light-yellow oil. MS m/z: 372 [M+H]⁺.

Step 2: rac-3-[(3aR,7aR)-octahydro-1H-pyrrolo[3,4-c]pyridin-2-yl]-4-(trifluoromethyl)pyridine hydrochloride: Followed the general procedure B using rac-tert-butyl (3aR,7aS)-2-[4-(trifluoromethyl)pyridin-3-yl]-octahydro-1H-pyrrolo[3,4-c]pyridine-5-carboxylate (25 mg) as starting material to give the crude product rac-3-[(3aR,7aR)-octahydro-1H-pyrrolo[3,4-c]pyridin-2-yl]-4-(trifluoromethyl)pyridine hydrochloride (25 mg). MS m/z: 272 [M+H]⁺.

Step 3: rac-3-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-4-(trifluoromethyl)pyridine: Followed the general procedure A using rac-3-[(3aR,7aR)-octahydro-1H-pyrrolo[3,4-c]pyridin-2-yl]-4-(trifluoromethyl)pyridine hydrochloride (25 mg, 92 umol, 1.0 equiv.) 6-chloro-1-(2,2-difluoroethyl)-1H-pyrazolo[3,4-b]pyrazine (20.1 mg, 92 umol, 1.0 equiv) as starting materials to afford rac-3-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-4-(trifluoromethyl)pyridine (9 mg, 21%) as an off-white solid. ¹H NMR (500 MHz, DMSO-d₆) δ 8.47 (s, 1H), 8.36 (s, 1H), 8.13 (s, 1H), 8.03 (d, J=5.1 Hz, 1H), 7.43 (d, J=5.1 Hz, 1H), 6.43 (tt, J=54.9, 3.8 Hz, 1H), 4.69 (tdd, J=15.0, 3.8, 2.2 Hz, 2H), 4.07 (ddd, J=13.5, 6.5, 3.9 Hz, 1H), 3.97 (dd, J=13.9, 5.8 Hz, 1H), 3.80 (dd, J=13.9, 4.4 Hz, 1H), 3.63-3.52 (m, 2H), 3.49 (t, J=8.5 Hz, 1H), 3.37 (dd, J=9.6, 4.4 Hz, 1H), 3.27 (dd, J=9.8, 6.6 Hz, 1H), 2.64-2.54 (m, 2H), 1.87-1.81 (m, 1H), 1.61-1.54 (m, 1H). MS m/z: 454 [M+H]⁺.

### rac-6-[(3aR,7aS)-2-[4-(trifluoromethyl)pyridin-2-yl]-octahydro-1H-pyrrolo[3,4-c]pyridine-5-carbonyl]-1-(oxetan-3-yl)-1H-indole (16)

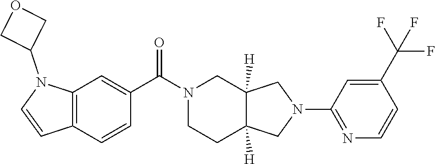 [SMILES: O=C(c1ccc2ccn(C3COC3)c2c1)N1CC[C@@H]2CN(c3cc(C(F)(F)F)ccn3)C[C@@H]2C1]

A mixture of 1-(oxetan-3-yl)-1H-indole-6-carboxylic acid (20 mg, 92 μmol, 1.0 equiv.) and HATU (38.5 mg, 101 μmol, 1.1 equiv.) were added into DMF (1.00 mL) followed by DIPEA (48.1 μL, 276 μmol, 3 equiv.) and rac-2-[(3aR,7aR)-octahydro-1H-pyrrolo[3,4-c]pyridin-2-yl]-4-(trifluoromethyl)pyridine (15 mg, 92 μmol, 1.0 equiv.) at room temperature. The mixture was stirred at room temperature for 16 h. The reaction was monitored by LCMS. The mixture was diluted with water (15 mL), and extracted with EtOAc (15 mL×2). The organic layer was combined, washed with brine, dried, evaporated, and purified with Combi-flash (4 g silico gel column), eluted with Hexane/EtOAc. The fractions were collected, and concentrated to afford rac-6-[(3aR,7aS)-2-[4-(trifluoromethyl)pyridin-2-yl]-octahydro-1H-pyrrolo[3,4-c]pyridine-5-carbonyl]-1-[(oxetan-3-yl)methyl]-1H-indole as a white solid. ¹H NMR (500 MHz, DMSO-d₆) δ 8.28 (d, J=5.2 Hz, 1H), 7.88 (d, J=3.3 Hz, 1H), 7.67 (s, 1H), 7.62 (d, J=8.1 Hz, 1H), 7.14-7.04 (m, 1H), 6.79 (d, J=4.6 Hz, 1H), 6.64 (s, 1H), 6.61 (dd, J=3.3, 0.8 Hz, 1H), 5.83 (p, J=7.5 Hz, 1H), 5.06-5.03 (m, 2H), 4.93-4.90 (m, 2H), 4.03-3.37 (m, 6H), 3.33-3.12 (m, 2H), 2.52 (br s, 2H, overlapped with DMSO solvent peak), 1.74 (br s, 1H), 1.49 (br s, 1H). MS m/z: 454 [M+H]⁺.

### rac-2-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-3-(trifluoromethyl)pyridine (17)

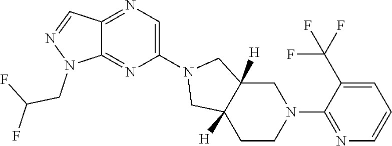 [SMILES: FC(F)Cn1ncc2ncc(N3C[C@H]4CCN(c5ncccc5C(F)(F)F)C[C@H]4C3)nc21]

Followed the general procedure A using rac-6-[(3aR,7aR)-octahydro-1H-pyrrolo[3,4-c]pyridin-2-yl]-1-(2,2-difluoroethyl)-1H-pyrazolo[3,4-b]pyrazine hydrochloride (70 mg, 203 μmol, 1.0 equiv.) and 2-bromo-3-(trifluoromethyl)pyridine (45.9 mg, 203 μmol, 1.0 equiv) as the starting materials to afford rac-2-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-3-(trifluoromethyl)pyridine (15 mg, 16%) as a colorless oil. ¹H NMR (500 MHz, DMSO-d₆) δ 8.53 (dd, J=4.8, 1.8 Hz, 1H), 8.12 (s, 1H), 8.10-8.04 (m, 2H), 7.21 (dd, J=8.0, 5.5 Hz, 1H), 6.45 (tt, J=55.0, 4.0 Hz, 1H), 4.67 (td, J=14.7, 3.7 Hz, 2H), 3.69 (br s, 2H), 3.64-3.54 (m, 2H), 3.36-3.32 (m, 1H), 3.29-3.23 (m, 2H), 3.05 (ddd, J=12.4, 8.8, 3.2 Hz, 1H), 2.63 (br s, 2H), 1.89-1.81 (m, 1H), 1.71-1.64 (m, 1H). MS m/z: 454 [M+H]⁺.

### rac-2-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-5-(trifluoromethyl)pyridine (18)

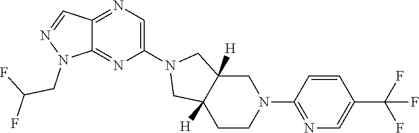 [SMILES: FC(F)Cn1ncc2ncc(N3C[C@H]4CCN(c5ccc(C(F)(F)F)cn5)C[C@H]4C3)nc21]

Followed the general procedure A using rac-6-[(3aR,7aR)-octahydro-1H-pyrrolo[3,4-c]pyridin-2-yl]-1-(2,2-difluoroethyl)-1H-pyrazolo[3,4-b]pyrazine hydrochloride (30 mg, 87 μmol, 1.0 equiv.) and 2-bromo-5-(trifluoromethyl)pyridine (19.7 mg, 87 μmol, 1.0 equiv) as starting materials to afford rac-2-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-5-(trifluoromethyl)pyridine (23.5 mg, 60%) as an off-white solid. ¹H NMR (500 MHz, DMSO-d₆) δ 8.39 (dt, J=2.8, 0.9 Hz, 1H), 8.11 (s, 1H), 8.06 (s, 1H), 7.77 (dd, J=9.2, 2.6 Hz, 1H), 6.99 (d, J=9.1 Hz, 1H), 6.43 (tt, J=54.9, 3.9 Hz, 1H), 4.66 (td, J=15.0, 3.9 Hz, 2H), 3.96-3.81 (m, 2H), 3.71-3.66 (m, 3H), 3.60 (dd, J=10.9, 4.7 Hz, 1H), 3.51 (ddd, J=12.7, 8.2, 3.5 Hz, 1H), 3.33-3.27 (m, 1H), 2.58 (br s, 2H), 1.86-1.71 (m, 1H), 1.52 (d, J=12.3 Hz, 1H). m/z: 454 [M+H]⁺.

### rac-2-[(3aR,7aS)-2-[4-(trifluoromethyl)pyridin-2-yl]-octahydro-1H-pyrrolo[3,4-c]pyridin-5-yl]-6-(1,3,4-thiadiazol-2-yl)pyrazine (19)

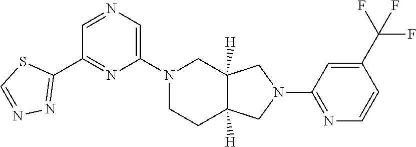 [SMILES: FC(F)(F)c1ccnc(N2C[C@H]3CCN(c4cncc(-c5nncs5)n4)C[C@H]3C2)c1]

Followed the general procedure D using rac-2-[(3aR,7aR)-octahydro-1H-pyrrolo[3,4-c]pyridin-2-yl]-4-(trifluoromethyl)pyridine hydrochloride (11 mg, 55 μmol, 1.0 equiv) 2-chloro-6-(1,3,4-thiadiazol-2-yl)pyrazine (11 mg, 55 μmol, 1.0 equiv) as starting materials to afford rac-2-[(3aR,7aS)-2-[4-(trifluoromethyl)pyridin-2-yl]-octahydro-1H-pyrrolo[3,4-c]pyridin-5-yl]-6-(1,3,4-thiadiazol-2-yl)pyrazine (5.3 mg, 22%) as a yellow solid. ¹H NMR (500 MHz, DMSO-d₆) δ 9.72 (s, 1H), 8.59 (s, 1H), 8.54 (s, 1H), 8.25 (d, J=5.2 Hz, 1H), 6.77 (dd, J=5.3, 1.5 Hz, 1H), 6.63 (d, J=1.6 Hz, 1H), 3.97 (ddd, J=13.4, 6.7, 3.8 Hz, 1H), 3.88 (dd, J=13.9, 5.5 Hz, 1H), 3.75 (dd, J=13.9, 4.2 Hz, 1H), 3.60-3.43 (m, 4H), 3.29-3.21 (m, 1H), 2.67-2.53 (m, 2H), 1.88-1.58 (m, 1H), 1.61-1.49 (m, 1H). MS m/z: 435 [M+H]⁺.

### rac-5-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-2-(trifluoromethyl)pyridine (20)

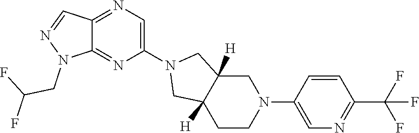 [SMILES: FC(F)Cn1ncc2ncc(N3C[C@H]4CCN(c5ccc(C(F)(F)F)nc5)C[C@H]4C3)nc21]

Step 1: rac-tert-butyl (3aR,7aR)-5-[6-(trifluoromethyl)pyridin-3-yl]-octahydro-1H-pyrrolo[3,4-c]pyridine-2-carboxylate: Followed the general procedure C using rac-tert-butyl (3aR,7aR)-octahydro-1H-pyrrolo[3,4-c]pyridine-2-carboxylate (50 mg, 221 μmol, 1.0 equiv) and 5-bromo-2-(trifluoromethyl)pyridine (49.9 mg, 221 μmol, 1.0 equiv) as the starting materials to afford rac-tert-butyl (3aR,7aR)-5-[6-(trifluoromethyl)pyridin-3-yl]-octahydro-1H-pyrrolo[3,4-c]pyridine-2-carboxylate (63 mg, 77%) as a light-yellow oil. MS m/z: 372 [M+H]⁺.

Step 2: rac-5-[(3aR,7aS)-octahydro-1H-pyrrolo[3,4-c]pyridin-5-yl]-2-(trifluoromethyl)pyridine hydrochloride: Followed the general procedure B using rac-tert-butyl (3aR,7aR)-5-[6-(trifluoromethyl)pyridin-3-yl]-octahydro-1H-pyrrolo[3,4-c]pyridine-2-carboxylate (63 mg) as starting material to give the crude product rac-5-[(3aR,7aS)-octahydro-1H-pyrrolo[3,4-c]pyridin-5-yl]-2-(trifluoromethyl)pyridine hydrochloride (56 mg). MS m/z: 272 [M+H]⁺.

Step 3: rac-5-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-2-(trifluoromethyl)pyridine: Followed the general procedure A using rac-5-[(3aR,7aS)-octahydro-1H-pyrrolo[3,4-c]pyridin-5-yl]-2-(trifluoromethyl)pyridine hydrochloride (56 mg, 182 μmol, 1.0 equiv.) and 6-chloro-1-(2,2-difluoroethyl)-1H-pyrazolo[3,4-b]pyrazine (45 mg, 182 μmol, 1.0 equiv) as the starting materials to afford Preparation of rac-5-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-2-(trifluoromethyl)pyridine (55 mg, 59%) as an off-white solid. ¹H NMR (500 MHz, DMSO-d₆) δ 8.45 (d, J=2.9 Hz, 1H), 8.12 (s, 1H), 8.07 (s, 1H), 7.62 (d, J=8.8 Hz, 1H), 7.45 (dd, J=8.9, 2.9 Hz, 1H), 6.44 (tt, J=54.9, 3.9 Hz, 1H), 4.66 (td, J=14.9, 3.8 Hz, 2H), 3.71 (td, J=10.6, 7.0 Hz, 2H), 3.59 (dtd, J=16.2, 8.6, 7.9, 4.5 Hz, 2H), 3.55-3.48 (m, 2H), 3.43 (dd, J=11.2, 5.9 Hz, 1H), 3.34-3.28 (m, 1H), 2.63-2.58 (m, 2H), 1.89-1.84 (m, 1H), 1.64-1.58 (m, 1H). MS m/z: 454 [M+H]⁺.

### rac-4-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-2-(trifluoromethyl)pyridine (21)

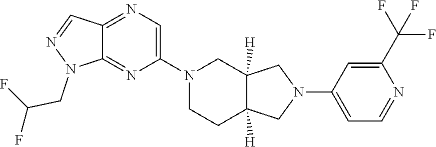 [SMILES: FC(F)Cn1ncc2ncc(N3CC[C@@H]4CN(c5ccnc(C(F)(F)F)c5)C[C@@H]4C3)nc21]

Followed the general procedure C using rac-6-[(3aR,7aS)-octahydro-1H-pyrrolo[3,4-c]pyridin-5-yl]-1-(2,2-difluoroethyl)-1H-pyrazolo[3,4-b]pyrazine hydrochloride (20 mg, 58 μmol, 1.0 equiv) 4-bromo-2-(trifluoromethyl)pyridine (13.1 mg, 58 μmol, 1.0 equiv) as the starting materials to afford rac-4-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-2-(trifluoromethyl)pyridine (4.4 mg, 17%) as a colorless oil. ¹H NMR (500 MHz, DMSO-d₆) δ 8.47 (s, 1H), 8.18 (d, J=5.8 Hz, 1H), 8.13 (s, 1H), 6.78 (s, 1H), 6.64-6.62 (m, 1H), 6.44 (tt, J=54.9, 3.8 Hz, 1H), 4.70 (tdd, J=15.1, 3.8, 2.4 Hz, 2H), 4.05-4.01 (m, 1H), 3.97-3.91 (m, 1H), 3.82 (d, J=13.4 Hz, 1H), 3.60 (br s, 1H), 3.51-3.45 (m, 2H), 3.16 (dd, J=10.6, 6.0 Hz, 1H), 2.68-2.54 (m, 2H), 2.51 (m, 1H), 1.91-1.77 (m, 1H), 1.57-1.50 (m, 1H). MS m/z: 454 [M+H]⁺.

### rac-3-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-4-(trifluoromethyl)pyridine (22)

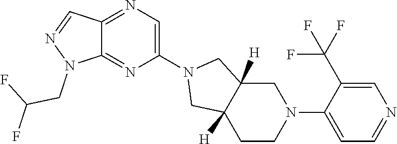 [SMILES: FC(F)Cn1ncc2ncc(N3C[C@H]4CCN(c5ccncc5C(F)(F)F)C[C@H]4C3)nc21]

Step 1: rac-tert-butyl (3aR,7aR)-5-[3-(trifluoromethyl)pyridin-4-yl]-octahydro-1H-pyrrolo[3,4-c]pyridine-2-carboxylate: Followed the general procedure C using rac-tert-butyl (3aR,7aR)-octahydro-1H-pyrrolo[3,4-c]pyridine-2-carboxylate (50 mg, 221 μmol, 1.0 equiv) and 4-bromo-3-(trifluoromethyl)pyridine (49.9 mg, 221 μmol, 1.0 equiv) as starting materials to afford rac-tert-butyl (3aR,7aR)-5-[3-(trifluoromethyl)pyridin-4-yl]-octahydro-1H-pyrrolo[3,4-c]pyridine-2-carboxylate (16 mg, 20%) as a light-yellow oil. MS m/z: 372 [M+H]⁺.

Step 2: rac-4-[(3aR,7aS)-octahydro-1H-pyrrolo[3,4-c]pyridin-5-yl]-3-(trifluoromethyl)pyridine hydrochloride: Follow the general procedure B using rac-tert-butyl (3aR,7aR)-5-[3-(trifluoromethyl)pyridin-4-yl]-octahydro-1H-pyrrolo[3,4-c]pyridine-2-carboxylate (16 mg) as the starting material to give the crude product rac-4-[(3aR,7aS)-octahydro-1H-pyrrolo[3,4-c]pyridin-5-yl]-3-(trifluoromethyl)pyridine hydrochloride (17 mg). MS m/z: 272 [M+H]⁺.

Step 3: rac-3-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-4-(trifluoromethyl)pyridine: Followed the general procedure A using rac-4-[(3aR,7aS)-octahydro-1H-pyrrolo[3,4-c]pyridin-5-yl]-3-(trifluoromethyl)pyridine hydrochloride (19 mg, 70 umol, 1.0 equiv.) and 6-chloro-1-(2,2-difluoroethyl)-1H-pyrazolo[3,4-b]pyrazine (15.3 mg, 70 umol, 1.0 equiv) as starting materials to afford rac-4-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-3-(trifluoromethyl)pyridine (12 mg, 38%) as an off-white solid. ¹H NMR (500 MHz, DMSO-d₆) δ 8.69 (s, 1H), 8.58 (d, J=5.7 Hz, 1H), 8.12 (s, 1H), 8.08 (s, 1H), 7.28 (d, J=5.8 Hz, 1H), 6.58-6.30 (m, 1H), 4.67 (td, J=14.8, 3.8 Hz, 2H), 3.70 (br s, 2H), 3.60 (dd, J=11.0, 4.6 Hz, 1H), 3.54 (dd, J=11.0, 6.5 Hz, 1H), 3.31-3.21 (m, 2H), 3.18 (dd, J=12.6, 5.7 Hz, 1H), 3.03 (ddd, J=12.2, 8.5, 3.3 Hz, 1H), 2.72-2.52 (m, 2H), 1.91-1.87 (m, 1H), 1.72-1.66 (m, 1H). MS m/z: 454 [M+H]⁺.

### rac-4-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-2-(trifluoromethyl)pyridine (23)

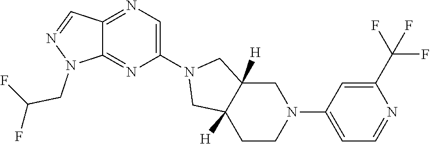 [SMILES: FC(F)Cn1ncc2ncc(N3C[C@H]4CCN(c5ccnc(C(F)(F)F)c5)C[C@H]4C3)nc21]

Step 1: rac-tert-butyl (3aR,7aR)-5-[2-(trifluoromethyl)pyridin-4-yl]-octahydro-1H-pyrrolo[3,4-c]pyridine-2-carboxylate: Followed the general procedure C using rac-tert-butyl (3aR,7aR)-octahydro-1H-pyrrolo[3,4-c]pyridine-2-carboxylate (50 mg, 221 μmol, 1.0 equiv) and 4-bromo-2-(trifluoromethyl)pyridine (49.9 mg, 221 μmol, 1.0 equiv) as the starting materials to afford rac-tert-butyl (3aR,7aR)-5-[2-(trifluoromethyl)pyridin-4-yl]-octahydro-1H-pyrrolo[3,4-c]pyridine-2-carboxylate (53 mg, 65%) as a colorless oil. MS m/z: 372 [M+H]⁺.

Step 2: rac-4-[(3aR,7aS)-octahydro-1H-pyrrolo[3,4-c]pyridin-5-yl]-2-(trifluoromethyl)pyridine hydrochloride: Followed the general procedure B using rac-tert-butyl (3aR,7aR)-5-[2-(trifluoromethyl)pyridin-4-yl]-octahydro-1H-pyrrolo[3,4-c]pyridine-2-carboxylate (53 mg) as starting material to give the crude product rac-4-[(3aR,7aS)-octahydro-1H-pyrrolo[3,4-c]pyridin-5-yl]-2-(trifluoromethyl)pyridine hydrochloride (54 mg). MS m/z: 272 [M+H]⁺.

Step 3: rac-4-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-2-(trifluoromethyl)pyridine: Followed the general procedure A using rac-4-[(3aR,7aS)-octahydro-1H-pyrrolo[3,4-c]pyridin-5-yl]-2-(trifluoromethyl)pyridine hydrochloride (54 mg, 199 μmol, 1.0 equiv.) and 6-chloro-1-(2,2-difluoroethyl)-1H-pyrazolo[3,4-b]pyrazine (43.5 mg, 199 μmol, 1.0 equiv) as the starting materials to afford rac-4-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-2-(trifluoromethyl)pyridine (27 mg, 30%) as an off-white solid. ¹H NMR (500 MHz, DMSO-d₆) δ 8.24 (d, J=6.0 Hz, 1H), 8.12 (s, 1H), 8.06 (s, 1H), 7.24 (d, J=2.6 Hz, 1H), 7.06 (dd, J=6.1, 2.6 Hz, 1H), 6.44 (tt, J=54.9, 3.9 Hz, 1H), 4.66 (td, J=14.9, 3.9 Hz, 2H), 3.77-3.53 (m, 6H), 3.46-3.34 (m, 2H), 2.59 (br s, 2H), 1.90-1.79 (m, 1H), 1.61-1.55 (m, 1H). MS m/z: 454 [M+H]⁺.

### rac-3-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-4-(trifluoromethyl)pyridine (24)

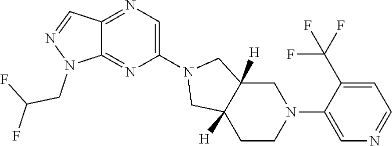 [SMILES: FC(F)Cn1ncc2ncc(N3C[C@H]4CCN(c5cnccc5C(F)(F)F)C[C@H]4C3)nc21]

Step 1: rac-tert-butyl (3aR,7aR)-5-[4-(trifluoromethyl)pyridin-3-yl]-octahydro-1H-pyrrolo[3,4-c]pyridine-2-carboxylate: Followed the general procedure C using rac-tert-butyl (3aR,7aR)-octahydro-1H-pyrrolo[3,4-c]pyridine-2-carboxylate (50 mg, 221 μmol, 1.0 equiv) and 3-bromo-4-(trifluoromethyl)pyridine (49.9 mg, 221 μmol, 1.0 equiv) as the starting materials to afford rac-tert-butyl (3aR,7aR)-5-[4-(trifluoromethyl)pyridin-3-yl]-octahydro-1H-pyrrolo[3,4-c]pyridine-2-carboxylate (13 mg, 16%) as a colorless oil. MS m/z: 372 [M+H]⁺.

Step 2: rac-3-[(3aR,7aS)-octahydro-1H-pyrrolo[3,4-c]pyridin-5-yl]-4-(trifluoromethyl)pyridine hydrochloride: Follow the general procedure B using rac-tert-butyl (3aR,7aR)-5-[4-(trifluoromethyl)pyridin-3-yl]-octahydro-1H-pyrrolo[3,4-c]pyridine-2-carboxylate (13 mg) as starting material to give the crude product rac-4-[(3aR,7aS)-octahydro-1H-pyrrolo[3,4-c]pyridin-5-yl]-2-(trifluoromethyl)pyridine hydrochloride (17 mg). MS m/z: 272 [M+H]⁺.

Step 3: rac-3-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-4-(trifluoromethyl)pyridine: Followed the general procedure A using rac-3-[(3aR,7aS)-octahydro-1H-pyrrolo[3,4-c]pyridin-5-yl]-4-(trifluoromethyl)pyridine hydrochloride (17 mg, 63 umol, 1.0 equiv.) and 6-chloro-1-(2,2-difluoroethyl)-1H-pyrazolo[3,4-b]pyrazine (13.7 mg, 63 umol, 1.0 equiv) as the starting materials to afford rac-3-[(3aR,7aR)-2-[1-(2,2-difluoroethyl)-1H-pyrazolo[3,4-b]pyrazin-6-yl]-octahydro-1H-pyrrolo[3,4-c]pyridin-5-yl]-4-(trifluoromethyl)pyridine (9.5 mg, 33%) as an off-white solid. ¹H NMR (500 MHz, DMSO-d₆) δ 8.86 (s, 1H), 8.57 (dd, J=5.2, 0.9 Hz, 1H), 8.12 (s, 1H), 8.08 (br s, 1H), 7.66 (d, J=5.0 Hz, 1H), 6.43 (tt, J=54.9, 3.9 Hz, 1H), 4.67 (td, J=14.9, 3.8 Hz, 2H), 3.67-3.61 (m, 4H), 3.26-3.14 (m, 1H), 3.05 (q, J=5.5, 5.0 Hz, 2H), 2.92 (ddd, J=11.8, 8.4, 3.3 Hz, 1H), 2.63 (p, J=1.8 Hz, 2H), 1.90-1.85 (m, 1H), 1.70-1.64 (m, 1H). MS m/z: 454 [M+H]⁺.

### rac-3-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-4-(trifluoromethyl)pyridine (25)

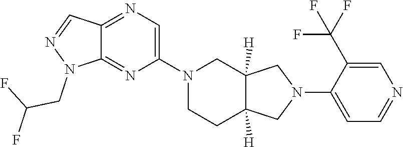 [SMILES: FC(F)Cn1ncc2ncc(N3CC[C@@H]4CN(c5ccncc5C(F)(F)F)C[C@@H]4C3)nc21]

Step 1: rac-tert-butyl (3aR,7aS)-2-[3-(trifluoromethyl)pyridin-4-yl]-octahydro-1H-pyrrolo[3,4-c]pyridine-5-carboxylate: Followed the general procedure C using rac-tert-butyl (3aR,7aS)-octahydro-1H-pyrrolo[3,4-c]pyridine-5-carboxylate (50 mg, 221 μmol, 1.0 equiv) and 4-bromo-3-(trifluoromethyl)pyridine (49.9 mg, 221 μmol, 1.0 equiv) as the starting materials to afford rac-tert-butyl (3aR,7aS)-2-[3-(trifluoromethyl)pyridin-4-yl]-octahydro-1H-pyrrolo[3,4-c]pyridine-5-carboxylate (26 mg, 32%) as a colorless oil. MS m/z: 372 [M+H]⁺.

Step 2: rac-4-[(3aR,7aR)-octahydro-1H-pyrrolo[3,4-c]pyridin-2-yl]-3-(trifluoromethyl)pyridine hydrochloride: Follow the general procedure B using rac-tert-butyl (3aR,7aS)-2-[3-(trifluoromethyl)pyridin-4-yl]-octahydro-1H-pyrrolo[3,4-c]pyridine-5-carboxylate (25 mg) as the starting material to give the crude product rac-4-[(3aR,7aR)-octahydro-1H-pyrrolo[3,4-c]pyridin-2-yl]-3-(trifluoromethyl)pyridine hydrochloride (25 mg). MS m/z: 272 [M+H]⁺.

Step 3: rac-3-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-4-(trifluoromethyl)pyridine: Followed the general procedure A using rac-4-[(3aR,7aR)-octahydro-1H-pyrrolo[3,4-c]pyridin-2-yl]-3-(trifluoromethyl)pyridine hydrochloride (25 mg, 92 μmol, 1.0 equiv.) and 6-chloro-1-(2,2-difluoroethyl)-1H-pyrazolo[3,4-b]pyrazine (20.1 mg, 92 μmol, 1.0 equiv) as the starting materials to afford rac-4-[(3aR,7aS)-5-[1-(2,2-difluoroethyl)-1H-pyrazolo[3,4-b]pyrazin-6-yl]-octahydro-1H-pyrrolo[3,4-c]pyridin-2-yl]-3-(trifluoromethyl)pyridine (9.5 mg, 33%) as an off-white solid. ¹H NMR (500 MHz, DMSO-d₆) δ 8.48 (s, 1H), 8.47 (br s, 1H), 8.23 (br s, 1H), 8.13 (s, 1H), 6.76 (d, J=6.0 Hz, 1H), 6.44 (tt, J=54.9, 3.8 Hz, 1H), 4.70 (tdd, J=15.0, 3.8, 2.2 Hz, 2H), 4.10 (ddd, J=13.6, 6.4, 3.9 Hz, 1H), 4.02 (dd, J=14.0, 5.5 Hz, 1H), 3.75 (dd, J=14.0, 4.3 Hz, 1H), 3.61 (dd, J=10.3, 6.4 Hz, 1H), 3.57-3.46 (m, 2H), 3.39 (dd, J=10.3, 4.3 Hz, 1H), 3.28 (dd, J=10.4, 7.1 Hz, 1H), 2.63-2.54 (m, 2H), 1.85-1.79 (m, 1H), 1.53-1.46 (m, 1H). MS m/z: 454 [M+H]⁺.

### Example 26: 3-{[(7R,8aS)-2-[1-(2,2-difluoroethyl)-1H-pyrazolo[3,4-b]pyrazin-6-yl]-octahydropyrrolo[1,2-a]pyrazin-7-yl]oxy}-2-(trifluoromethyl)pyridine

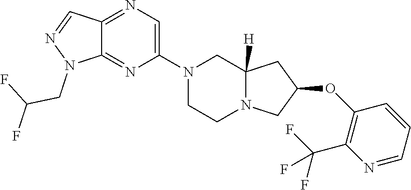 [SMILES: FC(F)Cn1ncc2ncc(N3CCN4C[C@H](Oc5cccnc5C(F)(F)F)C[C@H]4C3)nc21]

Followed the General procedure A to afford the desired product as a white solid (15 mg, 57%). ¹H NMR (500 MHz, DMSO-d₆) δ 8.48 (s, 1H), 8.27 (dd, J=4.5, 1.1 Hz, 1H), 8.15 (s, 1H), 7.80 (d, J=8.5 Hz, 1H), 7.69 (dd, J=8.6, 4.5 Hz, 1H), 6.44 (tt, J=54.9, 3.9 Hz, 1H), 5.17 (dq, J=9.3, 6.0, 4.1 Hz, 1H), 4.70 (td, J=14.9, 3.7 Hz, 3H), 4.59-4.50 (m, 1H), 3.74 (dd, J=9.8, 6.6 Hz, 1H), 3.12-3.00 (m, 2H), 2.75 (dd, J=12.5, 10.4 Hz, 1H), 2.38-2.20 (m, 3H), 2.02-1.90 (m, 2H). MS m/z: 470 [M+H]⁺.

### Example 27: 3-{[(7R,8aS)-2-{5-methyl-6-phenyl-5H-pyrrolo[2,3-b]pyrazine-7-carbonyl}-octahydropyrrolo[1,2-a]pyrazin-7-yl]oxy}-2-(trifluoromethyl)pyridine

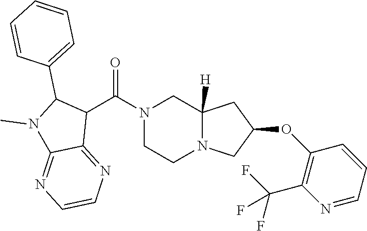 [SMILES: CN1c2nccnc2C(C(=O)N2CCN3C[C@H](Oc4cccnc4C(F)(F)F)C[C@H]3C2)C1c1ccccc1]

Followed the General procedure F to afford the desired product as a yellow oil (13 mg, 45%). ¹H NMR (500 MHz, CDCl₃) δ 8.51 (s, 1H), 8.32 (d, J=2.6 Hz, 1H), 8.28 (t, J=4.2 Hz, 1H), 7.54 (d, J=2.6 Hz, 5H), 7.46-7.40 (m, 1H), 7.28-7.20 (m, 1H), 5.02-4.74 (m, 2H), 3.84 (d, J=6.0 Hz, 3H), 3.76-3.58 (m, 2H), 3.20-2.85 (m, 2H), 2.80-2.50 (m, 2H), 2.46-2.14 (m, 2H), 1.75-1.60 (m, 2H). MS m/z: 523 [M+H]⁺.

### Example 28: 6-[(7R,8aS)-7-{[2-(trifluoromethyl)pyridin-3-yl]oxy}-octahydropyrrolo[1,2-a]pyrazine-2-carbonyl]-1H-indole

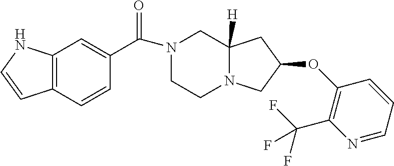 [SMILES: O=C(c1ccc2cc[nH]c2c1)N1CCN2C[C@H](Oc3cccnc3C(F)(F)F)C[C@H]2C1]

Followed the General procedure F to afford the desired product as a colorless oil (8 mg, 33.4%). MS m/z: 431 [M+H]⁺.

### Example 29: 2-({2-[1-(2,2-difluoroethyl)-1H-pyrazolo[3,4-b]pyrazin-6-yl]-octahydrocyclopenta[c]pyrrol-5-yl}oxy)-6-(trifluoromethyl)pyridine

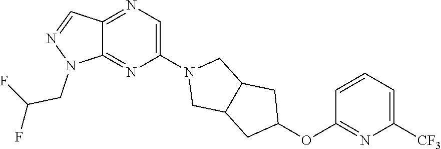 [SMILES: FC(F)Cn1ncc2ncc(N3CC4CC(Oc5cccc(C(F)(F)F)n5)CC4C3)nc21]

Followed the General procedure A to afford the desired product as a colorless oil (27 mg, 76%). ¹H NMR (500 MHz, DMSO-d₆) δ 8.12 (s, 1H), 8.10 (s, 1H), 7.88 (t, J=7.9 Hz, 1H), 7.43 (d, J=7.3 Hz, 1H), 6.92 (d, J=8.4 Hz, 1H), 6.44 (tt, J=54.9, 3.9 Hz, 1H), 5.40 (tt, J=6.8, 5.2 Hz, 1H), 4.67 (td, J=14.9, 3.9 Hz, 2H), 3.85-3.75 (m, 2H), 3.61 (dd, J=11.4, 3.6 Hz, 2H), 2.95-2.85 (m, 2H), 2.46 (qd, J=8.1, 6.7 Hz, 2H), 1.75 (dt, J=14.0, 5.0 Hz, 2H). MS m/z: 455 [M+H]⁺.

### Example 30: 2-({2-[1-(2,2-difluoroethyl)-1H-pyrazolo[3,4-b]pyrazin-6-yl]-octahydrocyclopenta[c]pyrrol-5-yl}oxy)-4-(trifluoromethyl)pyridine

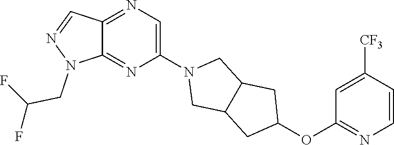 [SMILES: FC(F)Cn1ncc2ncc(N3CC4CC(Oc5cc(C(F)(F)F)ccn5)CC4C3)nc21]

Followed the General procedure A to afford the desired product as a colorless oil (21 mg, 57%). MS m/z: 455 [M+H]⁺.

### Example 31: 3-({2-[1-(2,2-difluoroethyl)-1H-pyrazolo[3,4-b]pyrazin-6-yl]-octahydrocyclopenta[c]pyrrol-5-yl}oxy)-2-(trifluoromethyl)pyridine

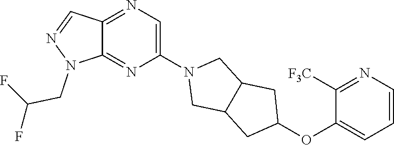 [SMILES: FC(F)Cn1ncc2ncc(N3CC4CC(Oc5cccnc5C(F)(F)F)CC4C3)nc21]

Followed the General procedure A to afford the desired product as a colorless oil (26 mg, 50%). ¹H NMR (500 MHz, DMSO-d₆) δ 8.21 (dd, J=4.5, 1.1 Hz, 1H), 8.12 (s, 1H), 8.06 (s, 1H), 7.79 (d, J=8.6 Hz, 1H), 7.67 (dd, J=8.6, 4.5 Hz, 1H), 6.43 (tt, J=55.0, 3.9 Hz, 1H), 5.15 (tt, J=6.6, 3.4 Hz, 1H), 4.66 (td, J=14.9, 3.9 Hz, 2H), 3.88 (dd, J=11.2, 8.2 Hz, 2H), 3.53 (dd, J=11.3, 4.1 Hz, 2H), 2.96 (dq, J=8.7, 4.5 Hz, 2H), 2.44 (ddd, J=14.5, 8.2, 6.3 Hz, 2H), 1.82 (dt, J=14.4, 3.6 Hz, 2H). MS m/z: 455 [M+H]⁺.

### Example 32: (3aR,5S,6aS)—N-[1-(2,2-difluoroethyl)-1H-pyrazolo[3,4-b]pyrazin-6-yl]-2-[6-(trifluoromethyl)pyridin-2-yl]-octahydrocyclopenta[c]pyrrol-5-amine

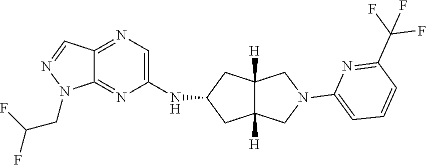 [SMILES: FC(F)Cn1ncc2ncc(N[C@@H]3C[C@@H]4CN(c5cccc(C(F)(F)F)n5)C[C@@H]4C3)nc21]

Followed the General procedure A to afford the desired product as a colorless oil (20 mg, 61%). ¹H NMR (500 MHz, DMSO-d₆) δ 8.03 (s, 1H), 7.95 (d, J=6.9 Hz, 1H), 7.88 (s, 1H), 7.70 (t, J=7.9 Hz, 1H), 6.98 (d, J=7.2 Hz, 1H), 6.79 (d, J=8.6 Hz, 1H), 6.43 (tt, J=54.9, 3.8 Hz, 2H), 4.66 (td, J=15.0, 3.9 Hz, 2H), 4.35 (qd, J=9.2, 7.1 Hz, 1H), 3.58 (dd, J=10.9, 7.1 Hz, 2H), 3.44 (dd, J=10.9, 3.2 Hz, 2H), 2.81 (dq, J=11.4, 7.7, 5.8 Hz, 2H), 2.45 (t, J=6.3 Hz, 2H), 1.43 (ddd, J=12.6, 9.1, 6.9 Hz, 2H). MS m/z: 454 [M+H]⁺.

### Example 33: (3aR,5S,6aS)—N-[1-(2,2-difluoroethyl)-1H-pyrazolo[3,4-b]pyrazin-6-yl]-2-[4-(trifluoromethyl)pyridin-2-yl]-octahydrocyclopenta[c]pyrrol-5-amine

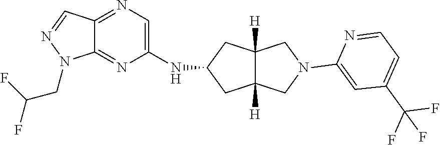 [SMILES: FC(F)Cn1ncc2ncc(N[C@@H]3C[C@@H]4CN(c5cc(C(F)(F)F)ccn5)C[C@@H]4C3)nc21]

Followed the General procedure A to afford the desired product as a white solid (14 mg, 43%). ¹H NMR (500 MHz, DMSO-d₆) δ 8.30 (d, J=5.2 Hz, 1H), 8.03 (s, 1H), 7.95 (d, J=6.9 Hz, 1H), 7.88 (s, 1H), 6.82 (dd, J=5.2, 1.5 Hz, 1H), 6.73 (s, 1H), 6.43 (tt, J=54.9, 3.8 Hz, 2H), 4.66 (td, J=15.0, 3.8 Hz, 2H), 4.32 (h, J=8.2 Hz, 1H), 3.59 (dd, J=10.8, 7.2 Hz, 2H), 3.49 (dd, J=11.1, 3.2 Hz, 2H), 2.80 (dd, J=9.4, 5.4 Hz, 2H), 2.48-2.42 (m, 2H), 1.42 (ddd, J=12.7, 9.1, 6.9 Hz, 2H). MS m/z: 454 [M+H]⁺.

### Example 34: (3aR,5R,6aS)—N-[1-(2,2-difluoroethyl)-1H-pyrazolo[3,4-b]pyrazin-6-yl]-2-[6-(trifluoromethyl)pyridin-2-yl]-octahydrocyclopenta[c]pyrrol-5-amine

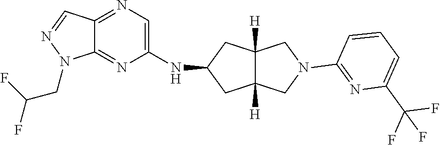 [SMILES: FC(F)Cn1ncc2ncc(N[C@H]3C[C@@H]4CN(c5cccc(C(F)(F)F)n5)C[C@@H]4C3)nc21]

Followed the General procedure A to afford the desired product as a colorless oil (18 mg, 55%). ¹H NMR (500 MHz, DMSO-d₆) δ 8.03 (s, 1H), 7.93 (s, 1H), 7.93 (q, J=7.5, 6.4 Hz, 1H), 7.70 (t, J=7.9 Hz, 1H), 6.98 (d, J=7.2 Hz, 1H), 6.78 (d, J=8.6 Hz, 1H), 6.40 (tt, J=54.9, 3.8 Hz, 2H), 4.62 (td, J=15.0, 3.9 Hz, 2H), 4.40 (h, J=6.4 Hz, 1H), 3.67 (dd, J=10.9, 7.7 Hz, 2H), 3.33-3.28 (m, 2H), 2.97 (dt, J=7.9, 4.2 Hz, 2H), 2.01 (ddd, J=13.1, 6.5, 3.9 Hz, 2H), 1.89 (ddd, J=13.4, 7.8, 5.7 Hz, 2H). MS m/z: 454 [M+H]⁺.

### Example 35: (3aR,5R,6aS)—N-[1-(2,2-difluoroethyl)-1H-pyrazolo[3,4-b]pyrazin-6-yl]-2-[4-(trifluoromethyl)pyridin-2-yl]-octahydrocyclopenta[c]pyrrol-5-amine

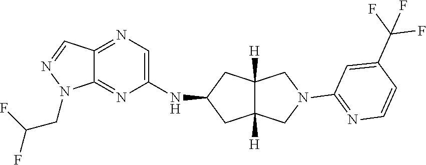 [SMILES: FC(F)Cn1ncc2ncc(N[C@H]3C[C@@H]4CN(c5cc(C(F)(F)F)ccn5)C[C@@H]4C3)nc21]

Followed the General procedure A to afford the desired product as a white solid (16 mg, 49%). ¹H NMR (500 MHz, DMSO-d₆) δ 8.30 (d, J=5.2 Hz, 1H), 8.03 (s, 1H), 7.93 (s, 1H), 7.91 (d, J=6.4 Hz, 1H), 6.82 (dd, J=5.2, 1.5 Hz, 1H), 6.74-6.68 (m, 1H), 6.40 (tt, J=55.0, 3.9 Hz, 1H), 4.62 (td, J=15.0, 3.9 Hz, 2H), 4.40 (h, J=6.4 Hz, 1H), 3.69 (dd, J=10.9, 7.9 Hz, 2H), 3.36-3.33 (m, 2H), 2.97 (dq, J=8.1, 4.3 Hz, 2H), 2.12-1.96 (m, 2H), 1.94-1.78 (m, 2H). MS m/z: 454 [M+H]⁺.

### Example 36: (3aR,5S,6aS)—N-[1-(2,2-difluoroethyl)-1H-pyrazolo[3,4-b]pyrazin-6-yl]-2-[2-(trifluoromethyl)pyridin-3-yl]-octahydrocyclopenta[c]pyrrol-5-amine

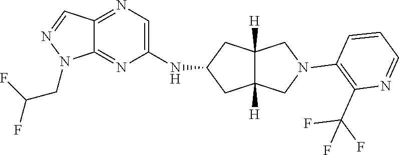 [SMILES: FC(F)Cn1ncc2ncc(N[C@@H]3C[C@@H]4CN(c5cccnc5C(F)(F)F)C[C@@H]4C3)nc21]

Followed the General procedure A to afford the desired product as a light-yellow solid (8 mg, 24%). ¹H NMR (500 MHz, DMSO-d₆) δ 8.30 (dd, J=4.5, 1.3 Hz, 1H), 8.03 (s, 1H), 7.97 (d, J=6.7 Hz, 1H), 7.94 (s, 1H), 7.78 (dd, J=8.3, 1.3 Hz, 1H), 7.60 (dd, J=8.4, 4.4 Hz, 1H), 6.44 (tt, J=54.9, 3.8 Hz, 2H), 4.66 (td, J=15.0, 3.9 Hz, 2H), 4.12 (td, J=11.1, 5.6 Hz, 1H), 3.16-3.06 (m, 3H), 2.73 (d, J=5.6 Hz, 2H), 2.45 (dt, J=13.3, 7.1 Hz, 2H), 1.38 (q, J=11.4 Hz, 2H). MS m/z: 454 [M+H]⁺.

### Example 37: (3aR,5R,6aS)—N-[1-(2,2-difluoroethyl)-1H-pyrazolo[3,4-b]pyrazin-6-yl]-2-[2-(trifluoromethyl)pyridin-3-yl]-octahydrocyclopenta[c]pyrrol-5-amine

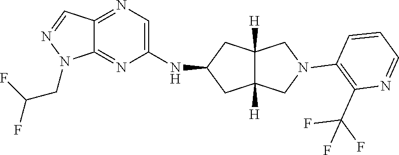 [SMILES: FC(F)Cn1ncc2ncc(N[C@H]3C[C@@H]4CN(c5cccnc5C(F)(F)F)C[C@@H]4C3)nc21]

Followed the General procedure A to afford the desired product as a white powder (12 mg, 37%). ¹H NMR (500 MHz, DMSO-d₆) δ 8.31 (dd, J=4.4, 1.3 Hz, 1H), 8.03 (s, 1H), 7.92 (s, 1H), 7.84-7.75 (m, 2H), 7.61 (dd, J=8.4, 4.4 Hz, 1H), 6.41 (tt, J=55.0, 4.0 Hz, 2H), 4.66 (td, J=14.7, 4.0 Hz, 2H), 4.53 (h, J=7.1 Hz, 1H), 3.18 (dd, J=9.2, 6.7 Hz, 2H), 3.08 (dd, J=9.5, 1.8 Hz, 2H), 2.89 (s, 2H), 1.98 (ddd, J=12.7, 6.7, 2.7 Hz, 2H), 1.83 (dt, J=12.8, 7.9 Hz, 2H). MS m/z: 454 [M+H]⁺.

### Example 38: (3aR,5S,6aS)—N-[1-(2,2-difluoroethyl)-1H-pyrazolo[3,4-b]pyrazin-6-yl]-2-[3-(trifluoromethyl)pyridin-2-yl]-octahydrocyclopenta[c]pyrrol-5-amine

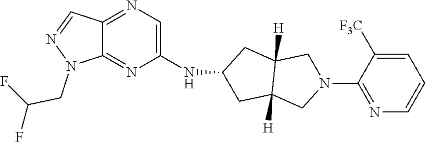 [SMILES: FC(F)Cn1ncc2ncc(N[C@@H]3C[C@@H]4CN(c5ncccc5C(F)(F)F)C[C@@H]4C3)nc21]

Followed the General procedure A to afford the desired product as a white solid (20 mg, 61%). ¹H NMR (500 MHz, DMSO-d₆) δ 8.38 (dd, J=4.7, 1.8 Hz, 1H), 8.03 (s, 1H), 7.97 (d, J=7.0 Hz, 1H), 7.97-7.94 (m, 1H), 7.89 (s, 1H), 6.89 (dd, J=7.8, 4.7 Hz, 1H), 6.43 (tt, J=54.9, 3.9 Hz, 1H), 4.66 (td, J=15.0, 3.8 Hz, 2H), 4.32-4.19 (m, 1H), 3.57-3.50 (m, 4H), 2.73 (dq, J=11.0, 7.4, 5.5 Hz, 2H), 2.44 (dt, J=12.4, 7.2 Hz, 2H), 1.38 (ddd, J=12.5, 10.0, 7.3 Hz, 2H). MS m/z: 454 [M+H]⁺.

### Example 39: (3aR,5S,6aS)—N-[1-(2,2-difluoroethyl)-1H-pyrazolo[3,4-b]pyrazin-6-yl]-2-[6-(trifluoromethyl)pyridin-3-yl]-octahydrocyclopenta[c]pyrrol-5-amine

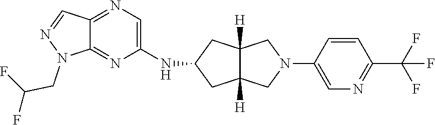 [SMILES: FC(F)Cn1ncc2ncc(N[C@@H]3C[C@@H]4CN(c5ccc(C(F)(F)F)nc5)C[C@@H]4C3)nc21]

Followed the General procedure A to afford the desired product as a white solid (21 mg, 64%). ¹H NMR (500 MHz, DMSO-d₆) δ 8.11 (d, J=2.8 Hz, 1H), 8.04 (s, 1H), 7.95 (d, J=6.8 Hz, 1H), 7.88 (s, 1H), 7.61 (d, J=8.7 Hz, 1H), 7.08 (dd, J=8.8, 2.9 Hz, 1H), 6.43 (tt, J=54.9, 3.8 Hz, 1H), 4.66 (td, J=15.0, 3.9 Hz, 2H), 4.32 (ddt, J=16.8, 9.5, 7.2 Hz, 1H), 3.48 (dd, J=10.2, 7.2 Hz, 2H), 3.38 (dd, J=10.3, 3.0 Hz, 2H), 2.85 (dq, J=11.3, 7.5, 5.6 Hz, 2H), 2.47 (t, J=8.1 Hz, 2H), 1.43 (ddd, J=12.8, 9.5, 7.2 Hz, 2H). MS m/z: 454 [M+H]⁺.

### Example 40: (3aR,5S,6aS)—N-[1-(2,2-difluoroethyl)-1H-pyrazolo[3,4-b]pyrazin-6-yl]-2-[5-(trifluoromethyl)pyridin-2-yl]-octahydrocyclopenta[c]pyrrol-5-amine

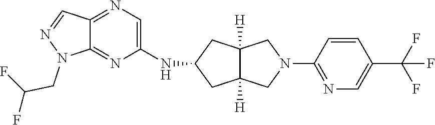 [SMILES: FC(F)Cn1ncc2ncc(N[C@H]3C[C@@H]4CN(c5ccc(C(F)(F)F)cn5)C[C@@H]4C3)nc21]

Followed the General procedure A to afford the desired product as a light-yellow oil (26 mg, 79%). ¹H NMR (500 MHz, DMSO-d₆) δ 8.39 (dt, J=2.7, 1.0 Hz, 1H), 8.03 (s, 1H), 7.93 (s, 1H), 7.92 (d, J=6.4 Hz, 1H), 7.76 (dd, J=9.0, 2.6 Hz, 1H), 6.61 (d, J=9.0 Hz, 1H), 6.40 (tt, J=55.0, 3.9 Hz, 1H), 4.62 (td, J=15.0, 3.9 Hz, 2H), 4.40 (h, J=6.3 Hz, 1H), 3.71 (dd, J=11.1, 7.6 Hz, 2H), 3.39-3.34 (m, 2H), 2.97 (dq, J=8.1, 4.4 Hz, 2H), 2.06-1.97 (m, 2H), 1.89 (ddd, J=13.4, 8.0, 5.8 Hz, 2H). MS m/z: 454 [M+H]⁺.

### Example 41: (3aR,5S,6aS)—N-[1-(2,2-difluoroethyl)-1H-pyrazolo[3,4-b]pyrazin-6-yl]-2-[3-(trifluoromethyl)pyridin-2-yl]-octahydrocyclopenta[c]pyrrol-5-amine

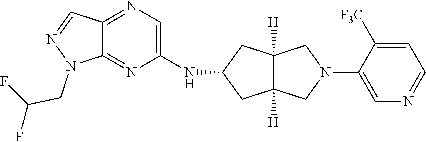 [SMILES: FC(F)Cn1ncc2ncc(N[C@H]3C[C@@H]4CN(c5cnccc5C(F)(F)F)C[C@@H]4C3)nc21]

Followed the General procedure A to afford the desired product as a white solid (23 mg, 70%). ¹H NMR (500 MHz, DMSO-d₆) δ 8.40 (dd, J=4.8, 1.8 Hz, 1H), 8.03 (s, 1H), 7.97 (dd, J=7.8, 1.8 Hz, 1H), 7.92 (s, 1H), 7.86 (d, J=6.7 Hz, 1H), 6.93 (dd, J=7.8, 4.7 Hz, 1H), 6.40 (tt, J=55.0, 4.0 Hz, 1H), 4.62 (td, J=14.8, 4.0 Hz, 2H), 4.43 (h, J=6.9 Hz, 1H), 3.56 (dd, J=10.8, 7.0 Hz, 2H), 3.42 (dd, J=10.9, 2.6 Hz, 2H), 2.88 (dt, J=7.5, 3.6 Hz, 2H), 1.99 (ddd, J=9.3, 8.0, 2.8 Hz, 2H), 1.82 (dt, J=12.6, 7.5 Hz, 2H). MS m/z: 454 [M+H]⁺.

### Example 42: (3aR,5S,6aS)—N-[1-(2,2-difluoroethyl)-1H-pyrazolo[3,4-b]pyrazin-6-yl]-2-[6-(trifluoromethyl)pyridin-3-yl]-octahydrocyclopenta[c]pyrrol-5-amine

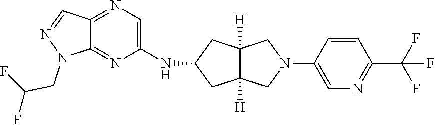 [SMILES: FC(F)Cn1ncc2ncc(N[C@H]3C[C@@H]4CN(c5ccc(C(F)(F)F)nc5)C[C@@H]4C3)nc21]

Followed the General procedure A to afford the desired product as a light-yellow solid (25 mg, 76%). ¹H NMR (500 MHz, DMSO-d₆) δ 8.09 (d, J=2.8 Hz, 1H), 8.03 (s, 1H), 7.94 (s, 1H), 7.91 (d, J=6.3 Hz, 1H), 7.61 (d, J=8.7 Hz, 1H), 7.07 (dd, J=8.7, 2.8 Hz, 1H), 6.40 (tt, J=54.9, 3.8 Hz, 1H), 4.63 (td, J=15.0, 3.8 Hz, 2H), 4.39 (h, J=6.3 Hz, 1H), 3.63-3.52 (m, 2H), 3.28-3.21 (m, 2H), 3.00 (h, J=4.3 Hz, 2H), 2.04-1.98 (m, 2H), 1.91 (ddd, J=13.5, 8.0, 6.0 Hz, 2H). MS m/z: 454 [M+H]⁺.

### Example 43: rac-2-[(3aR,6aS)-5-[1-(2,2-difluoroethyl)-1H-pyrazolo[3,4-b]pyrazin-6-yl]-octahydropyrrolo[3,4-c]pyrrol-2-yl]-4-(trifluoromethyl)pyridine

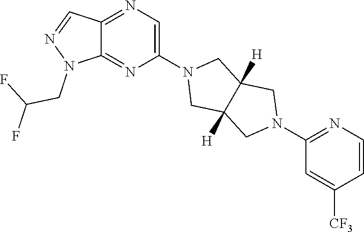 [SMILES: FC(F)Cn1ncc2ncc(N3C[C@H]4CN(c5cc(C(F)(F)F)ccn5)C[C@H]4C3)nc21]

Followed the General procedure A to afford the desired product as a white solid (18 mg, 45%). ¹H NMR (500 MHz, DMSO-d₆) δ 8.30 (d, J=5.2 Hz, 1H), 8.13 (s, 1H), 8.10 (s, 1H), 6.82 (d, J=7.0 Hz, 1H), 6.69 (s, 1H), 6.58-6.32 (m, 1H), 4.68 (td, J=14.9, 3.9 Hz, 2H), 3.90 (dd, J=11.3, 7.3 Hz, 2H), 3.76 (dd, J=11.0, 7.2 Hz, 2H), 3.62-3.53 (m, 2H), 3.52-3.44 (m, 2H), 3.26-3.16 (m, 2H). MS m/z: 440 [M+H]⁺.

### Example 44: rac-2-[(3aR,6aR)-5-[1-(2,2-difluoroethyl)-1H-pyrazolo[3,4-b]pyrazin-6-yl]-octahydropyrrolo[3,4-c]pyrrol-2-yl]-4-(trifluoromethyl)pyridine

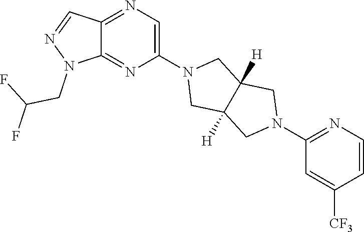 [SMILES: FC(F)Cn1ncc2ncc(N3C[C@@H]4CN(c5cc(C(F)(F)F)ccn5)C[C@H]4C3)nc21]

Followed the General procedure A to afford the desired product as a white solid (6 mg, 29%). ¹H NMR (500 MHz, DMSO-d₆) δ 8.31 (d, J=5.2 Hz, 1H), 8.14 (s, 1H), 8.13 (s, 1H), 6.82 (dd, J=5.3, 1.4 Hz, 1H), 6.70 (s, 1H), 6.45 (tt, J=54.9, 3.9 Hz, 1H), 4.69 (td, J=15.0, 3.9 Hz, 2H), 3.99 (t, J=8.0 Hz, 2H), 3.86 (s, 2H), 3.50-3.37 (m, 2H), 3.27 (t, J=10.0 Hz, 2H), 2.52 (s, 2H). MS m/z: 440 [M+H]⁺.

### Example 45: rac-2-[(3aR,6aS)-5-[1-(2,2-difluoroethyl)-1H-pyrazolo[3,4-b]pyrazin-6-yl]-octahydropyrrolo[3,4-c]pyrrol-2-yl]-6-(trifluoromethyl)pyridine

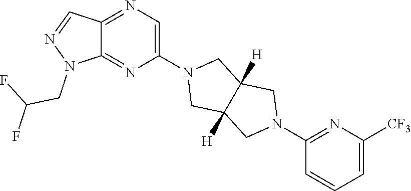 [SMILES: FC(F)Cn1ncc2ncc(N3C[C@H]4CN(c5cccc(C(F)(F)F)n5)C[C@H]4C3)nc21]

Followed the General procedure A to afford the desired product as a colorless oil (17 mg, 42%). ¹H NMR (500 MHz, DMSO-d₆) δ 8.12 (s, 1H), 8.09 (s, 1H), 7.70 (ddd, J=8.7, 7.2, 0.8 Hz, 1H), 6.98 (d, J=7.2 Hz, 1H), 6.75 (d, J=8.6 Hz, 1H), 6.45 (tt, J=55.0, 3.9 Hz, 1H), 4.67 (td, J=14.9, 3.9 Hz, 1H), 3.89 (dd, J=11.3, 7.3 Hz, 2H), 3.74 (dd, J=10.9, 7.2 Hz, 2H), 3.64-3.49 (m, 2H), 3.43 (dd, J=11.1, 3.7 Hz, 2H), 3.20 (s, 2H). MS m/z: 440 [M+H]⁺.

### Example 46: rac-2-[(3aR,6aR)-5-[1-(2,2-difluoroethyl)-1H-pyrazolo[3,4-b]pyrazin-6-yl]-octahydropyrrolo[3,4-c]pyrrol-2-yl]-6-(trifluoromethyl)pyridine

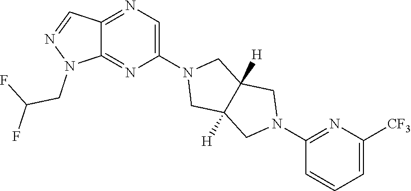 [SMILES: FC(F)Cn1ncc2ncc(N3C[C@@H]4CN(c5cccc(C(F)(F)F)n5)C[C@H]4C3)nc21]

Followed the General procedure A to afford the desired product as a white solid (27 mg, 67%). ¹H NMR (500 MHz, DMSO-d₆) δ 8.14 (s, 1H), 8.12 (s, 1H), 7.72 (dd, J=8.6, 7.2 Hz, 1H), 6.98 (d, J=7.2 Hz, 1H), 6.77 (d, J=8.6 Hz, 1H), 6.45 (tt, J=54.9, 3.9 Hz, 1H), 4.69 (td, J=15.0, 3.9 Hz, 2H), 3.99 (t, J=7.9 Hz, 2H), 3.82 (br s, 2H), 3.42 (br s, 2H), 3.26 (dd, J=10.7, 9.3 Hz, 2H), 2.52 (br s, 2H). MS m/z: 440 [M+H]⁺.

### Example 47: rac-5-[(3aR,6aS)-5-[1-(2,2-difluoroethyl)-1H-pyrazolo[3,4-b]pyrazin-6-yl]-octahydropyrrolo[3,4-c]pyrrol-2-yl]-2-(trifluoromethyl)pyridine

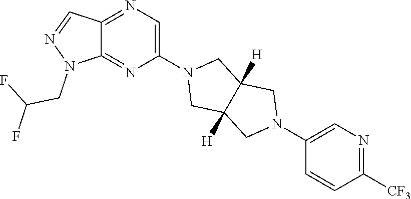 [SMILES: FC(F)Cn1ncc2ncc(N3C[C@H]4CN(c5ccc(C(F)(F)F)nc5)C[C@H]4C3)nc21]

Followed the General procedure A to afford the desired product as a white solid (20 mg, 48%). ¹H NMR (500 MHz, DMSO-d₆) δ 8.13 (s, 1H), 8.11 (s, 1H), 8.04 (d, J=2.8 Hz, 1H), 7.60 (d, J=8.8 Hz, 1H), 7.01 (dd, J=8.8, 2.9 Hz, 1H), 6.45 (tt, J=54.9, 3.9 Hz, 1H), 4.67 (td, J=14.9, 3.9 Hz, 2H), 3.90 (dd, J=11.3, 7.3 Hz, 2H), 3.67 (dd, J=10.2, 7.1 Hz, 2H), 3.57 (dd, J=11.2, 3.7 Hz, 2H), 3.38 (dd, J=10.5, 3.6 Hz, 2H), 3.24 (s, 2H). MS m/z: 440 [M+H]⁺.

### Example 48: rac-5-[(3aR,6aR)-5-[1-(2,2-difluoroethyl)-1H-pyrazolo[3,4-b]pyrazin-6-yl]-octahydropyrrolo[3,4-c]pyrrol-2-yl]-2-(trifluoromethyl)pyridine

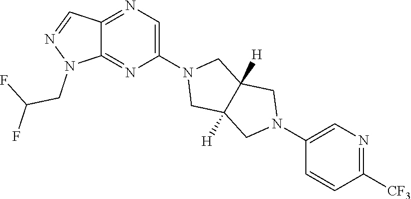 [SMILES: FC(F)Cn1ncc2ncc(N3C[C@@H]4CN(c5ccc(C(F)(F)F)nc5)C[C@H]4C3)nc21]

Followed the General procedure A to afford the desired product as a white solid (12 mg, 53%). ¹H NMR (500 MHz, DMSO-d₆) δ 8.14 (s, 1H), 8.13 (s, 1H), 8.08 (d, J=2.8 Hz, 1H), 7.61 (d, J=8.7 Hz, 1H), 7.04 (dd, J=8.8, 2.9 Hz, 1H), 6.46 (tt, J=54.9, 3.9 Hz, 1H), 4.69 (td, J=14.9, 3.9 Hz, 2H), 4.01 (dd, J=9.6, 6.1 Hz, 2H), 3.68 (dd, J=8.6, 6.3 Hz, 2H), 3.37 (br s, 2H), 3.27 (dd, J=10.5, 8.7 Hz, 2H), 2.57 (br s, 2H). MS m/z: 440 [M+H]⁺.

### Example 49: rac-3-[(3aR,6aS)-5-[1-(2,2-difluoroethyl)-1H-pyrazolo[3,4-b]pyrazin-6-yl]-octahydropyrrolo[3,4-c]pyrrol-2-yl]-2-(trifluoromethyl)pyridine

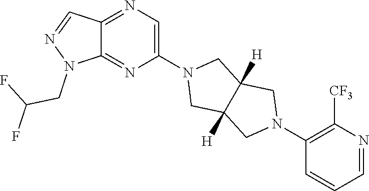 [SMILES: FC(F)Cn1ncc2ncc(N3C[C@@H]4CN(c5cccnc5C(F)(F)F)C[C@@H]4C3)nc21]

Followed the General procedure A to afford the desired product as a colorless oil (28 mg, 70%). ¹H NMR (500 MHz, DMSO-d₆) δ 8.16 (dd, J=4.3, 1.3 Hz, 1H), 8.15 (s, 1H), 8.13 (s, 1H), 7.62 (dd, J=8.6, 1.3 Hz, 1H), 7.52 (dd, J=8.6, 4.3 Hz, 1H), 6.60-6.33 (m, 1H), 4.69 (td, J=14.9, 3.9 Hz, 2H), 3.91 (dd, J=11.4, 7.5 Hz, 2H), 3.62-3.51 (m, 4H), 3.29 (dd, J=9.8, 3.2 Hz, 2H), 3.23-3.12 (m, 2H). MS m/z: 440 [M+H]⁺.

### Example 50: rac-3-[(3aR,6aR)-5-[1-(2,2-difluoroethyl)-1H-pyrazolo[3,4-b]pyrazin-6-yl]-octahydropyrrolo[3,4-c]pyrrol-2-yl]-2-(trifluoromethyl)pyridine

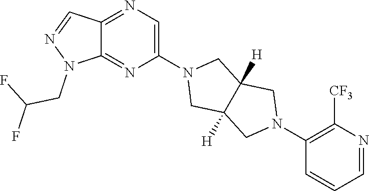 [SMILES: FC(F)Cn1ncc2ncc(N3C[C@@H]4CN(c5cccnc5C(F)(F)F)C[C@H]4C3)nc21]

Followed the General procedure A to afford the desired product as a white solid (21 mg, 52%). ¹H NMR (500 MHz, DMSO-d₆) δ 8.14 (s, 1H), 8.11 (s, 1H), 8.06 (dd, J=3.4, 2.0 Hz, 1H), 7.51-7.45 (m, 2H), 6.46 (tt, J=54.9, 3.9 Hz, 1H), 4.68 (td, J=14.9, 3.9 Hz, 2H), 3.98 (s, 2H), 3.54-3.34 (m, 8H). MS m/z: 440 [M+H]⁺.

### Example 51: (3aR,5S,6aS)—N-[1-(2,2-difluoroethyl)-1H-pyrazolo[3,4-b]pyrazin-6-yl]-2-[5-(trifluoromethyl)pyridin-2-yl]-octahydrocyclopenta[c]pyrrol-5-amine

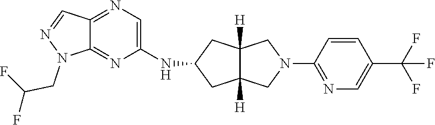 [SMILES: FC(F)Cn1ncc2ncc(N[C@@H]3C[C@@H]4CN(c5ccc(C(F)(F)F)cn5)C[C@@H]4C3)nc21]

Followed the General procedure A to afford the desired product as a colorless oil (20 mg, 61%). ¹H NMR (500 MHz, DMSO-d₆) δ 8.39 (dt, J=2.7, 1.0 Hz, 1H), 8.03 (s, 1H), 7.96 (d, J=6.9 Hz, 1H), 7.88 (s, 1H), 7.76 (dd, J=9.1, 2.6 Hz, 1H), 6.62 (d, J=9.0 Hz, 1H), 6.43 (tt, J=54.9, 3.8 Hz, 1H), 4.66 (td, J=15.0, 3.9 Hz, 2H), 4.33 (ddt, J=16.7, 9.3, 7.4 Hz, 1H), 3.68-3.57 (m, 2H), 3.52-3.42 (m, 2H), 2.81 (dd, J=8.3, 4.7 Hz, 2H), 2.49-2.40 (m, 2H), 1.46-1.40 (m, 2H). MS m/z: 454 [M+H]⁺.

### Example 52: (3aR,5S,6aS)—N-[1-(2,2-difluoroethyl)-1H-pyrazolo[3,4-b]pyrazin-6-yl]-2-[5-(trifluoromethyl)pyridin-3-yl]-octahydrocyclopenta[c]pyrrol-5-amine

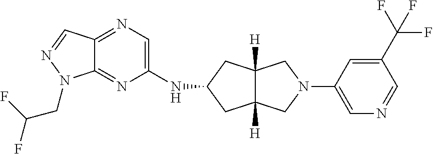 [SMILES: FC(F)Cn1ncc2ncc(N[C@@H]3C[C@@H]4CN(c5cncc(C(F)(F)F)c5)C[C@@H]4C3)nc21]

Followed the General procedure A to afford the desired product as a white solid (12 mg, 37%). ¹H NMR (500 MHz, DMSO-d₆) δ 8.28 (d, J=2.7 Hz, 1H), 8.20-8.14 (m, 1H), 8.04 (s, 1H), 7.94 (d, J=6.9 Hz, 1H), 7.88 (s, 1H), 7.20 (t, J=2.4 Hz, 1H), 6.43 (tt, J=54.9, 3.8 Hz, 1H), 4.66 (td, J=15.0, 3.9 Hz, 2H), 4.30 (tq, J=9.8, 7.2 Hz, 1H), 3.41 (qd, J=10.1, 5.1 Hz, 4H), 2.83 (tt, J=7.8, 3.9 Hz, 2H), 2.49-2.43 (m, 2H), 1.42 (ddd, J=12.7, 9.7, 7.3 Hz, 2H). MS m/z: 454 [M+H]⁺.

### Example 53: (3aR,5S,6aS)—N-[1-(2,2-difluoroethyl)-1H-pyrazolo[3,4-b]pyrazin-6-yl]-2-[5-(trifluoromethyl)pyridin-3-yl]-octahydrocyclopenta[c]pyrrol-5-amine

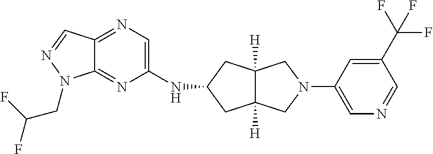 [SMILES: FC(F)Cn1ncc2ncc(N[C@H]3C[C@@H]4CN(c5cncc(C(F)(F)F)c5)C[C@@H]4C3)nc21]

Followed the General procedure A to afford the desired product as a off-white solid (8 mg, 24%). ¹H NMR (500 MHz, DMSO-d₆) δ 8.28 (d, J=2.7 Hz, 1H), 8.19 (s, 1H), 8.04 (s, 1H), 7.94 (s, 1H), 7.91 (d, J=6.4 Hz, 1H), 7.19 (t, J=2.4 Hz, 1H), 6.54-6.28 (m, 1H), 4.63 (td, J=15.0, 3.8 Hz, 2H), 4.44-4.35 (m, 1H), 3.58-3.52 (m, 2H), 3.28-3.23 (m, 2H), 3.05-2.95 (m, 2H), 2.04-1.97 (m, 2H), 1.95-1.86 (m, 2H). MS m/z: 454 [M+H]⁺.

### Example 54: (3aR,5S,6aS)—N-[1-(2,2-difluoroethyl)-1H-pyrazolo[3,4-b]pyrazin-6-yl]-N-methyl-2-[5-(trifluoromethyl)pyridin-3-yl]-octahydrocyclopenta[c]pyrrol-5-amine

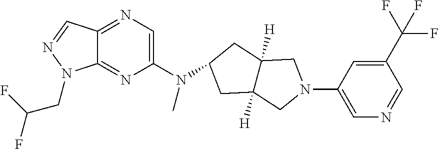 [SMILES: CN(c1cnc2cnn(CC(F)F)c2n1)[C@H]1C[C@@H]2CN(c3cncc(C(F)(F)F)c3)C[C@@H]2C1]

Followed the General procedure A to afford the desired product as a light-yellow solid (18 mg, 87%). ¹H NMR (500 MHz, DMSO-d₆) δ 8.36 (s, 1H), 8.28 (d, J=2.7 Hz, 1H), 8.18 (d, J=1.8 Hz, 1H), 8.11 (s, 1H), 7.19 (t, J=2.4 Hz, 1H), 6.37 (tt, J=55.0, 3.9 Hz, 1H), 5.13 (p, J=8.5 Hz, 1H), 4.61 (td, J=14.9, 3.9 Hz, 2H), 3.59 (dd, J=10.0, 7.9 Hz, 2H), 3.27 (dd, J=10.2, 3.7 Hz, 2H), 3.07 (s, 3H), 2.99 (s, 2H), 2.10-2.00 (m, 2H), 1.90-1.79 (m, 2H). MS m/z: 468 [M+H]⁺.

### Example 55: rac-(3aR,6aS)-2-[1-(2,2-difluoroethyl)-1H-pyrazolo[3,4-b]pyrazin-6-yl]-5-[4-(trifluoromethyl)pyridin-2-yl]-octahydropyrrolo[3,4-c]pyrrol-1-one

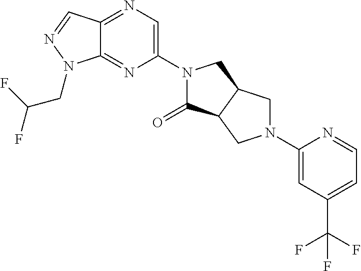 [SMILES: O=C1[C@@H]2CN(c3cc(C(F)(F)F)ccn3)C[C@@H]2CN1c1cnc2cnn(CC(F)F)c2n1]

Followed the General procedure C to afford the desired product as a white solid (12 g, 55%). ¹H NMR (500 MHz, DMSO-d₆) δ 9.66 (s, 1H), 8.50 (s, 1H), 8.32 (d, J=5.2 Hz, 1H), 6.87 (dd, J=5.3, 1.5 Hz, 1H), 6.77 (s, 1H), 6.53 (tt, J=54.6, 3.6 Hz, 2H), 4.88 (td, J=15.1, 3.6 Hz, 2H), 4.29-4.19 (m, 1H), 4.13 (dd, J=11.6, 1.6 Hz, 1H), 4.01 (d, J=9.6 Hz, 1H), 3.90 (dd, J=11.0, 8.9 Hz, 1H), 3.73-3.63 (m, 2H), 3.43 (dd, J=11.1, 6.7 Hz, 1H), 3.33 (s, 1H). MS m/z: 454 [M+H]⁺.

### Example 56: rac-2-[(3aR,6aR)-4-[1-(2,2-difluoroethyl)-1H-pyrazolo[3,4-b]pyrazin-6-yl]-octahydropyrrolo[3,2-b]pyrrol-1-yl]-4-(trifluoromethyl)pyridine

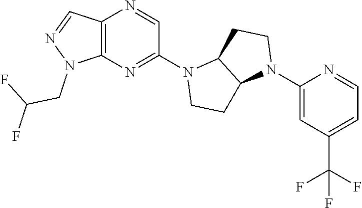 [SMILES: FC(F)Cn1ncc2ncc(N3CC[C@H]4[C@@H]3CCN4c3cc(C(F)(F)F)ccn3)nc21]

Followed the General procedure A to afford the desired product as a light-yellow oil (23 mg, 77%). ¹H NMR (500 MHz, DMSO-d₆) δ 8.35 (d, J=5.2 Hz, 1H), 8.20 (s, 1H), 8.17 (s, 1H), 6.87 (dd, J=5.2, 1.5 Hz, 1H), 6.79 (s, 1H), 6.46 (tt, J=54.9, 3.8 Hz, 1H), 4.84 (td, J=5.9, 3.3 Hz, 1H), 4.80-4.76 (m, 1H), 4.71 (td, J=15.1, 3.8 Hz, 2H), 3.90 (ddd, J=11.6, 8.2, 3.8 Hz, 1H), 3.73 (q, J=7.2, 3.8 Hz, 1H), 3.51 (td, J=10.8, 9.9, 7.2 Hz, 1H), 3.45-3.39 (m, 1H), 2.36-2.16 (m, 4H). MS m/z: 440 [M+H]⁺.

### Example 57: rac-2-[(3aR,6aR)-4-[1-(2,2-difluoroethyl)-1H-pyrazolo[3,4-b]pyrazin-6-yl]-octahydropyrrolo[3,2-b]pyrrol-1-yl]-6-(trifluoromethyl)pyridine

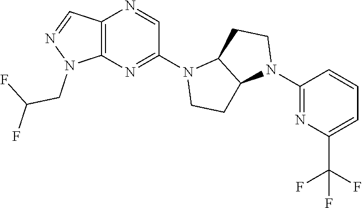 [SMILES: FC(F)Cn1ncc2ncc(N3CC[C@H]4[C@@H]3CCN4c3cccc(C(F)(F)F)n3)nc21]

Followed the General procedure A to afford the desired product as a white solid (28 mg (75%)). ¹H NMR (500 MHz, DMSO-d₆) δ 8.19 (s, 1H), 8.17 (s, 1H), 7.75 (ddd, J=8.3, 7.4, 0.8 Hz, 1H), 7.03 (d, J=7.2 Hz, 1H), 6.87 (d, J=8.6 Hz, 1H), 6.46 (tt, J=54.9, 3.8 Hz, 1H), 4.84 (q, J=5.1, 4.6 Hz, 1H), 4.71 (td, J=15.0, 3.8 Hz, 3H), 3.89 (d, J=9.2 Hz, 1H), 3.72 (s, 1H), 3.51 (q, J=9.0 Hz, 1H), 3.44-3.37 (m, 1H), 2.35-2.15 (m, 4H). MS m/z: 440 [M+H]⁺.

### Example 58: rac-2-[(3aR,6aR)-1-[1-(2,2-difluoroethyl)-1H-pyrazolo[3,4-b]pyrazin-6-yl]-octahydropyrrolo[3,4-b]pyrrol-5-yl]-4-(trifluoromethyl)pyridine

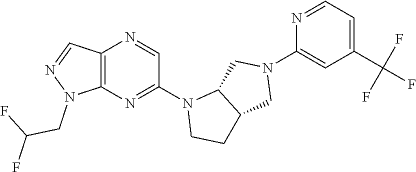 [SMILES: FC(F)Cn1ncc2ncc(N3CC[C@H]4CN(c5cc(C(F)(F)F)ccn5)C[C@H]43)nc21]

Followed the General procedure A to afford the desired product as a light-yellow solid (30 mg, 80%). ¹H NMR (500 MHz, DMSO-d₆) δ 8.28 (d, J=5.2 Hz, 1H), 8.16 (d, J=1.7 Hz, 2H), 6.82 (dd, J=5.2, 1.5 Hz, 1H), 6.70 (s, 1H), 6.46 (tt, J=54.9, 3.8 Hz, 1H), 4.80-4.65 (m, 3H), 3.94 (s, 1H), 3.78 (t, J=6.8 Hz, 2H), 3.73 (dd, J=11.1, 8.1 Hz, 1H), 3.63 (d, J=11.8 Hz, 1H), 3.54-3.45 (m, 1H), 3.23 (s, 1H), 2.21 (dd, J=12.9, 7.0 Hz, 1H), 1.97 (dd, J=12.3, 6.1 Hz, 1H). MS m/z: 440 [M+H]⁺.

### Example 59: rac-2-[(3aR,6aR)-1-[1-(2,2-difluoroethyl)-1H-pyrazolo[3,4-b]pyrazin-6-yl]-octahydropyrrolo[3,4-b]pyrrol-5-yl]-6-(trifluoromethyl)pyridine

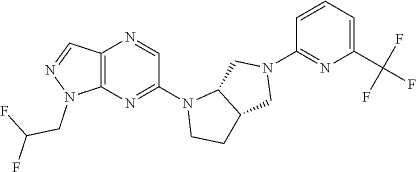 [SMILES: FC(F)Cn1ncc2ncc(N3CC[C@H]4CN(c5cccc(C(F)(F)F)n5)C[C@H]43)nc21]

Followed the General procedure A to afford the desired product as a white solid (33 mg, 88%). ¹H NMR (500 MHz, DMSO-d₆) δ 8.17 (d, J=2.0 Hz, 2H), 7.69 (t, J=7.9 Hz, 1H), 6.98 (d, J=7.2 Hz, 1H), 6.76 (d, J=8.6 Hz, 1H), 6.46 (tt, J=55.0, 3.8 Hz, 1H), 4.83-4.63 (m, 3H), 3.94 (s, 1H), 3.84-3.75 (m, 2H), 3.72 (dd, J=11.1, 8.1 Hz, 1H), 3.57 (d, J=11.8 Hz, 1H), 3.45 (dd, J=11.1, 5.0 Hz, 1H), 3.22 (s, 1H), 2.21 (dd, J=13.0, 6.9 Hz, 1H), 2.05-1.94 (m, 1H). MS m/z: 440 [M+H]⁺.

### Example 60: rac-2-[(3aR,6aR)-5-[1-(2,2-difluoroethyl)-1H-pyrazolo[3,4-b]pyrazin-6-yl]-octahydropyrrolo[2,3-c]pyrrol-1-yl]-4-(trifluoromethyl)pyridine

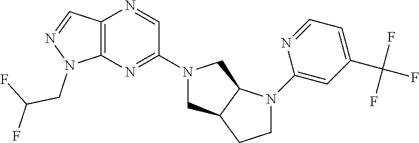 [SMILES: FC(F)Cn1ncc2ncc(N3C[C@@H]4CCN(c5cc(C(F)(F)F)ccn5)[C@@H]4C3)nc21]

Followed the General procedure A to afford the desired product as a white solid (21 mg, 78%). ¹H NMR (500 MHz, DMSO-d₆) δ 8.36 (d, J=5.2 Hz, 1H), 8.13 (s, 1H), 8.10 (s, 1H), 6.88 (dd, J=5.3, 1.5 Hz, 1H), 6.76 (s, 1H), 6.43 (tt, J=54.9, 3.8 Hz, 1H), 4.69 (s, 1H), 4.66 (td, J=15.1, 4.2 Hz, 2H), 3.99 (dd, J=12.1, 6.5 Hz, 1H), 3.86 (dd, J=11.4, 8.1 Hz, 1H), 3.75-3.55 (m, 4H), 3.21 (d, J=7.1 Hz, 1H), 2.20 (dq, J=13.9, 7.2, 6.4 Hz, 1H), 1.98 (tt, J=12.5, 5.9 Hz, 1H). MS m/z: 440 [M+H]⁺.

### Example 61: rac-2-[(3aR,6aR)-5-[1-(2,2-difluoroethyl)-1H-pyrazolo[3,4-b]pyrazin-6-yl]-octahydropyrrolo[2,3-c]pyrrol-1-yl]-6-(trifluoromethyl)pyridine

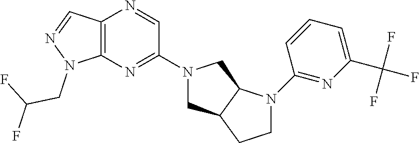 [SMILES: FC(F)Cn1ncc2ncc(N3C[C@@H]4CCN(c5cccc(C(F)(F)F)n5)[C@@H]4C3)nc21]

Followed the General procedure A to afford the desired product as a white solid (25 mg, 93%). ¹H NMR (500 MHz, DMSO-d₆) δ 8.13 (s, 1H), 8.08 (s, 1H), 7.75 (t, J=7.9 Hz, 1H), 7.03 (d, J=7.2 Hz, 1H), 6.84 (d, J=8.6 Hz, 1H), 6.43 (tt, J=54.9, 3.8 Hz, 1H), 4.65 (td, J=15.9, 15.4, 4.3 Hz, 3H), 3.99 (dd, J=12.3, 6.5 Hz, 1H), 3.87 (dd, J=11.3, 8.1 Hz, 1H), 3.73-3.53 (m, 4H), 3.22 (q, J=6.7 Hz, 1H), 2.19 (dq, J=14.0, 7.1 Hz, 1H), 1.96 (dq, J=12.4, 6.1 Hz, 1H). MS m/z: 440 [M+H]⁺.

### Example 62: rac-(3aR,6aS)-2-[1-(2,2-difluoroethyl)-1H-pyrazolo[3,4-b]pyrazin-6-yl]-5-[6-(trifluoromethyl)pyridin-2-yl]-octahydropyrrolo[3,4-c]pyrrol-1-one

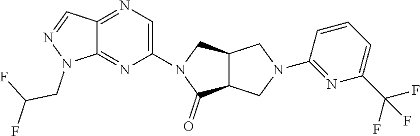 [SMILES: O=C1[C@@H]2CN(c3cccc(C(F)(F)F)n3)C[C@@H]2CN1c1cnc2cnn(CC(F)F)c2n1]

Followed the General procedure C to afford the desired product as a white solid (22 mg, 77%). ¹H NMR (500 MHz, DMSO-d₆) δ 9.66 (s, 1H), 8.50 (s, 1H), 7.73 (dd, J=8.8, 7.4 Hz, 1H), 7.02 (d, J=7.2 Hz, 1H), 6.83 (d, J=8.6 Hz, 1H), 6.53 (tt, J=54.6, 3.7 Hz, 1H), 4.88 (td, J=15.1, 3.6 Hz, 2H), 4.24 (dd, J=11.5, 6.2 Hz, 1H), 4.16 (dd, J=11.5, 1.4 Hz, 1H), 4.01-3.83 (m, 2H), 3.72-3.61 (m, 2H), 3.40-3.37 (m, 1H), 3.34-3.29 (m, 1H). MS m/z: 454 [M+H]⁺.

### Example 63: 1-(2,2-difluoroethyl)-6-(5-(3-(trifluoromethyl)pyridin-2-yl)-4,5,6,7-tetrahydro-2H-pyrazolo[4,3-c]pyridin-2-yl)-1H-pyrazolo[3,4-b]pyrazine

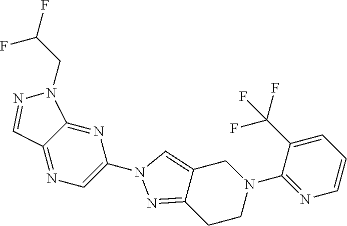 [SMILES: FC(F)Cn1ncc2ncc(-n3cc4c(n3)CCN(c3ncccc3C(F)(F)F)C4)nc21]

Step 1: tert-butyl 2-(1-(2,2-difluoroethyl)-1H-pyrazolo[3,4-b]pyrazin-6-yl)-2,4,6,7-tetrahydro-5H-pyrazolo[4,3-c]pyridine-5-carboxylate; tert-butyl 1-(1-(2,2-difluoroethyl)-1H-pyrazolo[3,4-b]pyrazin-6-yl)-1,4,6,7-tetrahydro-5H-pyrazolo[4,3-c]pyridine-5-carboxylate: To a stirred solution of 6-chloro-1-(2,2-difluoroethyl)-1H-pyrazolo[3,4-b]pyrazine (250 mg, 1.15 mmol, 1.00 equiv.) and tert-butyl 1,3a,4,6,7,7a-hexahydro-5H-pyrazolo[4,3-c]pyridine-5-carboxylate (309 mg, 1.38 mmol, 1.20 equiv.) in DMF (3 mL) was added Cs₂CO₃ (1.12 g, 3.39 mmol, 3.00 equiv.). Desired product could be detected by LCMS. The mixture was diluted with EtOAc (15 mL), washed with water and brine, dried, evaporated, and purified with Combi-flash (40 g silico gel column), eluted with gradient of Hex:EtOAc. The fractions were collected, and concentrated to give the crude mixture. The crude mixture was further purified by prep-HPLC to give tert-butyl 1-(1-(2,2-difluoroethyl)-1H-pyrazolo[3,4-b]pyrazin-6-yl)-2,4,6,7-tetrahydro-5H-pyrazolo[4,3-c]pyridine-5-carboxylate (60 mg, 12%) and tert-butyl 1-(1-(2,2-difluoroethyl)-1H-pyrazolo[3,4-b]pyrazin-6-yl)-1,4,6,7-tetrahydro-5H-pyrazolo[4,3-c]pyridine-5-carboxylate (200 mg, 43%) as off-white solids. MS m/z: 406 [M+H]⁺.

Step 2: 1-(2,2-difluoroethyl)-6-(4,5,6,7-tetrahydro-2H-pyrazolo[4,3-c]pyridin-2-yl)-1H-pyrazolo[3,4-b]pyrazine hydrochloride: Follow the general procedure B using tert-butyl 2-(1-(2,2-difluoroethyl)-1H-pyrazolo[3,4-b]pyrazin-6-yl)-2,4,6,7-tetrahydro-5H-pyrazolo[4,3-c]pyridine-5-carboxylate (60 mg, 0.148 mmol, 1.00 equiv.) as the starting material to give the crude product 1-(2,2-difluoroethyl)-6-(4,5,6,7-tetrahydro-2H-pyrazolo[4,3-c]pyridin-2-yl)-1H-pyrazolo[3,4-b]pyrazine hydrochloride (60 mg). MS m/z: 306 [M+H]⁺.

Step 3: 1-(2,2-difluoroethyl)-6-(5-(3-(trifluoromethyl)pyridin-2-yl)-4,5,6,7-tetrahydro-2H-pyrazolo[4,3-c]pyridin-2-yl)-1H-pyrazolo[3,4-b]pyrazine: To a stirred solution of 1-(2,2-difluoroethyl)-6-(4,5,6,7-tetrahydro-2H-pyrazolo[4,3-c]pyridin-2-yl)-1H-pyrazolo[3,4-b]pyrazine hydrochloride (60 mg, 0.148 mmol, 1.00 equiv.) and 2-bromo-3-(trifluoromethyl)pyridine (39.2 mg, 0.178 mmol, 1.2 equiv.) in dioxane (1 mL) were added Na₂CO₃ (47.0 mg, 0.444 mmol, 3.00 equiv.) and Pd-PEPPSI-IPentCl 2-methylpyridine (o-picoline) (12.1 mg, 0.015 mmol, 0.10 equiv.). The resulting mixture was stirred for overnight at 100° C. under nitrogen atmosphere. The reaction was monitored by LCMS. The mixture was filtered through Celite, and washed with EtOAc (10 mL). The organic layer was combined, evaporated, and purified with a silico gel column, eluted with gradient of Hexane/EtOAc. The fractions were collected, and concentrated to give 1-(2,2-difluoroethyl)-6-(5-(3-(trifluoromethyl)pyridin-2-yl)-4,5,6,7-tetrahydro-2H-pyrazolo[4,3-c]pyridin-2-yl)-1H-pyrazolo[3,4-b]pyrazine (11 mg, 16.5%) as a white solid. ¹H NMR (400 MHz, DMSO-d₆) δ 9.29 (s, 1H), 8.61-8.50 (m, 2H), 8.13-8.10 (m, 1H), 7.87 (s, 1H), 7.23-7.19 (m, 1H), 6.68-6.40 (m, 1H), 5.00-4.89 (m, 2H), 4.38 (s, 2H), 3.63-3.60 (m, 2H), 3.51-3.49 (m, 2H). MS m/z: 451 [M+H]⁺.

### Example 64: 1-(2,2-difluoroethyl)-6-(5-(3-(trifluoromethyl)pyridin-2-yl)-4,5,6,7-tetrahydro-1H-pyrazolo[4,3-c]pyridin-1-yl)-1H-pyrazolo[3,4-b]pyrazine

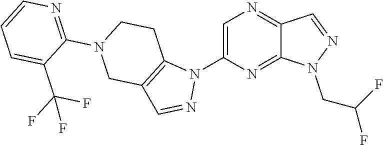 [SMILES: FC(F)Cn1ncc2ncc(-n3ncc4c3CCN(c3ncccc3C(F)(F)F)C4)nc21]

Step 1: 1-(2,2-difluoroethyl)-6-(4,5,6,7-tetrahydro-1H-pyrazolo[4,3-c]pyridin-1-yl)-1H-pyrazolo[3,4-b]pyrazine hydrochloride: Follow the general procedure B using tert-butyl 1-(1-(2,2-difluoroethyl)-1H-pyrazolo[3,4-b]pyrazin-6-yl)-1,4,6,7-tetrahydro-5H-pyrazolo[4,3-c]pyridine-5-carboxylate (60 mg, 0.148 mmol, 1.00 equiv.) as the starting material to give the crude product 1-(2,2-difluoroethyl)-6-(4,5,6,7-tetrahydro-1H-pyrazolo[4,3-c]pyridin-1-yl)-1H-pyrazolo[3,4-b]pyrazine hydrochloride (60 mg). MS m/z: 306 [M+H]⁺

Step 2: 1-(2,2-difluoroethyl)-6-(5-(3-(trifluoromethyl)pyridin-2-yl)-4,5,6,7-tetrahydro-1H-pyrazolo[4,3-c]pyridin-1-yl)-1H-pyrazolo[3,4-b]pyrazine: To a stirred solution of 1-(2,2-difluoroethyl)-6-(4,5,6,7-tetrahydro-2H-pyrazolo[4,3-c]pyridin-1-yl)-1H-pyrazolo[3,4-b]pyrazine hydrochloride (60 mg, 0.148 mmol, 1.00 equiv.) and 2-bromo-3-(trifluoromethyl)pyridine (39.2 mg, 0.178 mmol, 1.2 equiv.) in dioxane (1 mL) were added Na₂CO₃ (47.0 mg, 0.444 mmol, 3.00 equiv.) and Pd-PEPPSI-IPentCl 2-methylpyridine (o-picoline) (12.1 mg, 0.015 mmol, 0.10 equiv.). The resulting mixture was stirred for overnight at 100° C. under nitrogen atmosphere. The reaction was monitored by LCMS. The mixture was filtered through Celite, and washed with EtOAc (10 mL). The organic layer was combined, evaporated, and purified with a silico gel column, eluted with gradient of Hexane/EtOAc. The fractions were collected, and concentrated to give 1-(2,2-difluoroethyl)-6-(5-(3-(trifluoromethyl)pyridin-1-yl)-4,5,6,7-tetrahydro-2H-pyrazolo[4,3-c]pyridin-2-yl)-1H-pyrazolo[3,4-b]pyrazine (20 mg, 18.9%) as a white solid. ¹H NMR (400 MHz, Methanol-d₄) δ 9.30 (s, 1H), 8.56 (s, 1H), 8.53-8.49 (m, 1H), 8.35 (s, 1H), 8.06-8.04 (m, 1H), 7.19-7.16 (m, 1H), 6.51-6.22 (m, 2H), 4.48 (s, 2H), 3.65-3.62 (m, 2H), 3.07-3.04 (m, 2H). MS m/z: 451 [M+H]⁺.

### Example 65: 5-(1-(2,2-difluoroethyl)-1H-pyrazolo[3,4-b]pyrazin-6-yl)-2-(6-(trifluoromethyl)pyridin-2-yl)octahydro-3H-pyrrolo[3,4-c]pyridin-3-one

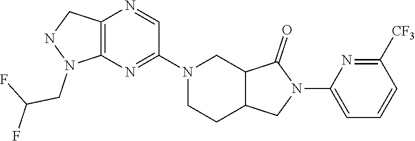 [SMILES: O=C1C2CN(c3cnc4c(n3)N(CC(F)F)NC4)CCC2CN1c1cccc(C(F)(F)F)n1]

Step 1: 5-(1-(2,2-difluoroethyl)-1H-pyrazolo[3,4-b]pyrazin-6-yl)octahydro-3H-pyrrolo[3,4-c]pyridin-3-one: Followed the general procedure E using 6-chloro-1-(2,2-difluoroethyl)-1H-pyrazolo[3,4-b]pyrazine (50 mg, 0.229 mmol, 1.00 equiv.) and octahydro-3H-pyrrolo[3,4-c]pyridin-3-one (35.3 mg, 0.252 mmol, 1.10 equiv.) as the starting materials to give 5-(1-(2,2-difluoroethyl)-1H-pyrazolo[3,4-b]pyrazin-6-yl)octahydro-3H-pyrrolo[3,4-c]pyridin-3-one (50 mg, 67%). MS m/z: 323 [M+H]⁺.

Step 2: 5-(1-(2,2-difluoroethyl)-1H-pyrazolo[3,4-b]pyrazin-6-yl)-2-(6-(trifluoromethyl)pyridin-2-yl)octahydro-3H-pyrrolo[3,4-c]pyridin-3-one: Followed the general procedure E using 5-(1-(2,2-difluoroethyl)-1H-pyrazolo[3,4-b]pyrazin-6-yl)octahydro-3H-pyrrolo[3,4-c]pyridin-3-one (50 mg, 0.155 mmol, 1.00 equiv.) and 2-fluoro-6-(trifluoromethyl)pyridine (30.7 mg, 0.186 mmol, 1.20 equiv.) as the starting materials to give 5-(1-(2,2-difluoroethyl)-1H-pyrazolo[3,4-b]pyrazin-6-yl)-2-(6-(trifluoromethyl)pyridin-2-yl)octahydro-3H-pyrrolo[3,4-c]pyridin-3-one (7 mg, 9.7%). MS m/z: 323 [M+H]⁺. ¹H NMR (400 MHz, DMSO-d₆) δ 8.54-8.45 (m, 2H), 8.13 (s, 1H), 8.05 (t, J=8.0 Hz, 1H), 7.62 (d, J=7.4 Hz, 1H), 6.44 (tt, J=55.0, 3.8 Hz, 1H), 4.69 (td, J=15.1, 3.6 Hz, 3H), 4.35 (d, J=12.9 Hz, 1H), 4.04 (dt, J=10.6, 4.4 Hz, 1H), 3.84 (d, J=11.1 Hz, 1H), 3.54 (dt, J=13.9, 3.9 Hz, 1H), 3.24-3.08 (m, 2H), 2.77 (t, J=8.4 Hz, 1H, 2.07-1.99 (m, 1H), 1.55-1.41 (m, 1H). MS m/z: 468 [M+H]⁺.

### Example 66: 5-(1-(2,2-difluoroethyl)-1H-pyrazolo[3,4-b]pyrazin-6-yl)-2-(6-(trifluoromethyl)pyridin-2-yl)octahydro-6H-pyrrolo[3,4-c]pyridin-6-one

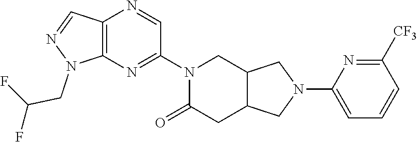 [SMILES: O=C1CC2CN(c3cccc(C(F)(F)F)n3)CC2CN1c1cnc2cnn(CC(F)F)c2n1]

Step 1: tert-butyl 5-(hydroxyimino)hexahydrocyclopenta[c]pyrrole-2(1H)-carboxylate: To a stirred mixture of tert-butyl 5-oxohexahydrocyclopenta[c]pyrrole-2(1H)-carboxylate (200 mg, 0.800 mmol, 1.00 equiv.) and hydroxylamine hydrochloride (66.9 mg, 0.96 mmol, 1.2 equiv.) in EtOH: H₂O=10:1 (5 mL) was added K₂CO₃ (220 mg, 1.60 mmol, 2.0 equiv.) in portions at room temperature. The resulting mixture was stirred for overnight at 80° C. Desired product could be detected by LCMS. The resulting mixture was purified by reverse phase flash with the following conditions (column, C18 silica gel; mobile phase, MeCN in water, 30% to 60% gradient in 15 min; detector, UV 220 nm.) to afford tert-butyl 5-(hydroxyimino)hexahydrocyclopenta[c]pyrrole-2(1H)-carboxylate (140 mg, 72%) as a colorless oil. MS m/z: 241 [M+H]⁺.

Step 2: tert-butyl 6-oxooctahydro-2H-pyrrolo[3,4-c]pyridine-2-carboxylate: To a stirred solution of tert-butyl 5-(hydroxyimino)hexahydrocyclopenta[c]pyrrole-2(1H)-carboxylate (140 mg, 0.583 mmol, 1.00 equiv.) in dioxane (2.00 mL) was added Thionyl chloride (104 mg, 0.874 mmol, 1.50 equiv.) dropwise at 0° C. under N₂ atmosphere. The resulting mixture was stirred for 4 h at room temperature. Desired product could be detected by LCMS. The resulting mixture was concentrated under vacuum, neutralized with saturated NaHCO₃ aq., and was extracted with DCM (3×10 mL). The combined organic layers were washed with water and brine, dried, evaporated, and purified by reversed phase Combi-flash with the following conditions: column, C18 silica gel; mobile phase, MeCN in water, 30% to 60% gradient in 15 min; detector, UV 220 nm. This resulted in tert-butyl 6-oxooctahydro-2H-pyrrolo[3,4-c]pyridine-2-carboxylate (60 mg, 42%) as a colorless oil. MS m/z: 241 [M+H]⁺.

Step 3: tert-butyl 5-(1-(2,2-difluoroethyl)-1H-pyrazolo[3,4-b]pyrazin-6-yl)-6-oxooctahydro-2H-pyrrolo[3,4-c]pyridine-2-carboxylate: To a stirred solution of tert-butyl 6-oxooctahydro-2H-pyrrolo[3,4-c]pyridine-2-carboxylate (60 mg, 0.250 mmol, 1.00 equiv.) and 6-chloro-1-(2,2-difluoroethyl)-1H-pyrazolo[3,4-b]pyrazine (65.4 mg, 0.30 mmol, 1.20 equiv.) in dioxane (1 mL) were added Cs₂CO₃ (246 mg, 0.750 mmol, 3.00 equiv.) and Xphos Pd G3 (21.1 mg, 0.025 mmol, 0.10 equiv.). The resulting mixture was stirred for overnight at 100° C. under nitrogen atmosphere. The reaction was monitored by LCMS. The mixture was filtered through Celite, and washed with EtOAc (10 mL). The organic layer was combined, evaporated, and purified with a silico gel column, eluted with gradient of DCM/MeOH. The fractions were collected, and concentrated to give tert-butyl 5-(1-(2,2-difluoroethyl)-1H-pyrazolo[3,4-b]pyrazin-6-yl)-6-oxooctahydro-2H-pyrrolo[3,4-c]pyridine-2-carboxylate (50 mg, 47%) as a white solid. MS m/z: 423 [M+H]⁺.

Step 4: 5-(1-(2,2-difluoroethyl)-1H-pyrazolo[3,4-b]pyrazin-6-yl)octahydro-6H-pyrrolo[3,4-c]pyridin-6-one hydrochloride: Follow the general procedure B using tert-butyl 5-(1-(2,2-difluoroethyl)-1H-pyrazolo[3,4-b]pyrazin-6-yl)-6-oxooctahydro-2H-pyrrolo[3,4-c]pyridine-2-carboxylate (50 mg) as the starting material to give the crude product 5-(1-(2,2-difluoroethyl)-1H-pyrazolo[3,4-b]pyrazin-6-yl)octahydro-6H-pyrrolo[3,4-c]pyridin-6-one hydrochloride (50 mg). MS m/z: 323 [M+H]⁺.

Step 5: 5-(1-(2,2-difluoroethyl)-1H-pyrazolo[3,4-b]pyrazin-6-yl)-2-(6-(trifluoromethyl)pyridin-2-yl)octahydro-6H-pyrrolo[3,4-c]pyridin-6-one: Followed the general procedure E using 5-(1-(2,2-difluoroethyl)-1H-pyrazolo[3,4-b]pyrazin-6-yl)octahydro-6H-pyrrolo[3,4-c]pyridin-6-one hydrochloride (50 mg, 0.118 mmol, 1.00 equiv.) and 2-fluoro-6-(trifluoromethyl)pyridine (23.2 mg, 0.142 mmol, 1.20 equiv.) as the starting materials to give 5-(1-(2,2-difluoroethyl)-1H-pyrazolo[3,4-b]pyrazin-6-yl)-2-(6-(trifluoromethyl)pyridin-2-yl)octahydro-6H-pyrrolo[3,4-c]pyridin-6-one (5.8 mg, 10.5%) as a white solid. ¹H NMR (400 MHz, DMSO-d₆) δ 9.11 (s, 1H), 8.48 (s, 1H), 7.71 (t, J=8.0 Hz, 1H), 7.00 (d, J=7.3 Hz, 1H), 6.80 (d, J=8.6 Hz, 1H), 6.52 (tt, J=54.6, 3.6 Hz, 1H), 4.91 (td, J=15.3, 3.6 Hz, 2H), 4.31 (dd, J=13.6, 4.2 Hz, 1H), 4.15 (dd, J=13.6, 5.4 Hz, 1H), 3.85-3.70 (m, 2H), 3.44-3.39 (m, 4H), 3.03 (br, 2H), 2.89 (dd, J=15.8, 6.1 Hz, 1H), 2.66 (dd, J=15.8, 5.0 Hz, 1H). MS m/z: 468 [M+H]⁺.

### Example 67: 5-(1-(2,2-difluoroethyl)-1H-pyrazolo[3,4-b]pyrazin-6-yl)-2-(6-(trifluoromethyl)pyridin-2-yl)octahydro-1H-pyrrolo[3,4-c]pyridin-1-one

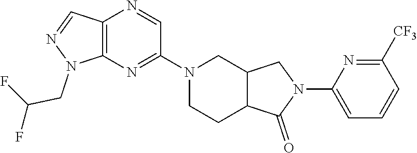 [SMILES: O=C1C2CCN(c3cnc4cnn(CC(F)F)c4n3)CC2CN1c1cccc(C(F)(F)F)n1]

Step 1: octahydro-1H-pyrrolo[3,4-c]pyridin-1-one: To the solution of 2,3-dihydro-1H-pyrrolo[3,4-c]pyridin-1-one (100 mg, 0.746 mmol, 1 equiv.) in 2,2,2-trifluoroethanol (3 mL) was added Pd/C (20 mg, 10% Pd on carbon, wetted with water). The mixture was hydrogenated at 60° C. under 50 atm of hydrogen pressure overnight. Desired product could be detected by LCMS. The reaction system was filtrated through celite, and the filtrate was concentrated to give product octahydro-1H-pyrrolo[3,4-c]pyridin-1-one (100 mg, 96%) as a colorless oil. MS m/z: 141 [M+H]⁺.

Step 2: 5-(1-(2,2-difluoroethyl)-1H-pyrazolo[3,4-b]pyrazin-6-yl)octahydro-1H-pyrrolo[3,4-c]pyridin-1-one: Followed the general procedure E using octahydro-1H-pyrrolo[3,4-c]pyridin-1-one (100 mg, 0.713 mmol, 1 equiv.) and 6-chloro-1-(2,2-difluoroethyl)-1H-pyrazolo[3,4-b]pyrazine (202.71 mg, 0.927 mmol, 1.3 equiv.) as the starting materials to give 5-(1-(2,2-difluoroethyl)-1H-pyrazolo[3,4-b]pyrazin-6-yl)octahydro-1H-pyrrolo[3,4-c]pyridin-1-one (150 mg, 65%) as a white solid. MS m/z: 323 [M+H]⁺.

Step 3: 5-(1-(2,2-difluoroethyl)-1H-pyrazolo[3,4-b]pyrazin-6-yl)-2-(6-(trifluoromethyl)pyridin-2-yl)octahydro-1H-pyrrolo[3,4-c]pyridin-1-one: Followed the general procedure E using 5-(1-(2,2-difluoroethyl)-1H-pyrazolo[3,4-b]pyrazin-6-yl)octahydro-1H-pyrrolo[3,4-c]pyridin-1-one (100 mg, 0.310 mmol, 1 equiv.) and 2-fluoro-6-(trifluoromethyl)pyridine (61.46 mg, 0.372 mmol, 1.2 equiv.) as the starting materials, Cs₂CO₃ (303.26 mg, 0.930 mmol, 3 equiv.) as the base to give 5-(1-(2,2-difluoroethyl)-1H-pyrazolo[3,4-b]pyrazin-6-yl)-2-(6-(trifluoromethyl)pyridin-2-yl)octahydro-1H-pyrrolo[3,4-c]pyridin-1-one (22 mg, 15%) as a white solid. ¹H NMR (400 MHz, DMSO-d₆) δ 8.64-8.62 (m, 1H), 8.45 (s, 1H), 8.16-8.08 (m, 2H), 7.65-7.63 (m, 1H), 6.59-6.30 (m, 1H), 4.74-4.66 (m, 2H), 4.41-4.36 (m, 1H), 4.15-4.12 (m, 1H), 4.06-4.01 (m, 1H), 3.81-3.78 (m, 1H), 3.31-3.23 (m, 2H), 3.14-3.09 (m, 1H), 2.80 (s, 1H), 2.11-2.02 (m, 1H), 2.01-1.94 (m, 1H), 1.23 (s, 1H). MS m/z: 468 [M+H]⁺.

### Example 68: 1-(2,2-difluoroethyl)-6-((3aR,7aS)-2-(5-(trifluoromethyl)pyridin-2-yl)octahydro-5H-pyrrolo[3,4-c]pyridin-5-yl)-1H-pyrazolo[3,4-b]pyrazine, Assumed; Example 69: 1-(2,2-difluoroethyl)-6-((3aS,7aR)-2-(5-(trifluoromethyl)pyridin-2-yl)octahydro-5H-pyrrolo[3,4-c]pyridin-5-yl)-1H-pyrazolo[3,4-b]pyrazine

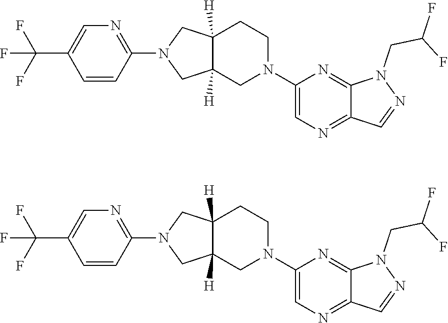 [SMILES: FC(F)Cn1ncc2ncc(N3CC[C@@H]4CN(c5ccc(C(F)(F)F)cn5)C[C@@H]4C3)nc21 ; FC(F)Cn1ncc2ncc(N3CC[C@H]4CN(c5ccc(C(F)(F)F)cn5)C[C@H]4C3)nc21]

1-(2,2-difluoroethyl)-6-(rac-(3aS,7aR)-3a,7a-dimethyl-2-(5-(trifluoromethyl)pyridin-2-yl)octahydro-5H-pyrrolo[3,4-c]pyridin-5-yl)-1H-pyrazolo[3,4-b]pyrazine (150 mg) was purified by Chiral-HPLC using following conditions: Column: CHIRALPAK IA-3, 4.6*50 mm, 3 m; Mobile Phase A: Hex (0.1% DEA): EtOH=50:50; Flow rate: 1 mL/min; Gradient: 0% B to 0% B to afford 1-(2,2-difluoroethyl)-6-((3aR,7aS)-2-(5-(trifluoromethyl)pyridin-2-yl)octahydro-5H-pyrrolo[3,4-c]pyridin-5-yl)-1H-pyrazolo[3,4-b]pyrazine, assumed (60.8 mg, 40.5%) and 1-(2,2-difluoroethyl)-6-((3aS,7aR)-2-(5-(trifluoromethyl)pyridin-2-yl)octahydro-5H-pyrrolo[3,4-c]pyridin-5-yl)-1H-pyrazolo[3,4-b]pyrazine (63.8 mg, 42.5%) as white solids. ¹H NMR (400 MHz, DMSO-d₆) δ 8.48 (s, 1H), 8.37-8.32 (m, 1H), 8.12 (s, 1H), 7.72 (dd, J=9.0, 2.6 Hz, 1H), 6.66-6.23 (m, 2H), 4.69 (td, J=15.0, 3.9 Hz, 2H), 4.10-4.02 (m, 1H), 3.98 (dd, J=13.6, 5.3 Hz, 1H), 3.82 (dd, J=13.8, 4.1 Hz, 1H), 3.64-3.53 (m, 3H), 3.49-3.45 (m, 1H), 3.26-3.21 (m, 1H), 2.60 (s, 2H), 1.87-1.82 (m, 1H), 1.58-1.50 (m, 1H). MS m/z: 454 [M+H]⁺.

### Example 70: 1-(2,2-difluoroethyl)-N-((3aR,5S,7aS)-2-(6-(trifluoromethyl)pyridin-2-yl)octahydro-1H-isoindol-5-yl)-1H-pyrazolo[3,4-b]pyrazin-6-amine, Assumed; Example 71: 1-(2,2-difluoroethyl)-N-((3aR,5R,7aS)-2-(6-(trifluoromethyl)pyridin-2-yl)octahydro-1H-isoindol-5-yl)-1H-pyrazolo[3,4-b]pyrazin-6-amine, Assumed

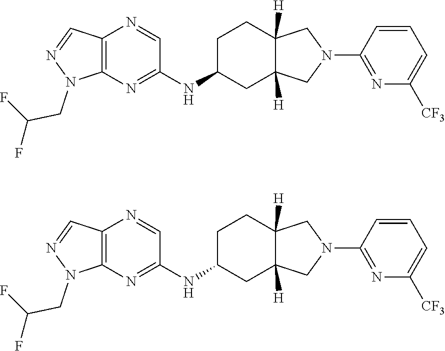

Step 1: tert-butyl 5-aminooctahydro-2H-isoindole-2-carboxylate: To a stirred solution of tert-butyl 5-oxooctahydro-2H-isoindole-2-carboxylate (750 mg, 3.13 mmol, 1.00 equiv.) in MeOH (8 mL) was added ammonium acetate (723 mg, 9.39 mol, 3.00 equiv.) at 0° C., and allowed to warm up to room temperature and stirred for 30 min. Sodium cyanoborohydride (393 mg, 6.26 mmol, 2.00 equiv.) was then added to the mixture at 0° C., and the mixture was heated to 60° C. for 30 min. The reaction was monitored by LCMS. The mixture was quenched with water and concentrated. The residue was purified by reverse phase Combi-flash with the following conditions: column, C18 silica gel; mobile phase, MeCN in water, 30% to 60% gradient in 15 min; detector, UV 220 nm. This resulted in tert-butyl 5-aminooctahydro-2H-isoindole-2-carboxylate (470 mg, 62%) as a colorless oil. MS m/z: 241 [M+H]⁺.

Step 2: tert-butyl 5-((1-(2,2-difluoroethyl)-1H-pyrazolo[3,4-b]pyrazin-6-yl)amino)octahydro-2H-isoindole-2-carboxylate: Followed the general procedure E using 6-chloro-1-(2,2-difluoroethyl)-1H-pyrazolo[3,4-b]pyrazine (427 mg, 1.95 mmol, 1.00 equiv.) and tert-butyl 5-aminooctahydro-2H-isoindole-2-carboxylate (470 mg, 1.95 mmol, 1.00 equiv.) as the starting materials to give tert-butyl 5-((1-(2,2-difluoroethyl)-1H-pyrazolo[3,4-b]pyrazin-6-yl)amino)octahydro-2H-isoindole-2-carboxylate (390 mg, 47%) as a white solid. MS m/z: 423 [M+H]⁺.

Step 3: 1-(2,2-difluoroethyl)-N-(octahydro-1H-isoindol-5-yl)-1H-pyrazolo[3,4-b]pyrazin-6-amine hydrochloride: Follow the general procedure B using tert-butyl 5-((1-(2,2-difluoroethyl)-1H-pyrazolo[3,4-b]pyrazin-6-yl)amino)octahydro-2H-isoindole-2-carboxylate (390 mg) as the starting material to give the crude product 1-(2,2-difluoroethyl)-N-(octahydro-1H-isoindol-5-yl)-1H-pyrazolo[3,4-b]pyrazin-6-amine hydrochloride (350 mg). MS m/z: 323 [M+H]⁺.

Step 4: 1-(2,2-difluoroethyl)-N-((3aR,5S,7aS)-2-(6-(trifluoromethyl)pyridin-2-yl)octahydro-1H-isoindol-5-yl)-1H-pyrazolo[3,4-b]pyrazin-6-amine; 1-(2,2-difluoroethyl)-N-((3aR,5R,7aS)-2-(6-(trifluoromethyl)pyridin-2-yl)octahydro-1H-isoindol-5-yl)-1H-pyrazolo[3,4-b]pyrazin-6-amine: Followed the general procedure E using 1-(2,2-difluoroethyl)-N-(octahydro-1H-isoindol-5-yl)-1H-pyrazolo[3,4-b]pyrazin-6-amine hydrochloride (120 mg, 0.335 mmol, 1.00 equiv.) and 2-fluoro-6-(trifluoromethyl)pyridine (66.3 mg, 0.402 mmol, 1.20 equiv.) as the starting materials to give the crude mixture. The crude mixture was further purified by prep-HPLC to give 1-(2,2-difluoroethyl)-N-((3aR,5S,7aS)-2-(6-(trifluoromethyl)pyridin-2-yl)octahydro-1H-isoindol-5-yl)-1H-pyrazolo[3,4-b]pyrazin-6-amine, assumed (37 mg, 23.6%) and 1-(2,2-difluoroethyl)-N-((3aR,5R,7aS)-2-(6-(trifluoromethyl)pyridin-2-yl)octahydro-1H-isoindol-5-yl)-1H-pyrazolo[3,4-b]pyrazin-6-amine, assumed (25 mg, 15.9%) as white solids.

1-(2,2-difluoroethyl)-N-((3aR,5S,7aS)-2-(6-(trifluoromethyl)pyridin-2-yl)octahydro-1H-isoindol-5-yl)-1H-pyrazolo[3,4-b]pyrazin-6-amine, assumed. ¹H NMR (400 MHz, DMSO-d₆) δ 8.02 (s, 1H), 7.87 (s, 1H), 7.73-7.63 (m, 2H), 6.94 (d, J=7.2 Hz, 1H), 6.72 (d, J=8.6 Hz, 1H), 6.61-6.25 (m, 1H), 4.65 (td, J=15.0, 3.9 Hz, 2H), 3.84 (d, J=4.1 Hz, 1H), 3.54-3.44 (m, 3H), 3.27-3.24 (m, 1H), 2.42 (s, 1H), 2.04-1.77 (m, 5H), 1.47-1.39 (m, 1H), 1.21-1.10 (m, 1H). MS m/z: 468 [M+H]⁺.

1-(2,2-difluoroethyl)-N-((3aR,5R,7aS)-2-(6-(trifluoromethyl)pyridin-2-yl)octahydro-1H-isoindol-5-yl)-1H-pyrazolo[3,4-b]pyrazin-6-amine, assumed. ¹H NMR (400 MHz, DMSO-d₆) δ 8.03 (s, 1H), 7.98 (s, 1H), 7.73 (d, J=6.9 Hz, 1H), 7.69 (t, J=8.0 Hz, 1H), 6.95 (d, J=7.2 Hz, 1H), 6.72 (d, J=8.6 Hz, 1H), 6.42 (tt, 1H), 4.66 (td, 2H), 4.09-4.05 (m, 1H), 3.60-3.54 (m, 1H), 3.52-3.44 (m, 1H), 3.44-3.35 (m, 2H), 2.64 (s, 1H), 2.32-2.27 (m, 1H), 2.05-1.97 (m, 2H), 1.86-1.78 (m, 1H), 1.73-1.62 (m, 1H), 1.49-1.34 (m, 2H). MS m/z: 468 [M+H]⁺.

### Example 72: 1-(2,2-difluoroethyl)-N-((3aR,5S,7aS)-2-(4-(trifluoromethyl)pyridin-2-yl)octahydro-1H-isoindol-5-yl)-1H-pyrazolo[3,4-b]pyrazin-6-amine, Assumed; Example 73: 1-(2,2-difluoroethyl)-N-((3aR,5R,7aS)-2-(4-(trifluoromethyl)pyridin-2-yl)octahydro-1H-isoindol-5-yl)-1H-pyrazolo[3,4-b]pyrazin-6-amine, Assumed

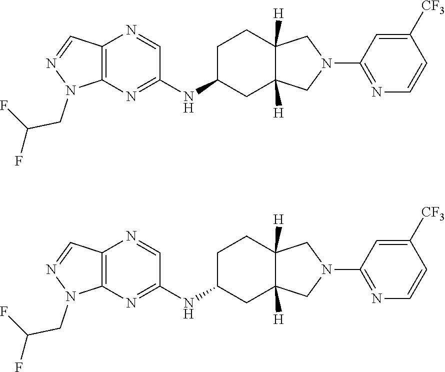

Step 1: 1-(2,2-difluoroethyl)-N-((3aR,5S,7aS)-2-(4-(trifluoromethyl)pyridin-2-yl)octahydro-1H-isoindol-5-yl)-1H-pyrazolo[3,4-b]pyrazin-6-amine; 1-(2,2-difluoroethyl)-N-((3aR,5R,7aS)-2-(4-(trifluoromethyl)pyridin-2-yl)octahydro-1H-isoindol-5-yl)-1H-pyrazolo[3,4-b]pyrazin-6-amine: Followed the general procedure E using 1-(2,2-difluoroethyl)-N-(octahydro-1H-isoindol-5-yl)-1H-pyrazolo[3,4-b]pyrazin-6-amine hydrochloride (60 mg, 0.167 mmol, 1.00 equiv.) and 2-fluoro-4-(trifluoromethyl)pyridine (33.1 mg, 0.201 mmol, 1.20 equiv.) as the starting materials to give the crude mixture. The crude mixture was further purified by prep-HPLC to give 1-(2,2-difluoroethyl)-N-((3aR,5S,7aS)-2-(4-(trifluoromethyl)pyridin-2-yl)octahydro-1H-isoindol-5-yl)-1H-pyrazolo[3,4-b]pyrazin-6-amine, assumed (22.6 mg, 28.9%) and 1-(2,2-difluoroethyl)-N-((3aR,5R,7aS)-2-(4-(trifluoromethyl)pyridin-2-yl)octahydro-1H-isoindol-5-yl)-1H-pyrazolo[3,4-b]pyrazin-6-amine, assumed (6 mg, 7.7%) as white solids.

1-(2,2-difluoroethyl)-N-((3aR,5S,7aS)-2-(4-(trifluoromethyl)pyridin-2-yl)octahydro-1H-isoindol-5-yl)-1H-pyrazolo[3,4-b]pyrazin-6-amine, assumed ¹H NMR (400 MHz, DMSO-d₆) δ 8.28 (d, J=5.2 Hz, 1H), 8.02 (s, 1H), 7.88 (s, 1H), 7.65 (d, J=7.3 Hz, 1H), 6.78 (dd, J=5.2, 1.5 Hz, 1H), 6.65 (s, 1H), 6.44 (tt, J=55.0, 3.9 Hz, 1H), 4.65 (td, J=14.9, 3.9 Hz, 2H), 3.92-3.76 (m, 1H), 3.50 (dd, J=10.4, 5.4 Hz, 2H), 3.43-3.33 (m, 2H), 2.45-2.37 (m, 1H), 2.00-1.76 (m, 5H), 1.50-1.37 (m, 1H), 1.26-1.10 (m, 1H). MS m/z: 468 [M+H]⁺.

1-(2,2-difluoroethyl)-N-((3aR,5R,7aS)-2-(4-(trifluoromethyl)pyridin-2-yl)octahydro-1H-isoindol-5-yl)-1H-pyrazolo[3,4-b]pyrazin-6-amine, assumed ¹H NMR (400 MHz, DMSO-d₆) δ 8.29 (d, J=5.2 Hz, 1H), 8.03 (s, 1H), 7.98 (s, 1H), 7.73 (d, J=7.1 Hz, 1H), 6.78 (dd, 1H), 6.64 (s, 1H), 6.43 (tt, J=54.9, 3.8 Hz, 1H), 4.65 (td, 2H), 4.09-4.04 (m, 1H), 3.59-3.54 (m, 1H), 3.53-3.38 (m, 3H), 2.65-2.63 (m, 1H), 2.34-2.29 (m, 1H), 2.04-1.96 (m, 2H), 1.85-1.78 (m, 1H), 1.73-1.61 (m, 1H), 1.50-1.34 (m, 2H). MS m/z: 468 [M+H]⁺.

### Example 74: 1-(2,2-difluoroethyl)-N-((3aR,5S,7aS)-2-(2-(trifluoromethyl)pyridin-3-yl)octahydro-1H-isoindol-5-yl)-1H-pyrazolo[3,4-b]pyrazin-6-amine, Assumed; Example 75: 1-(2,2-difluoroethyl)-N-((3aR,5R,7aS)-2-(2-(trifluoromethyl)pyridin-3-yl)octahydro-1H-isoindol-5-yl)-1H-pyrazolo[3,4-b]pyrazin-6-amine, Assumed

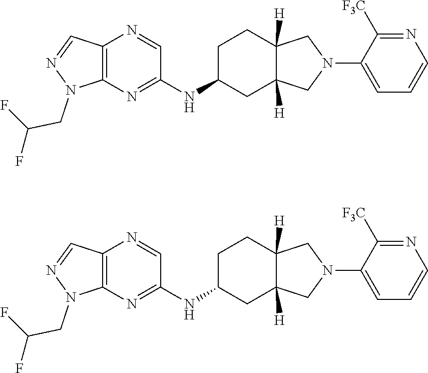

Step 1: 1-(2,2-difluoroethyl)-N-((3aR,5S,7aS)-2-(2-(trifluoromethyl)pyridin-3-yl)octahydro-1H-isoindol-5-yl)-1H-pyrazolo[3,4-b]pyrazin-6-amine; 1-(2,2-difluoroethyl)-N-((3aR,5R,7aS)-2-(2-(trifluoromethyl)pyridin-3-yl)octahydro-1H-isoindol-5-yl)-1H-pyrazolo[3,4-b]pyrazin-6-amine: To a stirred solution of 1-(2,2-difluoroethyl)-N-(octahydro-1H-isoindol-5-yl)-1H-pyrazolo[3,4-b]pyrazin-6-amine hydrochloride (60 mg, 0.167 mmol, 1.00 equiv.) and 3-iodo-2-(trifluoromethyl)pyridine (54.6 mg, 0.201 mmol, 1.20 equiv.) in dioxane (1 mL) were added Cs₂CO₃ (164 mg, 0.501 mmol, 3.00 equiv) and Xphos Pd G3 (14.3 mg, 0.017 mmol, 0.10 equiv). The resulting mixture was stirred for overnight at 100° C. under nitrogen atmosphere. The reaction was monitored by LCMS. The mixture was filtered through Celite, and washed with EtOAc (10 mL). The organic layer was combined, evaporated, and purified with a silico gel column, eluted with gradient of Hexane/EtOAc. The fractions were collected, and concentrated to give crude mixture. The crude mixture was further purified by prep-HPLC to give 1-(2,2-difluoroethyl)-N-((3aR,5S,7aS)-2-(2-(trifluoromethyl)pyridin-3-yl)octahydro-1H-isoindol-5-yl)-1H-pyrazolo[3,4-b]pyrazin-6-amine, assumed (2.7 mg, 3.5%) and 1-(2,2-difluoroethyl)-N-((3aR,5R,7aS)-2-(2-(trifluoromethyl)pyridin-3-yl)octahydro-1H-isoindol-5-yl)-1H-pyrazolo[3,4-b]pyrazin-6-amine, assumed (4 mg, 5.1%) as white solids.

1-(2,2-difluoroethyl)-N-((3aR,5S,7aS)-2-(2-(trifluoromethyl)pyridin-3-yl)octahydro-1H-isoindol-5-yl)-1H-pyrazolo[3,4-b]pyrazin-6-amine, assumed ¹H NMR (400 MHz, DMSO-d₆) δ 8.06-8.00 (m, 2H), 7.97 (s, 1H), 7.78 (d, J=7.2 Hz, 1H), 7.45 (d, J=2.7 Hz, 2H), 6.40 (tt, 1H), 4.65 (td, J=14.9, 4.0 Hz, 2H), 4.12-4.07 (m, 1H), 3.63-3.48 (m, 3H), 3.18-3.10 (m, 1H), 2.64-2.60 (m, 1H), 2.31-2.27 (m, 1H), 2.05-1.94 (m, 2H), 1.83 (dd, J=11.3, 5.4 Hz, 1H), 1.65-1.53 (m, 1H), 1.50-1.33 (m, 2H). MS m/z: 468 [M+H]⁺.

1-(2,2-difluoroethyl)-N-((3aR,5R,7aS)-2-(2-(trifluoromethyl)pyridin-3-yl)octahydro-1H-isoindol-5-yl)-1H-pyrazolo[3,4-b]pyrazin-6-amine, assumed ¹H NMR (400 MHz, DMSO-d₆) δ 8.02 (d, J=3.7 Hz, 2H), 7.88 (s, 1H), 7.73 (d, J=7.4 Hz, 1H), 7.49-7.40 (m, 2H), 6.44 (tt, J=55.0, 3.8 Hz, 1H), 4.65 (td, J=15.0, 3.9 Hz, 2H), 3.83 (d, J=9.5 Hz, 1H), 3.67 (dd, J=9.7, 5.3 Hz, 1H), 3.58 (t, J=10.2 Hz, 1H), 3.19 (t, J=9.0 Hz, 1H), 3.01 (d, J=9.6 Hz, 1H), 2.46-2.35 (m, 2H), 1.98-1.89 (m, 2H), 1.87-1.77 (m, 2H), 1.49-1.38 (m, 1H), 1.26-1.12 (m, 1H). MS m/z: 468 [M+H]⁺.

### Example 76: 1-(2,2-difluoroethyl)-N-((3aR,5s,6aS)-2-(6-methoxypyrazin-2-yl)octahydrocyclopenta[c]pyrrol-5-yl)-1H-pyrazolo[3,4-b]pyrazin-6-amine

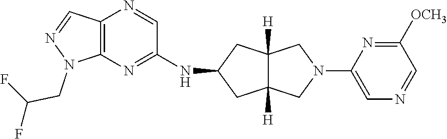 [SMILES: COc1cncc(N2C[C@H]3C[C@H](Nc4cnc5cnn(CC(F)F)c5n4)C[C@H]3C2)n1]

Step 1: 1-(2,2-difluoroethyl)-N-((3aR,5s,6aS)-2-(6-methoxypyrazin-2-yl)octahydrocyclopenta[c]pyrrol-5-yl)-1H-pyrazolo[3,4-b]pyrazin-6-amine: Followed the general procedure E using 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (60 mg, 0.194 mmol, 1.00 equiv.) and 2-chloro-5-methoxypyrazine (33.5 mg, 0.233 mmol, 1.20 equiv.) as the starting materials to give 1-(2,2-difluoroethyl)-N-((3aR,5s,6aS)-2-(5-(trifluoromethyl)pyrazin-2-yl)octahydrocyclopenta[c]pyrrol-5-yl)-1H-pyrazolo[3,4-b]pyrazin-6-amine (21.8 mg, 27.0%) as an off-white solid. ¹H NMR (400 MHz, DMSO-d₆) δ 8.04 (s, 1H), 7.93 (d, J=11.9 Hz, 2H), 7.50 (s, 1H), 7.42 (s, 1H), 6.42 (s, OH), 4.70-4.57 (m, 2H), 4.40 (q, J=6.5 Hz, 1H), 3.84 (s, 3H), 3.67 (t, J=8.9 Hz, 2H), 2.97 (s, 2H), 2.00 (m, J=11.1, 5.0 Hz, 2H), 1.90 (m, J=13.2, 6.5 Hz, 2H). MS m/z: 417.10 [M+H]⁺.

### Example 77: 1-(2,2-difluoroethyl)-N-((3aR,5s,6aS)-2-(5-methoxypyrazin-2-yl)octahydrocyclopenta[c]pyrrol-5-yl)-1H-pyrazolo[3,4-b]pyrazin-6-amine

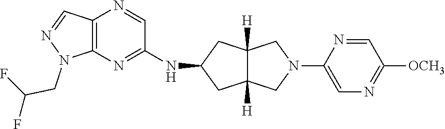 [SMILES: COc1cnc(N2C[C@H]3C[C@H](Nc4cnc5cnn(CC(F)F)c5n4)C[C@H]3C2)cn1]

Step 1: 1-(2,2-difluoroethyl)-N-((3aR,5s,6aS)-2-(5-methoxypyrazin-2-yl)octahydrocyclopenta[c]pyrrol-5-yl)-1H-pyrazolo[3,4-b]pyrazin-6-amine: To a stirred solution of 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (60 mg, 0.194 mmol, 1.00 equiv.) and 2-bromo-5-methoxypyrazine (44 mg, 0.233 mmol, 1.20 equiv.) in dioxane (1 mL) were added Na₂CO₃ (61.7 mg, 0.582 mmol, 3.00 equiv.) and Xphos Pd G3 (16.0 mg, 0.019 mmol, 0.10 equiv.). The resulting mixture was stirred for overnight at 100° C. under nitrogen atmosphere. The reaction was monitored by LCMS. The mixture was filtered through Celite, and washed with EtOAc (10 mL). The organic layer was combined, evaporated, and purified with a silico gel column, eluted with gradient of Hexane/EtOAc. The fractions were collected, and concentrated to give 1-(2,2-difluoroethyl)-N-((3aR,5s,6aS)-2-(5-methoxypyrazin-2-yl)octahydrocyclopenta[c]pyrrol-5-yl)-1H-pyrazolo[3,4-b]pyrazin-6-amine (4.3 mg, 5.3%) as a white solid. ¹H NMR (400 MHz, DMSO-d₆) δ 8.03 (s, 1H), 7.94 (s, 1H), 7.91-7.85 (m, 2H), 7.58 (d, J=1.5 Hz, 1H), 6.39 (tt, 1H), 4.63 (td, J=15.1, 3.9 Hz, 2H), 4.38 (q, J=6.4 Hz, 1H), 3.80 (s, 3H), 3.60-3.55 (m, 2H), 3.25-3.22 (m, 2H), 2.98-2.93 (m, 2H), 2.06-1.96 (m, 2H), 1.92-1.83 (m, 2H). MS m/z: 417[M+H]⁺.

### Example 78: 1-(2,2-difluoroethyl)-N-((3aR,5s,6aS)-2-(6-(trifluoromethyl)pyrazin-2-yl)octahydrocyclopenta[c]pyrrol-5-yl)-1H-pyrazolo[3,4-b]pyrazin-6-amine

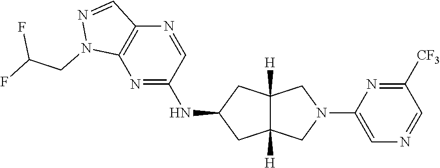 [SMILES: FC(F)Cn1ncc2ncc(N[C@H]3C[C@@H]4CN(c5cncc(C(F)(F)F)n5)C[C@@H]4C3)nc21]

Step 1: 1-(2,2-difluoroethyl)-N-((3aR,5s,6aS)-2-(6-(trifluoromethyl)pyrazin-2-yl)octahydrocyclopenta[c]pyrrol-5-yl)-1H-pyrazolo[3,4-b]pyrazin-6-amine: Followed the general procedure E using 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (60 mg, 0.194 mmol, 1.00 equiv.) and 2-fluoro-6-(trifluoromethyl)pyrazine (38.8 mg, 0.233 mmol, 1.20 equiv.) as the starting materials to give 1-(2,2-difluoroethyl)-N-((3aR,5s,6aS)-2-(6-(trifluoromethyl)pyrazin-2-yl)octahydrocyclopenta[c]pyrrol-5-yl)-1H-pyrazolo[3,4-b]pyrazin-6-amine (23.4 mg, 27.0%) as an off-white solid. ¹H NMR (400 MHz, DMSO-d₆) δ 8.32 (s, 1H), 8.19 (s, 1H), 8.03 (s, 1H), 7.96-7.88 (m, 2H), 6.81-6.12 (m, 1H), 4.69-4.56 (m, 2H), 4.48-4.35 (m, 1H), 3.81-3.71 (m, 2H), 3.45-3.37 (m, 2H), 3.03-2.94 (m, 2H), 2.10-1.98 (m, 2H), 1.96-1.85 (m, 2H). MS m/z: 455 [M+H]⁺.

### Example 79: 1-(2,2-difluoroethyl)-N-((3aR,5s,6aS)-2-(5-(trifluoromethyl)pyrazin-2-yl)octahydrocyclopenta[c]pyrrol-5-yl)-1H-pyrazolo[3,4-b]pyrazin-6-amine

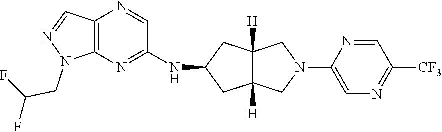 [SMILES: FC(F)Cn1ncc2ncc(N[C@H]3C[C@@H]4CN(c5cnc(C(F)(F)F)cn5)C[C@@H]4C3)nc21]

Step 1: tert-butyl 2-(5-fluoro-4-(trifluoromethyl)pyridin-2-yl)-2,8-diazaspiro[4.5]decane-8-carboxylate: Followed the general procedure E using tert-butyl (3aR,5s,6aS)-5-aminohexahydrocyclopenta[c]pyrrole-2(1H)-carboxylate (1 g, 4.41 mmol, 1.00 equiv.) and 6-chloro-1-(2,2-difluoroethyl)-1H-pyrazolo[3,4-b]pyrazine (1.06 g, 4.85 mmol, 1.10 equiv.) as the starting materials to give tert-butyl (3aR,5s,6aS)-5-((1-(2,2-difluoroethyl)-1H-pyrazolo[3,4-b]pyrazin-6-yl)amino)hexahydrocyclopenta[c]pyrrole-2(1H)-carboxylate (1.14 g, 63%) as a white solid. MS m/z: 409 [M+H]⁺.

Step 2: 1-(2,2-difluoroethyl)-N-((3aR,5s,6aS)-octahydrocyclopenta[c]pyrrol-5-yl)-1H-pyrazolo[3,4-b]pyrazin-6-amine hydrochloride: Followed the general procedure B using tert-butyl (3aR,5s,6aS)-5-((1-(2,2-difluoroethyl)-1H-pyrazolo[3,4-b]pyrazin-6-yl)amino)hexahydrocyclopenta[c]pyrrole-2(1H)-carboxylate (1.14 g) as the starting material to give the crude product 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (1 g). MS m/z: 309 [M+H]⁺.

Step 3: 1-(2,2-difluoroethyl)-N-((3aR,5s,6aS)-2-(5-(trifluoromethyl)pyrazin-2-yl)octahydrocyclopenta[c]pyrrol-5-yl)-1H-pyrazolo[3,4-b]pyrazin-6-amine: Followed the general procedure E using 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (60 mg, 0.194 mmol, 1.00 equiv.) and 2-chloro-5-(trifluoromethyl)pyrazine (42.7 mg, 0.233 mmol, 1.20 equiv.) as the starting materials to give 1-(2,2-difluoroethyl)-N-((3aR,5s,6aS)-2-(5-(trifluoromethyl)pyrazin-2-yl)octahydrocyclopenta[c]pyrrol-5-yl)-1H-pyrazolo[3,4-b]pyrazin-6-amine (36 mg, 40.0%) as an off-white solid. ¹H NMR (400 MHz, DMSO-d₆) δ 8.48 (s, 1H), 8.11 (d, J=1.4 Hz, 1H), 8.03 (s, 1H), 7.92 (d, J=12.0 Hz, 2H), 6.62-6.18 (m, 1H), 4.69-4.56 (m, 2H), 4.46-4.36 (m, 1H), 3.84-3.75 (m, 2H), 3.49-3.41 (m, 2H), 3.00 (s, 2H), 2.07-1.99 (m, 2H), 1.96-1.86 (m, 2H). MS m/z: 455 [M+H]⁺.

### Example 80: 1-(2,2-difluoroethyl)-N-((3aR,5s,6aS)-2-(3-(trifluoromethyl)pyrazin-2-yl)octahydrocyclopenta[c]pyrrol-5-yl)-1H-pyrazolo[3,4-b]pyrazin-6-amine

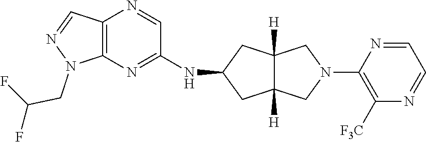 [SMILES: FC(F)Cn1ncc2ncc(N[C@H]3C[C@@H]4CN(c5nccnc5C(F)(F)F)C[C@@H]4C3)nc21]

Step 1: 1-(2,2-difluoroethyl)-N-((3aR,5s,6aS)-2-(6-(trifluoromethyl)pyrazin-2-yl)octahydrocyclopenta[c]pyrrol-5-yl)-1H-pyrazolo[3,4-b]pyrazin-6-amine: Followed the general procedure E using 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (60 mg, 0.194 mmol, 1.00 equiv.) and 2-fluoro-3-(trifluoromethyl)pyrazine (38.8 mg, 0.233 mmol, 1.20 equiv.) as the starting materials to give 1-(2,2-difluoroethyl)-N-((3aR,5s,6aS)-2-(3-(trifluoromethyl)pyrazin-2-yl)octahydrocyclopenta[c]pyrrol-5-yl)-1H-pyrazolo[3,4-b]pyrazin-6-amine (21.0 mg, 23.8%) as an off-white solid. ¹H NMR (400 MHz, DMSO-d₆) δ 8.42 (d, J=2.3 Hz, 1H), 8.08 (d, J=2.2 Hz, 1H), 8.03 (s, 1H), 7.96-7.86 (m, 2H), 6.69-6.18 (m, 1H), 4.68-4.55 (m, 2H), 4.47-4.34 (m, 1H), 3.73-3.64 (m, 2H), 3.53-3.44 (m, 2H), 2.96-2.88 (m, 2H), 2.05-1.95 (m, 2H), 1.86 (s, 1H), 1.87-1.79 (m, 1H). MS m/z: 455.20 [M+H]⁺.

### Example 81: 1-(2,2-difluoroethyl)-N-((3aR,5s,6aS)-2-(3-methoxypyrazin-2-yl)octahydrocyclopenta[c]pyrrol-5-yl)-1H-pyrazolo[3,4-b]pyrazin-6-amine

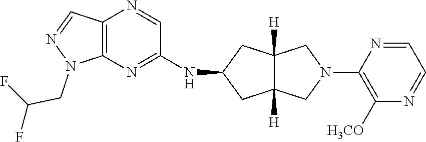 [SMILES: COc1nccnc1N1C[C@H]2C[C@H](Nc3cnc4cnn(CC(F)F)c4n3)C[C@H]2C1]

Step 1: 1-(2,2-difluoroethyl)-N-((3aR,5s,6aS)-2-(3-methoxypyrazin-2-yl)octahydrocyclopenta[c]pyrrol-5-yl)-1H-pyrazolo[3,4-b]pyrazin-6-amine: Followed the general procedure E using 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (60 mg, 0.194 mmol, 1.00 equiv.) and 2-chloro-3-methoxypyrazine (33.5 mg, 0.233 mmol, 1.20 equiv.) as the starting materials to give 1-(2,2-difluoroethyl)-N-((3aR,5s,6aS)-2-(3-methoxypyrazin-2-yl)octahydrocyclopenta[c]pyrrol-5-yl)-1H-pyrazolo[3,4-b]pyrazin-6-amine (28 mg, 34.7%) as a white solid. ¹H NMR (400 MHz, DMSO-d₆) δ 8.03 (s, 1H), 7.94 (s, 1H), 7.87 (d, J=6.4 Hz, 1H), 7.69-7.60 (m, 1H), 7.40 (dd, J=16.6, 3.7 Hz, 1H), 6.42 (tt, J=55.0, 4.0 Hz, 1H), 4.63 (td, J=14.9, 3.8 Hz, 2H), 4.40 (p, J=6.6 Hz, 1H), 3.89 (s, 3H), 3.73 (dd, J=11.3, 6.7 Hz, 2H), 3.56 (s, OH), 3.51-3.48 (m, 2H), 3.42 (s, OH), 2.87 (dq, J=8.4, 4.1 Hz, 2H), 2.05-1.92 (m, 2H), 1.91-1.80 (m, 2H). MS m/z: 417 [M+H]⁺.

### Example 82: 1-(2,2-difluoroethyl)-N-((3aR,5s,6aS)-2-(6-(trifluoromethyl)pyridazin-3-yl)octahydrocyclopenta[c]pyrrol-5-yl)-1H-pyrazolo[3,4-b]pyrazin-6-amine

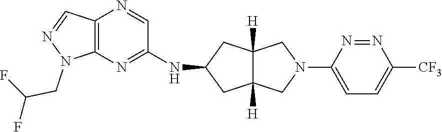 [SMILES: FC(F)Cn1ncc2ncc(N[C@H]3C[C@@H]4CN(c5ccc(C(F)(F)F)nn5)C[C@@H]4C3)nc21]

Step 1: 1-(2,2-difluoroethyl)-N-((3aR,5s,6aS)-2-(6-(trifluoromethyl)pyridazin-3-yl)octahydrocyclopenta[c]pyrrol-5-yl)-1H-pyrazolo[3,4-b]pyrazin-6-amine: Followed the general procedure E using 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (60 mg, 0.195 mmol, 1.00 equiv.) and 3-bromo-6-(trifluoromethyl)pyridazine (52.7 mg, 0.233 mmol, 1.20 equiv.) as the starting materials to give 1-(2,2-difluoroethyl)-N-((3aR,5s,6aS)-2-(6-(trifluoromethyl)pyridazin-3-yl)octahydrocyclopenta[c]pyrrol-5-yl)-1H-pyrazolo[3,4-b]pyrazin-6-amine (25.3 mg, 29%) as a white solid. ¹H NMR (400 MHz, DMSO-d₆) δ 8.03 (s, 1H), 7.96-7.89 (m, 2H), 7.78 (d, J=9.5 Hz, 1H), 7.05 (d, J=9.5 Hz, 1H), 6.40 (tt, J=55.0, 3.8 Hz, 1H), 4.69-4.56 (m, 2H), 4.48-4.38 (m, 1H), 3.81 (t, J=9.4 Hz, 2H), 3.46 (d, J=10.9 Hz, 2H), 3.02 (d, J=4.0 Hz, 2H), 2.08-2.01 (m, 2H), 1.96-1.88 (m, 2H). MS m/z: 455 [M+H]⁺.

### Example 83: 1-(2,2-difluoroethyl)-N-((3aR,5s,6aS)-2-(5-(trifluoromethyl)pyridazin-3-yl)octahydrocyclopenta[c]pyrrol-5-yl)-1H-pyrazolo[3,4-b]pyrazin-6-amine

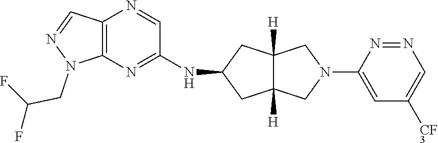 [SMILES: FC(F)Cn1ncc2ncc(N[C@H]3C[C@@H]4CN(c5cc(C(F)(F)F)cnn5)C[C@@H]4C3)nc21]

Step 1: 1-(2,2-difluoroethyl)-N-((3aR,5s,6aS)-2-(5-(trifluoromethyl)pyridazin-3-yl)octahydrocyclopenta[c]pyrrol-5-yl)-1H-pyrazolo[3,4-b]pyrazin-6-amine: Followed the general procedure E using 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (60 mg, 0.195 mmol, 1.00 equiv.) and 3-chloro-5-(trifluoromethyl)pyridazine (42.7 mg, 0.233 mmol, 1.20 equiv.) as the starting materials to give 1-(2,2-difluoroethyl)-N-((3aR,5s,6aS)-2-(5-(trifluoromethyl)pyridazin-3-yl)octahydrocyclopenta[c]pyrrol-5-yl)-1H-pyrazolo[3,4-b]pyrazin-6-amine (38.8 mg, 43.7%) as a white solid. ¹H NMR (400 MHz, DMSO-d₆) δ 8.83 (d, J=1.8 Hz, 1H), 8.03 (s, 1H), 7.96-7.89 (m, 2H), 7.22 (s, 1H), 6.40 (tt, J=54.9, 3.8 Hz, 1H), 4.62 (td, J=15.0, 3.9 Hz, 2H), 4.48-4.35 (m, 1H), 3.79 (dd, J=11.2, 7.5 Hz, 2H), 3.47 (dd, J=11.3, 3.5 Hz, 2H), 3.02 (dt, J=8.0, 4.2 Hz, 2H), 2.10-2.00 (m, 2H), 2.00-1.93 (m, 1H), 1.93-1.86 (m, 1H). MS m/z: 455 [M+H]⁺.

### Example 84: 1-(2,2-difluoroethyl)-N-((3aR,5s,6aS)-2-(4-(trifluoromethyl)pyridazin-3-yl)octahydrocyclopenta[c]pyrrol-5-yl)-1H-pyrazolo[3,4-b]pyrazin-6-amine

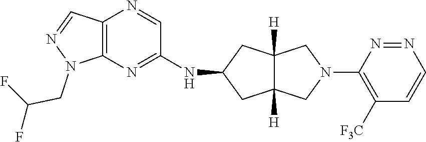 [SMILES: FC(F)Cn1ncc2ncc(N[C@H]3C[C@@H]4CN(c5nnccc5C(F)(F)F)C[C@@H]4C3)nc21]

Step 1: 1-(2,2-difluoroethyl)-N-((3aR,5s,6aS)-2-(4-(trifluoromethyl)pyridazin-3-yl)octahydrocyclopenta[c]pyrrol-5-yl)-1H-pyrazolo[3,4-b]pyrazin-6-amine: Followed the general procedure E using 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (60 mg, 0.195 mmol, 1.00 equiv.) and 3-chloro-4-(trifluoromethyl)pyridazine (42.7 mg, 0.233 mmol, 1.20 equiv.) as the starting materials to give 1-(2,2-difluoroethyl)-N-((3aR,5s,6aS)-2-(4-(trifluoromethyl)pyridazin-3-yl)octahydrocyclopenta[c]pyrrol-5-yl)-1H-pyrazolo[3,4-b]pyrazin-6-amine (6.5 mg, 6.1%) as a white solid. ¹H NMR (400 MHz, DMSO-d₆) δ 8.88 (d, J=5.0 Hz, 1H), 8.03 (s, 1H), 7.95-7.87 (m, 2H), 7.81 (d, J=5.0 Hz, 1H), 6.40 (tt, J=55.1, 4.0 Hz, 1H), 4.62 (td, J=14.8, 4.0 Hz, 2H), 4.50-4.37 (m, 1H), 3.74 (dd, J=10.9, 7.3 Hz, 2H), 3.56 (dd, J=11.1, 2.7 Hz, 2H), 2.96 (td, J=6.7, 3.2 Hz, 2H), 2.10-1.99 (m, 2H), 1.92-1.80 (m, 2H). MS m/z: 455 [M+H]⁺.

### Example 85: 1-(2,2-difluoroethyl)-N-((3aR,5s,6aS)-2-(4-(trifluoromethyl)pyrimidin-2-yl)octahydrocyclopenta[c]pyrrol-5-yl)-1H-pyrazolo[3,4-b]pyrazin-6-amine

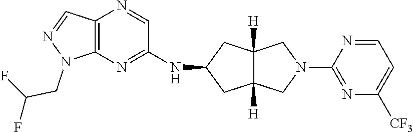 [SMILES: FC(F)Cn1ncc2ncc(N[C@H]3C[C@@H]4CN(c5nccc(C(F)(F)F)n5)C[C@@H]4C3)nc21]

Step 1: 1-(2,2-difluoroethyl)-N-((3aR,5s,6aS)-2-(4-(trifluoromethyl)pyrimidin-2-yl)octahydrocyclopenta[c]pyrrol-5-yl)-1H-pyrazolo[3,4-b]pyrazin-6-amine: Followed the general procedure E using 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (60 mg, 0.195 mmol, 1.00 equiv.) and 2-chloro-4-(trifluoromethyl)pyrimidine (42.7 mg, 0.233 mmol, 1.20 equiv.) as the starting materials to give 1-(2,2-difluoroethyl)-N-((3aR,5s,6aS)-2-(4-(trifluoromethyl)pyrimidin-2-yl)octahydrocyclopenta[c]pyrrol-5-yl)-1H-pyrazolo[3,4-b]pyrazin-6-amine (22.4 mg, 25.2%) as a white solid. ¹H NMR (400 MHz, DMSO-d₆) δ 8.67 (d, J=4.8 Hz, 1H), 8.03 (s, 1H), 7.95-7.89 (m, 2H), 7.01 (d, J=4.9 Hz, 1H), 6.40 (tt, J=55.0, 3.9 Hz, 1H), 4.62 (td, J=15.0, 3.9 Hz, 2H), 4.47-4.34 (m, 1H), 3.84-3.75 (m, 2H), 3.47-3.40 (m, 2H), 3.03-2.89 (m, 2H), 2.07-1.97 (m, 2H), 1.93-1.81 (m, 2H). MS m/z: 455 [M+H]⁺.

### Example 86: 1-(2,2-difluoroethyl)-N-((3aR,5s,6aS)-2-(5-(trifluoromethyl)pyrimidin-2-yl)octahydrocyclopenta[c]pyrrol-5-yl)-1H-pyrazolo[3,4-b]pyrazin-6-amine

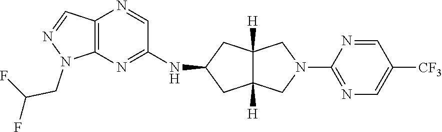 [SMILES: FC(F)Cn1ncc2ncc(N[C@H]3C[C@@H]4CN(c5ncc(C(F)(F)F)cn5)C[C@@H]4C3)nc21]

Step 1: 1-(2,2-difluoroethyl)-N-((3aR,5s,6aS)-2-(5-(trifluoromethyl)pyrimidin-2-yl)octahydrocyclopenta[c]pyrrol-5-yl)-1H-pyrazolo[3,4-b]pyrazin-6-amine: Followed the general procedure E using 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (60 mg, 0.195 mmol, 1.00 equiv.) and 2-chloro-5-(trifluoromethyl)pyrimidine (42.7 mg, 0.233 mmol, 1.20 equiv.) as the starting materials to give 1-(2,2-difluoroethyl)-N-((3aR,5s,6aS)-2-(5-(trifluoromethyl)pyrimidin-2-yl)octahydrocyclopenta[c]pyrrol-5-yl)-1H-pyrazolo[3,4-b]pyrazin-6-amine (34.6 mg, 52.7%) as a white solid. ¹H NMR (400 MHz, DMSO-d₆) δ 8.69 (s, 2H), 8.04 (s, 1H), 7.96-7.90 (m, 2H), 6.41 (tt, 1H), 4.62 (td, J=15.0, 3.8 Hz, 2H), 4.45-4.35 (m, 1H), 3.83 (dd, J=12.2, 7.2 Hz, 2H), 3.49 (dd, J=12.1, 3.6 Hz, 2H), 3.02-2.95 (m, 2H), 2.07-1.96 (m, 2H), 1.94-1.83 (m, 2H). MS m/z: 455 [M+H]⁺.

### Example 87: 1-(2,2-difluoroethyl)-N-((3aR,5s,6aS)-2-(2-(trifluoromethyl)pyrimidin-4-yl)octahydrocyclopenta[c]pyrrol-5-yl)-1H-pyrazolo[3,4-b]pyrazin-6-amine

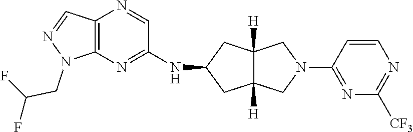 [SMILES: FC(F)Cn1ncc2ncc(N[C@H]3C[C@@H]4CN(c5ccnc(C(F)(F)F)n5)C[C@@H]4C3)nc21]

Step 1: 1-(2,2-difluoroethyl)-N-((3aR,5s,6aS)-2-(2-(trifluoromethyl)pyrimidin-4-yl)octahydrocyclopenta[c]pyrrol-5-yl)-1H-pyrazolo[3,4-b]pyrazin-6-amine: Followed the general procedure E using 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (60 mg, 0.195 mmol, 1.00 equiv.) and 4-bromo-2-(trifluoromethyl)pyrimidine (52.7 mg, 0.233 mmol, 1.20 equiv.) as the starting materials to give 1-(2,2-difluoroethyl)-N-((3aR,5s,6aS)-2-(2-(trifluoromethyl)pyrimidin-4-yl)octahydrocyclopenta[c]pyrrol-5-yl)-1H-pyrazolo[3,4-b]pyrazin-6-amine (34.6 mg, 39.0%) as a white solid. ¹H NMR (400 MHz, DMSO-d₆) δ 8.30 (d, J=6.2 Hz, 1H), 8.03 (s, 1H), 7.96-7.88 (m, 2H), 6.74 (d, J=6.2 Hz, 1H), 6.40 (tt, J=55.0, 3.9 Hz, 1H), 4.63 (td, J=15.0, 3.9 Hz, 2H), 4.41 (h, J=6.3 Hz, 1H), 3.75 (d, J=35.0 Hz, 2H), 3.51-3.32 (m, 2H), 2.98 (s, 2H), 2.07-1.97 (m, 2H), 1.93-1.87 (m, 2H). MS m/z: 455 [M+H]⁺.

### Example 88: 1-(2,2-difluoroethyl)-N-((3aR,5s,6aS)-2-(2-(trifluoromethyl)pyrimidin-5-yl)octahydrocyclopenta[c]pyrrol-5-yl)-1H-pyrazolo[3,4-b]pyrazin-6-amine

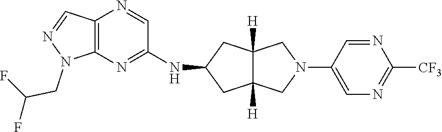 [SMILES: FC(F)Cn1ncc2ncc(N[C@H]3C[C@@H]4CN(c5cnc(C(F)(F)F)nc5)C[C@@H]4C3)nc21]

Step 1: 1-(2,2-difluoroethyl)-N-((3aR,5s,6aS)-2-(2-(trifluoromethyl)pyrimidin-5-yl)octahydrocyclopenta[c]pyrrol-5-yl)-1H-pyrazolo[3,4-b]pyrazin-6-amine: Followed the general procedure E using 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (60 mg, 0.195 mmol, 1.00 equiv.) and 5-chloro-2-(trifluoromethyl)pyrimidine (42.7 mg, 0.233 mmol, 1.20 equiv.) as the starting materials to give 1-(2,2-difluoroethyl)-N-((3aR,5s,6aS)-2-(2-(trifluoromethyl)pyrimidin-5-yl)octahydrocyclopenta[c]pyrrol-5-yl)-1H-pyrazolo[3,4-b]pyrazin-6-amine (36.2 mg, 40.8%) as a white solid. ¹H NMR (400 MHz, DMSO-d₆) δ 8.30 (d, J=6.2 Hz, 1H), 8.03 (s, 1H), 7.96-7.88 (m, 2H), 6.74 (d, J=6.2 Hz, 1H), 6.40 (tt, J=55.0, 3.9 Hz, 1H), 4.63 (td, J=15.0, 3.9 Hz, 2H), 4.41 (h, J=6.3 Hz, 1H), 3.75 (d, J=35.0 Hz, 2H), 3.51-3.32 (m, 2H), 2.98 (s, 2H), 2.07-1.97 (m, 2H), 1.93-1.87 (m, 2H). MS m/z: 455 [M+H]⁺.

### Example 89: 1-(2,2-difluoroethyl)-N-((3aR,5s,6aS)-2-(4-(trifluoromethyl)pyrimidin-5-yl)octahydrocyclopenta[c]pyrrol-5-yl)-1H-pyrazolo[3,4-b]pyrazin-6-amine

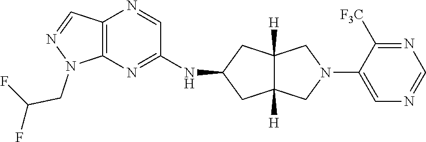 [SMILES: FC(F)Cn1ncc2ncc(N[C@H]3C[C@@H]4CN(c5cncnc5C(F)(F)F)C[C@@H]4C3)nc21]

Step 1: 1-(2,2-difluoroethyl)-N-((3aR,5s,6aS)-2-(4-(trifluoromethyl)pyrimidin-5-yl)octahydrocyclopenta[c]pyrrol-5-yl)-1H-pyrazolo[3,4-b]pyrazin-6-amine: Followed the general procedure E using 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (60 mg, 0.195 mmol, 1.00 equiv.) and 5-chloro-2-(trifluoromethyl)pyrimidine (42.7 mg, 0.233 mmol, 1.20 equiv.) as the starting materials to give 1-(2,2-difluoroethyl)-N-((3aR,5s,6aS)-2-(4-(trifluoromethyl)pyrimidin-5-yl)octahydrocyclopenta[c]pyrrol-5-yl)-1H-pyrazolo[3,4-b]pyrazin-6-amine (13 mg, 14.6%) as a white solid. ¹H NMR (400 MHz, DMSO-d₆) δ 8.83-8.74 (m, 2H), 8.04 (s, 1H), 7.93 (s, 1H), 7.85 (d, J=6.5 Hz, 1H), 6.40 (tt, 1H), 4.64 (t, 2H), 4.49-4.41 (m, 1H), 3.43-3.41 (m, 2H), 3.29 (s, 2H), 2.93 (s, 2H), 1.99 (d, J=8.7 Hz, 2H), 1.87 (d, J=13.5 Hz, 2H). MS m/z: 455 [M+H]⁺.

### Example 90: 1-(2,2-difluoroethyl)-N-((3aR,5s,6aS)-2-(6-(trifluoromethyl)pyrimidin-4-yl)octahydrocyclopenta[c]pyrrol-5-yl)-1H-pyrazolo[3,4-b]pyrazin-6-amine

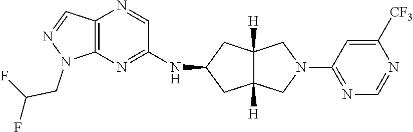 [SMILES: FC(F)Cn1ncc2ncc(N[C@H]3C[C@@H]4CN(c5cc(C(F)(F)F)ncn5)C[C@@H]4C3)nc21]

Step 1: 1-(2,2-difluoroethyl)-N-((3aR,5s,6aS)-2-(6-(trifluoromethyl)pyrimidin-4-yl)octahydrocyclopenta[c]pyrrol-5-yl)-1H-pyrazolo[3,4-b]pyrazin-6-amine: Followed the general procedure E using 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (60 mg, 0.194 mmol, 1.00 equiv.) and 4-bromo-6-(trifluoromethyl)pyrimidine (52.6 mg, 0.233 mmol, 1.20 equiv.) as the starting materials to give 1-(2,2-difluoroethyl)-N-((3aR,5s,6aS)-2-(6-(trifluoromethyl)pyrimidin-4-yl)octahydrocyclopenta[c]pyrrol-5-yl)-1H-pyrazolo[3,4-b]pyrazin-6-amine (41.0 mg, 46.4%) as an off-white solid. ¹H NMR (400 MHz, DMSO-d₆) δ 8.61 (s, 1H), 8.04 (s, 1H), 7.93 (d, J=5.0 Hz, 2H), 6.92 (s, 1H), 6.41 (t, J=3.8 Hz, OH), 4.63 (td, J=15.0, 3.9 Hz, 2H), 4.40 (h, J=6.4 Hz, 1H), 3.73 (s, 1H), 3.50 (d, J=10.9 Hz, 1H), 2.98 (s, 2H), 2.01 (m, J=12.3, 5.8 Hz, 2H), 1.90 (m, J=7.7, 7.3 Hz, 2H). MS m/z: 455 [M+H]⁺.

### Example 91: 1-(2,2-difluoroethyl)-N-((3aR,5s,6aS)-2-(5-(trifluoromethyl)pyrimidin-4-yl)octahydrocyclopenta[c]pyrrol-5-yl)-1H-pyrazolo[3,4-b]pyrazin-6-amine

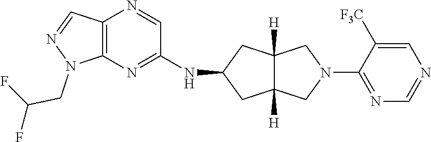 [SMILES: FC(F)Cn1ncc2ncc(N[C@H]3C[C@@H]4CN(c5ncncc5C(F)(F)F)C[C@@H]4C3)nc21]

Step 1: N-((3aR,5s,6aS)-2-(2-chloro-5-(trifluoromethyl)pyrimidin-4-yl)octahydrocyclopenta[c]pyrrol-5-yl)-1-(2,2-difluoroethyl)-1H-pyrazolo[3,4-b]pyrazin-6-amine: To a stirred solution of 1-(2,2-difluoroethyl)-N-((3aR,5s,6aS)-octahydrocyclopenta[c]pyrrol-5-yl)-1H-pyrazolo[3,4-b]pyrazin-6-amine hydrochloride (100 mg, 0.325 mmol, 1.00 equiv.) in DMF (3.00 mL) was added NaH (60% w/w, 14.3 mg, 0.358 mol, 1.10 equiv.) at 0° C., and allowed to warm up to room temperature and stirred for 15 min. 2,4-dichloro-5-(trifluoromethyl)pyrimidine (77.3 mg, 0.358 mmol, 1.10 equiv.) was added to the mixture, and the mixture was heated at room temperature for 3 h. The reaction was monitored by LCMS. The mixture was quenched with water, and extracted with EtOAc (20 mL×2). The organic layer was washed with brine, dried, filtered, evaporated, and purified by Combi-Flash to give N-((3aR,5s,6aS)-2-(2-chloro-5-(trifluoromethyl)pyrimidin-4-yl)octahydrocyclopenta[c]pyrrol-5-yl)-1-(2,2-difluoroethyl)-1H-pyrazolo[3,4-b]pyrazin-6-amine (50 mg, 34%) as a colorless oil. MS m/z: 489 [M+H]⁺.

Step 2: 1-(2,2-difluoroethyl)-N-((3aR,5s,6aS)-2-(5-(trifluoromethyl)pyrimidin-4-yl)octahydrocyclopenta[c]pyrrol-5-yl)-1H-pyrazolo[3,4-b]pyrazin-6-amine: To the solution of N-((3aR,5s,6aS)-2-(2-chloro-5-(trifluoromethyl)pyrimidin-4-yl)octahydrocyclopenta[c]pyrrol-5-yl)-1-(2,2-difluoroethyl)-1H-pyrazolo[3,4-b]pyrazin-6-amine (50 mg, 0.102 mmol, 1.00 equiv.) in 2,2,2-trifluoroethanol (3 mL) was added Pd/C (10 mg, 10% Pd on carbon, wetted with water). The resulting mixture was hydrogenated overnight at room temperature. Desired product could be detected by LCMS. The reaction system was filtrated through celite and the filtrate was concentrated. The residue was purified by Combi-flash (12 g silico gel column), eluted with DCM: MeOH. The fractions were collected, and concentrated to give product 1-(2,2-difluoroethyl)-N-((3aR,5s,6aS)-2-(5-(trifluoromethyl)pyrimidin-4-yl)octahydrocyclopenta[c]pyrrol-5-yl)-1H-pyrazolo[3,4-b]pyrazin-6-amine (12.6 mg, 27.1%) as a white solid. H NMR (300 MHz, DMSO-d₆) δ 8.71-8.59 (m, 2H), 8.04 (s, 1H), 7.97-7.88 (B, 2H), 6.40 (tt, J=55.1, 4.0 Hz, 1H), 4.62 (td, J=14.9, 4.0 Hz, 2H), 4.45-4.32 (m, 1H), 3.84 (dd, J=11.9, 6.9 Hz, 2H), 3.60 (dd, J=11.6, 3.2 Hz, 2H), 3.05-2.86 (m, 2H), 2.08-1.94 (m, 2H), 1.94-1.78 (m, 2H). MS m/z: 455 [M+H]⁺.

### Biological Assay Data and Procedures

Exemplary compounds were evaluated for activation of GCase in a live cell PFB assay in HELA cells (essentially as described in Ysselstein et al., “LRRK2 kinase activity regulates lysosomal glucocerebrosidase in neurons derived from Parkinson's disease patients” Nature Communications (2019) 10:5570). The results in Table 1 demonstrate that compounds of the disclosure are potent activators of GCase. EC₅₀ ranges: A: <10 μM; B: >10-50 μM; C: >50-100 μM; D: >100 μM.

TABLE 1
In vitro enzymatic EC₅₀ values for exemplary compounds

| Cmpd No. | GCase EC₅₀: (μM) |
|---|---|
| 1 | A |
| 2 | A |
| 3 | A |
| 4 | A |
| 5 | A |
| 6 | A |
| 7 | A |
| 8 | A |
| 9 | B |
| 10 | B |
| 11 | B |
| 12 | B |
| 13 | B |
| 14 | B |
| 15 | B |
| 16 | B |
| 17 | B |
| 18 | B |
| 19 | D |
| 20 | B |
| 21 | A |
| 22 | A |
| 23 | B |
| 24 | B |
| 25 | D |
| 26 | C |
| 27 | C |
| 28 | B |
| 29 | A |
| 30 | A |
| 31 | D |
| 32 | A |
| 33 | A |
| 34 | B |
| 35 | A |
| 36 | A |
| 37 | A |
| 38 | A |
| 39 | A |
| 40 | B |
| 41 | D |
| 42 | B |
| 43 | A |
| 44 | D |
| 45 | D |
| 46 | C |
| 47 | B |
| 48 | B |
| 49 | B |
| 50 | A |
| 51 | A |
| 52 | A |
| 53 | A |
| 54 | B |
| 55 | B |
| 56 | A |
| 57 | B |
| 58 | B |
| 59 | B |
| 60 | B |
| 61 | B |
| 62 | B |
| 63 | A |
| 64 | B |
| 65 | A |
| 66 | B |
| 67 | A |
| 68 | B |
| 69 | B |
| 70 | A |
| 71 | B |
| 72 | A |
| 73 | B |
| 74 | B |
| 75 | D |
| 76 | B |
| 77 | D |
| 78 | A |
| 79 | B |
| 80 | A |
| 81 | B |
| 82 | B |
| 83 | B |
| 84 | D |
| 85 | A |
| 86 | B |
| 87 | A |
| 88 | A |
| 89 | A |
| 90 | A |
| 91 | A |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

For reasons of completeness, various aspects of the present disclosure are set out in the following numbered clauses:

Clause 1. A compound of Formula (I):

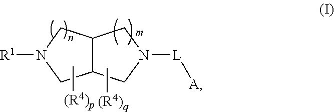

or a pharmaceutically acceptable salt thereof, wherein:
R¹ is substituted or unsubstituted heteroaryl, substituted or unsubstituted aryl, or a nitrogen protecting group;
L is a bond or —C(═O)—;
A is

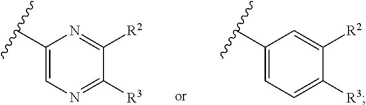

R² and R³ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl;
each R⁴ is independently hydrogen, halogen, substituted or unsubstituted alkyl, substituted or unsubstituted cycloalkyl, substituted or unsubstituted alkoxy, or two instances of R⁴ on the same carbon together with that carbon form a carbonyl;
p is 0, 1, 2, 3, or 4;
q is 0, 1, 2, 3, or 4;
m is 1 or 2; and
n is 1 or 2.

Clause 2. The compound of clause 1, or a pharmaceutically acceptable salt thereof, wherein: R¹ is substituted or unsubstituted heteroaryl, or a nitrogen protecting group.

Clause 3. The compound of clause 1 or 2, or a pharmaceutically acceptable salt thereof, wherein: R¹ is substituted or unsubstituted heteroaryl.

Clause 4. The compound of any of clauses 1-3, or a pharmaceutically acceptable salt thereof, wherein: R¹ is substituted or unsubstituted pyridinyl.

Clause 5. The compound of any of clauses 1-4, or a pharmaceutically acceptable salt thereof, wherein: R¹ is substituted pyridinyl.

Clause 6. The compound of any of clauses 1-5, or a pharmaceutically acceptable salt thereof, wherein: R¹ is pyridinyl substituted with halogen or haloalkyl.

Clause 7. The compound of any of clauses 1-6, or a pharmaceutically acceptable salt thereof, wherein: R¹ is pyridinyl substituted with haloalkyl.

Clause 8. The compound of any of clauses 1-7, or a pharmaceutically acceptable salt thereof, wherein: R¹ is pyridinyl substituted with trifluoromethyl.

Clause 9. The compound of any of clauses 1-8, or a pharmaceutically acceptable salt thereof, wherein: R¹ is

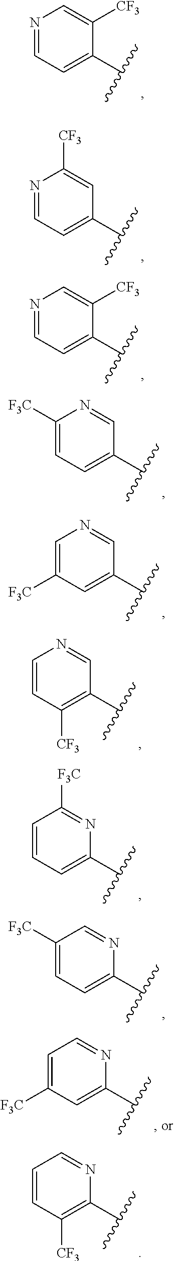

Clause 10. The compound of any of clauses 1-9, or a pharmaceutically acceptable salt thereof, wherein: R¹ is a nitrogen protecting group

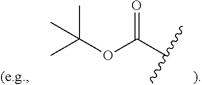 [SMILES: *C(=O)OC(C)(C)C]

Clause 11. The compound of any of clauses 1-10, or a pharmaceutically acceptable salt thereof, wherein: A is

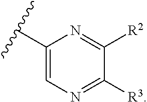

Clause 12. The compound of any of clauses 1-10, or a pharmaceutically acceptable salt thereof, wherein: A is

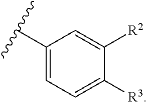

Clause 13. The compound of any of clauses 1-12, or a pharmaceutically acceptable salt thereof, wherein: R³ is hydrogen.

Clause 17. The compound of any of clauses 1-13, or a pharmaceutically acceptable salt thereof, wherein: R² and R³ together with the atoms to which they are attached form a substituted or unsubstituted heteroaryl.

Clause 18. The compound of any of clauses 1-17, or a pharmaceutically acceptable salt thereof, wherein: R² and R³ together with the atoms to which they are attached form a substituted or unsubstituted pyrrolyl or a substituted or unsubstituted pyrazolyl.

Clause 19. The compound of any of clauses 1-17, or a pharmaceutically acceptable salt thereof, wherein: R² and R³ together with the atoms to which they are attached form a substituted or unsubstituted pyrazolyl.

Clause 20. The compound of any of clauses 1-19, or a pharmaceutically acceptable salt thereof, wherein: A is

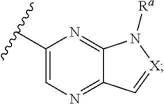

wherein X is N or CR^{a}; and each R^{a} is independently hydrogen, substituted or unsubstituted alkyl, or substituted or unsubstituted heterocyclyl.

Clause 21. The compound of any of clauses 1-20, or a pharmaceutically acceptable salt thereof, wherein: A is

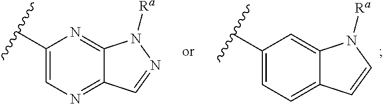

wherein R^{a} is hydrogen, substituted or unsubstituted alkyl, or substituted or unsubstituted heterocyclyl.

Clause 22. The compound of any of clauses 1-20, or a pharmaceutically acceptable salt thereof, wherein: A is

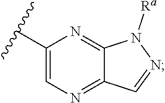

wherein R^{a} is substituted or unsubstituted alkyl or substituted or unsubstituted heterocyclyl.

Clause 23. The compound of any of clauses 1-22, or a pharmaceutically acceptable salt thereof, wherein: A is

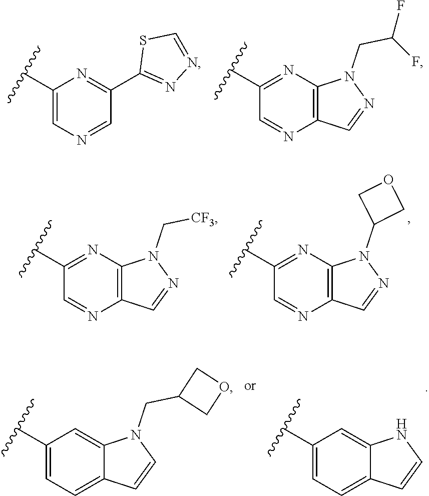

Clause 24. The compound of any of clauses 1-23, or a pharmaceutically acceptable salt thereof, wherein: p is 0.

Clause 25. The compound of any of clauses 1-24, or a pharmaceutically acceptable salt thereof, wherein: q is 0.

Clause 26. The compound of any of clauses 1-25, or a pharmaceutically acceptable salt thereof, wherein: m is 1.

Clause 27. The compound of any of clauses 1-25, or a pharmaceutically acceptable salt thereof, wherein: m is 2.

Clause 28. The compound of any of clauses 1-27, or a pharmaceutically acceptable salt thereof, wherein: n is 1.

Clause 29. The compound of any of clauses 1-27, or a pharmaceutically acceptable salt thereof, wherein: n is 2.

Clause 30. The compound of any of clauses 1-25, or a pharmaceutically acceptable salt thereof, wherein: m is 1; and n is 2.

Clause 31. The compound of any of clauses 1-25, or a pharmaceutically acceptable salt thereof, wherein: m is 2; and n is 1.

Clause 32. The compound of any of clauses 1-31, or a pharmaceutically acceptable salt thereof, wherein: L is a bond.

Clause 33. The compound of any of clauses 1-31, or a pharmaceutically acceptable salt thereof, wherein: L is —C(═O)—.

Clause 34. The compound of clause 1, wherein the compound is of formula (I-a-1):

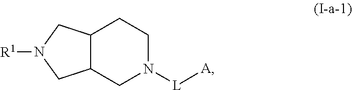

or a pharmaceutically acceptable salt thereof.

Clause 35. The compound of clause 1, wherein the compound is of formula (I-b-1):

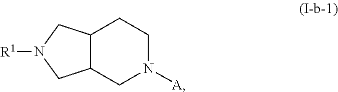

or a pharmaceutically acceptable salt thereof.

Clause 36. The compound of clause 1, wherein the compound is of formula (I-c-1):

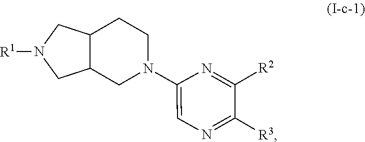

or a pharmaceutically acceptable salt thereof.

Clause 37. The compound of clause 1, wherein the compound is of formula (I-d-1):

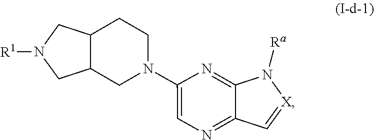

or a pharmaceutically acceptable salt thereof, wherein: X is N or CR^{a}; and each R^{a} is independently hydrogen, substituted or unsubstituted alkyl or substituted or unsubstituted heterocyclyl.

Clause 38. The compound of clause 1, wherein the compound is of formula (I-e-1):

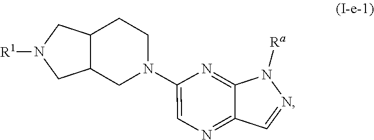

or a pharmaceutically acceptable salt thereof, wherein: R^{a} is hydrogen, substituted or unsubstituted alkyl or substituted or unsubstituted heterocyclyl.

Clause 39. The compound of clause 1, wherein the compound is of formula (I-f-1):

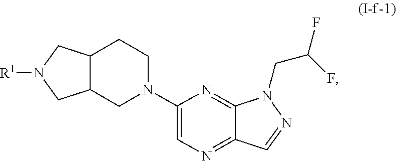

or a pharmaceutically acceptable salt thereof.

Clause 40. The compound of clause 1, wherein the compound is of formula (I-g-1):

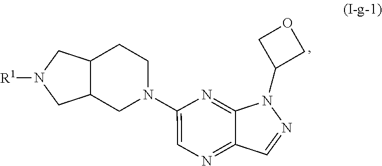

or a pharmaceutically acceptable salt thereof.

Clause 41. The compound of clause 1, wherein the compound is of formula (I-h-1):

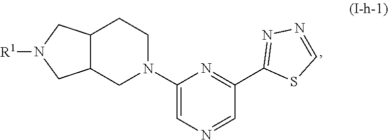

or a pharmaceutically acceptable salt thereof.

Clause 42. The compound of clause 1, wherein the compound is of formula (I-i-1):

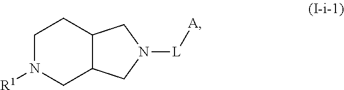

or a pharmaceutically acceptable salt thereof.

Clause 43. The compound of clause 1, wherein the compound is of formula (I-j-1):

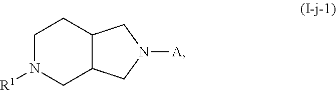

or a pharmaceutically acceptable salt thereof.

Clause 44. The compound of clause 1, wherein the compound is of formula (I-k-1):

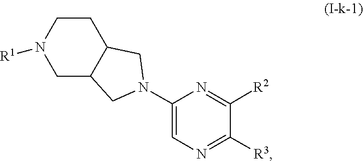

or a pharmaceutically acceptable salt thereof.

Clause 45. The compound of clause 1, wherein the compound is of formula (I-l-1):

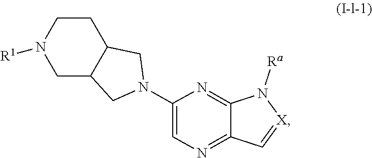

or a pharmaceutically acceptable salt thereof, wherein X is N or CR^{a}; and each R^{a} is independently hydrogen, substituted or unsubstituted alkyl, or substituted or unsubstituted heterocyclyl.

Clause 46. The compound of clause 1, wherein the compound is of formula (I-m-1):

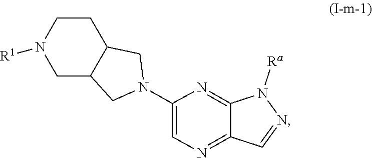

or a pharmaceutically acceptable salt thereof, wherein R^{a} is independently hydrogen, substituted or unsubstituted alkyl, or substituted or unsubstituted heterocyclyl.

Clause 47. The compound of clause 1, wherein the compound is of formula (I-n-1):

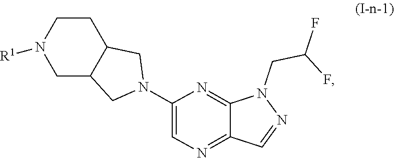

or a pharmaceutically acceptable salt thereof.

Clause 48. The compound of clause 1, wherein the compound is:

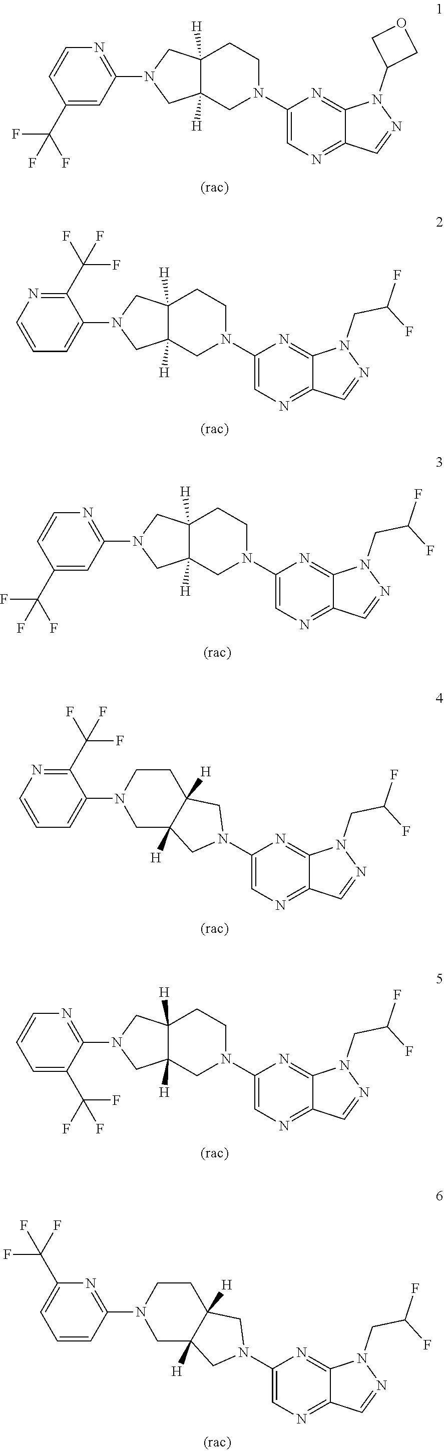 [SMILES: FC(F)(F)c1ccnc(N2C[C@H]3CCN(c4cnc5cnn(C6COC6)c5n4)C[C@H]3C2)c1 ; FC(F)Cn1ncc2ncc(N3CC[C@@H]4CN(c5cccnc5C(F)(F)F)C[C@@H]4C3)nc21 ; FC(F)Cn1ncc2ncc(N3CC[C@@H]4CN(c5cc(C(F)(F)F)ccn5)C[C@@H]4C3)nc21 ; FC(F)Cn1ncc2ncc(N3C[C@H]4CCN(c5cccnc5C(F)(F)F)C[C@H]4C3)nc21 ; FC(F)Cn1ncc2ncc(N3CC[C@H]4CN(c5ncccc5C(F)(F)F)C[C@H]4C3)nc21 ; FC(F)Cn1ncc2ncc(N3C[C@H]4CCN(c5cccc(C(F)(F)F)n5)C[C@H]4C3)nc21]

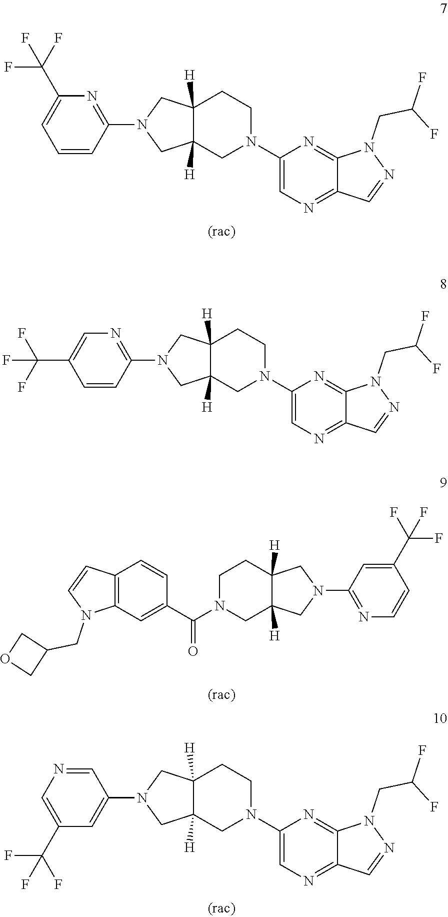 [SMILES: FC(F)Cn1ncc2ncc(N3CC[C@H]4CN(c5cccc(C(F)(F)F)n5)C[C@H]4C3)nc21 ; FC(F)Cn1ncc2ncc(N3CC[C@H]4CN(c5ccc(C(F)(F)F)cn5)C[C@H]4C3)nc21 ; O=C(c1ccc2ccn(CC3COC3)c2c1)N1CC[C@@H]2CN(c3cc(C(F)(F)F)ccn3)C[C@@H]2C1 ; FC(F)Cn1ncc2ncc(N3CC[C@@H]4CN(c5cncc(C(F)(F)F)c5)C[C@@H]4C3)nc21]

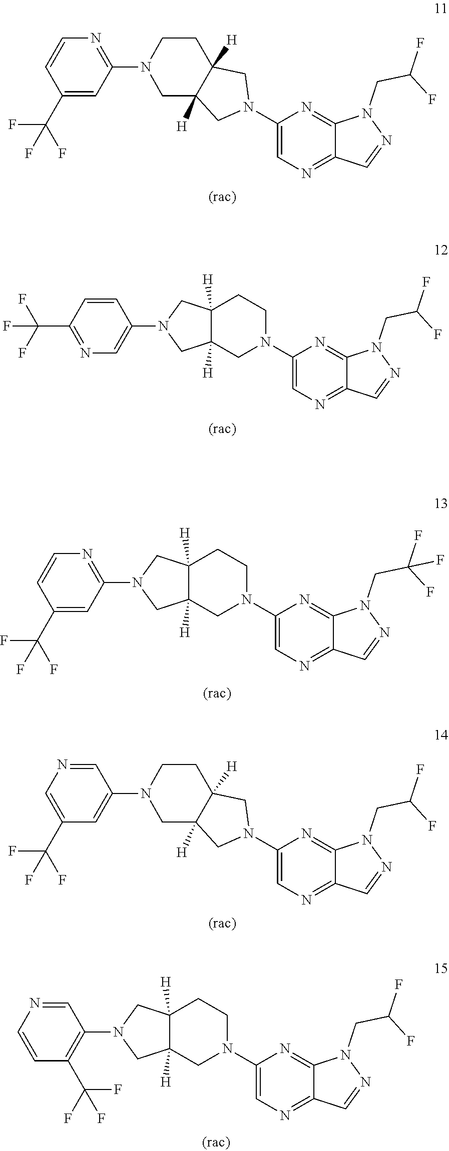 [SMILES: FC(F)Cn1ncc2ncc(N3C[C@H]4CCN(c5cc(C(F)(F)F)ccn5)C[C@H]4C3)nc21 ; FC(F)Cn1ncc2ncc(N3CC[C@@H]4CN(c5ccc(C(F)(F)F)nc5)C[C@@H]4C3)nc21 ; FC(F)(F)Cn1ncc2ncc(N3CC[C@@H]4CN(c5cc(C(F)(F)F)ccn5)C[C@@H]4C3)nc21 ; FC(F)Cn1ncc2ncc(N3C[C@H]4CN(c5cncc(C(F)(F)F)c5)CC[C@H]4C3)nc21 ; FC(F)Cn1ncc2ncc(N3CC[C@@H]4CN(c5cnccc5C(F)(F)F)C[C@@H]4C3)nc21]

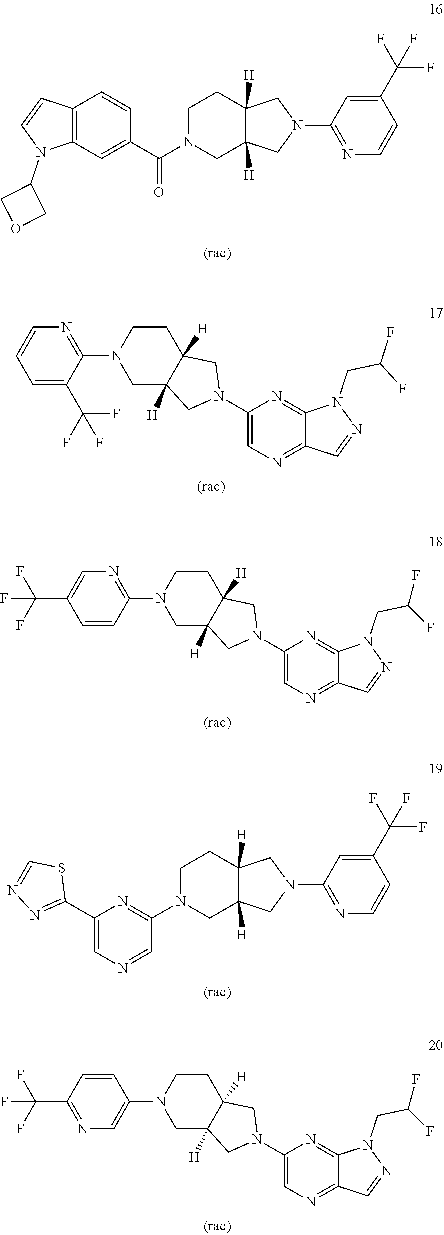 [SMILES: O=C(c1ccc2ccn(C3COC3)c2c1)N1CC[C@@H]2CN(c3cc(C(F)(F)F)ccn3)C[C@@H]2C1 ; FC(F)Cn1ncc2ncc(N3C[C@H]4CCN(c5ncccc5C(F)(F)F)C[C@H]4C3)nc21 ; FC(F)Cn1ncc2ncc(N3C[C@H]4CCN(c5ccc(C(F)(F)F)cn5)C[C@H]4C3)nc21 ; FC(F)(F)c1ccnc(N2C[C@H]3CCN(c4cncc(-c5nncs5)n4)C[C@H]3C2)c1 ; FC(F)Cn1ncc2ncc(N3C[C@H]4CN(c5ccc(C(F)(F)F)nc5)CC[C@H]4C3)nc21]

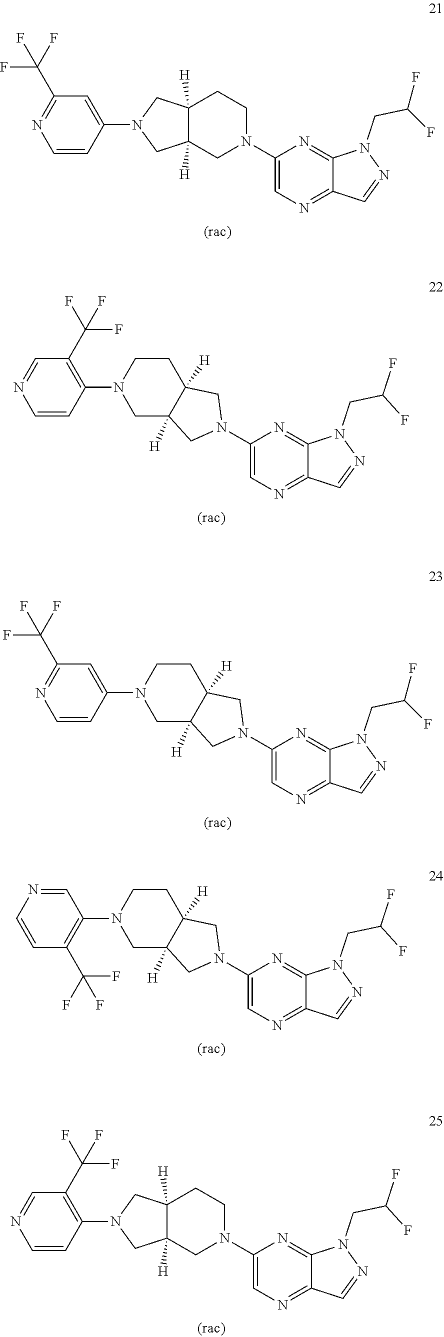 [SMILES: FC(F)Cn1ncc2ncc(N3CC[C@@H]4CN(c5ccnc(C(F)(F)F)c5)C[C@@H]4C3)nc21 ; FC(F)Cn1ncc2ncc(N3C[C@@H]4CCN(c5ccncc5C(F)(F)F)C[C@@H]4C3)nc21 ; FC(F)Cn1ncc2ncc(N3C[C@H]4CN(c5ccnc(C(F)(F)F)c5)CC[C@H]4C3)nc21 ; FC(F)Cn1ncc2ncc(N3C[C@@H]4CCN(c5cnccc5C(F)(F)F)C[C@@H]4C3)nc21 ; FC(F)Cn1ncc2ncc(N3CC[C@@H]4CN(c5ccncc5C(F)(F)F)C[C@@H]4C3)nc21]

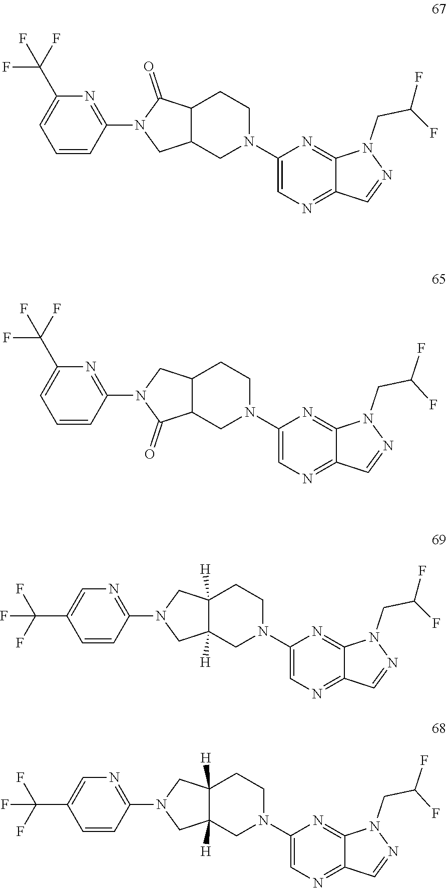 [SMILES: O=C1C2CCN(c3cnc4cnn(CC(F)F)c4n3)CC2CN1c1cccc(C(F)(F)F)n1 ; O=C1C2CN(c3cnc4cnn(CC(F)F)c4n3)CCC2CN1c1cccc(C(F)(F)F)n1 ; FC(F)Cn1ncc2ncc(N3CC[C@@H]4CN(c5ccc(C(F)(F)F)cn5)C[C@@H]4C3)nc21 ; FC(F)Cn1ncc2ncc(N3CC[C@H]4CN(c5ccc(C(F)(F)F)cn5)C[C@H]4C3)nc21]

or a pharmaceutically acceptable salt thereof.

Clause 49. A pharmaceutical composition comprising a compound of any of clauses 1-48, or a pharmaceutically acceptable salt thereof, and a pharmaceutically acceptable excipient.

Clause 50. A kit comprising a compound of any of clauses 1-48, or a pharmaceutically acceptable salt thereof, or a pharmaceutical composition of clause 49, and instructions for administering the compound or pharmaceutical composition to a subject in need thereof.

Clause 51. A method of treating a disease or disorder in a subject in need thereof, the method comprising administering an effective amount of a compound of any of clauses 1-48, or pharmaceutically acceptable salt thereof, or a pharmaceutical composition of clause 49.

Clause 52. The method of clause 51, wherein the disease or disorder is associated with glucocerebrosidase activity.

Clause 53. The method of clause 51 or 52, wherein the disease or disorder is a neurological disease or disorder.

Clause 54. The method of clause 53, wherein the neurological disease or disorder is Parkinson's disease or Gaucher's disease.

Clause 55. A method of activating glucocerebrosidase, the method comprising contacting glucocerebrosidase with an effective amount of a compound of any of clauses 1-48, or pharmaceutically acceptable salt thereof, or a pharmaceutical composition of clause 49.

Clause 56. The method of clause 55, wherein the contacting is in vitro.

Clause 57. The method of clause 55, wherein the contacting is in vivo.

Clause 58. A method of preparing the compound of clause 1, or a pharmaceutically acceptable salt thereof, the method comprising reacting a compound of Formula (A):

R¹—X (A),

or a salt thereof; with a compound of Formula (B):

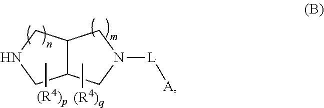

or a salt thereof; wherein:
X is a leaving group (e.g., halogen (e.g., —Cl, —Br—, —I)); and
R¹, R⁴, L, A, n, m, p, and q are as defined in clause 1.

Clause 59. The method of clause 58, further comprising reacting a compound of Formula (C):

X¹-L-A (C),

or a salt thereof; with a compound of Formula (D):

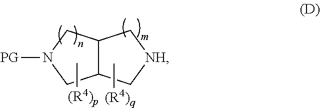

or a salt thereof; wherein:
R⁴, L, A, n, m, p, and q are as defined in clause 1;
X¹ is a leaving group (e.g., halogen (e.g., —Cl, —Br—, —I)); and
PG is a nitrogen protecting group (e.g., carbamate protecting group (e.g., BOC)).

Clause 60. A method of preparing the compound of clause 1, or a pharmaceutically acceptable salt thereof, the method comprising reacting a compound of Formula (C):

X¹-L-A (C),

or a salt thereof; with a compound of Formula (B-1):

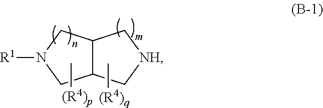

or a salt thereof; wherein:
X¹ is a leaving group (e.g., halogen (e.g., —Cl, —Br—, —I)); and
R¹, R⁴, L, A, n, m, p, and q are as defined in clause 1.

Clause 61. The method of clause 60, further comprising reacting a compound of Formula (A):

R¹—X (A),

or a salt thereof; with a compound of Formula (D-1):

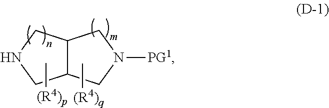

or a salt thereof; wherein:
R¹, R⁴, n, m, p, and q are as defined in clause 1;
X is a leaving group (e.g., halogen (e.g., —Cl, —Br—, —I)); and
PG¹ is a nitrogen protecting group (e.g., carbamate protecting group (e.g., BOC)).

What is claimed is:

1. A compound of Formula (I): 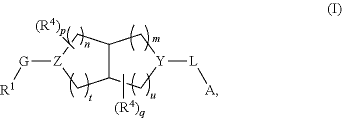
or a pharmaceutically acceptable salt thereof, wherein:
R¹ is substituted or unsubstituted heteroaryl, substituted or unsubstituted aryl, or a nitrogen protecting group;
G is a bond or —O—;
X¹ is N or CR⁴;
Y is N or CR⁴;
Z is N or CR⁴;
L is a bond, —NR^{A}—, or —C(═O)—;
A is 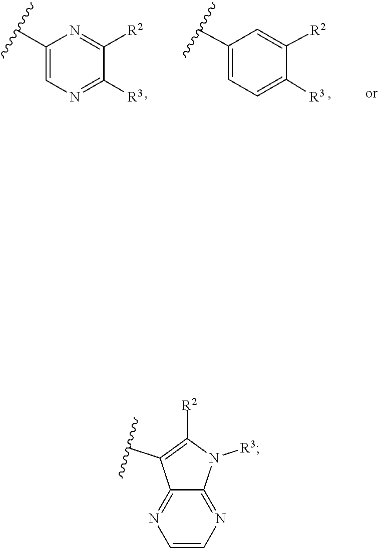
R² and R³ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl;
each R⁴ is independently hydrogen, halogen, substituted or unsubstituted alkyl, substituted or unsubstituted cycloalkyl, substituted or unsubstituted alkoxy, or two instances of R⁴ on the same carbon together with that carbon form a carbonyl;
R^{A} is hydrogen, substituted or unsubstituted alkyl, or a nitrogen protecting group;
p is 0, 1, 2, 3, or 4;
q is 0, 1, 2, 3, or 4;
t is 0, 1 or 2;
u is 0, 1 or 2;
m is 0, 1 or 2; and
n is 0, 1 or 2; provided that the sum of m and u is 2 or 3, and the sum of n and t is 2 or 3.

2. A compound of Formula (II): 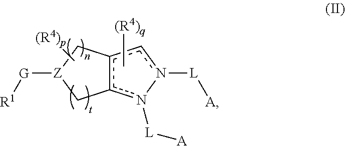
or a pharmaceutically acceptable salt thereof, wherein:
each [symbol] independently represents a single or double bond;
R¹ is substituted or unsubstituted heteroaryl, substituted or unsubstituted aryl, or a nitrogen protecting group;
G is a bond or —O—;
Z is N or CR⁴;
L is absent, a bond, —NR^{A}—, or —C(═O)—; 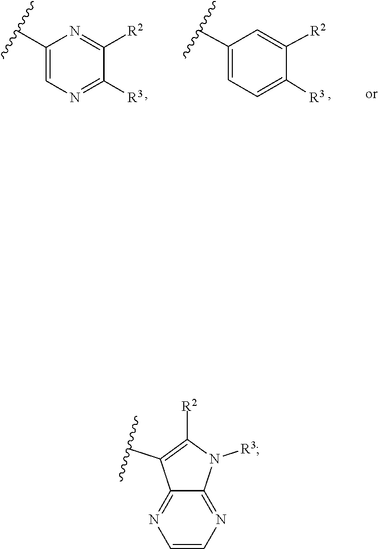
A is absent,
R² and R³ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl;
each R⁴ is independently hydrogen, halogen, substituted or unsubstituted alkyl, substituted or unsubstituted cycloalkyl, substituted or unsubstituted alkoxy, or two instances of R⁴ on the same carbon together with that carbon form a carbonyl;
R^{A} is hydrogen, substituted or unsubstituted alkyl, or a nitrogen protecting group;
p is 0, 1, 2, 3, or 4;
q is 0, 1, 2, or 3;
t is 1 or 2; and
n is 1 or 2; provided that one instance of -L-A is absent.

3. The compound of claim 1 or 2, or a pharmaceutically acceptable salt thereof, wherein:
R¹ is substituted or unsubstituted heteroaryl, or a nitrogen protecting group.

4. The compound of any of claims 1-3, or a pharmaceutically acceptable salt thereof, wherein:
R¹ is substituted or unsubstituted heteroaryl.

5. The compound of any of claims 1-4, or a pharmaceutically acceptable salt thereof, wherein:
R¹ is substituted or unsubstituted pyridinyl, substituted or unsubstituted pyrazinyl, substituted or unsubstituted pyrimidinyl, or substituted or unsubstituted pyridazinyl.

6. The compound of any of claims 1-5, or a pharmaceutically acceptable salt thereof, wherein:
R¹ is substituted or unsubstituted pyridinyl.

7. The compound of any of claims 1-6, or a pharmaceutically acceptable salt thereof, wherein:
R¹ is substituted pyridinyl.

8. The compound of any of claims 1-7, or a pharmaceutically acceptable salt thereof, wherein:
R¹ is pyridinyl substituted with halogen or haloalkyl.

9. The compound of any of claims 1-8, or a pharmaceutically acceptable salt thereof, wherein:
R¹ is pyridinyl substituted with haloalkyl.

10. The compound of any of claims 1-9, or a pharmaceutically acceptable salt thereof, wherein:
R¹ is pyridinyl substituted with trifluoromethyl.

11. The compound of any of claims 1-10, or a pharmaceutically acceptable salt thereof, wherein:
R¹ is

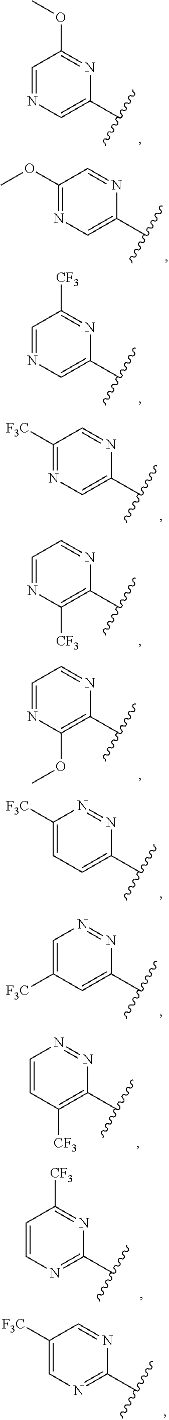

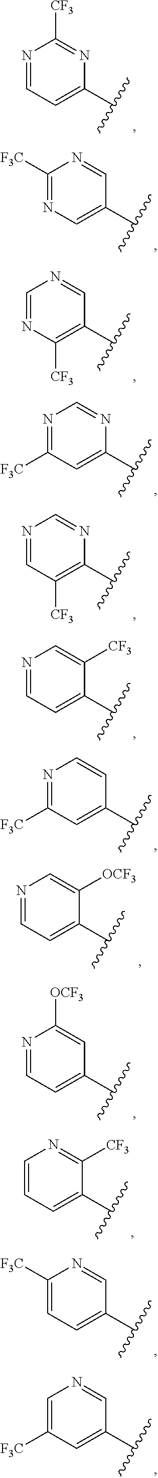

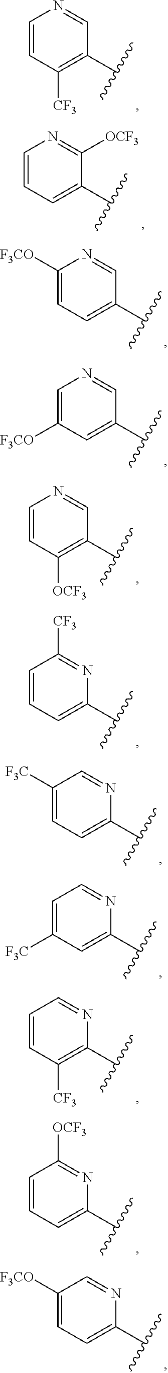

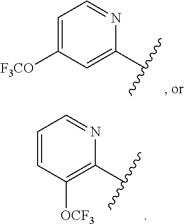 [SMILES: *c1cc(OC(F)(F)F)ccn1 ; *c1ncccc1OC(F)(F)F]

12. The compound of any of claims 1-11, or a pharmaceutically acceptable salt thereof, wherein:
R¹ is

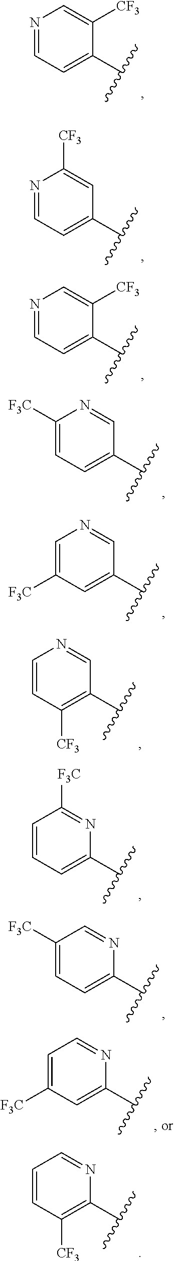

13. The compound of any of claims 1-3, or a pharmaceutically acceptable salt thereof, wherein:
R¹ is a nitrogen protecting group

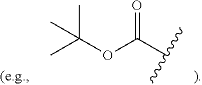 [SMILES: *C(=O)OC(C)(C)C]

14. The compound of any of claims 1-13, or a pharmaceutically acceptable salt thereof, wherein:
G is —O—.

15. The compound of any of claims 1-13, or a pharmaceutically acceptable salt thereof, wherein:
G is a bond.

16. The compound of any of claims 1-15, or a pharmaceutically acceptable salt thereof, wherein:
A is

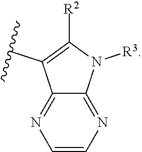

17. The compound of any of claims 1-15, or a pharmaceutically acceptable salt thereof, wherein:
A is

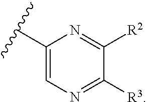

18. The compound of any of claims 1-15, or a pharmaceutically acceptable salt thereof, wherein:
A is

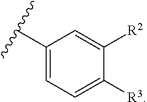

19. The compound of any of claims 1-18, or a pharmaceutically acceptable salt thereof, wherein:
R² and R³ are each independently hydrogen or substituted or unsubstituted heteroaryl; or R² and R³ together with the atoms to which they are attached form a substituted or unsubstituted aryl, or substituted or unsubstituted heteroaryl.

20. The compound of any of claims 1-19, or a pharmaceutically acceptable salt thereof, wherein:
R² is substituted or unsubstituted heteroaryl.

21. The compound of any of claims 1-20, or a pharmaceutically acceptable salt thereof, wherein:
R² is substituted or unsubstituted thiadiazolyl.

22. The compound of any of claims 1-20, or a pharmaceutically acceptable salt thereof, wherein:
R³ is hydrogen.

23. The compound of any of claims 1-19, or a pharmaceutically acceptable salt thereof, wherein:
R² and R³ together with the atoms to which they are attached form a substituted or unsubstituted heteroaryl.

24. The compound of any of claims 1-23, or a pharmaceutically acceptable salt thereof, wherein:
R² and R³ together with the atoms to which they are attached form a substituted or unsubstituted pyrrolyl or a substituted or unsubstituted pyrazolyl.

25. The compound of any of claims 1-24, or a pharmaceutically acceptable salt thereof, wherein:
R² and R³ together with the atoms to which they are attached form a substituted or unsubstituted pyrazolyl.

26. The compound of any of claims 1-24, or a pharmaceutically acceptable salt thereof, wherein:
A is 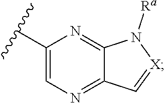
wherein X is N or CR^{a}; and each R^{a} is independently hydrogen, substituted or unsubstituted alkyl, or substituted or unsubstituted heterocyclyl.

27. The compound of any of claims 1-26, or a pharmaceutically acceptable salt thereof, wherein:
A is 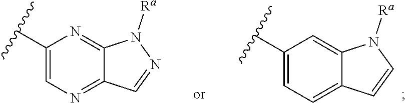
wherein R^{a} is hydrogen, substituted or unsubstituted alkyl, or substituted or unsubstituted heterocyclyl.

28. The compound of any of claims 1-27, or a pharmaceutically acceptable salt thereof, wherein:
A is 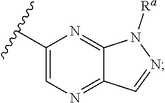
wherein R^{a} is substituted or unsubstituted alkyl or substituted or unsubstituted heterocyclyl.

29. The compound of any of claims 1-15, or a pharmaceutically acceptable salt thereof, wherein:
A is

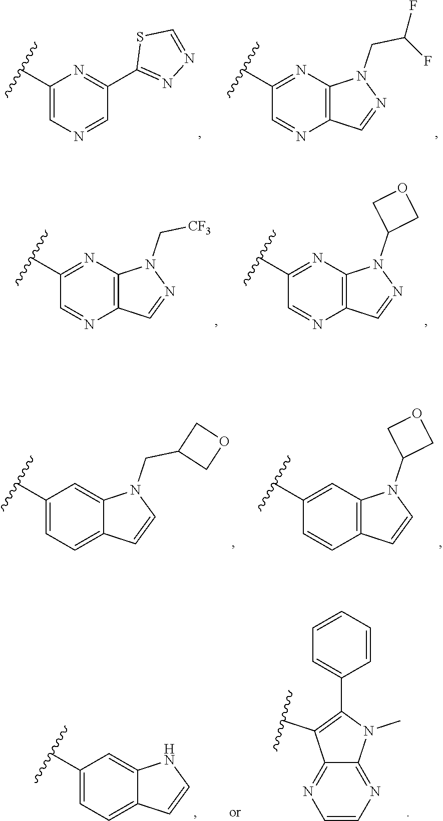

30. The compound of any of claims 1-15, or a pharmaceutically acceptable salt thereof, wherein:
A is

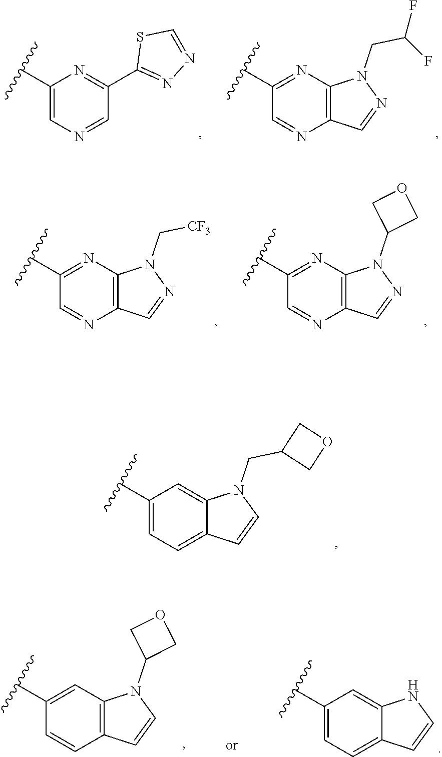

31. The compound of any of claims 1-30, or a pharmaceutically acceptable salt thereof, wherein:
A is

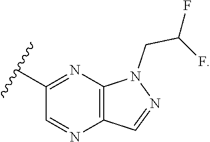 [SMILES: *c1cnc2cnn(CC(F)F)c2n1]

32. The compound of any of claims 1-31, or a pharmaceutically acceptable salt thereof, wherein:
p is 0.

33. The compound of any of claims 1-32, or a pharmaceutically acceptable salt thereof, wherein:
q is 0.

34. The compound of any of claims 1 or 3-33, or a pharmaceutically acceptable salt thereof, wherein:
m is 0.

35. The compound of any of claims 1 or 3-33, or a pharmaceutically acceptable salt thereof, wherein:
m is 1.

36. The compound of any of claims 1 or 3-33, or a pharmaceutically acceptable salt thereof, wherein:
m is 2.

37. The compound of any of claims 1 or 3-36, or a pharmaceutically acceptable salt thereof, wherein:
n is 0.

38. The compound of any of claims 1-36, or a pharmaceutically acceptable salt thereof, wherein:
n is 1.

39. The compound of any of claims 1-36, or a pharmaceutically acceptable salt thereof, wherein:
n is 2.

40. The compound of any of claims 1 or 3-39, or a pharmaceutically acceptable salt thereof, wherein:
t is 0.

41. The compound of any of claims 1-39, or a pharmaceutically acceptable salt thereof, wherein:
t is 1.

42. The compound of any of claims 1-39, or a pharmaceutically acceptable salt thereof, wherein:
t is 2.

43. The compound of any of claims 1 or 3-42, or a pharmaceutically acceptable salt thereof, wherein:
u is 0.

44. The compound of any of claims 1 or 3-42, or a pharmaceutically acceptable salt thereof, wherein:
u is 1.

45. The compound of any of claims 1 or 3-42, or a pharmaceutically acceptable salt thereof, wherein:
u is 2.

46. The compound of any of claims 1-45, or a pharmaceutically acceptable salt thereof, wherein:
Z is N.

47. The compound of any of claims 1-45, or a pharmaceutically acceptable salt thereof, wherein:
Z is CR⁴.

48. The compound of any of claims 1-45, or a pharmaceutically acceptable salt thereof, wherein:
Z is CH.

49. The compound of any of claims 1 or 3-48, or a pharmaceutically acceptable salt thereof, wherein:
Y is N.

50. The compound of any of claims 1 or 3-48, or a pharmaceutically acceptable salt thereof, wherein:
Y is CR⁴.

51. The compound of any of claims 1 or 3-48, or a pharmaceutically acceptable salt thereof, wherein:
Y is CH.

52. The compound of any of claims 1 or 3-51, or a pharmaceutically acceptable salt thereof, wherein:
X¹ is N.

53. The compound of any of claims 1 or 3-51, or a pharmaceutically acceptable salt thereof, wherein:
X¹ is CR⁴.

54. The compound of any of claims 1 or 3-51, or a pharmaceutically acceptable salt thereof, wherein:
X¹ is CH.

55. The compound of any of claims 1-54, or a pharmaceutically acceptable salt thereof, wherein:
L is a bond.

56. The compound of any of claims 1-54, or a pharmaceutically acceptable salt thereof, wherein:
L is —C(═O)—.

57. The compound of any of claims 1-54, or a pharmaceutically acceptable salt thereof, wherein:
L is —NR^{A}—.

58. The compound of claim 1, wherein the compound is of formula (I-1): 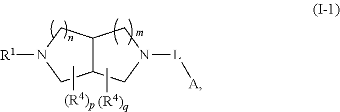
or a pharmaceutically acceptable salt thereof.

59. The compound of claim 1, wherein the compound is of formula (I-a-1): 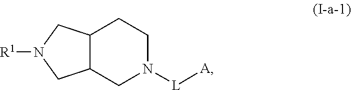
or a pharmaceutically acceptable salt thereof.

60. The compound of claim 1, wherein the compound is of formula (I-b-1): 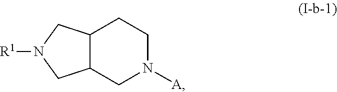
or a pharmaceutically acceptable salt thereof.

61. The compound of claim 1, wherein the compound is of formula (I-c-1): 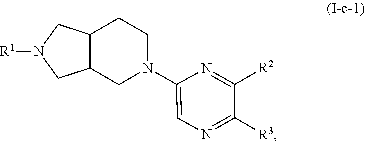
or a pharmaceutically acceptable salt thereof.

62. The compound of claim 1, wherein the compound is of formula (I-d-1): 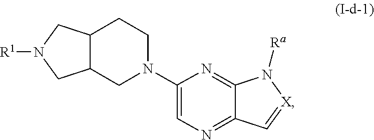
or a pharmaceutically acceptable salt thereof, wherein:
X is N or CR^{a}; and each R^{a} is independently hydrogen, substituted or unsubstituted alkyl or substituted or unsubstituted heterocyclyl.

63. The compound of claim 1, wherein the compound is of formula (I-e-1): 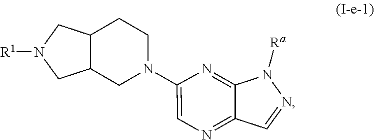
or a pharmaceutically acceptable salt thereof, wherein:
R^{a} is hydrogen, substituted or unsubstituted alkyl or substituted or unsubstituted heterocyclyl.

64. The compound of claim 1, wherein the compound is of formula (I-f-1): 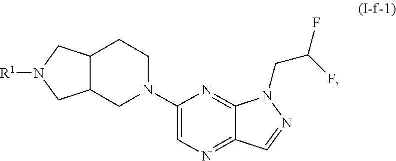
or a pharmaceutically acceptable salt thereof.

65. The compound of claim 1, wherein the compound is of formula (I-g-1): 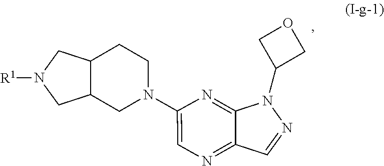
or a pharmaceutically acceptable salt thereof.

66. The compound of claim 1, wherein the compound is of formula (I-h-1): 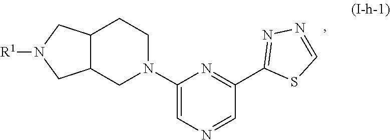
or a pharmaceutically acceptable salt thereof.

67. The compound of claim 1, wherein the compound is of formula (I-i-1): 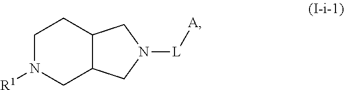
or a pharmaceutically acceptable salt thereof.

68. The compound of claim 1, wherein the compound is of formula (I-j-1): 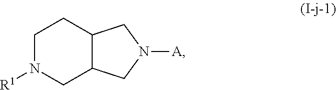
or a pharmaceutically acceptable salt thereof.

69. The compound of claim 1, wherein the compound is of formula (I-k-1): 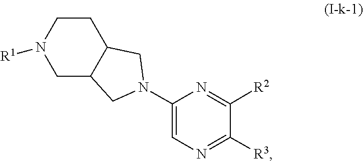
or a pharmaceutically acceptable salt thereof.

70. The compound of claim 1, wherein the compound is of formula (I-l-1): 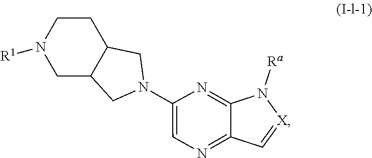
or a pharmaceutically acceptable salt thereof, wherein
X is N or CR^{a}; and each R^{a} is independently hydrogen, substituted or unsubstituted alkyl, or substituted or unsubstituted heterocyclyl.

71. The compound of claim 1, wherein the compound is of formula (I-m-1): 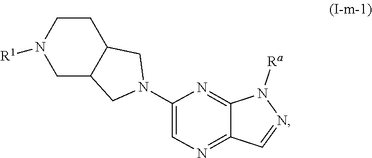
or a pharmaceutically acceptable salt thereof, wherein
R^{a} is independently hydrogen, substituted or unsubstituted alkyl, or substituted or unsubstituted heterocyclyl.

72. The compound of claim 1, wherein the compound is of formula (I-n-1): 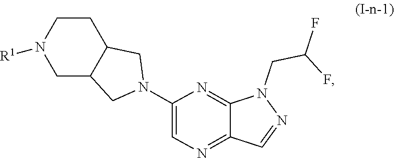
or a pharmaceutically acceptable salt thereof.

73. The compound of claim 1, wherein the compound is of formula (I-o): 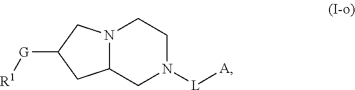
or a pharmaceutically acceptable salt thereof.

74. The compound of claim 1, wherein the compound is of formula (I-p): 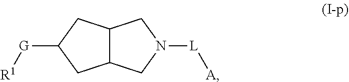
or a pharmaceutically acceptable salt thereof.

75. The compound of claim 1, wherein the compound is of formula (I-q): 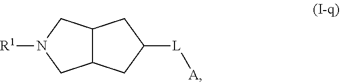
or a pharmaceutically acceptable salt thereof.

76. The compound of claim 1, wherein the compound is of formula (I-r): 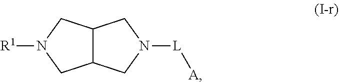
or a pharmaceutically acceptable salt thereof.

77. The compound of claim 1, wherein the compound is of formula (I-r-4): 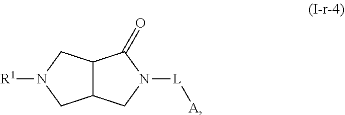
or a pharmaceutically acceptable salt thereof.

78. The compound of claim 1, wherein the compound is of formula (I-s): 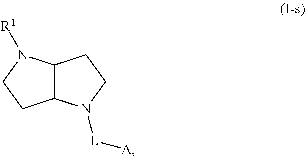
or a pharmaceutically acceptable salt thereof.

79. The compound of claim 1, wherein the compound is of formula (I-t): 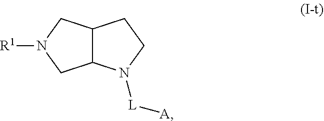
or a pharmaceutically acceptable salt thereof.

80. The compound of claim 1, wherein the compound is of formula (I-u): 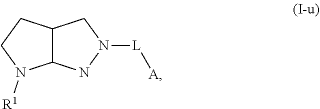
or a pharmaceutically acceptable salt thereof.

81. The compound of claim 1, wherein the compound is of formula (I-v): 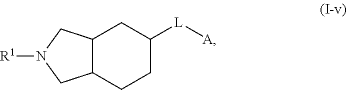
or a pharmaceutically acceptable salt thereof.

82. The compound of claim 1, wherein the compound is: 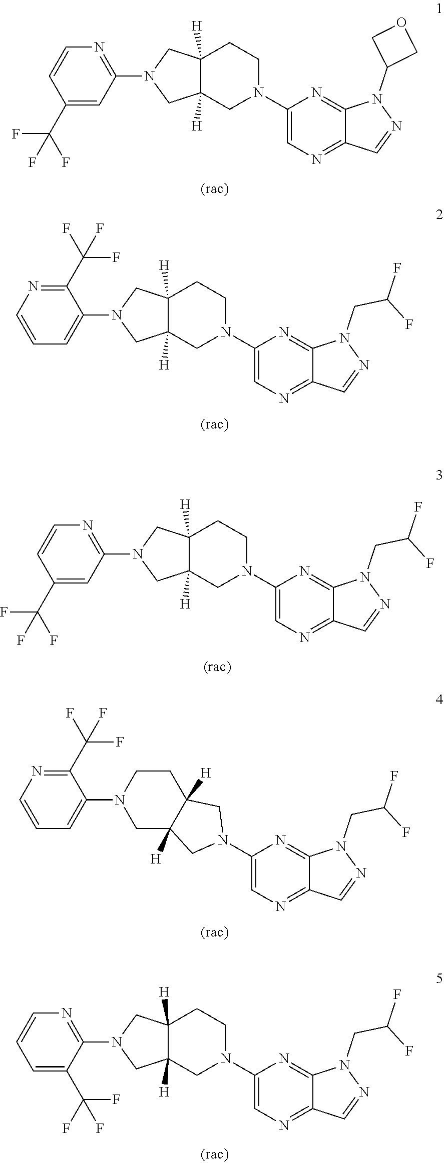 [SMILES: FC(F)(F)c1ccnc(N2C[C@H]3CCN(c4cnc5cnn(C6COC6)c5n4)C[C@H]3C2)c1 ; FC(F)Cn1ncc2ncc(N3CC[C@@H]4CN(c5cccnc5C(F)(F)F)C[C@@H]4C3)nc21 ; FC(F)Cn1ncc2ncc(N3CC[C@@H]4CN(c5cc(C(F)(F)F)ccn5)C[C@@H]4C3)nc21 ; FC(F)Cn1ncc2ncc(N3C[C@H]4CCN(c5cccnc5C(F)(F)F)C[C@H]4C3)nc21 ; FC(F)Cn1ncc2ncc(N3CC[C@H]4CN(c5ncccc5C(F)(F)F)C[C@H]4C3)nc21] 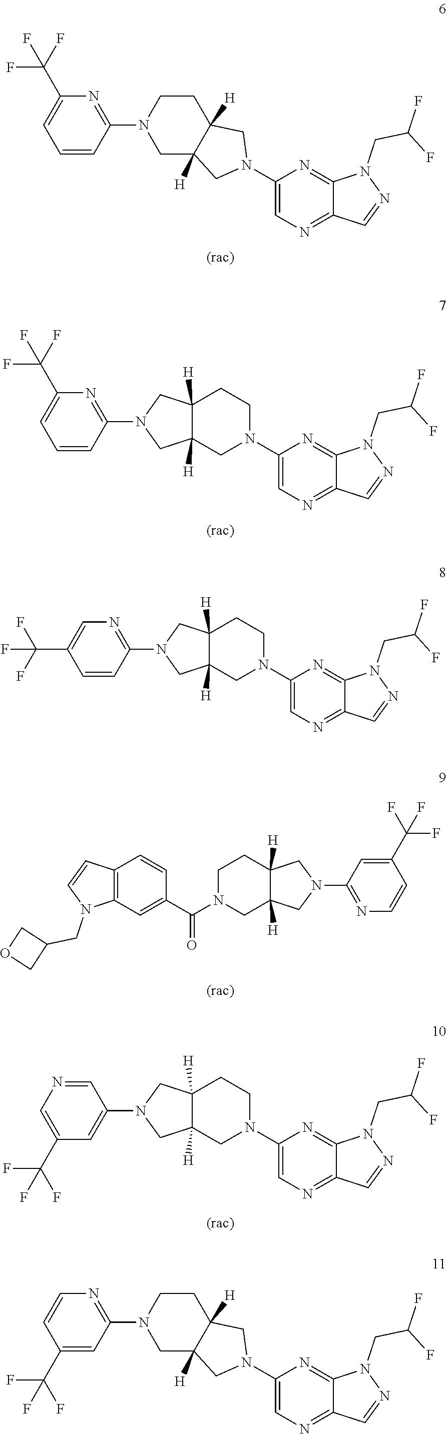 [SMILES: FC(F)Cn1ncc2ncc(N3C[C@H]4CCN(c5cccc(C(F)(F)F)n5)C[C@H]4C3)nc21 ; FC(F)Cn1ncc2ncc(N3CC[C@H]4CN(c5cccc(C(F)(F)F)n5)C[C@H]4C3)nc21 ; FC(F)Cn1ncc2ncc(N3CC[C@H]4CN(c5ccc(C(F)(F)F)cn5)C[C@H]4C3)nc21 ; O=C(c1ccc2ccn(CC3COC3)c2c1)N1CC[C@@H]2CN(c3cc(C(F)(F)F)ccn3)C[C@@H]2C1 ; FC(F)Cn1ncc2ncc(N3CC[C@@H]4CN(c5cncc(C(F)(F)F)c5)C[C@@H]4C3)nc21 ; FC(F)Cn1ncc2ncc(N3C[C@H]4CCN(c5cc(C(F)(F)F)ccn5)C[C@H]4C3)nc21] 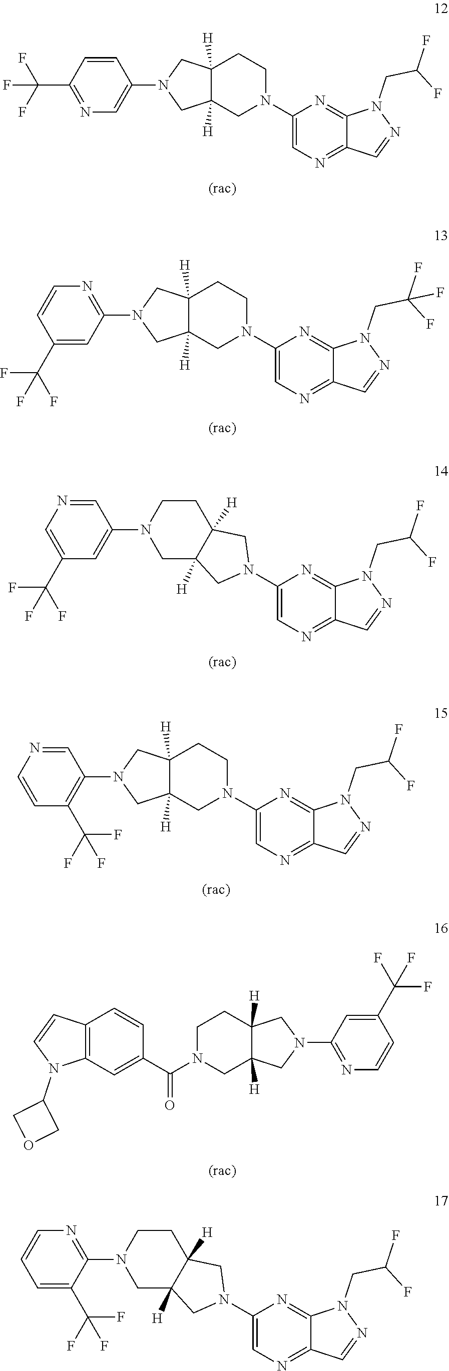 [SMILES: FC(F)Cn1ncc2ncc(N3CC[C@@H]4CN(c5ccc(C(F)(F)F)nc5)C[C@@H]4C3)nc21 ; FC(F)(F)Cn1ncc2ncc(N3CC[C@@H]4CN(c5cc(C(F)(F)F)ccn5)C[C@@H]4C3)nc21 ; FC(F)Cn1ncc2ncc(N3C[C@H]4CN(c5cncc(C(F)(F)F)c5)CC[C@H]4C3)nc21 ; FC(F)Cn1ncc2ncc(N3CC[C@@H]4CN(c5cnccc5C(F)(F)F)C[C@@H]4C3)nc21 ; O=C(c1ccc2ccn(C3COC3)c2c1)N1CC[C@@H]2CN(c3cc(C(F)(F)F)ccn3)C[C@@H]2C1 ; FC(F)Cn1ncc2ncc(N3C[C@H]4CCN(c5ncccc5C(F)(F)F)C[C@H]4C3)nc21] 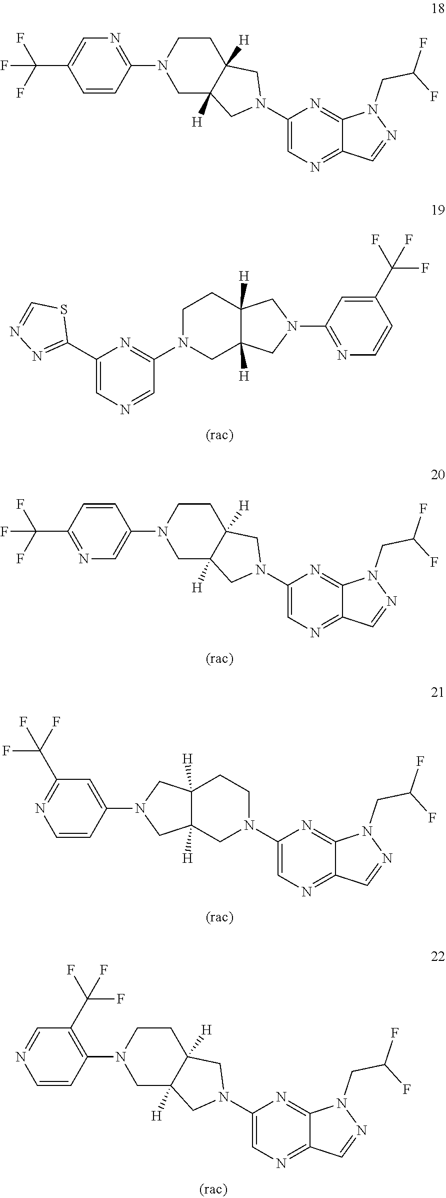 [SMILES: FC(F)Cn1ncc2ncc(N3C[C@H]4CCN(c5ccc(C(F)(F)F)cn5)C[C@H]4C3)nc21 ; FC(F)(F)c1ccnc(N2C[C@H]3CCN(c4cncc(-c5nncs5)n4)C[C@H]3C2)c1 ; FC(F)Cn1ncc2ncc(N3C[C@H]4CN(c5ccc(C(F)(F)F)nc5)CC[C@H]4C3)nc21 ; FC(F)Cn1ncc2ncc(N3CC[C@@H]4CN(c5ccnc(C(F)(F)F)c5)C[C@@H]4C3)nc21 ; FC(F)Cn1ncc2ncc(N3C[C@@H]4CCN(c5ccncc5C(F)(F)F)C[C@@H]4C3)nc21] 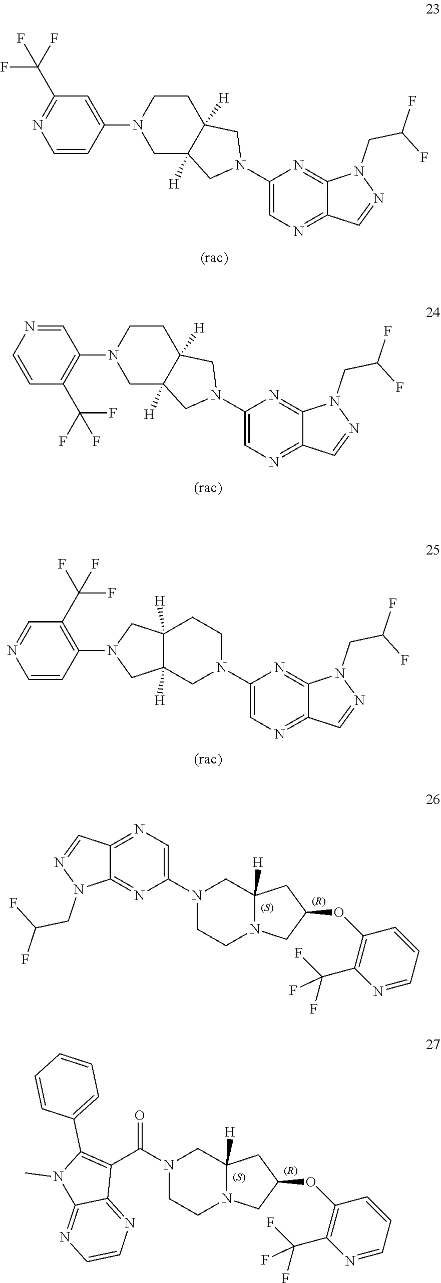 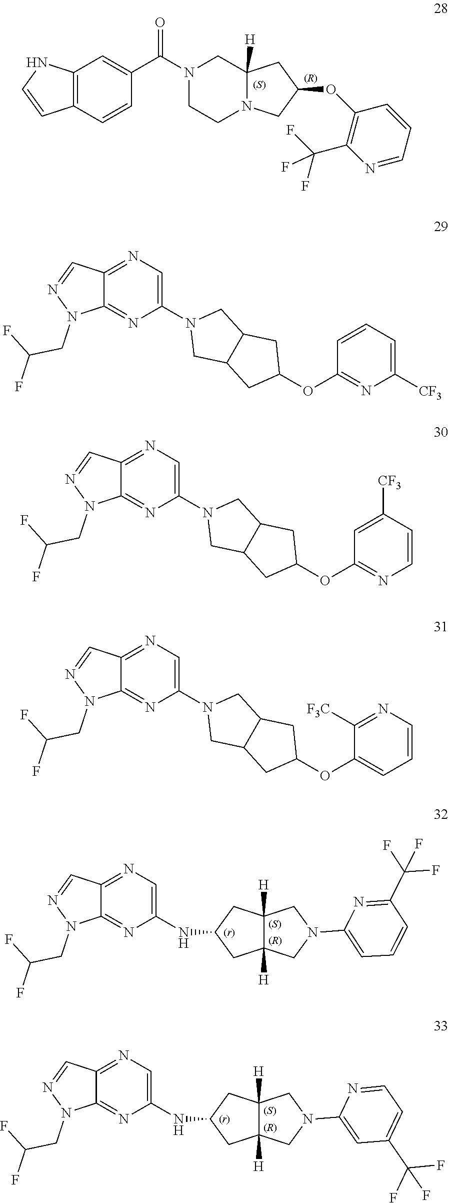 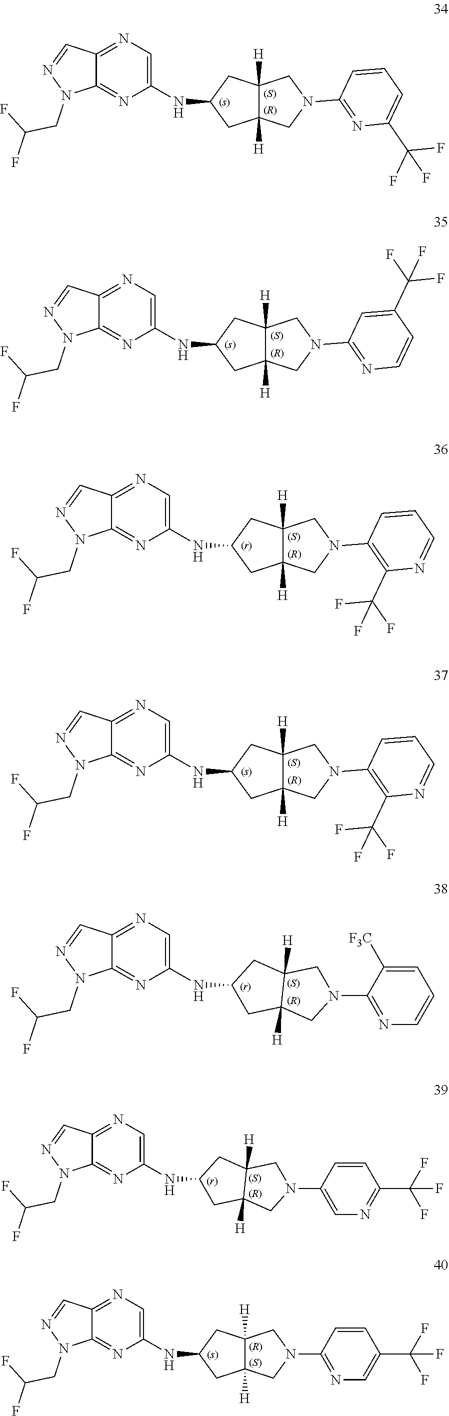 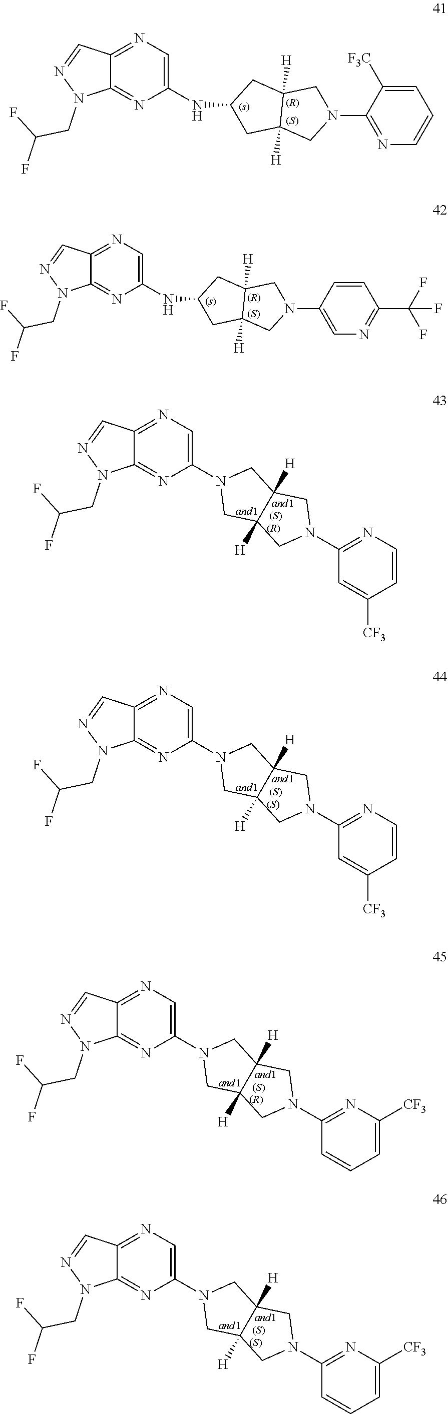 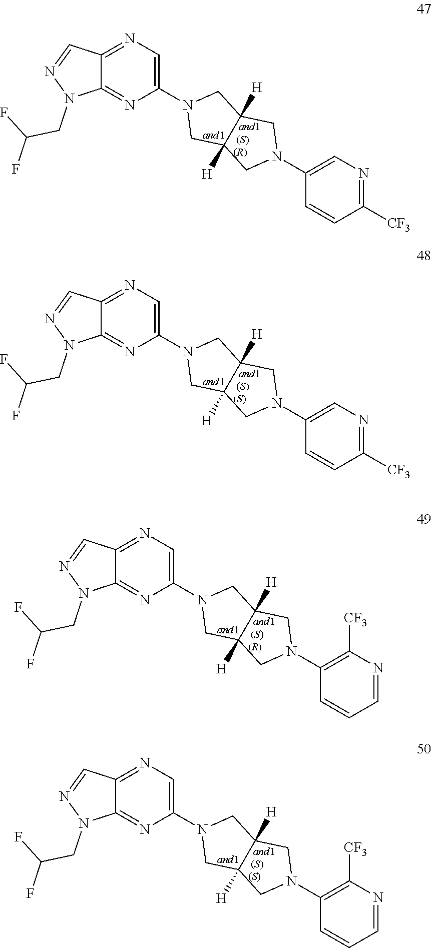 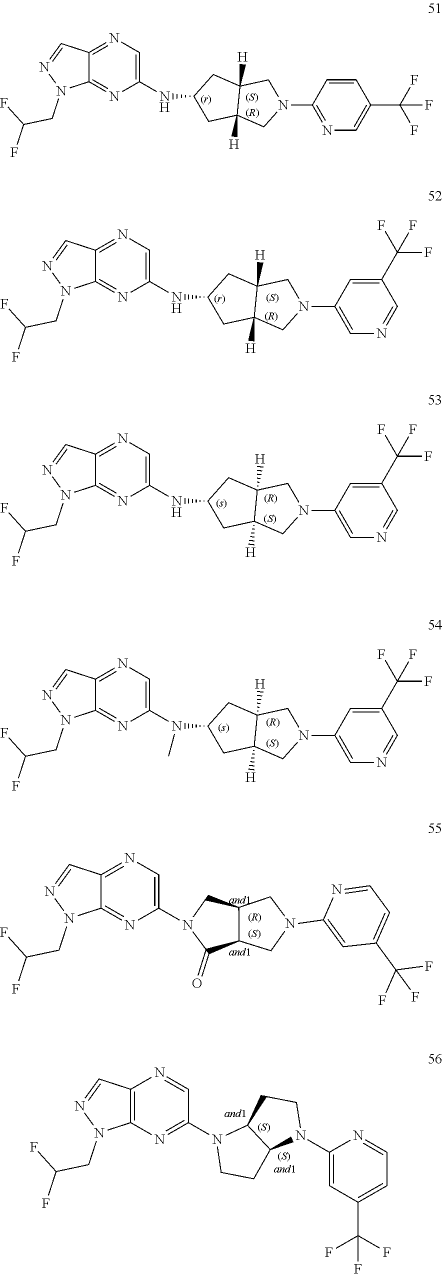 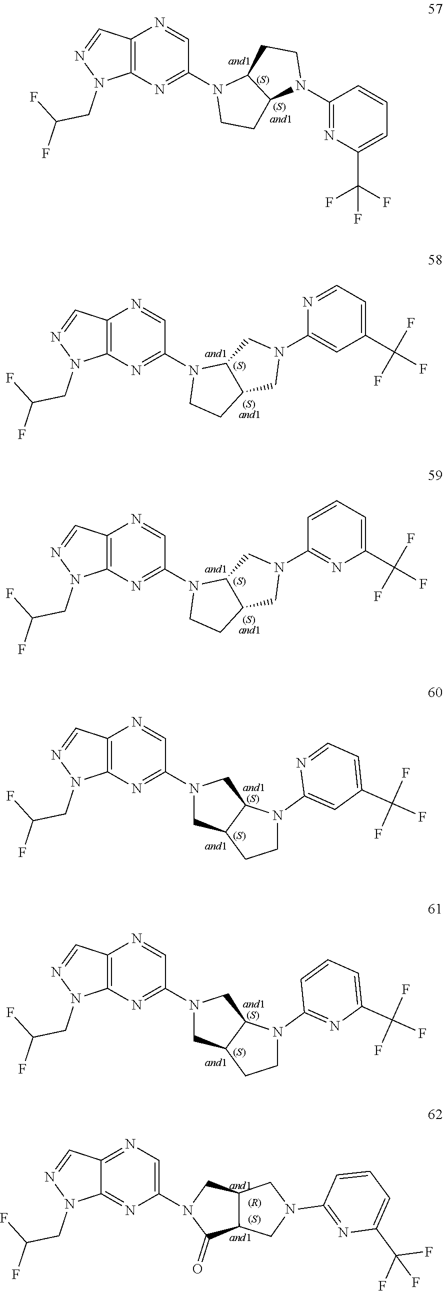 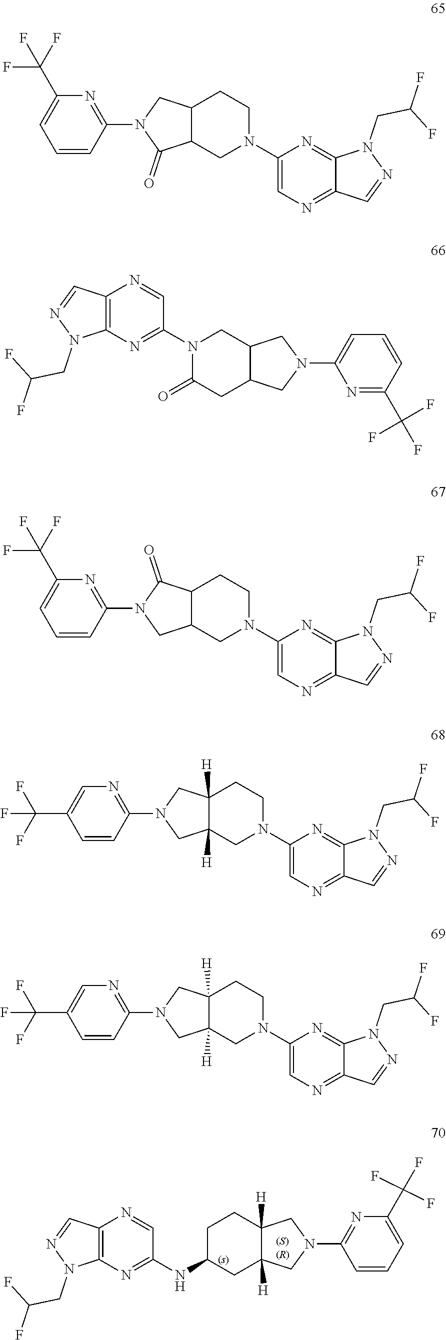 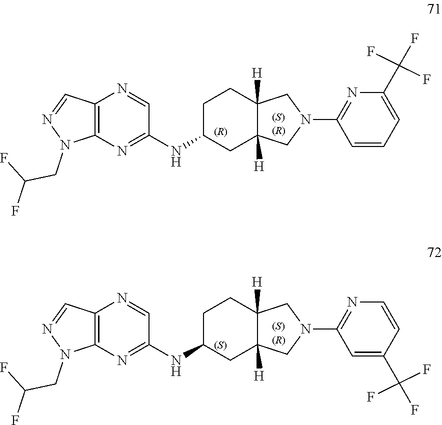 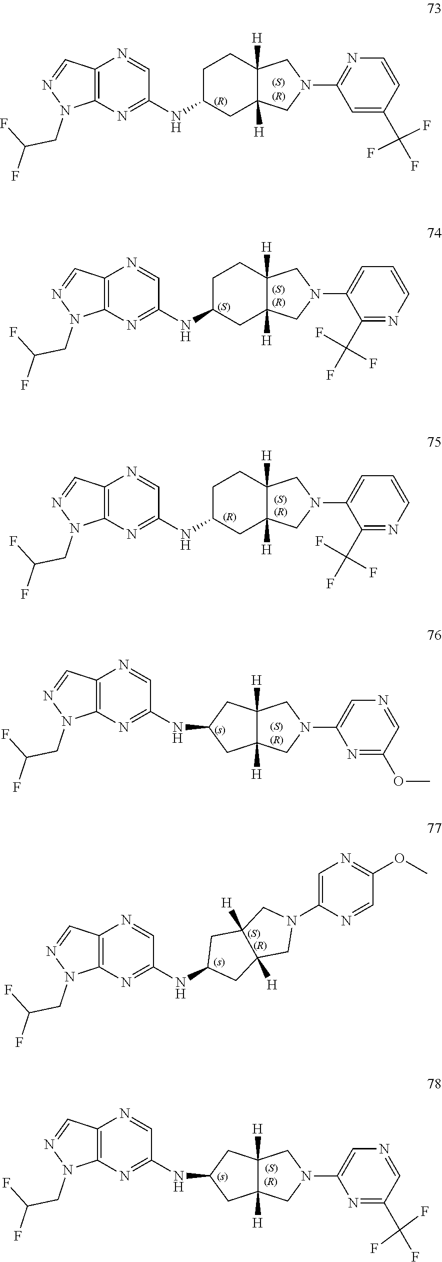 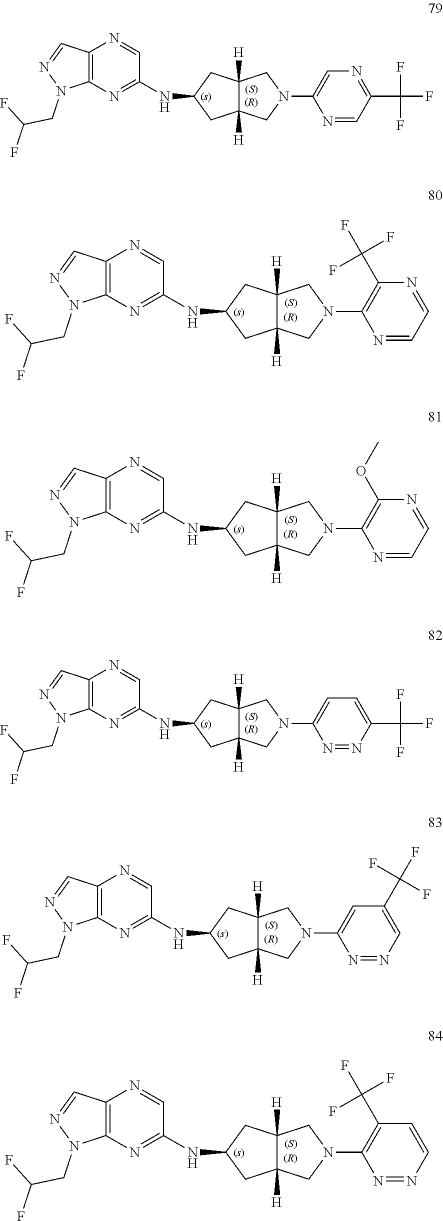 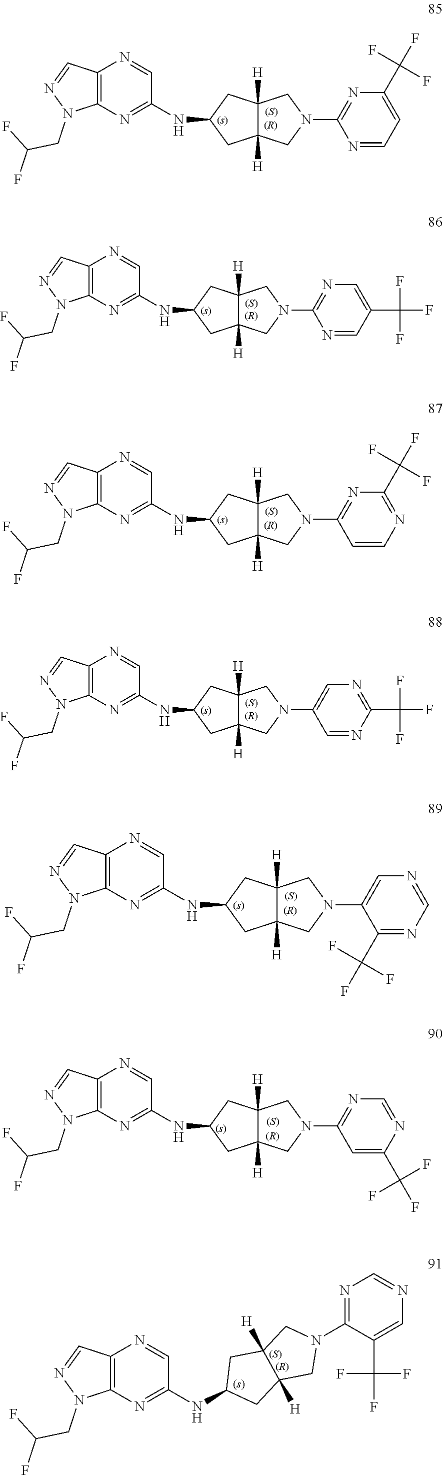
or a pharmaceutically acceptable salt thereof.

83. The compound of claim 2, wherein the compound is of formula (II-a): 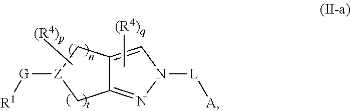
or a pharmaceutically acceptable salt thereof.

84. The compound of claim 2, wherein the compound is of formula (II-a-1): 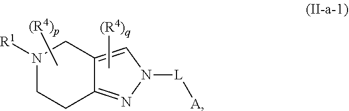
or a pharmaceutically acceptable salt thereof.

85. The compound of claim 1, wherein the compound is of formula (II-a-2): 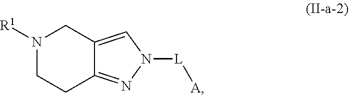
or a pharmaceutically acceptable salt thereof.

86. The compound of claim 2, wherein the compound is of formula (II-b): 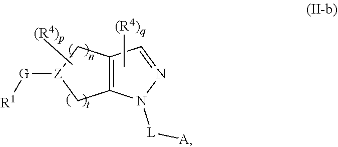
or a pharmaceutically acceptable salt thereof.

87. The compound of claim 2, wherein the compound is of formula (II-b-1): 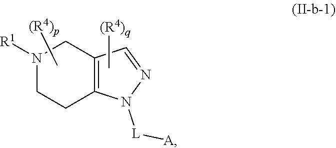
or a pharmaceutically acceptable salt thereof.

88. The compound of claim 1, wherein the compound is of formula (II-b-2): 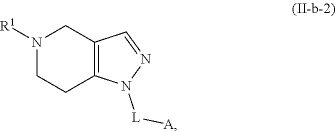
or a pharmaceutically acceptable salt thereof.

89. The compound of claim 1, wherein the compound is: 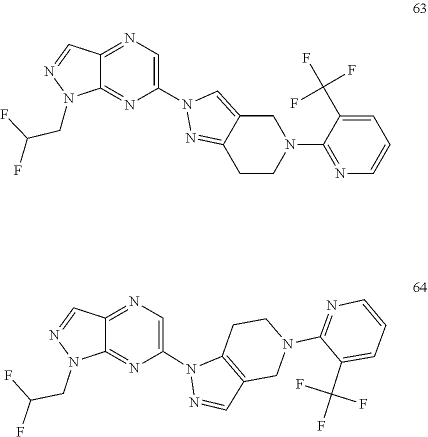 [SMILES: FC(F)Cn1ncc2ncc(-n3cc4c(n3)CCN(c3ncccc3C(F)(F)F)C4)nc21 ; FC(F)Cn1ncc2ncc(-n3ncc4c3CCN(c3ncccc3C(F)(F)F)C4)nc21]
or a pharmaceutically acceptable salt thereof.

90. A pharmaceutical composition comprising a compound of any of claims 1-89, or a pharmaceutically acceptable salt thereof, and a pharmaceutically acceptable excipient.

91. A kit comprising a compound of any of claims 1-89, or a pharmaceutically acceptable salt thereof, or a pharmaceutical composition of claim 90, and instructions for administering the compound or pharmaceutical composition to a subject in need thereof.

92. A method of treating a disease or disorder in a subject in need thereof, the method comprising administering an effective amount of a compound of any of claims 1-89, or pharmaceutically acceptable salt thereof, or a pharmaceutical composition of claim 90.

93. The method of claim 92, wherein the disease or disorder is associated with glucocerebrosidase activity.

94. The method of claim 92 or 93, wherein the disease or disorder is a neurological disease or disorder.

95. The method of claim 94, wherein the neurological disease or disorder is Parkinson's disease or Gaucher's disease.

96. A method of activating glucocerebrosidase, the method comprising contacting glucocerebrosidase with an effective amount of a compound of any of claims 1-89, or pharmaceutically acceptable salt thereof, or a pharmaceutical composition of claim 90.

97. The method of claim 96, wherein the contacting is in vitro.

98. The method of claim 96, wherein the contacting is in vivo.
